SEQUENCE LISTING (63 sequences)

SEQ ID NO: 1 - protein, 515 aa, Botrytis cinerea
  REGION 1..515: note = MISC_FEATURE - Botrytis cinera
  SITE 2: note = MISC_FEATURE - Xaa can be A or T
MXAQAIRPSISFEPGQTFDMSNPLLRTLRTSRPILSSSPSRISTKRFVSTSTETSPVLTRSKNLLVGTSIALALGFGYLYITDSRASVHQWLVVPALRAIYPDAEDAHHVGTKALKTLYSFGLHPRDRSFRQGNTDKADLSVEVWGQKLQNPIGTSAGLDKLAEVPDALFALGPAIVEVGGCTPLAQDGNPKPRVFRIPSQNALINRYGLNSKGADDMAMRLRERVRLYAYQNGLGVGEDAEKMVLNGEAGVPPGSLLDGKLLAVQVAKNKNTNGDDIEAVKRDYVHCVERLAPYADILVVNVSSPNTPGLRTLQKVEPLSRILEGVVGAAKSIDRKTKPRVMVKVSPDEDQDEQIEGIVEAVWRSGVDGIIVGNTTNRRDKLIPDDIKISARESRILEEQGGFSGPPMFERTLSLVKRYRTLLDRGPPQQETTPESNDNGLVSTIAEKAKSLTGNKANDQKVIFATGGITNGKQALEVLNAGANVAQVYTAMVYGGAGTITRIKTEMSNEISKK

SEQ ID NO: 2 - DNA, 1548 bp, synthetic construct
  misc_feature 1..1548: note = BOTRCI_DHODH_cDNA_KB708057.1_30538-30546,30690-3215 8,32250-32319
  source 1..1548: mol_type = other DNA
atggccgctcaagcaatccgtccctctatttccttcgaacccggacagacattcgatatgtcgaaccctctcttgcgcactctgcgtacatcgcgaccaattctctcctcttctccatcccgcatctctaccaaacgcttcgtatcaacctctaccgaaacatccccggttctgacccgatcgaagaatctcctcgttggaacctcgatagctctcgctctcggctttggctacttatatattactgattcccgagctagcgttcaccaatggcttgtggttcccgcactacgtgccatatatcccgatgccgaagatgcccatcatgtaggaactaaagctctcaaaacgctctactcattcggtctccatcctcgcgatcgaagcttcagacaaggcaatacagataaagcagatttaagcgtagaagtatggggccagaaattacaaaatccaattggcacatctgcggggcttgacaaattggcagaggttccagatgctttgtttgccttgggtcctgcgattgtcgaggttggcgggtgcacaccacttgcacaagatggaaacccaaaacctagagttttccgtataccgagtcagaatgctctgatcaatcgatatggacttaattcaaagggtgcagatgatatggctatgcgattgagggaacgggttcgtttatacgcatatcagaatggattgggtgtaggagaagatgctgagaagatggtcttgaatggagaagctggtgttcccccaggaagtttgttggacgggaaattattggcggtgcaagttgccaagaataaaaataccaacggtgatgacattgaggccgtcaaaagagactacgttcactgcgtcgagagattggctccatatgccgatatcttggtagtcaatgttagtagcccaaacacccctggcttgaggacattacaaaaggttgagccattatctcgaattttagaaggagtggtcggggctgccaaatccatagaccggaaaactaagcctagggttatggtcaaggtatcacccgatgaggatcaagacgagcaaatcgagggcatcgttgaagctgtctggagaagtggcgtagacggtatcattgtgggtaataccacaaaccgaagagacaagctcatcccagacgacatcaagatttctgccagagaaagcaggattctcgaagaacaaggaggattttctgggccaccaatgttcgagagaacattatctctggttaagagatacagaactctactcgatcgaggaccaccacaacaagaaaccactcctgaatcaaacgacaatggtcttgtctccacgatcgccgaaaaggcaaaatctctcacgggcaataaagcaaatgatcaaaaggtaatcttcgctactggtggcatcacgaatggaaaacaagcactagaagtcttgaatgcaggtgcaaatgtcgcccaagtttacacagcaatggtctatggaggtgccggcacaataactcgtataaaaactgagatgagcaacgagatttcaaaaaagtaa

SEQ ID NO: 3 - protein, 537 aa, Alternaria alternata
  REGION 1..537: note = MISC_FEATURE - ALTEAL_DHODH_XP_018389332
MNTAAHIRALRVRPSIPRCRPSHRALQHPTRRVTTATEAATNVRSTGTRTKNLVYGTLLALGGTFGYLYATDTRASIHEWLVVPALRLIYPDGEEAHHAGTHMLKVLHSFGLNPRERGVADSRGDLAVEVFGHTLNNPIGTSAGIDKGAEVPTPLFELGAGVVEVGAVTLLPQEGNPKPRVFRIPSQNALINRYGFNSEGAELVASRLRQRVREFAYHAGLGLDDDAERRVLDGEAGVPPGSLLKGRLMAVQVAKNKTTSENDIDAVVRDYATAVGHVGKYADILVVNVSSPNTPGLRTLQNVEPLTRLLSGVVGAVKKIDRKTKPVVMVKVSPDEDTEEQVSGICEAVWDSGVDGVIVGNTTKKRPDPLPKGYLLPESEAKLLLEQGGYSGPQTFERTLALVSRYRKALDEGPKHVAPPSDASSTSTGSSMTKPTLDLDQPAPTDLSSNVDDLSESAPTMTLHSTPKTAPAPNAPRKVIFATGGITNGRQAREILDAGASVAQVYTAMIYGGAGTISRIKQEMRDDAKEQKTITKR

SEQ ID NO: 4 - DNA, 1614 bp, synthetic construct
  misc_feature 1..1614: note = ALTEAL_DHODH_cDNA_XM_018536128.1
  source 1..1614: mol_type = other DNA
atgaacaccgccgcccacatccgtgccttgcgcgtgcggccctcgataccacgctgccgcccctctcaccgcgcgttgcagcaccccacaagacgtgtcacgacagccaccgaggccgcgaccaacgttcgatcgacgggcactcgcaccaagaacctcgtgtacggtacccttctggcgctcggtggtacgtttggctacctatacgccaccgacacgcgcgcaagcatccacgaatggctcgttgtgccagcactacgcctgatatacccagatggagaggaggcgcaccacgctggaacgcacatgctcaaggtgctgcatagcttcggcttgaaccccagagaacgtggcgtggctgattcacggggcgatctggctgtcgaggtctttggacacacactgaacaacccgattggaacatctgccggcatcgacaagggcgccgaggtaccaacaccgctattcgaacttggagcgggagtagtagaggtcggcgctgttacactactgccgcaagaaggcaacccaaaaccacgcgtcttcaggatccccagccagaacgcgctcatcaaccgctatggattcaattccgaaggcgccgagctggttgcttcgaggttacgacaacgtgtcagggagtttgcctaccacgccgggcttggactagatgacgacgcagagcgccgggtcctcgacggcgaagccggtgttccacctggttcgctgctcaagggacgtctcatggctgtccaggttgcgaagaacaagacgacctccgaaaacgatatcgatgctgtagtgcgcgactacgcgactgccgtgggccatgtcggaaagtacgctgatattctcgtcgtcaatgtctcctcacctaacacgcccggccttcgcactctccagaacgtcgagcccttgacacgactactctctggagttgtaggagcagtcaagaaaatcgatcgcaagacaaagcctgttgtcatggtgaaagtcagcccagatgaggacacagaagagcaggtatctggcatctgcgaggccgtctgggactcgggtgtcgacggcgtcatcgttggtaacacaacgaagaagcgaccagaccccctgcccaagggctacttgcttcctgaatcagaggcaaagctgcttctcgagcaaggcggatactcgggaccacaaaccttcgagcgcacacttgccctggtcagcaggtacagaaaagcactcgacgaaggccccaagcatgtcgcaccgccatctgacgcctcatccacctctaccggttcctcaatgaccaagccaacgctcgaccttgaccaaccggccccaacagacctctcctccaacgtcgacgacctttctgaatcggcacctaccatgaccctccattctacacccaagactgcacctgcaccaaacgcaccacgcaaggtcatattcgccacgggcggcatcacaaacggacgccaagcaagagagattctcgatgctggagcgagtgtggcacaggtatatactgccatgatctatggcggtgctggcacaatcagcaggatcaagcaggagatgagggatgacgcaaaggagcaaaagacaataacgaagcggtag

SEQ ID NO: 5 - protein, 548 aa, Alternaria solani
  REGION 1..548: note = MISC_FEATURE - ALTESO_DHODH_no_accession
MNTAAHIRALRVRPSIARCRPLHRQLQHPTRRATTATTATTATEAATNVRSAGTRTKNLVYGTLLALGGTFGYLYVTDTRASIHEWLVVPALRQIYPDGEEAHHAGTHMLKVLHSFGLNPRERGEADSRGDLAVQVFGHTLNNPIGTSAGIDKGAEVPTPLFELGAGVVEVGAVTLLPQEGNPKPRVFRIPSQNALINRYGFNSEGAELVASRLRQRVRQFAYHAGLGLDDDAERRVLDGEAGVPPGSLLKGRLLAVQVAKNKTTSENDIDAVVRDYATAVSHVGKYADILVVNVSSPNTPGLRTLQNVEPLTRLLSGVVGAVQKIDRKTKPAVMVKVSPDEDTEEQVSGICEAVWDSGVDGVIVGNTTKKRPDPLPKGYLLPESEAKLLLEQGGYSGPQTFERTLALVSRYRKALDEGPKHVALPSDAPTDDTPPPSTGSSKTKLTLDLDQPAPTPLSSNVDDLSESAPTITLHSTPKTAPAPDSPRKVIFATGGITNGRQARQVLDAGASVAQVYTAMIYGGAGTITRIKQEMRDDAKEQKTLTKR

SEQ ID NO: 6 - DNA, 1647 bp, synthetic construct
  misc_feature 1..1647: note = ALTESO_DHODH_cDNA_CP022024
  source 1..1647: mol_type = other DNA
atgaataccgccgcccatatccgtgccctgcgcgtgcggccctcgatagcacgctgccgccctttgcaccgccagttgcagcaccccacaagacgtgccacaaccgccacaaccgccacgacagccaccgaggccgcgaccaacgttcgatccgcaggcacgcgcaccaagaacctcgtctacggcacgcttctggcgctcggaggcacttttggctacctctacgtcaccgacacgcgcgcaagcatccacgaatggctcgttgtgcctgccctgcgccagatataccccgatggagaggaggcacatcacgcgggaacgcacatgctcaaggtgctgcatagcttcggcctgaaccctcgagagcgaggcgaggctgactcacggggcgatctggctgtccaagtctttggacacaccctgaacaacccaattggaacatctgcaggtattgacaagggcgccgaggtaccaacaccgctgttcgagctcggagcgggagtggtagaggtcggtgccgttacgctgctgccgcaagagggcaaccccaaaccacgcgtctttaggatccccagccagaacgctctcatcaaccgctatggattcaattctgaaggcgccgagctggtcgcttcaaggctgcgacaacgcgtcaggcagtttgcctaccacgccggacttgggctggatgacgatgcagagcgccgtgtcctcgacggcgaagctggtgttccaccgggatcgctgttaaaagggcgtcttttggcggtacaggtcgccaagaacaagacaacctctgaaaacgacatcgatgctgtagtgcgcgactacgcgactgccgtgagccatgtcggcaagtacgccgacatcctcgtcgtcaatgtctcctcacccaacacccccggcctccgcactctccagaacgtcgagcccttgacgcgcctactttctggcgtggtaggagcagtccagaaaatcgatcgcaagacaaagcccgccgtcatggtaaaagtcagcccagatgaggatacagaagagcaggtgtccggcatctgcgaggccgtctgggattcaggcgtcgacggtgtcatcgttggtaacacgaccaagaagcgaccagacccactgcccaagggctacttgcttcccgaatcagaggcaaaactgcttctcgagcaaggcggatactcgggaccacaaacctttgagcgcacacttgccctagtcagcaggtacagaaaagcactcgacgaaggccccaagcatgttgcactgccatctgacgcacccaccgatgacactccacccccctctaccggatcctcaaaaaccaagctaacgctcgaccttgaccaaccggctccaacaccactctcctccaacgtcgacgacctttccgaatcagcacctaccatcactcttcactctacacccaagactgcacctgcaccagactcaccacgcaaggttatattcgcgacgggcggcatcacaaacggacgccaagcaagacaggtgctcgatgccggagcgagtgtggcacaggtatatactgccatgatctatggcggtgcgggcacaatcaccaggatcaaacaggagatgagggatgacgcaaaggagcaaaagaccctgacgaaacggtag

SEQ ID NO: 7 - protein, 751 aa, Cercospora beticola
  REGION 1..751: note = MISC_FEATURE - CERCBE_DHODH_XP_023451964
MLATQKLLGASAPLVRQSAGLNRAQWRSVQRQWQRQLSLQSSRPRIAQHARNAQQRYQSTGEQAKEAADTAKAQAVKAGVATANTGSRFVAYVYGTGLVLLLSFGYSYATDTRASIHQFAVTPLVRLLFPDAEDAHHAGVRYIKAMHDFGLAPRERGNADKALGDLKVEVFGHILDNPLGTSAGIDKDAEIPDALFALGPAIVEVGGATPLAQEGNPKPRVFRIPSQNALINRYGLNSQGAEHMAIQLRERVRQFAYENGLGLEAESEQRILDGEAGVPPGSLVDGKLLAVQVAKNKTTPDNDIAAVAADYVFCVEHLAKYADILVVNVSSPNTPGLRDLQATKPLTAILSSVVDAAKSVDRKTKPAVMVKVSPDEDSDAQIEGICTAVWSSGVDGVIVGNTTKKRPEPIPKGYVMPADDVRTLTEVGGYSGPQIFERTLNLVKRYRSKLDQGPYDNQPADTQKAVQSPLDPLERGLIGDSTPENAQQHASQESIARDQQRLKPLTSEAEADSKRPLIELPERHTSEQPEAAKHVGKAAGPVATQPTDAAAPQNATQIGKASSPREAAEGAVQKVISVGESSTEAAKSELQSAASQAKDQAEQVTEKARASAEQFKAKAESAKDQAQSAAAQAKSQIDSATSDTKAKASELANKASSTAQSVATAAKDQFNHITSDPYAEVSAPKVDAPPVKDKVIFATGGITNGEQCLQILNAGASVCQVYTAMMYGGVGTITRMKTEMRAELRGKGKKE

SEQ ID NO: 8 - DNA, 2506 bp, synthetic construct
  misc_feature 1..2506: note = CERCBE_DHODH_NT_cDNA_XM_023596659.1
  source 1..2506: mol_type = other DNA
tccaacttcttgacgagtgaatcttcccaacaccggcacgatgctggccacgcagaagctgttgggcgcttctgcaccgctcgtgcggcagtctgcaggcctcaatcgcgcccaatggcgaagcgtgcagcgtcaatggcagcgccaactttcgctgcaatccagccgacctcgaatcgcccaacatgcccgaaatgcgcaacaacgataccaatcgactggtgaacaggcaaaagaggcagcagacacagcgaaagcgcaggctgtcaaggcgggagtagccaccgccaatacaggctctcgtttcgtcgcatatgtctacggaactggtcttgtcctcttgctgagcttcggctactcgtatgccacggatacacgcgccagcatccaccaattcgctgtgactcctctcgttcgcctactgttcccggacgccgaagacgcgcatcatgctggagtccgatacattaaggcaatgcacgactttggcttagctccacgagaacgcggaaatgcggataaagcgctgggagatttgaaggtcgaggtctttgggcacatcttagacaatccgctggggacatcagcaggaattgacaaggatgctgagattcctgacgcgttgtttgcgttggggcctgcgattgtggaagtgggaggcgcgacacctttggcgcaagaggggaaccctaagcccagagtctttcgaatacccagtcagaatgccttgatcaaccgatacggcctcaattcgcaaggagcggagcatatggcaatacagttgcgggaacgagtacgacagtttgcatatgagaacgggctagggcttgaagcagaatcagagcagaggatactggatggcgaagctggcgtgcctccgggaagtctggtggatggcaagttactggcagtacaagtggccaagaacaagacgacaccagataacgatatcgcggctgttgcagcggactacgtcttctgcgtagagcatctggccaaatacgccgacatcctggtcgtgaatgtatcgtcgcccaacacacctggcctgcgcgatttacaagcaacaaagccgctgacagctattctttcgtctgtcgtagatgcagccaagtctgtggaccgaaagacgaaacccgcagtcatggtcaaggtgtcgccagatgaggactcggatgcgcagatagaaggaatctgcactgcagtctggtcatcaggcgtcgatggagtcattgttggaaacacgaccaagaagcgacctgaaccaataccgaagggatacgtcatgcccgcagacgatgtgcgaacactcactgaagtcggaggatactctgggcctcaaatatttgagagaacactaaacctcgtcaagcgttatcgatcgaagctcgatcaaggaccgtacgacaatcagcctgcagacacacagaaggcagtccagagccccctcgatccacttgaacgtggtctaataggcgattcgacacctgaaaatgcccaacagcatgcatcacaagagagcatcgcccgcgatcagcaacgactaaagccgttgacgagcgaagcagaagccgacagcaagcgaccactgattgaacttcccgagcgacacacttccgagcagcctgaggcggcgaagcacgtcggaaaagctgctggtccagttgcgacacaacccaccgatgctgctgccccacagaatgcgacgcaaataggcaaggctagctcacccagagaagccgcggaaggagcagttcagaaggtgatttccgtcggtgaatccagcaccgaagccgccaagtcagaactgcaatccgcagcatcccaggccaaagaccaagcagagcaagtgacggagaaggcaagagccagtgccgaacaattcaaagccaaagcagaatccgctaaagaccaagcccaatccgccgcagctcaagcgaaatctcaaatcgactcggcgacatctgacaccaaagccaaagcatccgaactcgccaacaaggcatcctctaccgcccaatcagtcgctacagcagccaaagaccaattcaaccacatcacaagtgatccatatgccgaagtctcagcgcctaaggtcgacgctccacctgtcaaagacaaggttatctttgcgacgggaggcatcacgaatggcgagcagtgtttgcaaatcttgaatgcgggtgccagcgtgtgtcaggtgtatactgctatgatgtatggtggtgttgggacgattacgaggatgaagacggagatgagggcggagttgagagggaaggggaagaaggagtagacagggacagaggcagagaaatgtacagatccatggatgaagttcgtcccgtttgtatacatatcaaacatataccaaagatgtgttgtgcacgtggacatgcacctttgagggcatgatgaaagtagattttcaagaagcttttcgttgtgatagatgataatacaacagtagactgccctgtctgcagtcgcgccaccctcccagccc

SEQ ID NO: 9 - protein, 753 aa, Cercospora sojina
  REGION 1..753: note = MISC_FEATURE - CERSCO_DHODH_no_accession
MLATQKLLGASAPLVRQSAGLNRAQWRSVQRQWQRQLSLHSSGPRIAQHARNAPRRYQSTGEQAKEGAETAKAQAVKAGGATANTGSRFVAYVYGTGLVLVLSFGYLYATDTRASIHQFAVTPLVRLLFPDAEDAHHAGVRYIKALHDFGLAPRERGNADHALGDLKVEVFGHILDNPLGTSAGIDKDAEIPDALFALGPAIVEVGGATPLAQEGNPKPRVFRIPSQNALINRYGLNSRGAEHVAVQLRERVRQFAYENGLGLEAESEQRILDGEAGVPPGSLVDGKLLAVQVAKNKTTPDDDIAAVAADYVFCVEHLAKYADILVVNVSSPNTPGLRDLQATKPLTAILSSVVDAAKSVDRKTKPAVMVKVSPDEDSDAQIEGICTAVWSSGVDGVIVGNTTKKRPEPIPKGYVMPADDVRTLTEVGGYSGPQMFERTLNLVKRYRSKLDQGPHDNQPANTQKAVQSPLDPLERGFKGDSTPEDARQHASQESIARDQQRLKPLTSEAEADSKRSLIELPERHTSEQPEAAKHVGQAAAPASTQPTDAAAPQNATQIGKASSAREAAEGAVQKVISVGESNSSTEATKSGLRSAAAAAKDNAEQVADKARESAEQLKAKAESAKDQAQSAASQAKSQLDSATSDTKAKASELANKAASTAQSVATAAKDQFNHITSDPYAQVSAPKVEAPPVKDKVIFATGGITNGEQCLQILNAGASVCQVYTAMMYGGVGTITRMKGEMRAELRGQGKKE

SEQ ID NO: 10 - DNA, 2259 bp, synthetic construct
  misc_feature 1..2259: note = CERSCO_DHODH_cDNA_CP036211.1_684093-686351
  source 1..2259: mol_type = other DNA
atgctggccacgcagaagctgctgggcgcttctgcaccgctcgtgcggcaatctgcaggcctgaaccgcgcccaatggcgaagcgtgcagcggcaatggcagcgccaactttcgctgcactccagcggacctcgaatcgcccaacacgcccgaaatgcaccacgacgataccaatcgaccggagaacaggcgaaagagggcgcagaaacagcgaaggcgcaggcggtcaaggcgggaggggccacggccaacacaggctctcgtttcgtcgcatacgtctatggaacgggcctcgtcctcgtgctgagcttcgggtacctgtacgctacggacacacgcgccagcatccatcaatttgccgtgacccctctcgtccgtctgctgttcccagacgccgaagacgcgcatcacgcgggagtgcggtacatcaaagccctgcacgacttcggcctcgcgccgcgagaacgcggaaacgcggatcacgccttgggagatctgaaggtggaggtattcgggcacatcttggacaacccgctggggacatcggcgggtatcgataaggatgctgagattcctgacgcgttattcgcgttgggaccggcgatcgtggaagtgggcggcgcgacacctttggcacaggaagggaacccaaagcccagagtctttcgaatcccaagccaaaacgctttgatcaatcgatacggtctcaattcgcgaggagcagagcatgtggcagtacagttgcgggagcgagtgcggcagtttgcgtacgagaacggactggggctggaagcagaatcagaacagaggatcctggatggtgaagcgggcgtgccaccaggaagtctggtggatggcaagttactggcagtgcaagtggccaagaacaagacgacaccagacgacgacattgcggctgtcgcagcggattacgtcttctgtgtagaacatctggccaaatacgccgacattctggtcgtcaatgtctcatctcccaacactccggggttgcgcgatctccaagcaacaaagccactgacagcgattctttcgtctgtcgtcgatgccgccaagtctgtcgaccggaagacgaaaccggcagtcatggtcaaggtgtcgccagatgaggactcggatgcgcaaatagaaggcatctgcactgcagtctggtcgtctggtgtggatggagtcatcgtgggaaatacgaccaaaaagcgacctgaaccgataccaaagggttatgttatgcctgccgacgacgtgcgaacactcactgaagtcggaggatattccggccctcagatgtttgagcggacactgaatctcgtcaaacgataccgatcgaagctcgatcaagggccgcacgacaatcaacccgccaacacacagaaggcggttcagagtcctctcgatcccctcgagcgcggtttcaagggtgattctactcccgaggatgctcgacagcatgcatcacaagagagcatcgcgcgtgatcaacaaagactaaagccgttgacgagcgaggcggaagccgacagcaagcgatcgctgattgagctccctgagcgacacacttctgagcagcccgaggcggcgaagcacgttggtcaggctgctgctccagcttcgacgcaacctaccgatgctgctgctccacagaatgcgacgcagataggcaaggccagttcagccagagaagctgccgaaggagcagttcagaaggtcatttccgtgggcgaatcgaattccagtaccgaagccaccaagtcaggactgcgatccgcggcagctgcagccaaggacaatgcagagcaagtggcggacaaggccagagagagtgccgaacagctcaaagcgaaggcagagtccgccaaagaccaagctcaatccgctgcctctcaagcgaaatcacaactcgactcggcgacgtctgacaccaaagccaaagcctccgaactcgcaaacaaggccgcctctaccgcccaatcagtcgctacagcagccaaagaccaattcaatcacatcacaagtgatccatatgcccaagtctcggcgcccaaagtcgaagctccacctgtcaaagacaaggtgattttcgcaacgggaggcatcacgaatggagagcaatgtctgcagatactaaatgcgggtgcaagtgtgtgtcaggtgtatactgcgatgatgtacggtggtgtagggacaattaccaggatgaagggcgaaatgagagcggagttgagagggcaggggaagaaggaa

SEQ ID NO: 11 - protein, 553 aa, Bipolaris sorokiniana
  REGION 1..553: note = MISC_FEATURE - COCHSA_DHODH_XP_007699125
MMNSAASLRALRVRPSIPRHRPTHRSLLQTPTRYATTSTTATEAATNVRSAGTRTKNFIYGTLLAVGGTFGYLYVTDTRASIHQWLLVPALRVIYPDGEDAHHAGTRMLKVLHSFGINPRERGVADSQGDLAVEVFGHTLNNPIGTSAGIDKGAEVPTPLFDLGAGVVEVGAITLMPQEGNPKPRVFRIPSQNALINRYGFNSEGAELVASRLRQRVREFAYHAGLGLDEDAERRVLDGEAGVPPGSLHEGRLMAVQVAKNKSTSENDIESVVRDYATAVGYVGKYADVLVVNVSSPNTPGLRTLQNVEPLTKLLSGVVGAVKKIDRKTKPAVMVKVSPDEDSEEQISGICEAVWDSGVDGVIVGNTTKKRPDPLPKGYLLSQSEASILLEQGGYSGPQMFERTLALVKKYRKALDEGPKHITLPTQPKDIKASAGQDSGDESTKSSMTKPSLDVEQPAPKPASSDFDDISESAPLVTLQSTPKAAPAPDAPRKVIFATGGITNGRQAREVLDAGASVAQVYTAMIYGGAGTITRIKQEMRDDAKAQKTITKS

SEQ ID NO: 12 - DNA, 1903 bp, synthetic construct
  misc_feature 1..1903: note = COCHSA_DHODH_cDNA_XM_007700935.1
  source 1..1903: mol_type = other DNA
gttcaatcgttcacccgagcgattgcccagccattgaaagtcgcacgggctgcgcacgacactcgtgatgatgaacagtgccgccagcctgcgggctctgcgcgtccggccctcgataccgcgtcatcgcccgacccatcgctcgctgttacagacaccgacgagatatgctaccacatcgacaacggccaccgaagccgcgacaaatgtccgatcggctgggacccgcacgaagaacttcatttatggaactttgctggctgtggggggaacctttggatacctgtacgttacggacacgcgcgccagcatccaccaatggctccttgtacctgcgctgcgagtgatatacccagatggcgaggacgcacaccatgctggcactcggatgctcaaggtgctgcatagttttggaatcaaccctagggagcgtggcgtagccgactcgcagggtgatctagcagtcgaagtctttggtcacacattgaacaaccccattggaacatctgcgggcattgacaagggcgccgaggttccaacgccgctgttcgatctgggcgcgggcgtagttgaggtcggagctatcacattaatgccgcaagaaggcaaccccaagccgcgtgtctttaggatccccagccagaacgcgctcattaaccgctatggattcaactcagagggcgcagagctggttgcttcaagactgcgacaacgcgtgcgagagtttgcctaccatgccggcctggggctcgacgaggatgctgaacgacgtgtgctggatggcgaggctggtgtaccgcctggctcgcttcatgaaggacgcctcatggccgtgcaagtcgcaaagaacaagtcgacttcggagaacgacatagaatccgtggttcgcgactatgccactgctgttggctacgtgggaaagtatgcagatgtccttgtagtcaatgtctcgtcgcccaacacccccggcctccgcaccctccagaatgtcgaaccattgacaaagcttctctcgggtgtcgttggcgccgtcaagaagattgaccgtaagactaaacctgctgtcatggtcaaggtcagcccagacgaagactcggaagagcagatatcaggtatttgcgaggccgtgtgggactctggtgtcgatggtgtcattgttggtaacacaaccaagaagcggccagacccgcttcccaagggctaccttctgtcccaatcagaagcgagtattctacttgaacagggtggatactcgggccctcagatgtttgagcgcaccctagctctggtaaagaagtaccgaaaggcacttgatgaaggcccaaagcacatcaccctccccacgcagcctaaagacatcaaggcttctgctggacaagactcaggtgacgagtcaaccaagtcctcgatgaccaagccctccctcgatgtcgagcaacccgctcctaaacctgcatcctcagactttgatgacatctccgaatctgcacctcttgtcactctgcaatctacacccaaggccgcgcctgcaccagacgccccacgtaaggtcatttttgcaaccggaggaatcaccaatggccggcaggcacgtgaggttctcgatgctggtgcgagtgtagcacaagtctacactgccatgatttatggcggtgcaggcacaattaccaggatcaaacaggagatgagggacgatgccaaggcacaaaagaccattaccaagtcttagtgtacaaagcccagtagtgatgattggtgttagaccatatgtgttgtgtataccaagatttcaagtgtgtttactttagctaaaataaaaatagacgtgtaccatgttcccaaagaaagaaagaagtaatccgtgttccctagtttgcaactcgaaagttatatgtacaccagt

SEQ ID NO: 13 - protein, 498 aa, Colletotrichum graminicola
  REGION 1..498: note = MISC_FEATURE - COLLGR_DHODH_XP_008100212
MASICVRSGSVQTLLRTARNNTTAIRPRLPSNAPRRYISTGPGPSTESQPGSSLKAGLYGIALAGSLYISYLYVTDTRASIHRLSPMVMRFVWSDAEDAHHAGTSMMKTLYDLGLHIRERDRTLAEHPALATEVYGMQLSNPIGISAGLDKHADIPDALFALGAGIVEVGGCTPRPQDGNPRPRVFRVPTLDGMINRYGLNSRGADSMAITLRERVRKFARGVGATEQEVLDGEAGVPAGSLLPGRLLAVQIAKNKETDQKDVSAVAKDYVYCVQRLARYADILVVNVSSPNTPGLRDLQATEPLTRLLTAVVDEAAKVDRKVRPKVMVKVSPDEEEDAQVEGIVQAVCNSGVDGVIVGNTTNRRTGIVPPGVKLTAQEHKALQETGGYSGPAMYNRTLELVKRYRKKLDAKALQTGSAEEAAAIPSTDGSLSGKAHDLFEGKDTQRKVIFATGGITNGEQALNILNAGASVAMVYTGLTYGGPGTITRIKKEIKDQA

SEQ ID NO: 14 - DNA, 1497 bp, synthetic construct
  misc_feature 1..1497: note = COLLGR_DHODH_cDNA_XM_008102021
  source 1..1497: mol_type = other DNA
atggcctccatctgcgtgcgatccggcagtgtgcaaaccctcctccgcactgccagaaataacaccaccgccatccgcccgagactccctagcaatgcccctcgccgttatatttcgaccggccccggcccttccaccgaatcccagcccggctcgagtttgaaggccggcctctacggcattgccctcgccggctccctctatatctcctacctctacgtcaccgacacccgagcctcgatccaccgcctgtcgcccatggtgatgcgctttgtctggagcgacgccgaagacgcccaccatgctggcacgagcatgatgaagacactttacgacctgggattacacattcgcgaacgcgataggaccctagccgaacacccggccctcgccaccgaggtctacggcatgcagctctcgaaccccattggaatcagtgccggtctcgacaagcatgccgacatccccgatgccctctttgccctcggcgccggcatcgtcgaagtcggcggctgcaccccccggccgcaggacggtaacccccgcccgcgcgtcttccgcgtcccgaccctcgacggcatgatcaaccggtacggtctcaactcgcggggcgccgacagcatggcaattacgctcagggaacgcgtgcgaaagttcgctcgcggggtcggtgccacagagcaggaggttctcgacggcgaggctggtgtccctgcgggcagtctgctgcctggccgcctgctggccgtgcagatcgccaagaacaaggagacggaccagaaggacgtcagcgccgtggccaaggactacgtctactgcgtgcagcgcctggcgagatacgccgatatcctggtcgtcaatgtcagcagccccaacacccctggtctccgtgacctgcaggctaccgagccccttacgaggttgttgaccgccgtggttgacgaagccgccaaggttgaccgcaaggttcgccccaaggtcatggtcaaggtgtcccccgatgaagaagaagatgcccaggtcgagggaatcgtccaagccgtttgtaacagcggcgttgatggcgtcattgtcggcaacactaccaatcgccgtaccggcatcgttcccccgggcgttaagcttacggcacaagagcacaaggccctgcaggagacgggcggatactcaggccctgccatgtacaacaggacgctcgagctggtcaagaggtaccgcaagaagctcgacgccaaggccctgcagactggctcggccgaagaggcggctgccattccttccactgacggcagcctgtctggcaaggcccacgacctgtttgaaggtaaggacacccagagaaaggttatcttcgcgacgggaggcatcacgaatggagagcaagcgttaaacatcttgaatgctggtgcgagtgtggccatggtgtatacaggcttgacgtacggcggaccgggaacaatcaccaggataaagaaggaaatcaaggatcaggcgtaa

SEQ ID NO: 15 - protein, 496 aa, Colletotrichum obriculare
  REGION 1..496: note = MISC_FEATURE - COLLLA_DHODH_TDZ24597.1
MTSLFMRSGRMPYVLRAARNPTFRQSRLPSNNTLSRRHASTTSPSTDAASNLKLALYGTALAGSLYISYLYVTDTRASIHRVAPAILRFLYPDAEDAHHAGTTMMKTTYDLGLHIRERDRSLIQDPALASEVYGVNLWNPIGISAGLDKHADIPDALFALGAGVVEVGGCTPRPQEGNPKPRVFRVPSIDGMVNRYGLNSRGADSMAATLRERVRKFARKVGATEQEVLDGEAGVPAGSLLPGRLLAVQIAKNKETDERDPKAIAEDYVYCVQALAKYADILVVNVSSPNTPGLRDLQATEPLTKLLSAVVEEAAKVDRKVRPKVMVKVSPDEEEDTQVEGIVQAVCNSGVDGVIVGNTTKRRTGVIPKGVQLSAKEQKAFLETGGYSGPEMYGRTLQLVKKYRKKLDNQTLQSGSAEEAAAIPSVDGTLSGKLHDLVEGKDMPRKVIFATGGITNGEQALKILNAGANVAMVYTGLTYGGPGTITRIKRELRDQQ

SEQ ID NO: 16 - DNA, 1491 bp, synthetic construct
  misc_feature 1..1491: note = COLLLA_DHODH_cDNA_AMCV02000004
  source 1..1491: mol_type = other DNA
atgacctccctcttcatgcgctccgggcgcatgccgtacgtccttcgtgccgcaagaaatcccacttttcgccagtccagactccctagcaacaacaccctctccaggcgtcatgcttcgacgacctctccctcgaccgacgctgcctcgaacttgaagcttgctctctacggcactgcactggccggctcactctacatctcctacctctacgtcaccgacactcgtgcttctatccaccgggtcgctcctgctatactgcgcttcctctaccccgatgccgaagatgcccatcacgccggcacgactatgatgaagaccacgtacgacctgggcctgcatatccgcgaacgcgaccgctcgctcatccaggaccccgccctggcttccgaagtttatggcgtcaacttatggaaccccatcggaatcagcgctggcctcgacaagcacgccgacatccccgacgccctctttgctctcggtgccggtgtcgttgaggtcggtggctgcacaccacgccctcaggagggaaaccctaagccccgcgtctttcgcgtcccgagcatcgacggtatggtcaaccgctacggcctcaactcccgtggtgcggacagcatggctgccaccctccgcgagcgggtgcggaagttcgcccgcaaggttggtgctacggagcaagaggtactcgacggcgaggcgggcgttcccgcaggcagtctgcttcccggccgtctgcttgccgtgcagattgccaagaacaaggagacggacgagcgcgatcccaaggccattgccgaggactacgtctactgcgtgcaggccttggccaagtacgccgacatcctggtcgtcaacgtcagcagccccaacaccccgggtctgcgtgatcttcaggccacagagcctttgacgaaattgttgagtgctgtggttgaggaggctgccaaggtcgaccgcaaggtccgccccaaagtcatggtcaaggtatctcccgacgaggaagaggatactcaagtcgagggcatcgtccaggccgtctgcaacagtggcgtcgacggcgtcatcgtcggcaacaccaccaagcgtagaacgggcgtcatccccaagggtgtccagctttcggccaaggagcagaaggccttcctggaaacgggcggctactctggcccggagatgtacggaaggacgctgcagctcgtcaagaagtaccgcaagaagctcgacaaccagactctgcagtcgggctcggccgaggaggctgcggccatcccttccgtcgacggcacgctgtccggcaagctgcacgacttggtggaggggaaagacatgccgaggaaggtcatctttgcgactggcggtatcacgaacggagagcaggcgctgaagattctgaatgccggcgctaacgtggccatggtgtacacgggtttgacgtatggtggaccaggaaccatcaccaggataaagcgggaattgagggaccaacagtag

SEQ ID NO: 17 - protein, 559 aa, Corynespora cassicola
  REGION 1..559: note = MISC_FEATURE - CORYCA_DHODH_PSN63766.1
MNTAAQMRALRIRPSTLRSNLSRRSVFHPTRNASSTATDARSAATRTKNLVYGTALTFGLAFGYLYVTDTRASVHEWLVVPALRQIYPDGEDAHHAGTAILKGLHSFGLNPRERGNPDAAGDLAVEIFGHTLDNPIGTSAGIDKGAEVPTPLFDLGAAIVEVGAVTPYAQPGNDRPRVFRLPSQNALINRYGFNSEGAEKVALRLRQRIREFAYHVGLGMDEEAERKVLDGEAGVPPGSLRPGRLMAVQITKNKTTSEHDIDAVVRDYTYCVGQVGKYADILVVNVSSPNTPGHRALQNVEPLTKLLSGVVQAVKQVDRKTKPAIMVKVSPDEDSEEQVSGICEAVWDSGVDGIIVGNTTKKRPDPLPKGYLLPDHEASLLLEQGGYSGPQMFERTLDLVKRYRKAIDEGPKLPPPAPKLEKPAADQSNEGESDSSILEKLTPSTTPSEILDSAASSVPGASTTLPSESAPLSSNVDDLSESAPTLTVPPTPDAAPAPSTSGSRKVIFATGGITNGRQAREILDAGASVAQVYTALIYGGVGTISRIKEEMREEAKKGK

SEQ ID NO: 18 - DNA, 1677 bp, synthetic construct
  misc_feature 1..1677: note = CORYCA_DHODH_cDNA_JAQF01000000
  source 1..1677: mol_type = other DNA
atgaacacggccgctcagatgcgtgccttacgcatccgcccctcgaccctacgtagcaatctttcccgccgctcagttttccaccccacccgaaatgcgtcttcaacagcgacggatgcccgctcggccgcgacccggacgaagaatctcgtatacggaactgccttgacctttggcctggcttttggatatctctacgttacggatacgcgtgctagcgttcacgaatggcttgttgtgccggcattgcgtcagatctatcccgacggtgaagacgcgcaccatgccggcaccgccattttgaagggtctccatagcttcggcttgaaccccagggaacgcgggaaccctgatgcggctggagacctggcagtagagatttttggccacaccctggacaaccccattggaacctcggcgggtatcgataagggagccgaagtacctacgcctctcttcgacttgggagctgcgattgttgaggtgggagccgtgactccctacgcacagcccggaaacgataggcccagagtctttcgtcttccctcccagaacgcgctcattaaccgctacggattcaactcggaaggagctgagaaggtcgccctaagactgcgccagcgtatccgggagtttgcttaccatgtcggattgggaatggacgaagaggcagaacgcaaggtattggatggagaagctggcgttcccccgggttctctcaggccgggccgcctcatggccgtccaaattaccaagaacaagaccacctcggagcatgacattgatgccgtcgtccgagattacacctattgtgtcggtcaagttggtaaatacgccgacattctcgtagtgaacgtatccagccccaacacccctggccatcgtgcattgcagaatgtcgagcccctgaccaaactcctgagcggcgtggtccaggccgtgaagcaggttgacaggaagaccaagcccgctatcatggtcaaggtcagccccgatgaagactctgaagagcaggtttctggcatatgtgaggcagtgtgggattctggcgtcgacggaatcattgttggcaacaccaccaagaagcgacccgaccctcttcccaaggggtatctcctccccgaccacgaggccagccttctcctggaacagggcggctattccggtccacaaatgttcgagcggaccctggatctagtgaaacgctaccgcaaagccatcgacgaaggccccaagctccccccacctgcccccaagcttgaaaagcctgcagctgatcagagcaacgagggcgagtctgatagctccattctcgagaagctcaccccctcaactaccccttctgaaatcttggactcggccgcttcctccgtccccggcgcttccacaacgctcccctcggaatccgcacccctatcttccaacgttgacgacctcagcgaatccgcgcccaccctcactgttccccccacccctgacgctgctcctgcaccctcgacatcaggtagcaggaaggtcatcttcgctacgggtggcatcaccaacggccgccaggcccgtgagatcctcgatgcgggagcgagcgttgcgcaggtgtacactgcgctgatctacggcggtgtgggcacgattagcagaatcaaggaggagatgagggaggaggcgaagaaggggaaa

SEQ ID NO: 19 - protein, 472 aa, Fusarium culmorum
  REGION 1..472: note = MISC_FEATURE - FUSACU_DHODH_PTD01677.1
MSAALLRSRAAVSGTRIASSARPALPKSNLVQRRSASSSSSAGDGAGLRLALYGSATAAVLYGSYLYTTDTRAFFYRWVVPPLLRWAYPDAEDAHHAGTTALKVLHELNLNPRERDTTLNSPDLAVNVFGTEVQNPIAISAGLDKDGEVADALFDLGAGIVEIGGCTPLPQAGNPKPRVFRVRSLDGMVNRYGLNSRGADDMAIRLRDRLRRFARSLGLTERDVLDGNAKVPPGSLREGRLLAVQIAKNKETDERDEKAIAEDYVYCVRRLARFADLLVVNVSSPNTPGLRDLQATEPLTRLLSAVVDEAAKTDRKQKPKVMVKVSPDEDEDTQMEGIVEAVHRSGVDGVIVGNTTKRRDGVIPPGIKLTAQERQNLMETGGYSGPAMYSRTLDLVGRYRKMLDKHSFKASLGAKGGAQKVIFATGGITNGEQARQILNAGASVAMVYTGLTYGGPGTITRIKQELKGGVKN

SEQ ID NO: 20 - DNA, 1416 bp, synthetic construct
  misc_feature 1..1416: note = FUSACU_DHODH_cDNA_LT598662
  source 1..1416: mol_type = other DNA
atgtctgccgctctcctacgatcacgagctgccgtttctggcacccgaatcgcttcttccgcccgccctgctcttcccaagtcaaacctcgtccagcgccgcagcgcttcatcttcttcttccgctggtgacggagctggtctcaggctagctctctacggcagtgctaccgctgctgttctctacggctcatacctctacaccaccgatacaagagctttcttctaccgatgggtcgttcctccgctgctgcgctgggcctaccctgatgccgaggacgcccaccatgctggcaccacggccctaaaggtgctccatgaactcaacctcaacccccgcgagcgcgatactactctcaactctcccgacctggctgtcaatgtttttggaacagaggttcagaacccaattgctatctctgccggtctcgacaaggatggagaggtcgcagatgcgttgtttgaccttggagctggcattgtcgaaattggtggctgcacacccctccctcaagctggcaaccccaagcctcgagtgttccgcgttcgaagcctcgatggtatggtcaaccgatatggcctgaactctcgcggtgctgatgatatggctattcgattgagggaccgacttagacgatttgcccgatctcttggtctgactgaaagggatgtccttgacggaaacgccaaggtcccccctggaagtcttcgagagggccgacttctggctgttcagatcgccaagaataaggagacagacgagcgggacgagaaggctattgctgaggattacgtctactgtgttcgaagactcgcacgattcgctgatctgttggtcgtaaacgtcagcagccccaacactcccggtcttcgcgatctccaggccactgagcccctgacaaggctcctcagcgccgttgttgacgaggccgccaagaccgaccgaaagcaaaagcccaaggtcatggtcaaggtctcccctgacgaggacgaggacacacaaatggagggcatcgtcgaggctgttcaccgaagtggcgtcgacggagttattgtcggcaacacaaccaagcgacgagatggcgttatccccccgggcattaagcttactgctcaagagcgacagaacctcatggagactggtggttactccggtcctgccatgtacagccgcacactcgatcttgtcggccgatatcgcaagatgctagataaacactctttcaaggccagcctcggcgccaagggcggtgctcaaaaggtcatcttcgccacaggaggcatcaccaacggcgagcaagcacgacagatcctcaacgccggagctagcgtggcgatggtttacacagggttgacatatggcggtcccggcactatcaccaggatcaagcaggagttaaaaggtggtgtgaagaat

SEQ ID NO: 21 - protein, 469 aa, Fusarium oxysporum
  REGION 1..469: note = MISC_FEATURE - FUSAOX_DHODH_XP_018245138
MSAALFRSRAAVSGTRIASSARPAIPRSQLVQRRNASSSSSAGDGAALKLALYGTVGAVALYGSYLYATDTRAFFHRWVVPPFIRWAFPDAEDAHHAGTTAMKVTYEFNLHPRERDTTLDSPELAVNVFGTEVSNPIGISAGLDKDAEIPDVLFELGAGIVEVGGCTPLPQEGNPKPRVFRVRSLDGMVNRYGLNSRGADDMAIRLRDRLRRFARSLGVTEKDVLNGEANVPPGSLRKGRLLAVQIAKNKETDERDEKAIADDYVYCVRRLARYADILVVNVSSPNTPGLRDLQATEPLTRLLSAVVDEAAKTDRKQRPKVMVKVSPDEDEDTQMEGIVEAVHRSGVDGVIVGNTTKRRDGVVPEGIKLTAQEQKNLMETGGYSGPAMYSRTLDLVRRYRKLLDSRSFAATAGSPQKVIFATGGITNGEQARNILNAGASVAMIYTAMTYGGAGTVTRIKRELKDGGKN

SEQ ID NO: 22 - DNA, 2867 bp, synthetic construct
  misc_feature 1..2867: note = FUSAOX_DHODH_cDNA_XM_018387299.1
  source 1..2867: mol_type = other DNA
atgagaactcaattccagtctacgacgagcgttatcggcgtaataagctccttaaatcacgagacagaaatgaataagaaacaggtatatatcccagagttggcactccaggcgctgatgcgccttatatcttgctgtgtactgtgcatcacactgtctgatactgtgctctacaaccaagggtcggcaatttgtggatgcgttttgaagcttcacccgaacaacactatcgcgacccgtgaaaccattgggagctggcccggcccagcatcacttcccagccatgtctgccgctctcttcagatcacgagctgccgtctccggcacccgaattgcttcctccgctcgtcctgccattcctaggtcacagctcgtccagcgccgcaacgcatcatcttcctcttcggctggtgacggagctgctctcaagcttgctctctatggcactgttggtgctgttgctctatacggctcatacctctacgctaccgatacccgcgctttcttccaccgttgggtcgttcctccattcattcgctgggctttccccgacgctgaggacgctcatcatgccggcaccacagcgatgaaggtgacttatgagtttaaccttcaccctcgagagcgtgatacgaccctcgactctcctgaactcgccgtcaatgtatttggaactgaagtgtcgaacccaattggcatctctgctggtcttgacaaggatgctgagatcccagatgtgctatttgagcttggagctggtattgtcgaggttggcggctgcactcctcttcctcaagagggcaacccaaagcctcgagttttccgtgttcgcagccttgatggcatggtgaaccgatatggtctgaactctcgcggagccgacgacatggctattcgacttagggatagacttcgacgattcgcccgatctcttggtgtgactgagaaggatgttctcaatggagaggccaatgttccgcctggaagtctgcgcaagggccgtcttttggctgttcagatcgccaagaacaaggagacggacgagcgggatgagaaggctatcgccgatgactatgtatactgtgtgcgaagactagctcgctacgctgatattctggtcgtcaatgtcagcagccctaacacacctggtctgcgtgaccttcaggctaccgagcccctgacccggcttctcagcgctgtagttgacgaagctgccaagactgaccgaaagcaaaggcccaaggtcatggtcaaggtgtcacctgatgaggatgaggatactcaaatggagggtatcgttgaggctgttcatcgaagtggcgttgacggagtcatcgtgggcaacacaactaagagacgagatggtgttgttcccgagggtattaagctcactgctcaggagcaaaagaacctcatggagactggtggctactcaggacctgctatgtacagccgaacactcgaccttgttcgacgataccgcaagttgttagactcgcgctctttcgcggcaacagctggatcgccacagaaggtcatctttgctacaggaggcatcaccaatggcgagcaggcacgcaacattctcaacgctggcgcgagtgtagccatgatctacacagcaatgacatatggtggtgcgggtactgttaccaggatcaagcgggagttaaaagatggcggaaagaattagacatgctatagcaacatttcacgttttatttgttcgtcataggtataaaaatgggacaagttatgaagtttaaatggtttttatctgcttatcattttttttttttttttttttatatttgtctgtttttaatgtcaaaacccccagtactcaaaaggtccaaacttcagactcctgaagagtcatcagcttctccattttctagctttccttgattgggtccagccatatcaagtgacgaaggactgatcatccaaattggatgtcagcacgaaaactcctcaacttcaagaaacccagcgcgcaaggaggagtgacttaaaagccaggctccttgtcgagccactgacgcagagcgtcggcgtagcctcgaatctgggcctgaagagcagcaatgcgcttatcggcgtcactgatctccttcatggccaggtgaacattgtctggcactggatgcttgcaggcttgcgcaattgccgcctgtgcctccttcttgttgatgatgaggcgtagagcctcctccagagactgtaaggtcttcttacagtcgttaacccattcgcggcactgatttgccacattgccagcattggatatcgccttcttgttgctgatgctcgcgttgaccagatagttctggtctcgggtagtctccttgtacttcttaagcaggatttcatattcctgctggatttcctccagactccagctcttacactccccagaatccgggagtgtccaattttgcgttctcagtctcgccgccttcttttcttggctcttctcctgttggcgtactgacaagccccggagcatgccgcgaataccggtggcaggtgcaccgtggatggggtcctcgaaagggttctgggttgaggacatgatgttagaatttaagttaatgtgagagcataggtagggtttttattgatctctgagagacagagagttgggaatagagtactgtggcggtgaaaatgctgcgagaggtgttgcgagaggttgtggagagctttgctgtgcaagacaggtgtaggaagtaaatggtgtttggtgatgattgaatagataaggagatatcgtggctatttgtacctcaaattgaaatcatccgcagctctctaagccgtcccagttcgtgtgaagacattgattgtgtaaattctgttccatg

SEQ ID NO: 23 - protein, 472 aa, Fusarium graminearum
  REGION 1..472: note = MISC_FEATURE - GIBBZE_DHODH_XP_011328027.1
MSAALLRSRAAVSGTRIASSARPALPKSNLVQRRSASSSSSAGDGAGLRLALYGSAAAAVLYGSYLYTTDTRAFFYRWVVPPLLRWAYPDAEDAHHAGTTALKVLHELNLNPRERDTTLNSSDLAVNVFGTEVQNPIAISAGLDKDGEVADALFDLGAGIVEIGGCTPLPQAGNPKPRVFRVRSLDGMVNRYGLNSRGADDMAIRLRDRLRRFARSLGLTERDVLDGNAKVPPGSLREGRLLAVQIAKNKETDERDEKAIAEDYVYCVRRLARFADVLVVNVSSPNTPGLRDLQATEPLTRLLSAVVDEAAKTDRKQKPKVMVKVSPDEDEDTQMEGIVEAVHRSGVDGVIVGNTTKRRDGVIPPGIKLTAQERQNLMETGGYSGPAMYSRTLDLVGRYRKMLDKHSFKASLGAKGGAQKVIFATGGITNGEQARQILNAGASVAMVYTGLTYGGPGTITRIKQELKGGVKN

SEQ ID NO: 24 - DNA, 1419 bp, synthetic construct
  misc_feature 1..1419: note = GIBBZE_DHODH_cDNA_XM_011329725.1
  source 1..1419: mol_type = other DNA
atgtctgccgctctcctacgatcacgagctgccgtttctggcacccgaatcgcttcttccgcccgccctgctcttcccaagtcaaacctcgtccagcgccgcagcgcttcatcctcttcttccgccggtgatggagctggtctcaggttagctctctacggcagtgctgccgctgctgttctctacggttcatacctctacaccaccgatacaagagctttcttctaccgatgggtcgttcctccgctgctgcgctgggcctaccctgatgccgaggacgcccaccatgctggcaccacggccctaaaggtgctccatgaactcaacctcaacccccgcgagcgcgatactactctcaactcttccgacctggctgtcaatgtttttggaacagaggtccagaacccaattgctatctctgccggtctcgacaaggatggagaggtcgcagatgcgttgtttgaccttggagctggcattgtcgaaattggtggctgcacacccctccctcaagctggcaaccccaagcctcgagtgttccgcgttcgaagcctcgatggtatggtcaaccgatacggcctgaactctcgcggtgctgatgatatggctattcgattgagggaccgacttagacgatttgcccgatctcttggtctgactgaaagggatgtccttgacggaaacgccaaggtcccccctggaagtcttcgagagggccgactactggctgttcagatcgccaagaataaggagacggacgagcgggacgagaaggctattgctgaggattacgtctactgtgttcgaagactcgcacgattcgctgatgtgctggtcgtgaacgtcagcagccccaacactcccggtcttcgcgatctccaggccactgagcccctgacacggctcctcagcgccgttgttgacgaggccgccaagaccgaccgaaagcaaaagcccaaggtcatggtcaaggtctcccctgacgaggacgaggacacacaaatggagggcatcgtcgaggctgttcaccgaagtggtgtcgacggagttattgtcggcaacacaaccaagcgacgagatggtgttattcccccgggcattaagcttactgctcaggagcgacagaacctcatggagactggtggttactccggtcctgccatgtacagccgcacactcgatcttgtcggccgataccgcaagatgctagacaaacactctttcaaggccagcctcggcgccaagggcggtgctcaaaaggtcatcttcgccacaggaggcatcaccaacggcgagcaagcacggcagatcctcaacgctggagctagcgtagcgatggtctacacaggattgacatatggcggtcccggcactatcaccaggatcaagcaggagttaaaaggtggtgtgaagaattag

SEQ ID NO: 25 - protein, 797 aa, Plenodomus lingam
  REGION 1..797: note = MISC_FEATURE - LEPTMA_DHODH_XP_003834159.1
MPPAGNEYAAEQLKNIGNKCFKNGDYEQAEAHYSQAIQKNSANPLLFTNRANARLKLEKWEGVIDDSIRSIELLKDNMKAFFYLDETCPSIAILLSSQNECNAKMTTAQAQLAINHPNEALSSALMAYQLCTNSTQQTSNAATISALVLRCKKAKWDIRERERVRRRGGLLADLEAMLETQYKKDMDDIDARIEANEVTRVAGQEEKAERKSDFEKKRDDLRTAFAISDPENQQKREVPDYLVDGITFEIMHDPVVTKNGRSYERATLIEHLKRSPTDPLTRETLTIGDLRPNIALKEACTEFMEQNSGWVYDCEEVRMNVNPRLFRITNLSRYSLESQQQLHSHRRPYVLSAILIAMKNTAGMRNLRMRPVIPRCRPAQHLFQHPTRHATNATEAAAATVTATNVRSGGTRARNLVYGTILTVGLSFGYLYVTDTRASIHEWLVVPALRQIYPDGEEAHQAGTRILKALHNFGLNPRERGIADSQGDLTVPVFGHTLSNPIGTSAGIDKGAEIPTPLFELGAAVVEVGAITLLPQEGNPKPRVFRLPSQNALINRYGFNSEGAEHVATRLRQRVREFAYHKGLGMDEEAERAVLDGEAGVPPGSLLPGRLLAVQVAKNKTTSENDIEAVVRDYATTVGHVGKYADILVVNVSSPNTPGLRTLQNVEPLTRLLSGVVQAVKNIDRKTKPAIMVKVSPDEDSEEQISGICEAVWDSGVDGVIVGNTTKKRPDPLPRGYALPASEAKLLLEQGGYSGPQMFERTLALVTKYRKALDQGPKHPTFSTSSSTAATIADNSQ

SEQ ID NO: 26 - DNA, 2394 bp, synthetic construct
  misc_feature 1..2394: note = LEPTMA_DHODH_cDNA_XM_003834111.1
  source 1..2394: mol_type = other DNA
atgcccccggcaggcaacgagtatgctgcagaacagctgaaaaacatcgggaacaaatgcttcaagaatggcgactatgagcaagccgaggcacactactcccaggctatacagaagaactcagccaatccgttattattcacgaaccgggcaaacgcaagactgaagctggaaaaatgggagggcgtgattgatgatagtatccgcagtatcgagttactcaaggataatatgaaggccttcttctatttggatgaaacatgtccgtcgattgcgatattactctcttcccaaaatgaatgcaacgctaagatgacgacagcacaagctcaactcgcaatcaatcaccccaacgaagccctctccagtgccctcatggcctaccaactctgcacaaattccacgcagcaaacctcaaacgcggccacgatatctgctcttgtcctgcgttgcaaaaaagcaaagtgggatattcgcgaacgcgaacgcgtacggcggcgtggaggcttgctcgcagatctggaagccatgctcgagacccagtacaagaaagacatggatgacatcgatgcgcgcatcgaagctaacgaagtcacacgcgtagctggccaagaagagaaagcagagcgcaaaagtgattttgagaagaaacgagatgatctacgcactgcatttgccatttcagacccagaaaaccagcaaaaacgcgaagtgcctgattatctggtcgacggaatcacgtttgaaatcatgcacgatccggttgtaaccaaaaatggccgttcctacgagcgcgcaacactgattgagcatttgaagcgcagtccaactgatcccttgacgagggagacgctgacaatcggcgacctgaggccgaatatcgcgttgaaagaggcgtgtacagagttcatggaacagaacagtgggtgggtttatgattgtgaggaagtccgcatgaacgtcaacccgaggctgttccgcatcaccaacctctcgaggtacagcttagaatcccaacagcaattgcacagccatcgacggccttatgtgctgtccgccatacttattgccatgaaaaacactgcaggtatgcggaacctgcgcatgcgccccgtgataccacgttgccgccccgcacaacacctgtttcaacaccccacaagacacgcgaccaatgcaacggaagccgccgctgctaccgtgacagcgacgaatgttcgatcgggaggcacacgtgccagaaacctggtatacggtaccattctgactgtcggtctctcctttggctacctctacgtcaccgacacacgcgcaagcattcacgaatggcttgtcgtaccggccctacgacaaatatatcccgatggcgaagaggcgcatcaagcgggaacacgaatactcaaggctctccataattttgggctaaaccccagggagcgtggtatagctgattcacaaggcgatctgactgtaccagtctttggacacacattgagtaacccaattggcacatcggcgggtatcgacaagggcgctgagatccccacgcccttgttcgagctgggtgcggcggttgttgaggttggcgccattacattgttgccacaggagggcaatcccaagccacgtgtattcaggctcccgagccagaacgcgctcatcaaccgctatgggttcaactcagagggtgccgaacacgtagctacccgacttcgtcagcgcgttcgcgagtttgcttatcacaagggattgggcatggatgaagaggctgagcgggcggtcctcgatggagaagctggtgttccacccggctcattgctgccaggacgactgctggcagtccaagttgccaagaacaagaccacgtcggaaaacgacattgaggcagtcgttcgtgactatgcgaccactgtaggacatgttggaaagtacgccgacatcctggtagtaaatgtctcgtctcccaacacacccggcctgcgaacattacagaacgtggagccattgacacggctgctctcaggcgtcgtccaagccgtcaagaacatcgaccgcaagaccaaacccgccatcatggtcaaagtcagcccagacgaagattcagaggaacaaatctctggtatctgcgaagccgtttgggactcgggcgtagacggcgtcatcgtcggtaacacaacgaagaagcgaccagaccctttgcccagaggatacgcactgccagcgtctgaggccaaacttctgctagagcaaggcggatactctggaccacagatgtttgagcgcacacttgcgcttgtgaccaagtacagaaaagcactggaccagggaccaaaacatccaacgttctcaacgtcatcaagcactgcggcgacaatcgccgacaactctcaatag

SEQ ID NO: 27 - protein, 549 aa, Parastagonospora nodorum
  REGION 1..549: note = MISC_FEATURE - LEPTNO_DHODH_XP_001803781.1
MLSAANMRALRVRPSMLRVGASRSLTRHASTDAVIAPQTASNVRSAGTRSKNLIFGTVLTLGLSFGYLYVTDTRASIHEWLLVPALRQIYPDGEDAHHAGTAMLKALHSFGMNPRERGPGDSAGDLAVEVFGHTLANPIGTSAGIDKNAEVPTPLFELGPAIVEVGAVTLLPQEGNPKPRVFRIPSQNALINRYGFNSEGAELVATRLRQRVREFAYHSGLGLDEEAERKVLDGEAGVPPGSLKQGRLMAVQVAKNKTTSENDIDAVVRDYATSVSHVGKYADILVVNVSSPNTPGLRTLQNVEPLTRLLTGVVQAVNKIDRKTKPAIMVKVSPDEDSEEQVSGICEAVWDSGVDGVIVGNTTKKRPDPLPKGYLLPASEAQVLLEQGGYSGPQLFERTLALVSRYRKALDEGPKSVPSQQADSEGPNTSIIEKLQPSKQTGSSKTETTVEVGSASPALSSNVDDLQESAPTLTLHSKPVTAPAPKNSQKVIFATGGITNGRQAREILDAGASVAQVYTALIYGGAGTISRIKQEMRDDAKKQTTATKR

SEQ ID NO: 28 - DNA, 1650 bp, synthetic construct
  misc_feature 1..1650: note = LEPTNO_DHODH_cDNA_XM_001803729.1
  source 1..1650: mol_type = other DNA
atgctctcggccgctaacatgcgcgcccttcgagtgcgaccctcgatgctgcgtgtaggcgcttctcggagcctcacgagacatgcctccaccgatgccgtgatcgctccccaaaccgcgtccaacgttcgctcggccggcacgcgctccaagaacctcatcttcggcaccgtcctgactctcggcctctccttcggctacctctacgtcaccgacacccgcgcgagcatccacgaatggctccttgtaccggctctgcgccagatatatcccgatggtgaagacgctcatcacgcgggcacagccatgctcaaagcgttgcacagcttcggaatgaaccccagagaacgagggcctggtgactctgctggtgacttggcagtcgaggtctttggacacacgttggcgaaccctatcggaacctctgctggcattgacaagaatgccgaggtacccacaccactgttcgagctgggtccggcgatagtcgaggttggcgcagtcacgttgctgccgcaagaaggaaacccgaaaccacgtgtgttcaggatccccagccagaatgcgcttatcaaccgctacggcttcaactcagaaggcgccgaactggtagccacaaggctacgacagcgtgtcagggagtttgcataccactctggtctgggcctggacgaagaggccgagcgcaaggtgcttgacggcgaggctggcgtaccaccaggctcgctcaagcagggcagacttatggcagtccaggttgcgaagaacaagacaacatccgagaacgacatcgatgctgttgtgcgtgattacgcaacttcagtcagtcacgtcggcaagtatgccgacatcctggtggtcaacgtctcctcaccaaacactcccggtctccgcacactgcagaatgtagagccgctcacacgcttgcttaccggcgttgtacaggctgtcaacaagatcgatcgaaagaccaagcccgcaatcatggtcaaggtcagcccagatgaagactcagaagagcaggtctctggcatctgcgaggccgtttgggactcgggtgttgacggtgtcattgttggaaacacgaccaagaagcgacctgacccgttgccaaaaggctacctgctccccgcctcggaagcccaggtacttctcgagcagggcggatactcgggcccgcagttgttcgaacgcaccctagccctggtgagcaggtacaggaaagcgctggatgagggtccgaaatctgtaccgtcacagcaggctgatagcgaagggccaaacacaagcataatagaaaagctgcaaccatcaaagcagactggctcatcaaagaccgagaccactgtcgaagtcggatcagcttcacccgcactctcctccaacgtagacgacctacaagagtccgcgccaacacttaccctccattcaaaacccgtcacagcacccgcacccaaaaactctcagaaagtcatctttgccactggaggcattaccaacggacgacaagcacgcgagatcctcgatgccggagctagtgttgcgcaagtctataccgcgctcatctacggtggtgcaggcacgatcagcaggataaagcaggagatgagggacgatgcgaagaagcagacgacggctactaaacgttag

SEQ ID NO: 29 - protein, 520 aa, Monographella nivalis
  REGION 1..520: note = MISC_FEATURE - MONGNI_DHODH_in_house
MASTARLAGRLVATSTATARPRATLLANSRPVYRHSLQLLPRTARRHASSSSPASTSVSNLKIALWSSSIALSGYAVYLYATDTRSFIHRYVVPPVLRIAIPDAEDAHHAAVSALGTLYSLGLNPRERSRSADGPALSVSVFGEDLCNPIGISAGLDKHAEIPDALFALGAGVVEVGGCTPLAQPGNPKPRMFRVPGLDGLINRYGLNSHGADHMAMKLRERVRLYAKSVGLDEDGVLNGEANVPPGSLLPGRLLLVQIAKNKDTDGGNVDAVSRDYVYCVERLAKYADVLVVNVSSPNTPGLRDLQAVEPLSKILTAVVDEARRANRKTKPKVMVKVSPDEDDESQMRGIVQAIYSSGVDGVIVGNTTRDRLGVIPPGVKLDSRDTKAMAEVGGYSGPMMFNRTLDLVGRYRKMLDNYSRQVRQKETLDKEAVDASLAGIDGSVTGMVEDAALTHKGKQTQGPKVIFATGGITNGDQALRILNAGASVAMIYTSLVYGGSGTVTRIKSQMKEEIGSKEK

SEQ ID NO: 30 - DNA, 1563 bp, synthetic construct
  misc_feature 1..1563: note = MONGNI_DHODH_cDNA_inhouse
  source 1..1563: mol_type = other DNA
atggcctccactgcacgtcttgctgggcgccttgttgcgacgtcgactgcgaccgccagaccgcgggctactcttcttgccaacagccgtcccgtctaccgccacagcttgcagctgctacctcgcactgctcgccgccacgcttcgtcgtcgagtcctgcgtccacctccgtctcgaatctcaagattgccctctggtcgagttccattgccctttccggctacgccgtctacctgtatgccaccgacacgagatccttcatccaccgatacgtcgtcccgcccgtcctccgcatcgccattcccgacgccgaagatgcccatcatgcagccgtctctgccctaggcacgctctatagccttggcctcaaccctcgcgagcgctcacggtcagcagacggcccagccctctccgtctccgtcttcggtgaggacctgtgcaacccaatcggcatctccgcgggccttgacaagcatgccgagatccccgatgccctcttcgcactcggcgctggtgtcgtcgaggttggcggctgcacgcccctcgcccagcccggcaaccccaagccgcgcatgttccgcgtgcccggcctcgacggcctcatcaaccggtacggcctcaactcacatggcgccgaccacatggccatgaagctgcgcgagcgcgtgcgcttatatgcaaagtccgtcgggctcgacgaggacggtgtgctcaacggagaggccaatgtcccccccggaagtctgctcccgggacgcctgctgctcgtacagatcgccaagaacaaggacacggacggtggcaacgtcgacgcggtctcgcgcgactacgtttattgcgtggagcgcctcgccaagtatgctgacgtgctcgtcgtcaacgttagcagccctaatacgcctggcttgcgcgacctgcaggccgtcgagccgctcagcaagatcctcactgctgttgtcgacgaggcccgccgagcaaaccgcaagaccaagccaaaggtcatggtcaaggtctcgcccgacgaggacgacgagtcacagatgcgagggatcgtgcaggccatttactctagtggcgttgacggcgtcattgtaggcaacacaacgcgggaccggctcggtgtgattccacctggtgtcaagcttgacagcagggacacaaaggctatggccgaggttggcgggtactcgggccccatgatgtttaaccgcacactggaccttgtcggtcggtatcgcaagatgctcgacaattactcgcgccaggtccgacagaaggagacactcgacaaggaggctgtcgacgctagcctcgctggcatcgacggctcggtgacgggcatggtcgaggatgccgcgctgacgcacaagggcaagcagacacagggccccaaggtcatattcgcgacgggcggcatcaccaacggcgaccaggcgctcaggatcctcaacgcaggcgcgagcgtagccatgatctacacgtcactcgtgtacggcggctcgggcaccgtgacgcgaatcaagagccagatgaaggaggagattggctcgaaagaaaagtag

SEQ ID NO: 31 - protein, 446 aa, Pseudocercospora fijiensis
  REGION 1..446: note = MISC_FEATURE - MYCOFI_DHODH_XP_007930122.1
MLATQRLFAYIYGTGLVLLLSFGYLYITDTRASLHQYFVVPLIRLVYSDAEDAHHAGVRYIKALYDFNLHIRERGNQDKELGDLKVEVFGHVLHNPLGTSAGIDKDAEIPDALMALGPAVVEVGGTTPLPQLGNPKPRVFRIPSQNALINRYGLNSEGAAHVAAQLRERVRHFAYASGLGLGPDSEKLVLDGEAGVPPGSLVPGKLLAVQIAKNKTTPDADIAAVARDYVFCVDHLAKYADVLVVNVSSPNTPGLRDLQATAPLTAILSAVVDAAKSVDRKTKPAVMVKVSPDEDTDSQIEGICSAVWASGVDGVIVGNTTKKRPDPLPQGYVIPVKEAATLTEVGGYSGPQMFKRTLDLVKRYRINLDQGPQEDARDSTPSFSPASKVIFASGGITNGEQCLEILNAGASVCQIYTAMMYGGVGTVTRIKSEMREAIKGVRDKSS

SEQ ID NO: 32 - DNA, 1341 bp, synthetic construct
  misc_feature 1..1341: note = MYCOFI_DHODH_cDNA_XM_007931931.1
  source 1..1341: mol_type = other DNA
atgctggccacgcagaggctgtttgcgtacatctatggcactgggctggtgctcttgctctccttcggctacctctacatcaccgatacccgcgccagcctacatcaatactttgtcgtccctctcataaggcttgtctactcggatgcagaggatgcgcatcatgctggcgttcgctacatcaaagctctttatgacttcaatcttcacattcgggaacgaggcaaccaagataaagagctcggagatttgaaggtggaagtatttggccatgttctccacaacccactgggaacatccgcgggcatcgacaaagatgctgaaattccagatgcgctcatggcattgggacctgcagtcgtagaagttggaggcaccacgccactaccgcagctgggcaatccgaagcccagagtgtttagaatccctagccagaacgccttgatcaacaggtacggcttgaactctgaaggagcagcccatgtcgcagcacagcttcgtgaacgggtacggcactttgcgtatgccagcggccttgggctgggccctgactcggagaagctggttctcgatggcgaggccggtgtccctcctggcagtcttgtaccaggaaagctactggctgttcaaatcgcgaagaacaagacaactccagatgccgatattgcagcggtcgcaagagattacgtcttctgtgtcgaccatcttgccaaatacgctgacgtcttggttgtgaacgtgtcgtcgcccaacacgccaggactccgagatctacaagctactgctccattgactgccattttgtctgctgtagtagacgctgccaagtctgtagaccgaaagactaagccggccgtcatggtcaaggtgtcgccggacgaagataccgactcacagattgaaggtatttgctcagcagtctgggcttcgggtgtagacggagttatagttggcaacacaaccaagaaacgacccgacccactgcctcaaggatatgtcatacctgtgaaggaggcagcgacgcttacagaagtgggtggctattctggcccgcagatgttcaagcgtactctcgatctcgtgaagagatatcgcataaatctcgatcaaggcccacaagaggatgctcgagactcaactccaagcttttctccagcctcaaaagtcattttcgcctctggcggcatcaccaacggcgagcagtgcttggagattctaaatgcaggagcaagcgtctgccaaatttacactgcaatgatgtacggtggcgtcggcacggtgacgcgaatcaaatcagaaatgcgagaagctattaagggagttagagataaatcctcgtag

SEQ ID NO: 33 - protein, 514 aa, Pyricularia oryzae
  REGION 1..514: note = MISC_FEATURE - PYRIOR_DHODH_XP_003719157
MASHSRILRLSRSTPSFHNAGRLRFQPPRRHASTVPPSSETPVQSSVLSPGARVFLYSAATAVTAYATYLYATDTRANLLHRYIVPPLLRTLVPDAEDAHHFGTAAMKAMFNMGLHIRDRTVASDEPILGVTVFGRRLANPVGISAGLDKDAQIPDALFAVGPAVVEVGGITPHPQKGNPQPRVFRVPTLEGMVNRYGLNSAGADAVARTLRARLRSFARENGLTEADVLEGRAGVPVGSLAPGRLLCVQVAKAKETDEADIQAVARDYTRCVERLAPYADVLVVNVSSPNTPGLRDLQRSGPLASILSAVVGEAKAAAARCGRAADPPRVMVKVSPDEDSDAQVEGVVEAVWGSGVDGVIVGNTTKRRDVIPPRIVLSGKEVENLHETGGFSGPALFDRTMSLVAKYRKALDGHSFSSGAGRPAAAAVEAGANMEEVSAQAGAAAVVVPPPTKKERDEQKVIFATGGITNGEQALAVLNAGASVAMVYTGMVYGGAGTVTRIKGEMKKQLTQS

SEQ ID NO: 34 - DNA, 1545 bp, synthetic construct
  misc_feature 1..1545: note = PYRIOR_DHODH_cDNA_XM_003719109.1
  source 1..1545: mol_type = other DNA
atggcatctcactctcgcattctgcgtctctctcgctccacgccatcattccacaatgccggacgacttcgcttccagcccccacggcgacacgcctcgacagttcctccatcctccgaaacgcctgtgcaatcctctgttcttagcccaggcgcccgcgtcttcctctactcggcagcgacagcagtgactgcctatgcaacatacctatacgcgaccgacacccgcgccaacctcctgcaccggtacatcgtcccgccgctgctgcgtacccttgtccctgacgccgaggatgcccaccactttggcaccgcggccatgaaggccatgttcaacatgggactgcacatccgcgatcgcaccgtcgcgtcggacgagccgatcctaggcgtcaccgtcttcggccgcaggctagccaaccccgtcggcatcagcgccggcctcgacaaggatgcccagatccccgacgccctcttcgccgtcggcccagccgttgtcgaagtcggcggcatcacgccgcacccgcaaaagggcaacccgcagccccgcgtattccgcgtccccacgctcgagggcatggtcaaccgctacgggctcaacagcgcaggcgccgacgctgtcgcccgcaccctccgcgcccgtctgcggtcctttgcgcgcgaaaacggcctgaccgaggccgacgtgctggagggccgcgcgggcgtccccgtcggctccctggctcccggcaggctgctgtgcgtgcaggtggcaaaggccaaggagacggacgaggccgacatccaggcggtggccagggactacacgcgatgcgtcgagcggctcgcgccctacgccgacgtgctcgtcgtcaacgtcagcagccccaacacccccggtctgagggacttgcagcgctcgggcccgctggcaagcatcctgtcggctgttgtgggcgaggccaaggccgcggcggctcggtgcggcagggcggccgacccgcctcgggtcatggtgaaggtctcgcccgacgaggactcggacgcgcaggtcgagggcgtggtggaggctgtgtggggcagcggagtggacggcgtcatcgtcggcaacacgaccaagcgccgcgacgtcatccccccgcgcatagtgctgtccggcaaggaggttgagaacctgcacgagacgggcggcttctccggcccggccctgttcgaccgaacgatgtcgctagtggccaagtaccgcaaggcgctggatgggcactcgtttagctccggagccggccgtcctgccgcggcggcggtcgaggcgggtgctaatatggaggaggtctccgcacaggctggtgccgcggccgtcgttgttccgccgccgacgaagaaggagcgggacgagcagaaggtgatctttgccacgggcggtatcacgaatggggagcaggccctggcggttttgaatgcgggcgccagcgtggcgatggtgtacacggggatggtgtacggtggtgctggcacggtgacgaggataaagggcgagatgaagaagcagctgacgcagagctga

SEQ ID NO: 35 - protein, 551 aa, Pyrenophora teres
  REGION 1..551: note = MISC_FEATURE - PYRNTE_DHODH_EFQ86357.1
MRALRVRPSIIRPQLSPRHIQQPRRYAATATEAATNVRSAGTRTKNLIYGTVLTVGGTFGYLYATDTRASIHEWLVVPALRQIYPDGEEAHHAGTRMLKVLHSFGLNPRERGIADTQGDLAVEVFGHTLNNPIGTSAGIDKGAEIPTPLFELGAGVVEVGAVTLLPQEGNPKPRVFRLPSQNALINRYGFNSEGAELVASRLRQRVREFAYRVGLGLDEDAERRVLDGEAGVPPGSLLKGRLMAVQVAKNKTTSENDIDAVVRDYATAVGYVGKYADILVVNVSSPNTPGLRTLQNVEPLTRLLSGVVGAVKKIDRKTKPAIMVKVSPDEDSEEQIAGICEAVWDSGVDGVIVGNTTKKRPDPLPKGYLLPESEAKLLLEQGGYSGPQMFERTLALVSKYRKALDEGPKHMSPQDSKPVTGQSTGDEPSPSILEKLESSSSLQGSKTKPTLDLEQPSPKPLSSNIDDMSESAPKITLQATPKAAPAPDAPRKVIFATGGITNGRQAREILDAGASVAQVYTAMIYGGAGTISRIKQEMRDDANAQKTLTKR

SEQ ID NO: 36 - DNA, 1656 bp, synthetic construct
  misc_feature 1..1656: note = PYRNTE_DHODH_cDNA_GL537441.1:c28889-27234
  source 1..1656: mol_type = other DNA
atgcgtgccttgcgcgtacggccctcgatcatacgcccgcagctatcccctcgccatatccagcaacccaggagatatgctgcgacggcaaccgaagccgcgacaaatgttcgatctgcaggcacacgcacgaagaatctcatctacggcaccgttttgactgtgggtggcacctttggctacctgtacgcaaccgatacccgcgccagcatacacgaatggctcgttgtacctgcattacgacaaatatacccagacggcgaagaggcacaccacgctggcactcgcatgctcaaggtgctgcacagcttcggcttgaaccctagagaacgcgggatagcggatacacaaggcgacctcgccgttgaagtctttgggcacactctaaacaatccaattggaacatctgctggcattgacaagggtgctgagataccaacaccactgttcgagctgggagctggtgttgtggaggtcggagccgtcacattgctaccacaggaaggtaacccaaagcctcgcgtctttagactaccgagccagaacgccctcatcaatcgctatggctttaactcagagggcgcagagctggtggcttcgaggctacggcaacgcgtgagagaatttgcataccgcgtcggacttgggctagatgaagatgcggaacgacgagtgctggatggcgaggcgggtgtacctccaggatcgctactcaaaggacggcttatggctgtgcaagtcgcaaagaacaagacgacatcagagaacgacattgatgctgtagtacgcgactatgccactgcggtaggctacgttggaaagtatgcagatatcctggtggtcaacgtctcctcgcccaacaccccgggcctccgtacgctccaaaatgtcgagcctttgacacgactcctttccggtgtagtcggtgctgttaaaaagattgatcgcaagaccaagcccgctatcatggtcaaggtcagtccagacgaggactcggaagagcaaattgcaggtatttgtgaagctgtgtgggattcaggcgtggacggtgtcattgtcggcaacacgacgaagaagcgaccagaccctctgccaaagggctacctgctaccggaatccgaggccaagctactcctcgaacaaggtggatactcgggaccacagatgttcgaacgcacattggcactcgtgagcaagtaccgcaaagcacttgacgaaggccctaagcatatgtcgccacaagactccaaacccgtcaccggtcaaagcacaggtgacgaaccttcccccagcatcctagagaagcttgagtcctcatcctcgttacaaggctccaagacaaagcccactctagatctcgaacaaccatcacccaaaccgctatcttcgaatatcgatgacatgtcggaatccgcgcctaagattaccttacaagctacacccaaggccgcgccggcgccagatgctccccgcaaggttattttcgcaacgggtggcatcacgaatggccgtcaagccagagagatcctagacgcgggagcgagtgtggcgcaggtatatactgccatgatctatggaggcgcgggcacaattagcaggatcaagcaagagatgagggatgatgcaaatgcacagaagacattgacaaagcggtag

SEQ ID NO: 37 - protein, 551 aa, Pyrenophora tritici-repentis
  REGION 1..551: note = MISC_FEATURE - PYRNTR_DHODH_KAA8622403.1
MRALRVRPSIIRSQLSPRHIQQPRRYAATATEAATNVRSAGTRTKNLIYGTVLAVGGTFGYLYATDTRASIHEWLVVPALRHIYPDGEEAHHAGTRMLKVLHSFGMNPRERGIADTQGDLAVEVFGHTLNNPIGTSAGIDKGAEIPTPLFELGAGVVEVGAVTLLPQEGNPKPRVFRLPSQNALINRYGFNSEGAELVASRLRQRVREFAYRIGLGLDEDAERRVLDGEAGVPPGSLLKGRLMAVQVAKNKTTSENDIDAVVRDYATAVGYVGKYADILVVNVSSPNTPGLRTLQNVEPLTRLLSGVVSAVKKIDRKTKPAVMVKVSPDEDSESQIAGICEAVWDSGVDGVIVGNTTKKRPDPLPKGYLLPESEAKLLLEQGGYSGPQMFERTLALVSKYRKALDEGPKHVSPQDSKSVTGQSTGDEPSPSIVEKLEPLSSSQSSKTKATLDLDQPSPKPLSSNIDDISESAPKITLQATPKAAPAPDAPRKVIFATGGITNGRQAREILDAGASVAQVYTAMIYGGAGTISRIKQEMRDDANAQKTLTKR

SEQ ID NO: 38 - DNA, 1656 bp, synthetic construct
  misc_feature 1..1656: note = PYRNTR_DHODH_cDNA_SAXQ01000002.1_c685824-684169
  source 1..1656: mol_type = other DNA
atgcgtgcgttacgcgtacggccctcaatcatacgctcgcagctatcccctcgccatatacagcaacccaggagatatgctgcgacggcaaccgaagccgcgacaaatgttcgatctgcaggcacacgcacgaagaatctcatctacggtaccgttttggctgtgggtggcacctttggctacttgtacgcaaccgatacccgcgccagcatccacgaatggctcgttgtacctgcactacgacatatatacccagacggcgaagaggcacatcatgctggcactcgcatgctcaaagtgctgcacagcttcggcatgaaccccagagaacgtggaatagcggatacacaaggcgacctggctgttgaggtctttgggcacactctgaacaatccaattggaacatctgctggcatcgacaagggcgccgagataccaacaccactgttcgagctgggagctggtgttgtagaggttggagctgtcacattgctaccgcaggaaggtaacccaaagccccgcgtttttaggttaccaagtcagaacgcccttatcaaccgctatggcttcaactcggagggcgcagagctagtggcttcaaggctacggcaacgcgtgagagagtttgcataccgtattgggcttgggctggatgaggatgcggaacgacgagtgctggatggcgaggcaggtgtacctccaggatcgctactgaaaggacggcttatggctgtgcaagttgcaaagaacaagacgacatcggagaatgacattgatgctgtagtacgcgactatgccactgcggtaggctatgttggaaagtatgcagatatactggtggtcaacgtctcctcgcccaacaccccaggcctccgtaccctccaaaatgtcgagcctttgacacgacttctctcaggtgtagtcagtgctgttaagaagattgatcgcaagaccaagcccgctgtcatggtcaaggttagcccagatgaggactcggaatcacaaatcgcaggcatttgtgaagctgtgtgggattcaggcgtggatggtgtcattgtcggcaacacgacgaaaaagcggccagacccgttgccaaagggctacttactaccggaatccgaggccaagctcctcctcgagcaaggtggatactcaggcccacagatgttcgaacgcacattggcacttgtgagcaagtaccggaaagcacttgacgagggccctaagcatgtgtcgccacaagactctaaatccgtcaccggccaaagtacaggtgacgagccgtccccaagtatcgtagagaagctcgagcctttatcctcatcacaaagctctaagacgaaggccactctagatctcgatcaaccatcccccaaaccgctatcttccaacatcgatgacatttcggagtccgcgcctaagattaccttgcaagccacacccaaggctgcaccggcgccagatgctcctcgcaaggttattttcgcaacaggtggcattacaaatggccgtcaagccagagagatcctagacgcgggagcgagtgtggcacaggtgtataccgccatgatctatggaggcgcgggcacaattagcaggatcaagcaagagatgagggatgatgcaaatgcacagaagacgttgacaaagcggtag

SEQ ID NO: 39 - protein, 572 aa, Ramularia collo-cygni
  REGION 1..572: note = MISC_FEATURE - RAMUCC_DHODH_XP_023626237
MLERTAMSSLRASFSRQVNLSRQFSTRRPSPFLHLQPRRTPQTNSRRYESTTATVTRATTSAGSSVIAYIYGAGLVLVLSFGYLYVTDTRASIHQWLVVPTLRLIHSDPEEAHHSGIRYVKALYDFGLHPRERGNDDQKLGDLKVEVFGHVLDNPIGTSAGIDKNAEIPDALMALGPAIVEVGGATPLPQPGNPKPRVFRIPSQNALINRYGLNSEGAEHVAMQLRERVRSFAYSHGLGLGAEAERIVLDGEAQVPPGSMVPGKLLAVQVAKNKTTPDGDIAAIAKDYTFCVEHLAKYADILVVNVSSPNTPGLRDLQATGPLTTILTAVVNTAKSIDRKTKPAVMVKVSPDEDSDAQIEGICAAVWASGVDGVIVGNTTNKRPDTIPKGYVLPEKDTKILQNEIGGYSGPQMFERTLSLVKRYRSKLDNGPPEEPAEAPSKQKLPSPIPTEDHLSDASIERDMQRLKPEDTKEDLQPLIQIPDRHKTSTEQQASQDGAPISKTQSSRSSASKVLFATGGITNGEQCLQILNAGASVCQVYTAMMYGGVGTITRMKGEMRAAIKGQNGDKPL

SEQ ID NO: 40 - DNA, 1719 bp, synthetic construct
  misc_feature 1..1719: note = RAMUCC_DHODH_cDNA_XM_023770469.1
  source 1..1719: mol_type = other DNA
atgctggaaaggacggcaatgagctcgctgagagcgagcttcagccgacaagtcaacttgagtcggcaattctcgactcgacgaccctcaccgttcctccatctacaaccacgacggacaccacagaccaactcccgccgatacgagtccacaactgccacagtcactcgtgcaacgacctctgccggatcatctgtgatcgcgtacatctatggagctggtcttgtgctggtattgtcctttggctacctatacgtcaccgacactcgcgcctccattcatcaatggctcgtagtacccactctacgactgatccactccgatcctgaggaagcccaccactctggcattcgatacgtaaaagctttgtatgatttcggtctccatccccgcgagcgcggtaacgatgaccagaagctgggtgatctcaaggtggaagtctttggacatgtactggacaatcccattggcacctcagccggcattgataagaatgccgagattccagacgcgctcatggcattgggaccagccatagtcgaagttggaggtgctacgcctctgccacagcccggcaacccgaagccgakagtcttccgcattcccagtcaaaatgcactcatcaaccgttatgggttgaactcggaaggagcagagcatgtcgcaatgcagctacgggagcgtgtgcggtctttcgcctattcccacgggcttggtctgggagccgaggcggagcgcatcgttctcgatggcgaggcgcaggtcccacccggaagcatggtaccaggaaagcttctcgccgtgcaagttgcaaagaacaagactacacctgatggagatattgctgcaattgccaaggactacaccttttgtgtggagcacttggccaagtacgcagacattcttgtcgtaaatgtctcctcgcccaacacacccggactgagagatttgcaagccaccggacccctcaccactatcctcacggctgtggtcaatactgcaaagtcaatcgaccgaaagacgaagccggcagtcatggtcaaggtgtctccggacgaagacagtgatgcccagatcgagggtatttgcgcggcagtttgggccagtggcgtggatggtgtgatcgtgggtaacaccacaaacaagcggcctgataccataccgaagggatatgttcttccggagaaagacacgaagattctacaaaatgagattggtggctactctggaccacaaatgtttgagcgcaccctcagccttgtgaagagatatcggtccaagcttgacaacggccctccagaagagcccgcagaagcgccatcaaaacagaaattgcctagtcctattcccactgaggaccatctttcggatgcgagtatagagcgggacatgcagcgtctaaagccggaggataccaaggaggatttgcagccgctcatccagatccctgacaggcacaagacttctacagaacagcaagcatcacaagatggtgctccaatatccaaaactcagagctctagatcctcggcgtcaaaggttctgtttgctaccggaggcatcaccaacggcgagcagtgtctgcagattctgaacgcaggcgcaagtgtatgccaagtgtacactgcgatgatgtatggtggtgtaggaaccatcacgaggatgaagggagagatgcgagcggctatcaaaggtcagaatggtgacaagcccttgtaa

SEQ ID NO: 41 - protein, 544 aa, Rhynchosporium secalis
  REGION 1..544: note = MISC_FEATURE - RHYNSE_DHODH_CZT49653.1
MASTPLRLLRFSQNPTRRLLLAQSRSTFLPRAAVQFSRPVTTAAAEASSFVTRLRNLLYGTSLSLFAVFSYLYITDTRASVHQWLVVPALRWIYPDAEEAHHAGTKALKSLYSFNMHPRERGSRKEEVDLSITVYGQKLQNPIGTSAGIDKLAEIPDALFALGPAIVEVGGATPLPQDGNPKPRVFRLPSRNSMINRYGLNSKGADDMARRLRERVRAFAYRSGYGIGDEAEEFVLSGGAGVPPGSLVEGKLLLVQVAKNKDTKDGDIEAVKRDYVYCVERLAPYADVLVVNVSSPNTPGLRTLQRVEPLTRILEGVVAAAKSSRRKTPPKVMVKVSPDEDQDEQIEGIVEAIWKSGVDGIIVGNTTNRRDNIAPADHPLTQKEKEVLNEKGGLSGPHMFERTLSLVKRYRQLLDQGPPVESPEPQLTHQKQPAQLAAIPSPRDEISTKISNKLSATKDVVSTAVSAAVSGVNDSVSSVKESGRGEKHEEKVIFATGGITNGKQALEILNAGASVAQIYTALTYGGSGTVTRIKRERSEEVRRG

SEQ ID NO: 42 - DNA, 1635 bp, synthetic construct
  misc_feature 1..1635: note = RHYNSE_DHODH_cDNA_FJVC01000391.1_35744-37378
  source 1..1635: mol_type = other DNA
atggcgtctaccccattgcgcctcctgcgcttctcgcaaaatccaacccgacgcctcctcctcgctcaatcccgatcgacattcctcccgcgcgccgcggtacagttttctcgtcctgtaacaacagcagcagctgaagccagctctttcgtcactcggctcaggaacctgctgtacggcacctcactttccctcttcgccgtcttcagctatctctacattaccgacacgcgagcgagcgtacatcaatggctcgtagttcccgccttacgatggatttatcccgatgcagaggaggcacaccacgcaggcacgaaagcactaaaatctctatattccttcaacatgcatcctcgggaacgagggtctaggaaagaagaagtagatttgagtatcacggtctacggacagaaattgcaaaatccaattggcacttcagcgggtatcgataagttggctgagattccggatgcgttgttcgctctaggtccggcaattgtggaggttggaggagcgacgcctttgccgcaggacggcaatccaaagccgagagtcttcagattaccgagtcggaattcaatgattaatcgctatgggttaaattccaaaggagcggacgatatggctaggagactacgggagcgagtccgcgcatttgcgtaccgcagcggttatggcattggtgatgaagccgaggagtttgtgctaagcggtggtgcgggtgttcctccaggaagtttggtggaaggtaaactgctgttggtgcaggttgctaagaataaggatacaaaggatggggatattgaggctgtgaagagggattacgtttattgcgtcgagaggctggctccttatgcggatgtgctggtcgtaaatgttagtagtccgaacacgcctgggttgcggacactgcagagggtagaacctctgactaggatcttggaaggcgtggttgcagcagcgaaatcgtcgaggaggaaaacgccgccaaaggttatggttaaagtctcgccggatgaggatcaagatgagcagattgagggcattgtagaagctatttggaaaagtggtgtagatggaattattgtggggaatacgactaatcgacgagataacattgctccagcagaccaccctctgacccagaaggagaaggaagtgctaaacgagaagggaggcttatcaggcccacacatgttcgaaagaacactaagcctcgtaaagcgctatcgacaactcctggaccaaggaccaccagtcgagtccccagaaccacaactaacacatcaaaaacaacccgcccaactagccgccatcccatcaccccgcgatgaaatctctacgaaaatcagtaacaagctctcagcaaccaaagacgtcgtgtccaccgccgtttccgctgccgtttccggtgtcaacgattctgtaagcagcgtcaaagaatctggaaggggggagaaacatgaagagaaagttattttcgccactggaggtatcacgaatgggaagcaggcgttggagattctgaatgcgggcgcgagtgtcgcgcaaatttatacggcgcttacttatggaggaagtgggacggtgacgaggatcaaaagggagaggagtgaggaggttaggagggggtag

SEQ ID NO: 43 - protein, 476 aa, Sclerotinia sclerotiorum
  REGION 1..476: note = MISC_FEATURE - SCLESC_DHODH_APA13900.1
MSNLLLRTLRISRPSLSSSISRISTKRFASTTNETPTILTRSRNILFGTSIALTLGFGYLYITDTRASIHQWLVVPALRAIYSDAEEAHHMGTKALKSLYSFGFHPRDRSFRNGNTDKADLSVEVWEHKLQNPIGTSAGLDKLAEIPDALFAMGPAIVEVGGCTPLAQDGNPKPRVFRIPSQNALINRYGLNSKGADDMAMRLRERVRLYAYQNGLGVGEDAEKMVLNGEAGVPPGSLLDGKLLAVQVAKNKDTNGDDIEAVKRDYVYCVERLAPYADILVVNVSSPNTPGLRTLQKVEPLSRILEGVVGAAKSVDRKTKPRVMVKVSPDEDQDEQLEGIVEAIWRSGVDGVIVGNTTNRRDKLIPDDIKISARERRVLEEQGGFSGPPMFSRTLSLVKRYRALLDQGPPQDNIAQSSDSGLVSTIAEKAKSLTGNKESKGNNQKVIFATGGITNGKQALEILNAGANVAQVYTAL

SEQ ID NO: 44 - DNA, 1431 bp, synthetic construct
  misc_feature 1..1431: note = SCLESC_DHODH_cDNA_CP017824.1_2042520-2043950
  source 1..1431: mol_type = other DNA
atgtcaaaccttttgttgcgcactctgcgcatatcacggccatccctctcctcttctatatcccgcatatctaccaaacgcttcgcctcaactactaacgaaacacccacaatcctcacccgatcgaggaatatcctctttggaacctcgatagctctcacccttggatttggctacttatacatcaccgatacccgagctagcatccatcaatggcttgttgtccccgcattacgtgccatatattccgatgccgaagaagctcaccatatgggcaccaaagctctcaaaagtctctactcgttcggttttcaccctcgcgatcgcagtttcagaaatggtaatacagataaggcggatttgagcgtagaagtatgggagcataaattacaaaacccaattggtacatctgcgggacttgacaaattggcagagattccagatgctttgttcgctatgggtcctgcgatcgttgaggttggcggatgtacaccacttgctcaagatggaaatccaaaaccaagagtcttccgtatacctagtcaaaatgcgttgatcaatcgatatggcctcaattcgaagggtgcagatgatatggctatgcgattgagggaacgggttcggttatatgcatatcagaatgggctaggtgtgggtgaagatgccgagaagatggttcttaatggagaagctggtgttcctccgggaagtttattggatgggaagttattggcggttcaggtcgccaaaaataaggatacgaatggcgatgatatcgaggctgttaagagagactatgtttactgtgtggaaagattggctccatatgccgacatcttggtagttaatgtcagcagcccgaatacgcctggattgaggacattacagaaggttgagccattatcccgaatcttagaaggtgtggttggagctgccaaatctgtagacaggaaaacgaagcctagggtcatggtgaaagtatcacctgacgaggatcaagatgaacagcttgagggcatcgtcgaagctatatggcgaagtggcgtggatggtgtgatcgtgggcaatactacaaaccgaagggacaagctcatccccgatgatatcaaaatttctgccagagaacgcagagttctcgaagaacaaggaggattttctggaccaccaatgttctctcgaacattatctttggtaaagagatatcgagctttacttgaccaaggaccaccacaagataatattgctcaatcaagtgacagcggtcttgtctccactatcgctgaaaaggcaaaatccctcacaggaaataaagagtcgaagggtaataatcagaaagtgatttttgctactggtggcatcacgaatggaaagcaagctctggagatcttgaatgcaggcgcaaatgtagcacaggtttacacggcattgtaa

SEQ ID NO: 45 - protein, 599 aa, Zymoseptoria tritici
  REGION 1..599: note = MISC_FEATURE - SEPTTR_DHODH_XP_003852344.1
MKATAKSAARETEAAAKDAAESVEGAAKTLQREATADAEAAKDAAKTAARETESAAKNAARETESAATDAAETAKTAAQATKDAAVDTARSAADKAETAAKVATYGATSAGSKIIAWIYGTGVVLLLSFGFLYVTDTRASIHQYVVVPFLRYVHADPEEAHHAGIRYIKALYDFGLAPRERGDMDKKLGDLQIEVFGHVLDNPIGTSAGIDKNAEVPDALFSLGPAIVEVGGVTPLPQEGNPKPRVFRIPSQNALINRYGLNSEGAEHVAMQLRERVRMFAYHNGLGLGKEAEDMVLNGEAQVPPGSLVPGKMLLVQMAKNKITPDGDIEAIARDYTFCVEHLAKYADVLVVNVSSPNTPGLRDLQAVGPLTRILGAVVDSANACERKTKPKVMVKVSPDEDSEEQIEGICAAVWASGVDGVIVGNTTKKRPDTIPKGYELPEEEVRLLTEVGGYSGPQMFERTLSLVGRYRSKLDQGPQDEAEDAGEEGSTRDVAESALKMVTHTGGTPDERSEELSASGSSTPAGKKTKATKKEDLQPKVIFATGGITNGEQCLQILNAGASVCQVYTAMMYGGVGTVTRMKGEMRREMRAQGEKKN

SEQ ID NO: 46 - DNA, 1800 bp, synthetic construct
  misc_feature 1..1800: note = SEPTTR_DHODH_cDNA_XM_003852296.1
  source 1..1800: mol_type = other DNA
atgaaggctactgctaagagtgctgctcgtgagacggaggctgctgctaaggacgctgcggagagcgttgagggtgctgcgaagacgttacaacgcgaggcgacggctgacgctgaggctgcgaaggacgccgcgaagactgctgcacgcgaaacggaatctgctgcaaagaacgccgcccgcgaaaccgagtccgccgcaaccgatgccgcagagaccgcgaagacagccgctcaagcgacgaaagacgctgccgtggacaccgctcgctcagctgcagacaaagcagagaccgccgcgaaggtagctacctacggcgccacttcagccggcagcaagatcatcgcctggatctacggtaccggagtcgtgctcctcctctcctttggcttcctctacgtgaccgacactcgcgccagcatccaccaatacgtcgtcgttccgttccttcgctacgtccacgccgatcctgaagaggcgcatcacgcgggaattcgatacatcaaagctctctacgatttcggccttgctccgagggaaagaggtgacatggataagaagcttggagacttgcagattgaagtctttggacacgtcttggacaacccgattggaacgtccgcggggatcgataagaatgctgaggtcccggatgcattgttctcgcttggaccagcgatcgtggaagtcggaggtgtgacgcctttgccgcaggagggcaacccgaagccgagagtgttccgcattcccagccagaatgcgttgatcaaccggtatggattgaattcggaaggagcggaacatgttgccatgcaattgagggagagagtgcggatgttcgcatatcataatggactgggactgggcaaggaggcggaagacatggtgctgaatggagaggcgcaagtgcctccgggcagtctggtacccgggaagatgctgcttgtgcaaatggcgaagaacaaaatcacaccagatggtgacattgaagcgatcgcgagggactatacattctgcgttgagcacttggccaagtacgccgacgtgctggtcgtcaacgtcagttcgccgaatacaccaggactgagggacttgcaagctgtgggacctttgacgagaatcctgggagctgtggttgactcagcaaacgcgtgtgaacgtaagaccaagccaaaggtgatggtgaaggtatcgccagatgaggacagcgaggagcagattgagggtatctgcgcggcggtatgggcgagcggtgtcgatggagtgatcgtaggcaacacaacgaagaagaggccggacacgataccgaaaggatacgaacttccagaggaggaggtgaggctgctcactgaagttggtggatattctggcccgcaaatgttcgaacgtacactcagtcttgtgggaagatatcggtcgaagctggatcaaggtcctcaagacgaagctgaagatgcaggcgaggagggatcgacgcgagatgtggccgagagcgcgttgaagatggtcacacatactggtggaactccggacgagcgaagtgaggaactgtcggcatcgggatcatctactccagcaggaaagaagaccaaggcgacgaagaaagaggatcttcaacccaaagtcatcttcgcgacgggcggcatcaccaacggcgaacaatgtttgcagattttgaatgcgggcgcgagtgtgtgtcaggtgtatacggcgatgatgtatggtggggtggggactgtgacgaggatgaagggggagatgaggagggagatgagagcccaaggggagaagaagaattaa

SEQ ID NO: 47 - protein, 594 aa, Venturia inaequalis
  REGION 1..594: note = MISC_FEATURE - VENTIN_DHODH_KAE9964670.1
MTIRAPPVRLLCQAVRPIIRNAPLHATRNASTSSNPPLNRARNLIYGSAALAAVSLGFIYVTDTRASVHEHIAPPVLRLLFPDAEDAHHAGTRALKELYTFGAHPRERGAGDSDGKLSVEAFGHILANPIGISGGLDKNAEIIDPLLALGPAVVEVGGITPLPQAGNPQPRVFRIASQNALINRYGLNSEGADVVAMRLRQRVREYAYSHDFGIDENAERLVLDGQTGVPPGSLVPGRLLAVQIAKNKLTPDTDLDAVVWDHVYCVDRLGKYADILVVNVSSPNTPGLRSLQSKKPLTQILSAVVNAANAVDRKTKPVVMVKVSPDEDSETQIQGIVDAVYESGVSGVIVGNTTMKRPAASPAGYILPEKDGRALLETGGYSGPQLFSRTKALVAKYRKALDDGPSENPLESPPPSKPSQPAAPSAPDHPESSVVEKIEASVKRDEANLKPMDDKSATGQGHRGRELHTGLELPERSSENHPIIRLPERNNPLSSSDNKTSDVPAPVAINTQTPSEHSDPAPASQASGRSKAFATAPKVIFASGGITTGEEALEVLNAGASVAMMYTGIVYGGVGTVGRVKSEMRKVIDGQSKK

SEQ ID NO: 48 - DNA, 1785 bp, synthetic construct
  misc_feature 1..1785: note = VENTIN_DHODH_cDNA_WNWS01000666.1
  source 1..1785: mol_type = other DNA
atgaccatacgggcccctccagtgcggctcctttgccaggcagttcggccgataatacgcaatgcccccttacatgccaccagaaatgcctctacgtcctcgaatccgcctctgaaccgcgctcgcaacttgatatacggctctgctgcgctcgcagctgtgtctcttggcttcatatatgtcaccgacactcgtgcatcagtacatgaacacatagcaccgccagtgctccggttactgttccccgatgctgaagacgcccaccacgccggcacccgcgccctcaaagaactatacacattcggcgcacatcctcgcgaacgtggagctggagattcggatgggaaattaagcgttgaggcttttggtcatatcctcgcgaatccaattggcatatctggaggattggacaagaatgcagaaattattgatcctctccttgctctgggaccagctgtggtggaggttggaggaataacgcctttgccacaggcaggaaacccgcaaccacgagtcttccgaattgccagtcaaaatgctttgatcaaccggtatggcttgaactcagaaggtgcagatgttgtggctatgcgattgaggcagagggtgcgagagtacgcatattctcatgactttggcatagacgaaaatgcggaaagactcgtactcgatggtcagactggagtaccgcccggatcacttgttcctggtcgcctactcgctgttcagattgcaaagaacaaactcacgcccgatacagacttagacgctgttgtctgggatcacgtgtactgcgttgatcgattgggcaagtacgcagatatcttggttgtcaacgtctcgagtccgaatacaccgggactgagatcacttcaatctaagaagccattgacgcaaatcttgtccgcagttgtgaatgccgccaatgcagtggacaggaaaactaaaccagtcgtcatggtcaaagtgtcaccagacgaggattctgagactcaaattcaaggcattgttgatgctgtatacgaatctggcgttagtggtgtcatcgtcggaaatactacgatgaaaagaccggctgcctctccagcaggctacattttaccagagaaggatggacgagcactacttgagaccggcggatacagtggtccacagctattctcaaggacaaaggcgctagtcgccaaatacagaaaagcattggacgatggtccgagtgaaaatcccctagaatcaccaccaccatcgaaaccaagccaacccgccgcaccaagcgcacccgatcatcccgaatcttccgttgtcgagaaaatcgaagcctctgtcaaaagggacgaagctaacctcaaaccaatggacgacaaatcggcaacgggtcaaggtcatcgaggtcgcgaactccatactggtcttgagcttcccgagcgctcatcagaaaaccacccaatcatccggctacccgaacgaaacaacccattatcttcctctgacaacaagacatccgatgtcccagcaccagtggctattaacacacagacgccatccgagcactcagatcctgcgcccgcatctcaagcctccgggcgaagtaaagcattcgccacggcacccaaagtcatttttgcctctgggggcatcacgactggcgaagaagcattggaagttctcaatgcgggagccagtgttgctatgatgtacactggtatcgtctatggtggtgttggaaccgttggtcgggtcaagtcagagatgcggaaagtgattgatggtcagagcaagaaatag

SEQ ID NO: 49 - protein, 670 aa, Monilinia laxa
  REGION 1..670: note = MISC_FEATURE - MONILA_DHODH_KAB8292478.1
MSNPLLRTLRTSRPILSSSPSRISTKRFSSTNTETSTILTRSKNILFGTSIALSLGFGYLYITDTRASIHQWLVVPALRAIYSDAEEAHHVGTKALKTLYSFGLHPRDRSFRDGNTDKADLSVEVWGQRLQNPIGTSAGIDKLAEVPDALFAMGPAIVEVGGCTPLAQEGNPKPRVFRIPSQNALINRYGLNSKGADDMAMRLRERVRLYAYQNGLGVGEDAERKVLNGDAGVPPGSLVDGKLLAVQVAKNKDTNGDDIEAVKRDYVYCVERLAPYADILVVNVSSPNTPGLRTLQKVEPLSRILDGVVGAAKSVDRKTKPRVMVKVSPDEDQDEQLEGIVEAIWRSGVDGVIVGNTTNRRDKLIPDDIKISARESRILEEQGGFSGPPMFERTLSLVKRYRALLDQGPPQQDTTVQPNEDGVASTIAEKAKSLTVNKELKNNDQKVVFATGGITNGKQALEILNAGASVAQVYTALVYGGVGTITRIKTEISNEISKNIPSILPPLAYTSSLYAAISHPSHLIPSQLTFKSSTRKKDIIYICQRIEYLPLSDHVMFRSFVPFGYPSSFLFPYALVCYFAPFLAYTYLRISNPFVINNQILSFPEKPHKVHKFIKERRVSFSFLVEIMIMIKSPSKDQKTKNYIKFKCEVDHNTSNALPFPRRFVCVVVL

SEQ ID NO: 50 - DNA, 2013 bp, synthetic construct
  misc_feature 1..2013: note = MONILA_DHODH_cDNA_VIGI01000013.1
  source 1..2013: mol_type = other DNA
atgtcgaaccctttattgcgcactctgcgcacatcgcgtcccatcctctcctcttccccatcccgcatatctaccaaacgcttctcatcaaccaataccgaaacatccacaatcctaacccgttcgaagaatatcctttttggaacctcgatagctctctcccttggctttggttacctctacattaccgacactcgagctagcatacaccaatggctggtagttcccgcattacgtgccatatattctgatgccgaggaagctcatcatgtgggcaccaaagctcttaaaaccctctactcattcggtctccatcctcgtgatcgaagtttcagagatggcaatacggacaaggctgatttgagtgtcgaggtatggggacaaaggttacaaaatccaattggtacatctgcgggaattgacaaattggcagaggtcccggatgctttgtttgctatgggccctgcgattgttgaagtcggtggatgtacacctcttgctcaagagggaaatcctaagccgagagttttccgcatacctagtcagaatgctctgattaatagatatgggcttaattcgaagggtgcggatgatatggcgatgcgattgagggagcgagttcgtctatatgcataccagaatggattaggtgtgggtgaagatgctgagagaaaggtgttaaacggagatgcaggtgttcctccaggaagcttggtagatgggaagttattggcagttcaggttgctaagaacaaggatactaatggtgatgatattgaggctgttaagagagattatgtttactgcgtcgagagactggcaccatacgccgatatcttggtagtcaatgttagtagtccaaacacacctgggctgaggacattacaaaaggttgagccattatcccgaatcttagatggagtggttggggccgcaaaatctgttgatcggaaaacaaagcctagggttatggtcaaggtatcacctgatgaggatcaagatgaacagcttgaggggattgttgaagctatatggagaagtggtgtagatggtgtaattgtgggtaatactacaaaccgaagagataagctcattcccgatgatatcaaaatctctgccagagaaagtagaattcttgaagaacaaggaggcttttctgggccacccatgtttgagagaaccttatctttggttaagagatatagagcattacttgaccaagggccaccacaacaggatactactgtccaaccaaatgaggatggcgttgcctctactatcgccgagaaggcaaaatctctcacggtcaacaaagaattgaaaaacaacgaccaaaaagttgtgttcgctactggtggtatcacgaacggaaagcaagctttggagatcttgaatgcaggtgcaagtgttgctcaggtttacacggcattggtctatggaggcgttggcacaataactcggataaagaccgagataagcaacgagatttcgaaaaacattccatcgattcttccaccactggcatatacttcctcattatatgctgccatctctcacccatcccatcttatcccatctcagttaacattcaaatcctccacaagaaaaaaagatattatatatatttgccaaagaattgaataccttcctctttctgaccatgtaatgtttcgctcctttgttcctttcggctatccatcttcctttcttttcccctatgccctcgtttgttacttcgctccttttctcgcctatacttatctaaggatatccaatccatttgtaatcaataatcaaatattatcctttcccgagaagccacataaagttcataaattcataaaagagcgtcgggtatcattctcattcctcgtcgagattatgattatgattaaatcacctagtaaagatcaaaaaacaaaaaattatataaagttcaaatgcgaagtagatcataatacgtcaaatgcactgccgtttccgcgtcggtttgtttgcgtcgtggtattatga

SEQ ID NO: 51 - protein, 496 aa, Botrytis cinerea
  REGION 1..496: note = MISC_FEATURE - BOTRCI_DHODH_XP_024553432
MSNPLLRTLRTSRPILSSSPSRISTKRFVSTSTETSPVLTRSKNLLVGTSIALALGFGYLYITDSRASVHQWLVVPALRAIYPDAEDAHHVGTKALKTLYSFGLHPRDRSFRQGNTDKADLSVEVWGQKLQNPIGTSAGLDKLAEVPDALFALGPAIVEVGGCTPLAQDGNPKPRVFRIPSQNALINRYGLNSKGADDMAMRLRERVRLYAYQNGLGVGEDAEKMVLNGEAGVPPGSLLDGKLLAVQVAKNKNTNGDDIEAVKRDYVHCVERLAPYADILVVNVSSPNTPGLRTLQKVEPLSRILEGVVGAAKSIDRKTKPRVMVKVSPDEDQDEQIEGIVEAVWRSGVDGIIVGNTTNRRDKLIPDDIKISARESRILEEQGGFSGPPMFERTLSLVKRYRTLLDRGPPQQETTPESNDNGLVSTIAEKAKSLTGNKANDQKVIFATGGITNGKQALEVLNAGANVAQVYTAMVYGGAGTITRIKTEMSNEISKK

SEQ ID NO: 52 - skipped

SEQ ID NO: 53 - protein, 9 aa, synthetic construct
  REGION 1..9: note = Consensus Motif 2
  SITE 2: note = misc_feature - Xaa can be any naturally occurring amino acid
  SITE 4: note = misc_feature - Xaa can be any naturally occurring amino acid
  REGION 7..8: note = misc_feature - Xaa can be any naturally occurring amino acid
DXEXAHXXG

SEQ ID NO: 54 - protein, 10 aa, synthetic construct
  REGION 1..10: note = Consensus Motif 3
  REGION 2..4: note = misc_feature - Xaa can be any naturally occurring amino acid
  REGION 6..8: note = misc_feature - Xaa can be any naturally occurring amino acid
LXXXGXXXVE

SEQ ID NO: 55 - protein, 12 aa, synthetic construct
  REGION 1..12: note = Consensus Motif 4
  SITE 1: note = misc_feature - Xaa can be any naturally occurring amino acid
  SITE 3: note = misc_feature - Xaa can be any naturally occurring amino acid
  REGION 6..7: note = misc_feature - Xaa can be any naturally occurring amino acid
  SITE 10: note = misc_feature - Xaa can be any naturally occurring amino acid
XQXGNXXPRXFR

SEQ ID NO: 56 - protein, 7 aa, synthetic construct
  REGION 1..7: note = Consensus Motif 5
  SITE 5: note = misc_feature - Xaa can be any naturally occurring amino acid
NRYGXNS

SEQ ID NO: 57 - protein, 14 aa, synthetic construct
  REGION 1..14: note = Consensus Motif 6,
  REGION 1..3: note = misc_feature - Xaa can be any naturally occurring amino acid
  SITE 7: note = misc_feature - Xaa can be any naturally occurring amino acid
  REGION 10..11: note = misc_feature - Xaa can be any naturally occurring amino acid
  SITE 13: note = misc_feature - Xaa can be any naturally occurring amino acid
XXXYGGXGTXXRXK

SEQ ID NO: 58 - protein, 8 aa, synthetic construct
  REGION 1..8: note = Motif 1
WLVVPALR

SEQ ID NO: 59 - protein, 9 aa, synthetic construct
  REGION 1..9: note = Motif 2
DAEDAHHVG

SEQ ID NO: 60 - protein, 10 aa, synthetic construct
  REGION 1..10: note = Motif 3
LFALGPAIVE

SEQ ID NO: 61 - protein, 12 aa, synthetic construct
  REGION 1..12: note = Motif 4
AQDGNPKPRVFR

SEQ ID NO: 62 - protein, 7 aa, synthetic construct
  REGION 1..7: note = Motif 5
NRYGLNS

SEQ ID NO: 63 - protein, 14 aa, synthetic construct
  REGION 1..14: note = Motif 6
AMVYGGAGTITRIK

(19) United States
(12) Patent Application Publication
(10) Pub. No.: US 2026/0150841 A1
(43) Pub. Date: Jun. 4, 2026

(54) PYRIDINE COMPOUNDS FOR COMBATTING DIHYDROOROTATE-DEHYDROGENASE INHIBITOR-RESISTANT PHYTOPATHOGENIC FUNGI

(71) Applicant: BASF SE, Ludwigshafen (DE)

(72) Inventors: Bernd Mueller, Frankenthal (DE); Philipp Georg Werner Seeberger, Ludwigshafen (DE); Wassilios Grammenos, Ludwigshafen (DE); Benjamin Juergen Merget, Ludwigshafen (DE); Andreas Koch, Limburgerhof (DE); Annette Schuster, Limburgerhof (DE); Ronan Le Vezouet, Ludwigshafen (DE); Jan Klaas Lohmann, Ludwigshafen (DE); Amin Minakar, Beelan (DE); Tim Alexander Stoesser, Ludwigshafen (DE); Dorothee Sophia Ziegler, Ludwigshafen (DE); Aymane Selmani, Ludwigshafen (DE); Jochen Dietz, Ludwigshafen (DE)

(21) Appl. No.: 19/122,318

(22) PCT Filed: Oct. 20, 2023
(86) PCT No.: PCT/EP2023/079247

§ 371 Date: Apr. 18, 2025

(30) Foreign Application Priority Data
Oct. 20, 2022 (EP) 22202813.6

(51) Int. Cl. A01N 43/86; A01N 43/54; A01N 43/72; A01P 3/00; C12N 9/02

(52) U.S. Cl. CPC A01N 43/86; A01N 43/54; A01N 43/72; A01P 3/00; C12N 9/001; C12Y 103/05002

(57) ABSTRACT

The present invention relates to the use of compounds (I) where the variables are as defined in the claims and the description, for combatting dihydroorotate-dehydrogenase inhibitor-resistant phytopathogenic fungi, and to a method for combatting dihydroorotate-dehydrogenase inhibitor-resistant phytopathogenic fungi comprising treating the phytopathogenic fungi or the materials, plants, the soil or seeds that are infected or infested or are at risk of being infected or infested with said phytopathogenic fungi with at least one compound (I). The invention relates moreover to a mutated DHODH inhibitor resistant DHODH enzyme and to a nucleic acid molecule comprising at least one nucleotide sequence encoding a mutated DHODH enzyme.

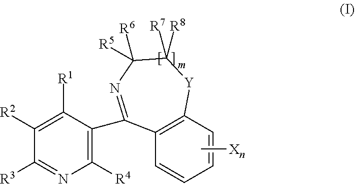

The present invention relates to the use of compounds (I) as defined below for combatting dihydroorotate-dehydrogenase inhibitor-resistant phytopathogenic fungi and to a method for combatting dihydroorotate-dehydrogenase inhibitor-resistant phytopathogenic fungi comprising treating the phytopathogenic fungi or the materials, plants, the soil or seeds that are infected or infested or are at risk of being infected or infested with said phytopathogenic fungi with at least one compound (I) as defined below. The invention relates moreover to a a mutated DHODH inhibitor resistant DHODH enzyme and toa nucleic acid molecule comprising at least one nucleotide sequence encoding a mutated DHODH enzyme.

### TECHNICAL BACKGROUND

Dihydroorotate dehydrogenase (DHODH; PyrE) is an enzyme involved in the de novo synthesis of pyrimidines, catalysing the oxidation of dihydroorotate to orotate. Two classes of DHODH have been described on the basis of differences in amino acid sequence; Class II DHODH are found in most fungi (including A. fumigatus and C. albicans), animals, plants, gram-negative bacteria and archeabacteria. These use an FMN molecule as a cofactor, and, in the case of humans and fungi, this is recycled by means of oxidation via a quinone cofactor from the respiratory chain. The human and fungal proteins are non-covalently associated with the mitochondrial inner membrane by an N-terminal trans-membrane domain. The quinone-binding pocket is adjacent to, but distinct from the catalytic site of the enzyme. Class I enzymes are found in gram-positive bacteria, trypanosomes, Saccharomyces cerevisiae, and closely related fungi such as other members of the genus Saccharomyces.

While DHODH protein has been described as potential antifungal targets for the medi-cal treatment of fungal infections (cf. for example WO 2009/133379) the applicability as target for combatting phytopathogens has not been described.

The present inventors have observed that the application of several known phytopathogen inhibitors results in the selection of phytopathogen mutants which show resistance to such inhibitors. They observed that resistance is caused by a mutated DHODH gene of the pathogen.

A first object of the present invention was to identify novel species of phytopathogenic fungi, in particular mutant strains containing a mutated form of their endogenous DHODH gene, and showing resistance against several prior art antifungal agents, in particular quinoline-type antifungal compounds. Such mutant strains or mutated DHODH enzymes may be applied as target for identifying suitable efficient phytopathogen inhibitors.

It was another object of the present invention to provide DHODH inhibitor compounds which are suitable for combatting such mutated phytopathogenic fungi which show resistance against such prior art phytopathogen inhibitors.

### SUMMARY OF THE INVENTION

This object is achieved by the use of pyridine compounds of the formula (I) as defined below.

Thus, in a first aspect, the invention relates to the use of compounds of the formula (I)

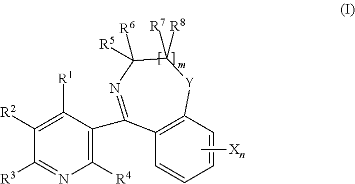

where
R¹ is hydrogen, halogen, CN, C₁-C₄-alkyl or C1-C₄-haloalkyl;
R² is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₁-C₆-alkoxy, C₂-C₆-alkenyloxy or C₂-C₆-alkynyloxy;
R³ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₁-C₆-alkoxy, C₂-C₆-alkenyloxy or C₂-C₆-alkynyloxy;

R⁴ is hydrogen, halogen, CN, C₁-C₄-alkyl or C₁-C₄-haloalkyl;

R⁵ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the 8 last-mentioned aliphatic or aromatic radicals are unsubstituted or carry 1, 2 or 3 substituents R^{5a}; where
each R^{5a} is independently halogen, CN, C₁-C₆-alkyl, C₁-C₆-haloalkyl or C₁-C₆-alkoxy;
R⁶ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the 8 last-mentioned aliphatic or aromatic radicals are unsubstituted or carry 1, 2 or 3 substituents R^{6a}; where
each R^{6a} is independently halogen, CN, C₁-C₆-alkyl, C₁-C₆-haloalkyl or C₁-C₆-alkoxy; or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing 1, 2 or 3 heteroatoms selected from O and S as ring members; where the carbocyclic or heterocyclic ring is unsubstituted or carries 1,2 or 3 substituents R⁵⁶; where
each R⁵⁶ is independently halogen, C₁-C₆-alkyl or C₁-C₆-haloalkyl;
R⁷ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the 8 last-mentioned aliphatic or aromatic radicals are unsubstituted or carry 1, 2 or 3 substituents R^{7a}; where
each R^{7a} is independently halogen, CN, C₁-C₆-alkyl, C₁-C₆-haloalkyl or C₁-C₆-alkoxy;
R⁸ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the 8 last-mentioned aliphatic or aromatic radicals are unsubstituted or carry 1, 2 or 3 substituents R^{8a}; where
each R^{8a} is independently halogen, CN, C₁-C₆-alkyl, C₁-C₆-haloalkyl or C₁-C₆-alkoxy;
or
R⁷ and R⁸, together with the carbon atom to which they are bound, form a 3-, 4-, 5- or 6-membered saturated carbocyclic ring or a 3-, 4-, 5- or 6-membered saturated heterocyclic ring containing 1, 2 or 3 heteroatoms selected from O and S as ring members;
each X is independently halogen, CN, C₁-C₆-alkyl, C₁-C₆-haloalkyl, C₂-C₆-alkenyl, C₂-C₆-alkynyl, C₃-C₆-cycloalkyl, C₁-C₆-alkoxy or C₁-C₆-haloalkoxy;
Y is O if m is 1; and is O or NR⁹ if m is 0;
where
R⁹ is hydrogen, CN, CH₂CN, CH(CH₃)CN, —CH(═O), —C(═O)C₁-C₆-alkyl, —C(═O)C₂-C₆-alkenyl, —C(═O)C₂-C₆-alkynyl, —C(═O)C₃-C₆-cycloalkyl, —C(═O)—N(H) C₁-C₄-alkyl, —C(═O)—N(C₁-C₄-alkyl)₂, C₁-C₆-alkyl, C₁-C₆-alkoxy, C₁-C₄-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl,
   —S(═O)₂-R^{9a}, five- or six-membered heteroaryl, aryl or benzyl; wherein heteroaryl contains 1, 2 or 3 heteroatoms selected from N, O and S as ring members; wherein aryl and the phenyl ring in benzyl are unsubstituted or carry 1, 2, 3, 4 or 5 substituents selected from the group consisting of CN, halogen, OH, C₁-C₄-alkyl, C₁-C₄-haloalkyl, C₁-C₄-alkoxy and C₁-C₄-haloalkoxy; wherein the phenyl ring in benzyl is unsubstituted or carries 1, 2 or 3 substituents selected from the group consisting of CN and halogen;
   wherein
   R^{9a} is C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the phenyl ring in the two last-mentioned radicals is unsubstituted or carries 1, 2 or 3 substituents each independently selected from the group consisting of halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl;
m is 0 or 1; and
n is 0, 1, 2 or 3;
or the N-oxides, tautomers, stereoisomers or agriculturally acceptable salts thereof,
for combatting dihydroorotate-dehydrogenase inhibitor-resistant phytopathogenic fungi.

The invention relates further to a method for combating dihydroorotate-dehydrogenase inhibitor-resistant phytopathogenic fungi comprising treating the phytopathogenic fungi or the materials, plants, the soil or seeds that are infected or infested or are at risk of being infected or infested with said phytopathogenic fungi with an effective amount of at least one compound of formula I, an N-oxide, a tautomer, a stereoisomer or an agriculturally acceptable salt thereof or with a composition comprising at least one compound of formula I, an N-oxide, a tautomer, a stereoisomer or an agriculturally acceptable salt thereof.

The method comprises in particular
a) identifying the phytopathogenic fungi as defined above or below, or the materials, plants, the soil or seeds that are at risk of being diseased from phytopathogenic fungi as defined above or below, and
b) treating said fungi or the materials, plants, the soil or seeds with an effective amount of at least one compound of formula I as defined above or below or an N-oxide, a tautomer, a stereoisomer or an agriculturally acceptable salt thereof or with an effective amount of a composition comprising at least one compound of formula I as defined above or below or an N-oxide, a tautomer, a stereoisomer or an agriculturally acceptable salt thereof.

The invention relates moreover to an agrochemical composition comprising at least one compound of formula I as defined above or below, an N-oxide, a tautomer, a stereoisomer or an agriculturally acceptable salt thereof and at least one auxiliary for the use for combatting dihydroorotate-dehydrogenase inhibitor resistant phytopathogenic fungi as defined above or below.

The invention relates moreover to novel DHODH mutants, corresponding coding sequences and organisms carrying the same as herein defined below in more detail.

### DESCRIPTION OF DRAWINGS

FIG. 1 shows a sequence alignment of endogenous DHODHs of a group of plant pathogenic fungi. The top row shows the native amino acid sequence of Botrytis cinerea DHODH (long version SEQ ID NO: 1). Analogous amino acid sequence positions may be easily derived there from. For example, amino acid sequence position V181 of Alternaria altemata DHODH (SEQ ID NO:3) as depicted in the second row of the alignment is analogous to position V195 of Botrytis cinerea DHODH (long version SEQ ID NO: 1).

FIG. 2 illustrates the results of modeling of different inhibitor molecules into the substrate pocket of Botrytis cinerea DHODH (native enzyme and V195F mutant). FIG. 2a shows the modeling results for a prior art quinoline-type inhibitor for the native enzyme (left) and the mutant V195F (right), and in particular the steric hindrance induced by the more bulky phenyl residue in position 195. FIG. 2b on the other hand illustrates that the pyridine—type residue of an inhibitor of the present invention fits much better into the substrate pocket of the V195F mutant and consequently allows to combat the V195F mutant resistant against the prior art quinoline-type inhibitor.

### DETAILED DESCRIPTION OF THE INVENTION

### General definitions

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine.

The term “alkyl” as used as such and in the alkyl moieties of alkoxy, alkylcarbonyl and the like is a saturated straight-chain or branched aliphatic hydrocarbon radicals having in general 1 to 6 (“C₁-C₆-alkyl”), 1 to 4 (“C₁-C₄-alkyl”), 1 to 3 (“C₁-C₃-alkyl”) or 1 or 2 (“C₁-C₂-alkyl”) carbon atoms. “C₁-C₂-Alkyl” is a saturated aliphatic hydrocarbon radical having 1 or 2 carbon atoms. “C₁-C₃-alkyl” is a saturated straight-chain or branched aliphatic hydrocarbon radical having 1 to3 carbon atoms. “C₁-C₄-Alkyl” is a saturated straight-chain or branched aliphatic hydrocarbon radical having 1 to 4 carbon atoms. “C₁-C₆-Alkyl” is a saturated straight-chain or branched aliphatic hydrocarbon radical having 1 to 6 carbon atoms. C₁-C₂-Alkyl is methyl or ethyl. Examples for C₁-C₃-alkyl are, in addition to those mentioned for C₁-C₂-alkyl, propyl and isopropyl. Examples for C₁-C₄-alkyl are, in addition to those mentioned for C₁-C₃-alkyl, butyl, 1-methylpropyl (sec-butyl), 2-methylpropyl (isobutyl) or 1,1-dimethylethyl (tert-butyl). Examples for C₁-C₆-alkyl are, in addition to those mentioned for C₁-C₄-alkyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethyl-butyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “haloalkyl”, which is also expressed as “halogenalkyl” or “alkyl which is partially or fully halogenated”, as used as such and in the haloalkyl moieties of haloalkoxy and the like indicates saturated straight-chain or branched aliphatic hydrocarbon radicals having in general 1 to 6 (“C₁-C₆-halo (gen) alkyl”), 1 to 4 (“C₁-C₄-halo (gen) alkyl”), 1 to 3 (“C₁-C₃-halo (gen) alkyl”) or 1 or 2 (“C₁-C₂-halo (gen) alkyl”) carbon atoms, where some or all of the hydrogen atoms in these groups are replaced by halogen atoms as mentioned above, in particular by fluorine, chlorine and/or bromine. “C₁-C₂-Haloalkyl” refers to alkyl groups having 1 or 2 carbon atoms (as mentioned above), where some or all of the hydrogen atoms in these groups are replaced by halogen atoms as mentioned above, in particular by fluorine, chlorine and/or bromine. “C₁-C₃-Haloalkyl” refers to straight-chain or branched alkyl groups having 1 to 3 carbon atoms (as mentioned above), where some or all of the hydrogen atoms in these groups are replaced by halogen atoms as mentioned above, in particular by fluorine, chlorine and/or bromine. “C₁-C₄-Haloalkyl” refers to straight-chain or branched alkyl groups having 1 to 4 carbon atoms (as mentioned above), where some or all of the hydrogen atoms in these groups are replaced by halogen atoms as mentioned above, in particular by fluorine, chlorine and/or bromine. “C₁-C₆-Haloalkyl” refers to straight-chain or branched alkyl groups having 1 to 6 carbon atoms (as mentioned above), where some or all of the hydrogen atoms in these groups are replaced by halogen atoms as mentioned above, in particular by fluorine, chlorine and/or bromine. Examples for C₁-C₂-haloalkyl are chloromethyl, bromomethyl, dichloromethyl, trichloromethyl, fluoromethyl, difluoromethyl, trifluoromethyl, chlorofluoromethyl, dichlorofluoromethyl, chlorodifluoro-methyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. Examples for C₁-C₃-haloalkyl are, in addition to those mentioned for C₁-C₂-haloalkyl, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, in addition to those mentioned for C₁-C₃-haloalkyl, 4-chlorobutyl and the like.

Strictly speaking, the term “alkenyl” indicates monounsaturated (i.e. containing one C-C double bond) straight-chain or branched aliphatic hydrocarbon radicals having in general 2 to 6 (“C₂-C₆-alkenyl”), 2 to 4 (“C₂-C₄-alkenyl”) or 2 to 3 (“C₂-C₃-alkenyl”)carbon atoms, where the C-C double bond can be in any position. As used in the present invention, the term encompasses however also “alkapolyenyl” groups, i.e. straight-chain or branched aliphatic hydrocarbon radicals having in general 4 to 6 (“C₄-C₆-alkapolyenyl”) carbon atoms, and two or three conjugated or isolated, but non-cumulated C-C double bonds. Examples for C₂-C₃-alkenyl in the strict sense (only 1 C-C double bond) are ethenyl, 1-propenyl, 2-propenyl or 1-methylethenyl. Examples for C₂-C₄-alkenyl in the strict sense (only 1 C-C double bond) are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl and 2-methyl-2-propenyl. Examples for C₂-C₆-alkenyl in the strict sense (only 1 C-C double bond) are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like. Examples for alkapolyenyl groups are buta-1,3-dien-1-yl, buta-1,3-dien-2-yl, penta-1,3-dien-1-yl, penta-1,3-dien-2-yl, penta-1,3-dien-3-yl, penta-1,3-dien-4-yl, penta-1,3-dien-5-yl, penta-1,4-dien-1-yl, penta-1,4-dien-2-yl, penta-1,4-dien-3-yl, and the like.

The term “haloalkenyl” as used herein, which is also expressed as “, “halogenalkenyl” or “alkenyl which is partially or fully halogenated”, refers to unsaturated straight-chain or branched hydrocarbon radicals having 2 to 3 (“C₂-C₃-halo (gen) alkenyl”), 2 to 4 (“C₂-C₄-halo (gen) alkenyl”) or 2 to 6 (“C₂-C₆-halo (gen) alkenyl”) carbon atoms and a double bond in any position (as mentioned above), where some or all of the hydrogen atoms in these groups are replaced by halogen atoms as mentioned above, in particular by fluorine, chlorine and bromine. Example are chlorovinyl, chloroallyl and the like.

The term “alkynyl” as used herein refers to straight-chain or branched hydrocarbon groups having 2 to 3 (“C₂-C₃-alkynyl”), 2 to 4 (“C₂-C₄-alkynyl”) or 2 to 6 (“C₂-C₆-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like.

The term “haloalkynyl” as used herein, which is also expressed as “halogen-alkynyl” or “alkynyl which is partially or fully halogenated”, refers to unsaturated straight-chain or branched hydrocarbon radicals having 2 to 3 (“C₂-C₃-halo (gen) alkynyl”), 2 to 4 (“C₂-C₄-halo (gen) alkynyl”) or 2 to 6 (“C₂-C₆-halo (gen) alkynyl”) carbon atoms and one or two triple bonds in any position (as mentioned above), where some or all of the hydrogen atoms in these groups are replaced by halogen atoms as mentioned above, in particular by fluorine, chlorine and bromine.

The term “cycloalkyl” as used herein refers to monocyclic saturated hydrocarbon radicals having 3 to 6 carbon atoms (“C₃-C₆-cycloalkyl”). Examples are cyclopropyl, cy-clobutyl, cyclopentyl and cyclohexyl.

The term “halocycloalkyl” as used herein, which is also expressed as “halogency-cloalkyl” or “cycloalkyl which is partially or fully halogenated”, refers to monocyclic saturated hydrocarbon groups having 3 to 6 (“C₃-C₆-halo (gen) cycloalkyl”) carbon ring members (as mentioned above) in which some or all of the hydrogen atoms are replaced by halogen atoms as mentioned above, in particular by fluorine, chlorine and bromine. Halogenated cyclopropyl is for example 1-fluorocyclopropyl, 2-fluorocyclopropyl, 1-chlorocyclopropyl, 2-chlorocyclopropyl, 1,1-difluorocyclopropyl, 1,2-difluorocyclopropyl, 2,2-difluorocyclopropyl, 1, 1,2,2-tetrafluorocyclopropyl and the like.

“Alkoxy”, also expressed as “O-alkyl”, is an alkyl group, as defined above, attached via an oxygen atom to the remainder of the molecule. “C₁-C₂-Alkoxy” is a C₁-C₂-alkyl group, as defined above, attached via an oxygen atom. “C₁-C₃-Alkoxy” is a C₁-C₃-alkyl group, as defined above, attached via an oxygen atom. “C₁-C₄-Alkoxy” is a C₁-C₄-alkyl group, as defined above, attached via an oxygen atom. “C₁-C₆-Alkoxy” is a C₁-C₆-alkyl group, as defined above, attached via an oxygen atom. C₁-C₂-Alkoxy is methoxy or ethoxy. C₁-C₃-Alkoxy is additionally, for example, n-propoxy and 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

“Haloalkoxy”, also expressed as “halogenalkoxy” or “O-halogenalkyl” is a haloalkyl group, as defined above, attached via an oxygen atom to the remainder of the molecule. “C₁-C₂-Halo (gen) alkoxy” is a C₁-C₂-haloalkyl group, as defined above, attached via an oxygen atom. “C₁-C₃-Halo (gen) alkoxy” is a C₁-C₃-haloalkyl group, as defined above, attached via an oxygen atom. “C₁-C₄-Halo (gen) alkoxy” is a C₁-C₄-haloalkyl group, as defined above, attached via an oxygen atom. “C₁-C₆-Halo (gen) alkoxy” is a C₁-C₆-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoro-methoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloro-ethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoro-propoxy, 3,3,3-trichloropropoxy, OCH₂-C₂F₅, OCF₂-C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

“Alkenyloxy”, also expressed as “O-alkenyl”, is an alkenyl group, as defined above, attached via an oxygen atom to the remainder of the molecule. “C₂-C₃-Alkenyloxy” is a C₂-C₃-alkenyl group, as defined above, attached via an oxygen atom. “C₂-C₄-Alkenyloxy” is a C₂-C₄-alkenyl group, as defined above, attached via an oxygen atom. “C₂-C₆-Alkenyloxy” is a C₂-C₆-alkenyl group, as defined above, attached via an oxygen atom. Examples for C₂-C₃-alkenyloxy are ethenyloxy, prop-1-en-1-yloxy, prop-1-en-2-yl oxy or prop-1-en-3-yloxy. Examples for C₂-C₄-alkenyloxy are, in addition to those mentioned above for C₂-C₃-alkenyloxy, but-1-en-1-yloxy, but-2-en-1-yloxy, but-3-en-1-yloxy, 1-methylprop-1-en-1-yloxy, 2-methylprop-1-en-1-yloxy, 1-methylprop-2-enyl-1-oxy or 2-methylprop-2-en-1-yloxy.

“Haloalkenyloxy”, also expressed as “halogenalkenyloxy” or “O-halogenalkenyl” is a haloalkenyl group, as defined above, attached via an oxygen atom to the remainder of the molecule. “C₂-C₃-Halo (gen) alkenyloxy” is a C₂-C₃-haloalkenyl group, as defined above, attached via an oxygen atom. “C₂-C₄-Halo (gen) alkenyloxy” is a C₂-C₄-haloalkenyl group, as defined above, attached via an oxygen atom. “C₂-C₆-Halo (gen) alkenyloxy” is a C₂-C₆-haloalkenyl group, as defined above, attached via an oxygen atom.

“Alkynyloxy”, also expressed as “O-alkynyl”, is an alkynyl group, as defined above, attached via an oxygen atom to the remainder of the molecule. “C₂-C₃-Alkynyloxy” is a C₂-C₃-alkynyl group, as defined above, attached via an oxygen atom. “C₂-C₄-Alkynyloxy” is a C₂-C₄-alkynyl group, as defined above, attached via an oxygen atom. “C₂-C₆-Alkynyloxy” is a C₂-C₆-alkynyl group, as defined above, attached via an oxygen atom. Examples for C₂-C₃-alkynyloxy are ethynyloxy, prop-1-yn-1-yloxy, prop-1-yn-2-yl oxy or prop-1-yn-3-yloxy. Examples for C₂-C₄-alkynyloxy are, in addition to those mentioned above for C₂-C₃-alkynyloxy, but-1-yn-1-yloxy, but-2-yn-1-yloxy, but-3-yn-1-yloxy, 1-methylprop-1-yn-1-yloxy, 2-methylprop-1-yn-1-yloxy, 1-methylprop-2-ynyl-1-oxy or 2-methylprop-2-yn-1-yloxy.

“Haloalkynyloxy”, also expressed as “halogenalkynyloxy” or “O-halogenylkenyl” is a haloalkynyl group, as defined above, attached via an oxygen atom to the remainder of the molecule. “C₂-C₃-Halo (gen) alkynyloxy” is a C₂-C₃-haloalkynyl group, as defined above, attached via an oxygen atom. “C₂-C₄-Halo (gen) alkynyloxy” is a C₂-C₄-haloalkynyl group, as defined above, attached via an oxygen atom. “C₂-C₆-Halo (gen) alkynyloxy” is a C₂-C₆-haloalkynyl group, as defined above, attached via an oxygen atom.

Oxo is a group ═O, i.e. a CH₂ group is replaced by C═O.

R⁵ and R⁶, together with the carbon atom to which they are bound, may form a 3-, 4-, 5- or 6-membered saturated carbocyclic ring or a 3-, 4-, 5- or 6-membered saturated heterocyclic ring containing 1, 2 or 3 heteroatoms selected from O and S as ring members. This ring is spirocyclically bound to the condensed ring system to which R⁵ and R⁶ are bound, the carbon atom to which R⁵ and R⁶ are bound being the spiro atom. The ring is generally monocyclic.

Examples for saturated three- to six-membered monocyclic carbocyclic rings formed by R⁵ and R⁶ together with the carbon atom to which they are bound are cyclo-propan-1,1-diyl, cyclobutan-1,1-diyl, cyclopentan-1,1-diyl or cyclohexan-1,1-diyl. 3-, 4-, 5- or 6-membered saturated heterocyclic ring containing 1, 2 or 3 heteroatoms selected from O and S as ring members formed by R⁵ and R⁶ together with the carbon atom to which they are bound are for example oxiran-2,2-diyl, thiiran-2,2-diyl, oxetan-2,2-diyl, oxetan-3,3,-diyl, thietan-2,2-diyl, thietan-3,3-diyl, tetrahydrofuran-2,2-diyl, tetrahydrofuran-3,3-diyl, tetrahydrothiophen-2,2-diyl, tetrahydrothiophen-3,3-diyl, 1,3-dioxolan-2,2-diyl, 1,3-dioxolan-4,4-diyl, 1,3-dithiolan-2,2-diyl, 1,3-dithiolan-4,4-diyl, 1,3-oxathiolan-2,2-diyl, 1,3-oxathiolan-4,4-diyl, 1,3-oxathiolan-5,5-diyl, tetrahydropyran-2,2-diyl, tetrahydropyran-3,3-diyl, tetrahydropyran-4,4-diyl, tetrahydrothiopyran-2,2-diyl, tetrahydrothiopyran-3,3-diyl, tetrahydrothiopyran-4,4-diyl, 1,3-dioxan-2,2-diyl, 1,3-dioxan-4,4-diyl, 1,3-dioxan-5,5-diyl, 1,4-dioxan-2,2-diyl, oxepan-2,2-,-3,3-or 4,4-diyl, hexahydro-1,3-dioxepin-2,2- ,-4,4-or 5,5-diyl, hexahydro-1,4-dioxepin-2,2- ,-5,5-or 6,6-diyl, oxocan-2,2-, 3,3-or 4,4-diyl, thiocan-2,2-, 3,3-or 4,4-diyl and the like.

R⁷ and R⁸, together with the carbon atom to which they are bound, may form a 3-, 4-, 5- or 6-membered saturated carbocyclic ring or a 3-, 4-, 5- or 6-membered saturated heterocyclic ring containing 1, 2 or 3 heteroatoms selected from O and S as ring members. This ring is spirocyclically bound to the condensed ring system to which R⁷ and R⁸ are bound, the carbon atom to which R⁷ and R⁸ are bound being the spiro atom. The ring is generally monocyclic.

Examples for saturated three- to six-membered monocyclic carbocyclic rings formed by R⁷ and R⁸ together with the carbon atom to which they are bound are cyclo-propan-1,1-diyl, cyclobutan-1,1-diyl, cyclopentan-1,1-diyl and cyclohexan-1,1-diyl.

Examples for saturated heterocyclic ring containing 1, 2 or 3 heteroatoms selected from O and S as ring members formed by R⁷ and R⁸ together with the carbon atom to which they are bound correspond to those given above for the rings formed by R⁵ and R⁶ together with the carbon atom to which they are bound.

“Aryl” is a mono-, bi-or polycyclic carbocyclic (i.e. without heteroatoms as ring members) aromatic radical. One example for a monocyclic aromatic radical is phenyl. In bicyclic aryl rings two aromatic rings are condensed, i.e. they share two vicinal C atoms as ring members. One example for a bicyclic aromatic radical is naphthyl. In polycyclic aryl rings, three or more rings are condensed. Examples for polycyclic aryl radicals are phenanthrenyl, anthracenyl, tetracenyl, 1H-benzo[a]phenalenyl, pyrenyl and the like. In the terms of the present invention “aryl” encompasses however also bi-or polycyclic radicals in which not all rings are aromatic, as long as at least one ring is; especially if the reactive site is on the aromatic ring (or on a functional group bound thereto). Examples are indanyl, indenyl, tetralinyl, 6,7,8,9-tetrahydro-5H-benzo[7]annu-lenyl, fluorenyl, 9,10-dihydroanthracenyl, 9,10-dihydrophenanthrenyl, 1H-benzo[a] phenalenyl and the like, and also ring systems in which not all rings are condensed, but for example spiro-bound or bridged, such as benzonorbornyl. In particular, the aryl group has 6 to 20, preferably 6 to 10 carbon atoms as ring members.

Examples for a 5- or 6-membered heteroaromatic ring (or heteroaryl) containing 1, 2 or 3 heteroatoms selected from N, O and S as ring members are 2-furyl, 3-furyl, 2-thienyl, 3-thienyl, 1-pyrrolyl, 2-pyrrolyl, 3-pyrrolyl, 2-oxazolyl, 4-oxazolyl, 5-oxazolyl, 3-isoxazolyl, 4-isoxazolyl, 5-isoxazolyl, 2-thiazolyl, 4-thiazolyl, 5 thiazolyl, 3-isothiazolyl, 4-isothiazolyl, 5-isothiazolyl, 17-pyrazolyl, 3-pyrazolyl, 4-pyrazolyl, 5-pyrazolyl, 1-imidazolyl, 2-imidazolyl, 4-imidazolyl, 5-imidazolyl, 1,2,3-triazol-1-yl, 1,2,3-triazol-2-yl, 1,2,3-triazol-4-yl, 1,2,4-triazol-1-yl, 1,2,4-triazol-2-yl, 1,2,4-triazol-3-yl, 1,3,4-triazol-1-yl, 1,3,4-triazol-2-yl, 1,2,5-oxadiazol-3-yl, 1,2,3-oxadiazol-4-yl, 1,2,3-oxadiazol-5-yl, 1,3,4-oxadi-azol-2-yl, 1,2,5-thiadiazol-3-yl, 1,2,3-thiadiazol-4-yl, 1,2,3-thiadiazol-5-yl, 1,3,4-thiadia-zol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

Salts of the compounds of the formula I are preferably agriculturally acceptable salts. They can be formed in a customary method, e.g. by reacting the compound with an acid of the anion in question or, if the compound I has also an acidic center, by reacting it with a suitable base. The present compounds I have at least two centers of basicity, namely the two basic nitrogen ring atoms, and thus the agriculturally acceptable salts are generally acid addition salts.

Suitable agriculturally acceptable salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and bar-ium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tet-raethylammonium, tetrabutylammonium, 2-hydroxyethylammonium, 2-(2-hydroxyeth-oxy) ethylammonium, bis(2-hydroxyethyl) ammonium, benzyltrimethylammonium and benzl-triethylammonium, furthermore phosphonium ions, sulfonium ions, preferably tri (C₁-C₄-alkyl) sulfonium, and sulfoxonium ions, preferably tri (C₁-C₄-alkyl) sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formula I with an acid of the corresponding anion, preferably of hydrochloric acid, hydrobromic acid, sulfuric acid, phos-phoric acid or nitric acid.

The term “steroisomers” relates to enantiomers and diastereomers including Z/E iso-mers.

Depending on the substitution pattern, the compounds of the formula (I) may have one or more stereogenic centers. Just by way of example, a stereogenic center in compounds (I) is the C atom carrying R⁵ and R⁶, provided of course that R⁵ and R⁶ have different meanings or provided that the ring formed by R⁵ and R⁶ together with said carbon atom has not rotary mirror axis. Also the C atom carrying R⁷ and R⁸, if present (i.e. if m≠0), is a stereogenic center, provided of course that R⁷ and R⁸ have different meanings or provided that the ring formed by R⁷ and R⁸ together with said carbon atom has not rotary mirror axis. The invention provides both the use of pure enantiomers, of pure diastereomers and of mixtures of enantiomers and/or of diastereomers.

In terms of the present invention, the term “pure enantiomer” is understood as a non-racemic mixture of a specific compound where the desired enantiomer is present in an enantiomeric excess of >90% ee. In terms of the present invention, the term “pure diastereomer” is understood as a mixture of the diastereomers of a specific compound, where the desired diastereomer is present in an amount of >90%, based on the total amount of diastereomers of said compound.

The term N-oxides relates to a form of compounds I in which at least one nitrogen atom is present in oxidized form (as NO). To be more precise, it relates to any compound I which has at least one tertiary nitrogen atom that is oxidized to an N-oxide moiety. N-oxides of compounds I can in particular be prepared by oxidizing e.g. the ring nitrogen atom of the pyridine ring and/or of any nitrogen-containing heterocyclic or heteroaromatic group present, such as for example the nitrogen atom vicinal to CR⁵R⁶, with a suitable oxidizing agent, such as peroxo carboxylic acids or other peroxides. The person skilled in the art knows if and in which positions compounds I may form N-oxides.

The compounds (I) may be present in form of different tautomers. For instance, if the ring carrying R⁵ and R⁶ is a lactam, i.e. if it contains an amide group as ring member (=unsubstituted, secondary nitrogen ring atom neighboured to a carbon ring atom carrying an oxo group), this ring moiety —N(H)—C(═O)— can be in equilibrium with its tauto-meric form —N═C(OH)—. This is for example the case if m is 0, R⁵ and R⁶ form together an oxo group, Y is NR⁹ and R⁹ is hydrogen.

### Biochemical Definitions

The term “dihydroorotate dehydrogenase” (abbreviated DHODH) refers to an enzyme of the class 2 of DHODHs (mitochondrial origin, FMN as cofactor) EC 1.3.5.2 which ox-idizes dihydroorotate to orotate while reducing Quinone as an oxidizing agent to Quinol.

The term “dihydroorotate-dehydrogenase inhibitor resistance” of a phytopathogenic fungus in the context of the present invention refers to the resistance against one or more prior art quinoline-type phytopathogen inhibitors as described in EP-A-2522658,EP-A-1736471 or WO2017/153380, in particular at least one compound of the formulae (1), (2) or (3):

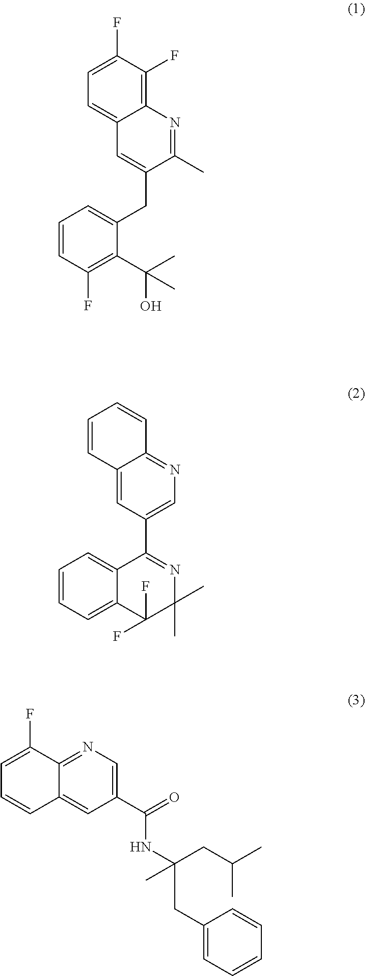

In particular, resistance is conferred by at least one mutation in the DHODH structural gene, resulting in at least one amino acid residue change, as for example addition, inversion, deviation or substitution, in particular deletion or substitution, more particularly substitution, of at least one amino acid residue of the endogenous non-mutated DHODH enzyme. Such mutation may for example induce a change in the primary, secondary, tertiary or quaternary protein structure of the enzyme which affects docking of an inhibitory molecule to the enzyme, for example into the substrate pocket of the enzyme.

The term “methods for combating phytopathogenic fungi containing a resistance conferring mutation in the DHODH gene” comprises applying compounds of formula I of the invention onto such resistant phytopathogenic fungus. It has been observed under field conditions that populations of phytopathogenic fungi apparently consisting of non-resistant strains can readily develop resistance. The compounds of the invention can be applied under such conditions, too, in order to prevent the formation of resistance and the spread of resistant strains altogether. In this regard it is useful that they have strong activity against non-resistant phytopathogenic fungi also.

The term “phytopathogenic fungus containing a resistance conferring mutation in the DHODH gene” is to be understood that at least 10% of the fungal isolates to be controlled contain a mutation in the endogenous DHODH gene conferring resistance to a prior art inhibitor as defined above, more preferably at least 30%, even more preferably at least 50%, and most preferably at least 75%, in particular between 90 and 100% of the fungal isolate to be controlled.

The term “resistance-inducing mutation” or “resistance-conferring mutation” describes the influence of a mutation introduced into the coding sequence of a particular enzyme, in the present matter of DHODH, as expressed in a phytopathogenic fungus, on the ability of a particular inhibitor substance or class of inhibitor substances, to combat said phytopathogen. By such mutation the inhibiting activity of such substance may be partially reduced or completely abolished. For example, the inhibitor concentration of such substance resulting in a 50% reduction of growth, for example expressed as IC₅₀ concentration as determined in the experimental section below, may be increased relative to the respective value observed for the non-mutated wild type strain of the phytopathogen under otherwise identical conditions. For example, the resistance may be increased by a factor of at least 2 (i.e. the respective IC₅₀ concentration is at least dou-bled), as for example increased by a factor in the range of 2 to 1exp6, or in particular 5 to 10.000, more particularly 10 to 5.000 or 50 to 1.000, or especially 100 to 500.

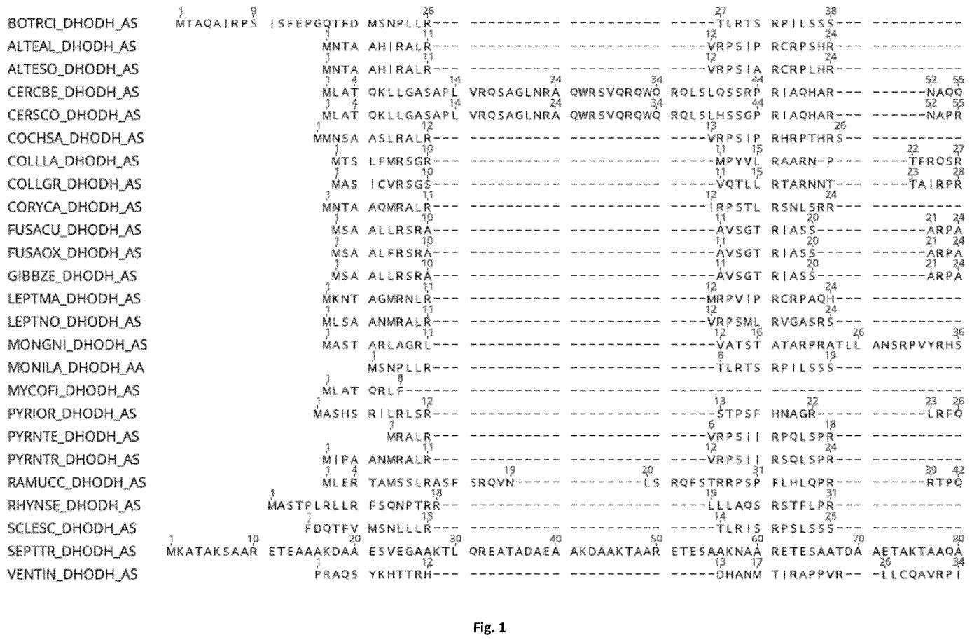

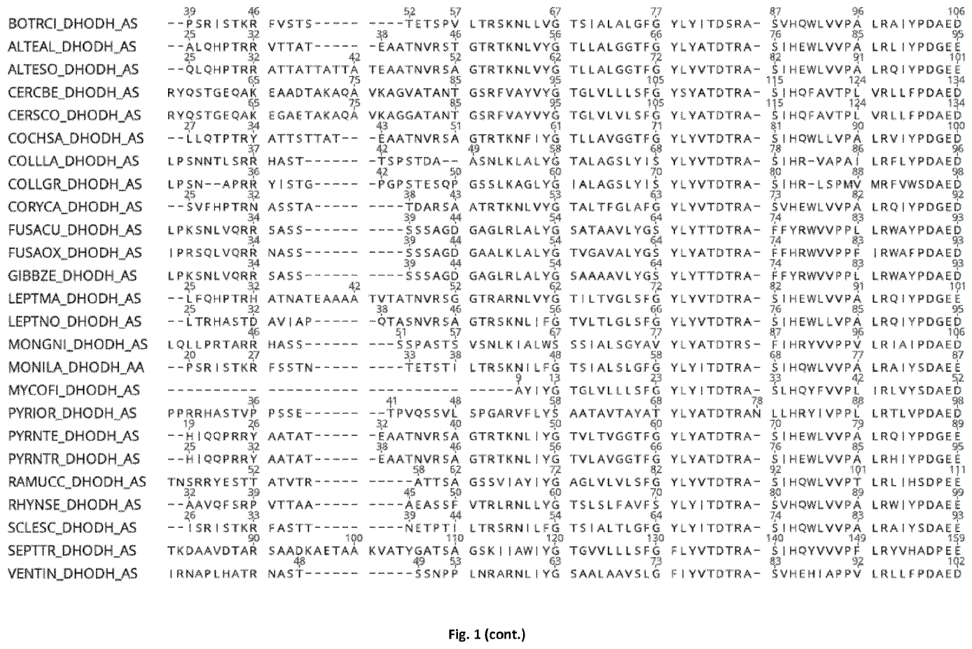

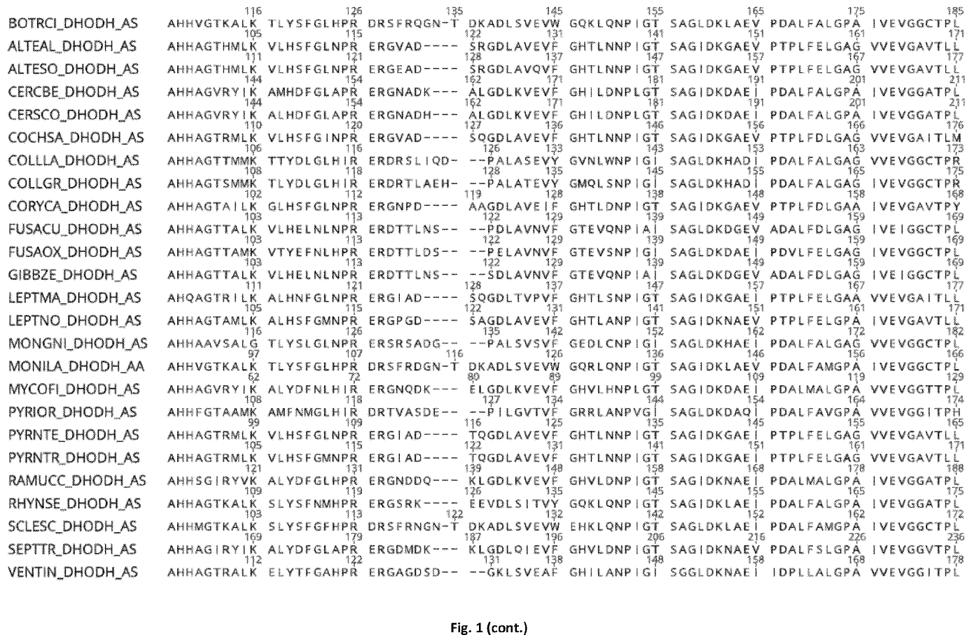

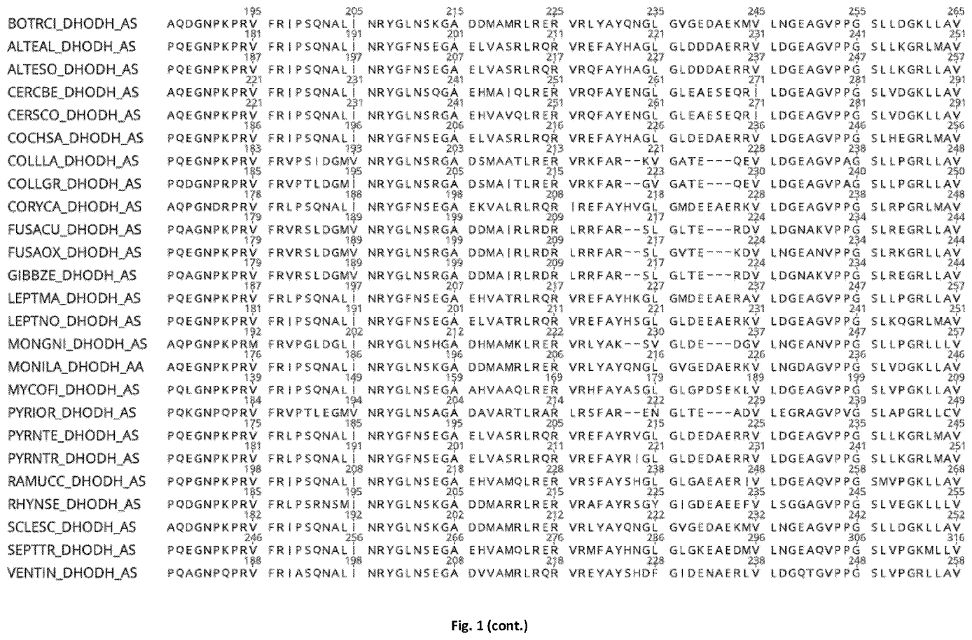

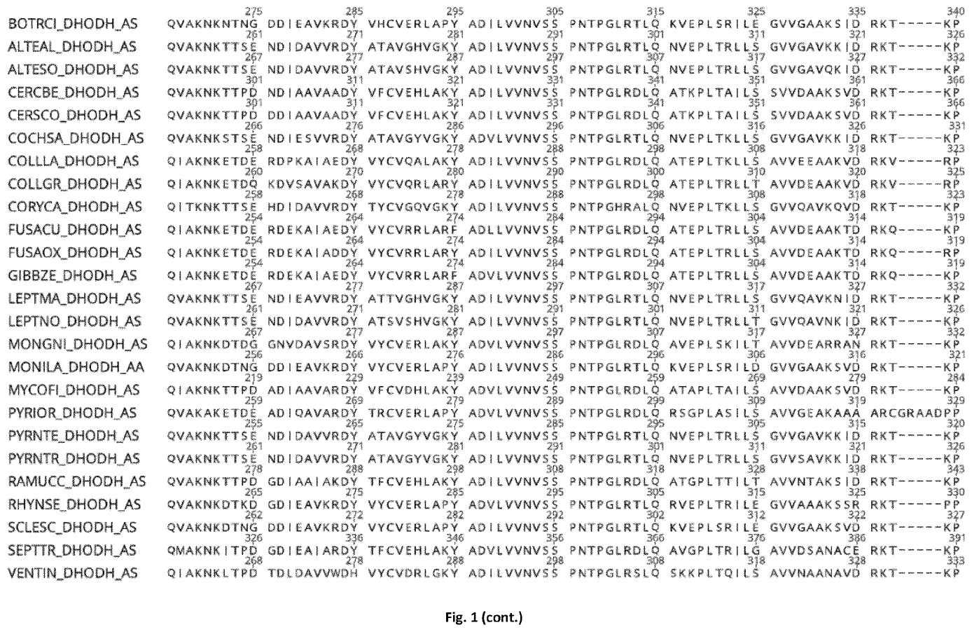

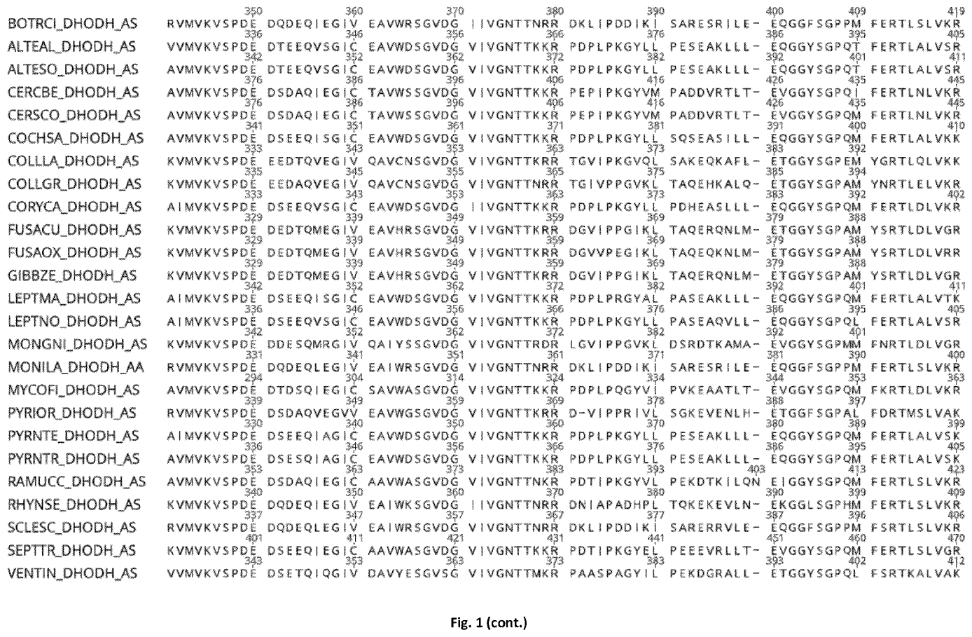

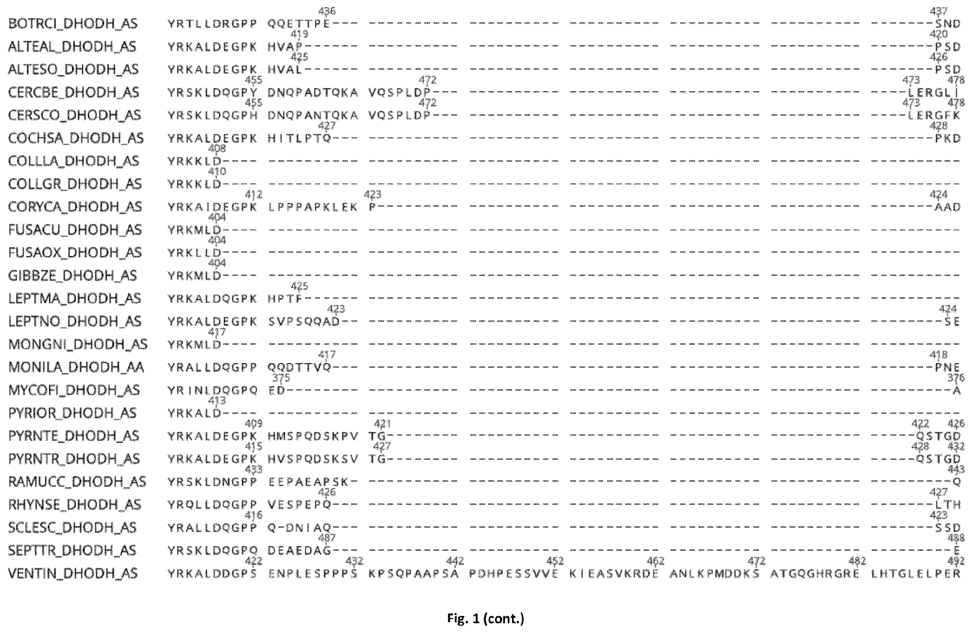

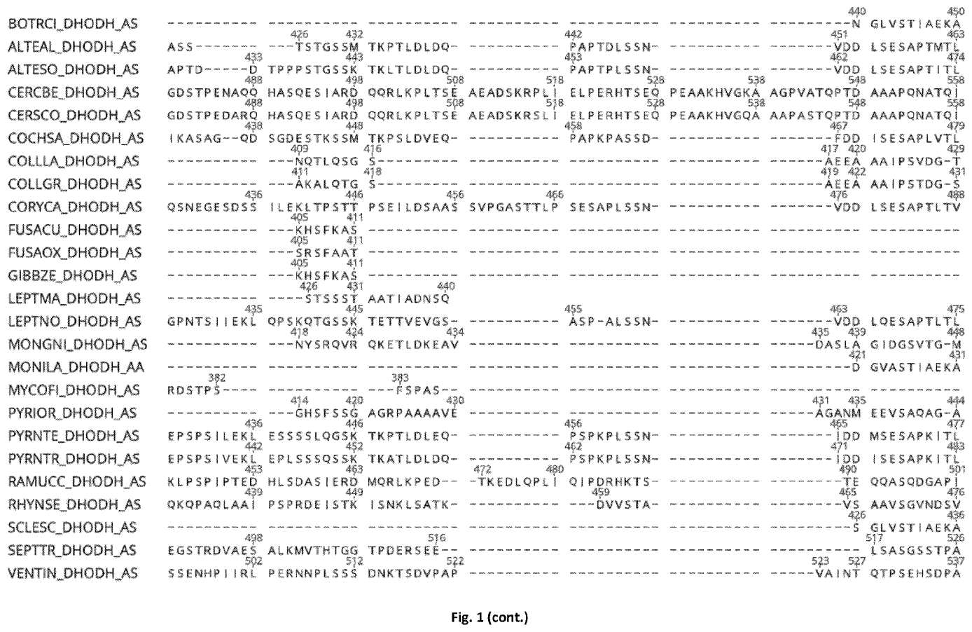

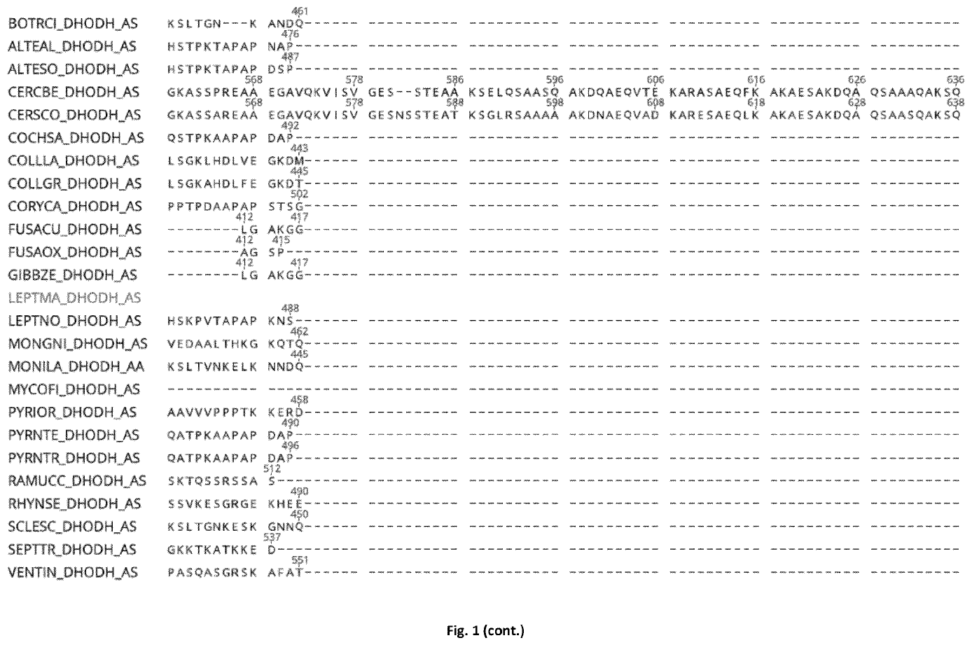

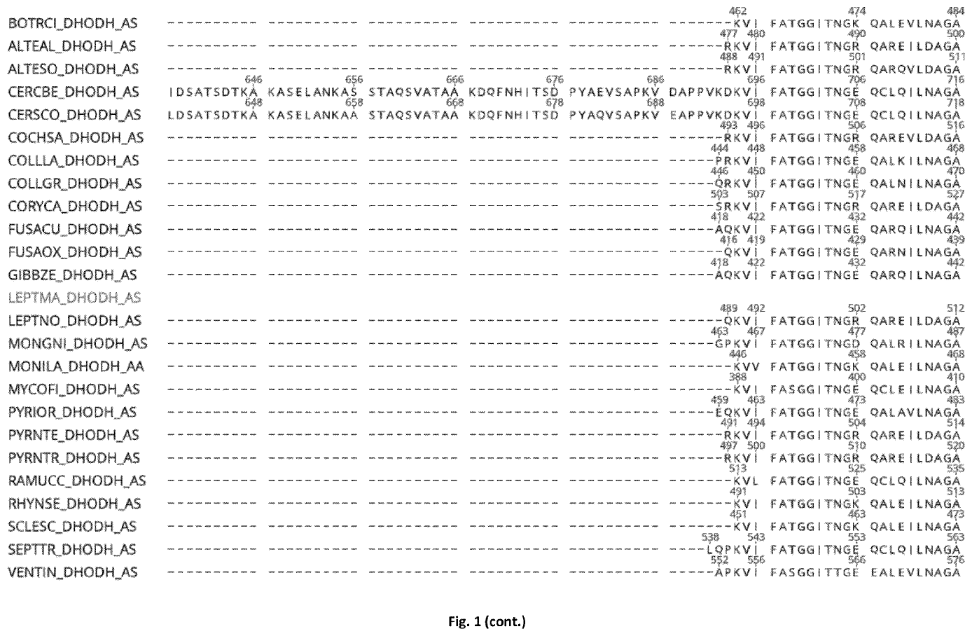

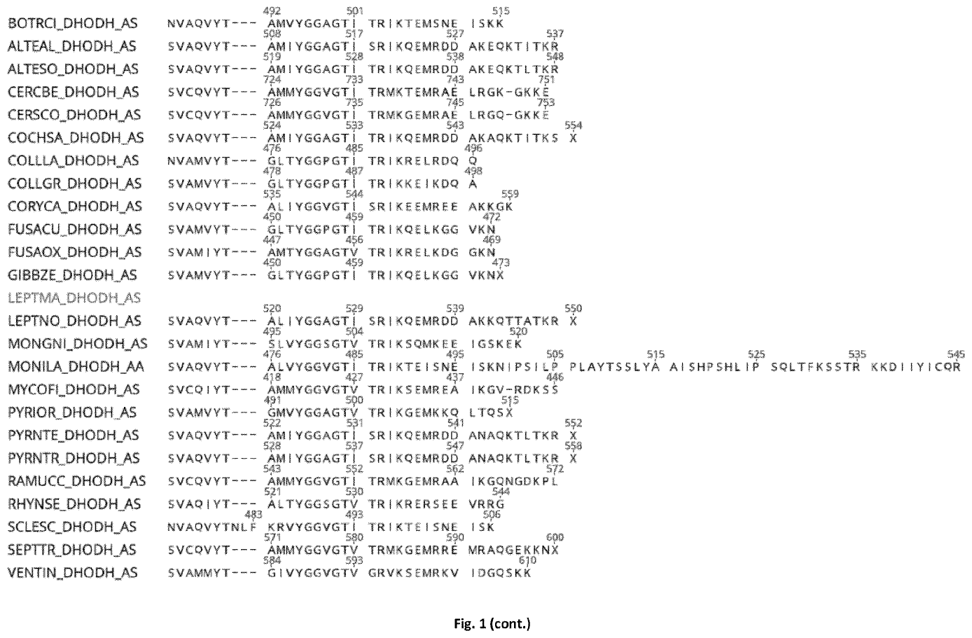

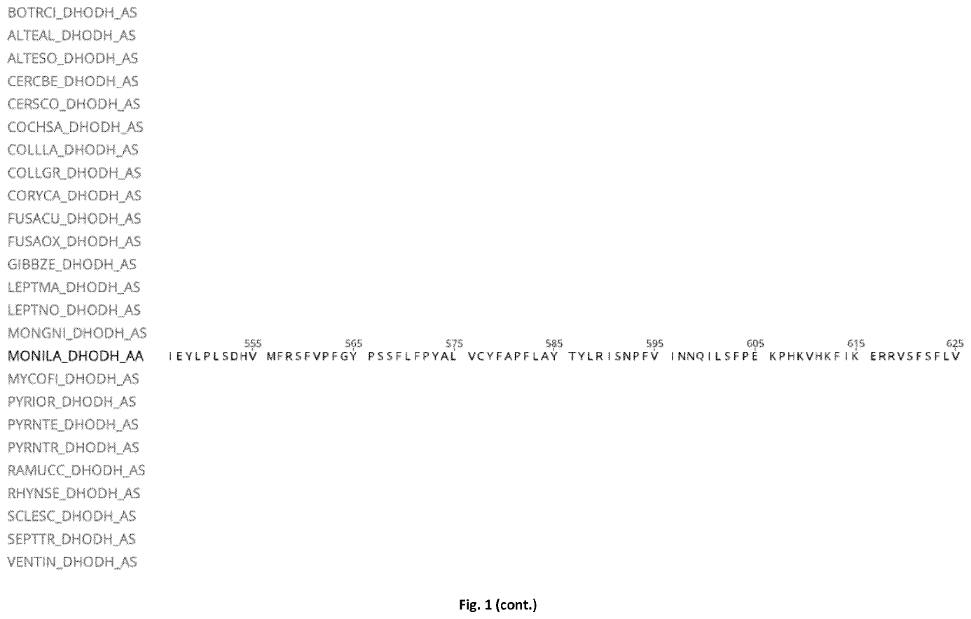

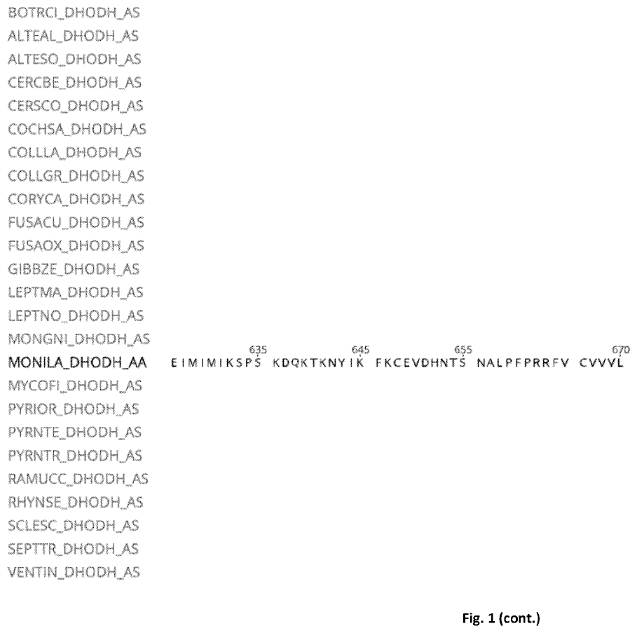

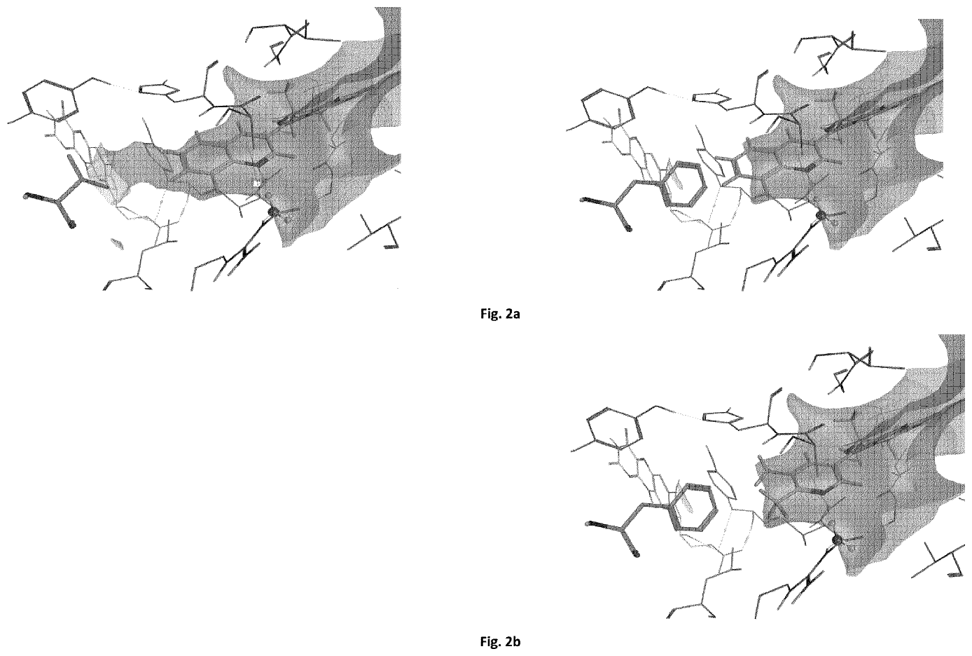

The term “analogous amino acid sequence position” refers to amino acid residues in different species of a particular type of protein, here in particular amino acid residues of DHODH enzymes isolated from different phytopathogenic fungi, which are located within the respective amino acid sequence in particular sequence motifs or consensus sequences or signatures, and which are predicted to have similar effects on a functional feature of the protein, as for example enzyme activity or substrate binding ability or inhibitor binding ability. Such an analogous amino acid sequence positions may be easily derived from so-called amino acid sequence alignments. An example thereof is depicted in FIG. 1 of the present application. Generating suitable sequence alignments is well within the scope of a person of ordinary skill in the art.

The term “motif” or “consensus sequence” or “signature” refers to a short conserved region in the sequence of optionally evolutionarily related proteins. Motifs are frequently highly conserved parts of domains, but may also include only part of the domain. A “protein family” is defined as a group of proteins that share a common evolutionary origin reflected by their related functions, similarities in sequence, or similar primary, secondary or tertiary structure. Proteins within protein families are usually homologous and have similar structure of conserved functional domains and motifs.

As used herein, the term “host cell” or “transformed cell” refers to a cell (or organism) altered to harbor at least one nucleic acid molecule, for instance, a recombinant gene encoding a desired protein or nucleic acid sequence which upon transcription yields at least one functional polypeptide of the present invention, in particular a DHODH enzyme or mutant thereof as defined herein. The host cell is particularly a bacterial cell, a fungal cell or a plant cell. The host cell may contain a recombinant gene or several genes, as for example organized as an operon, which has been integrated into the nu-clear or organelle genomes of the host cell. Alternatively, the host may contain the recombinant gene extra-chromosomally.

The term “organism” refers to any non-human multicellular or unicellular organism such as a plant, or a microorganism. Particularly, a microorganism is a bacterium, a yeast, an algae or a fungus.

The term “plant” includes plant cells including plant protoplasts, plant tissues, plant cell tissue cultures giving rise to regenerated plants, or parts of plants, or plant organs such as roots, stems, leaves, flowers, pollen, ovules, embryos, fruits and the like.

“Proteinogenic” amino acids comprise in particular (single-letter code): G, A, V, L, I, F, P, M, W, S, T, C, Y, N, Q, D, E, K, R and H.

### EMBODIMENTS

Unless specified otherwise, the below description of general and preferred embodiments of the invention relate both to the use and the method as well as to the composition of the invention. The remarks made below concerning preferred embodiments of the variables of the compounds of formula I, especially with respect to their substituents R¹, R², R³, R⁴, R⁵, R⁶, R⁷, R⁸, X, Y, m and n and any subgroups thereof, the fea-tures of the use and method according to the invention and of the composition of the invention are valid both on their own and, in particular, in every possible combination with each other.

### a) Compounds (I)

In a preferred embodiment, R¹ is hydrogen.

In a preferred embodiment, R⁴ is hydrogen.

In a preferred embodiment, both R¹ and R⁴ are hydrogen.

In a preferred embodiment, R² is halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₁-C₆-alkoxy, C₂-C₆-alkenyloxy or C₂-C₆-alkynyloxy. More preferably, R² is C₁-C₄-alkyl or C₁-C₄-haloalkyl, and is in particular C₁-C₄-alkyl.

In a preferred embodiment, R³ is halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₁-C₆-alkoxy, C₂-C₆-alkenyloxy or C₂-C₆-alkynyloxy. More preferably, R³ is C₁-C₄-alkyl or C₁-C₄-haloalkyl.

In a preferred embodiment, R² and R³, independently of each other, are C₁-C₄-alkyl or C₁-C₄-haloalkyl. More preferably, R² is C₁-C₄-alkyl and R³ is C₁-C₄-alkyl or C₁-C₄-haloalkyl.

In particular, R¹ and R⁴ are hydrogen and R² and R³, independently of each other, are C₁-C₄-alkyl or C₁-C₄-haloalkyl. More particularly, R¹ and R⁴ are hydrogen, R² is C₁-C₄-alkyl and R³ is C₁-C₄-alkyl or C₁-C₄-haloalkyl.

In a preferred embodiment,
R⁵ is hydrogen, C₁-C₆-alkyl, C₁-C₆-haloalkyl, phenyl or benzyl, where the 4 last-mentioned aliphatic or aromatic radicals are unsubstituted or carry 1 or 2 substituents R^{5a}; where
   each R^{5a} is independently halogen or C₁-C₆-alkoxy;
R⁶ is hydrogen, C₁-C₆-alkyl, C₁-C₆-haloalkyl, phenyl or benzyl, where the 4 last-mentioned aliphatic or aromatic radicals are unsubstituted or carry 1 or 2 substituents R^{6a}; where
   each R^{6a} is independently halogen or C₁-C₆-alkoxy;
or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶;where each R⁵⁶ is independently C₁-C₄-alkyl.

Even more preferably,
R⁵ and R⁶, independently of each other, are hydrogen, C₁-C₆-alkyl or phenyl;
or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶;where each R⁵⁶ is independently C₁-C₄-alkyl.

In particular,
R⁵ and R⁶, independently of each other, are hydrogen, C₁-C₆-alkyl or phenyl where in case that m is 0, at least one of R⁵ and R⁶ is not hydrogen;
or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶;where each R⁵⁶ is independently C₁-C₄-alkyl.

In a preferred embodiment,
R⁷ and R⁸, independently of each other, are hydrogen, C₁-C₄-alkyl or C₁-C₄-alkoxy-C₁-C₄-alkyl;
or
R⁷ and R⁸, together with the carbon atom to which they are bound, form a 3-, 4-, 5- or 6-membered saturated carbocyclic ring.

In a preferred embodiment, each X is independently halogen, CN, C₁-C₆-alkyl, C₁-C₆-haloalkyl, C₁-C₆-alkoxy or C₁-C₆-haloalkoxy; and is more preferably halogen, C₁-C₆-alkyl or C₁-C₆-alkoxy.
n is preferably 0, 1 or 2.

In one embodiment, Y is O and m is 0.

In another embodiment, Y is NR° and m is 0.

In yet another embodiment, Y is O and m is 1.

In a preferred embodiment, R⁹ is hydrogen, —CH(═O), —C(═O)C₁-C₆-alkyl, —C(═O)C₂-C₆-alkenyl, —C(═O)C₂-C₆-alkynyl, —C(═O)C₃-C₆-cycloalkyl, C₁-C₆-alkyl, C₁-C₄-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, —S(═O)₂-R^{9a}, wherein R^{9a} is C₁-C₆-alkyl, C₁-C₆-haloalkyl or phenyl, where the phenyl ring is unsubstituted or carries 1, 2 or 3 C₁-C₄-alkyl substituents; five-or six-membered heteroaryl or phenly; wherein heteroaryl contains 1, 2 or 3 heteroatoms selected from N, O and S as ring members; wherein phenyl is unsubstituted or carries 1, 2, 3, 4 or 5 substituents selected from the group consisting of CN, halogen, OH, C₁-C₄-alkyl, C₁-C₄-haloalkyl, C₁-C₄-alkoxy and C₁-C₄-haloalkoxy.

More preferably, R⁹ is hydrogen, C₁-C₄-alkyl, —C(═O)C₁-C₃-alkyl or —S(═O)₂-R^{9a}, wherein R^{9a} is C₁-C₆-alkyl, C₁-C₆-haloalkyl or phenyl, where the phenyl ring is unsubstituted or carries 1, 2 or 3 C₁-C₄-alkyl substituents.

In particular, in compounds (I),
R¹ is hydrogen;
R² is C₁-C₄-alkyl;
R³ is C₁-C₄-alkyl or C₁-C₄-haloalkyl;
R⁴ is hydrogen;
R⁵ is hydrogen, C₁-C₆-alkyl or phenyl;
R⁶ is hydrogen, C₁-C₆-alkyl or phenyl;
or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶;where each R⁵⁶ is independently C₁-C₄-alkyl;
R⁷ is hydrogen, C₁-C₄-alkyl or C₁-C₄-alkoxy-C₁-C₄-alkyl;
R⁸ is hydrogen, C₁-C₄-alkyl or C₁-C₄-alkoxy-C₁-C₄-alkyl;
or
R⁷ and R⁸, together with the carbon atom to which they are bound, form a 3-, 4-, 5- or 6-membered saturated carbocyclic ring;
each X is independently halogen, C₁-C₆-alkyl or C₁-C₆-alkoxy;
R⁹ is hydrogen, C₁-C₄-alkyl, —C(═O)C₁-C₃-alkyl or —S(═O)₂-R⁹a;
wherein
R^{9a} is C₁-C₆-alkyl, C₁-C₆-haloalkyl or phenyl, where the phenyl ring is unsubstituted or carries 1, 2 or 3 C₁-C₄-alkyl substituents; and
n is 0, 1 or 2.

More preferably,
R¹ is hydrogen;
R² is C₁-C₄-alkyl;
R³ is C₁-C₄-alkyl or C₁-C₄-haloalkyl;
R⁴ is hydrogen;
R⁵ is hydrogen, C₁-C₆-alkyl or phenyl;
R⁶ is hydrogen, C₁-C₆-alkyl or phenyl;
where in case that m is 0, at least one of R⁵ and R⁶ is not hydrogen;
or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶; where
   each R⁵⁶ is independently C₁-C₄-alkyl;
R⁷ is hydrogen, C₁-C₄-alkyl or C₁-C₄-alkoxy-C₁-C₄-alkyl;
R⁸ is hydrogen, C₁-C₄-alkyl or C₁-C₄-alkoxy-C₁-C₄-alkyl;
or
R⁷ and R⁸, together with the carbon atom to which they are bound, form a 3-, 4-, 5- or 6-membered saturated carbocyclic ring;
each X is independently halogen, C₁-C₆-alkyl or C₁-C₆-alkoxy;
R⁹ is hydrogen, C₁-C₄-alkyl, —C(═O)C₁-C₃-alkyl or —S(═O)₂-R⁹a;
wherein
   R^{9a} is C₁-C₆-alkyl, C₁-C₆-haloalkyl or phenyl, where the phenyl ring is unsubstituted or carries 1, 2 or 3 C₁-C₄-alkyl substituents; and
n is 0, 1 or 2.

In one embodiment, Y is O and m is 0. This results in compounds of the formula (I.A)

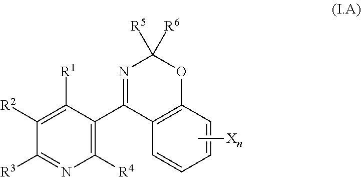

where R¹, R², R³, R⁴, R⁵, R⁶, X and n have one of the above general or, in particular, one of the above preferred meanings.

In particular, in compounds (I.A),
R¹ is hydrogen;
R² is C₁-C₄-alkyl;
R³ is C₁-C₄-alkyl or C₁-C₄-haloalkyl;
R⁴ is hydrogen;
R⁵ is hydrogen, C₁-C₆-alkyl or phenyl;
R⁶ is C₁-C₆-alkyl or phenyl;
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶; where
   each R⁵⁶ is independently C₁-C₄-alkyl;
each X is independently halogen, C₁-C₆-alkyl or C₁-C₆-alkoxy; and
n is 0, 1 or 2.

In another embodiment, Y is NR° and m is 0. This results in compounds of the formula (I.B)

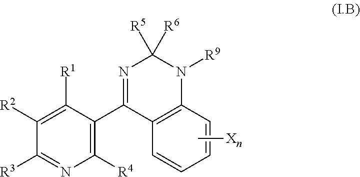

where R¹, R², R³, R⁴, R⁵, R⁶, R⁹, X and n have one of the above general or, in particular, one of the above preferred meanings.

In particular, in compounds (I.B),
R¹ is hydrogen;
R² is C₁-C₄-alkyl;
R³ is C₁-C₄-alkyl or C₁-C₄-haloalkyl;
R⁴ is hydrogen;
R⁵ is C₁-C₆-alkyl;
R⁶ is C₁-C₆-alkyl;
each X is independently halogen;
R⁹ is hydrogen, C₁-C₄-alkyl, —C(═O)C₁-C₃-alkyl or —S(═O)₂-R^{3a};
   wherein
   R⁹ is phenyl, where the phenyl ring is unsubstituted or carries 1, 2 or 3 C₁-C₄-alkyl substituents; and
n is 0, 1 or 2.

In another embodiment, Y is O and m is 1. This results in compounds of the formula (I.C)

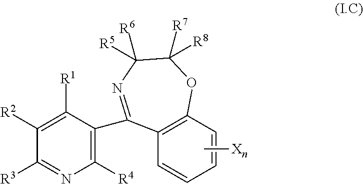

where R¹, R², R³, R⁴, R⁵, R⁶, X and n have one of the above general or, in particular, one of the above preferred meanings.

In particular, in compounds (I.C),
R¹ is hydrogen;
R² is C₁-C₄-alkyl;
R³ is C₁-C₄-alkyl or C₁-C₄-haloalkyl;
R⁴ is hydrogen;
R⁵ is hydrogen or C₁-C₆-alkyl;
R⁶ is hydrogen or C₁-C₆-alkyl;
or
R⁵ and R⁶ form together an oxo group (═O);
R⁷ is hydrogen, C₁-C₄-alkyl or C₁-C₄-alkoxy-C₁-C₄-alkyl;
R⁸ is hydrogen, C₁-C₄-alkyl or C₁-C₄-alkoxy-C₁-C₄-alkyl;
or
R⁷ and R⁸, together with the carbon atom to which they are bound, form a 3-, 4-, 5- or 6-membered saturated carbocyclic ring; and
n is 0.

Examples of preferred compounds are compounds of the following formulae I.A. 1 to I.A.6, I.B.1 to I.B.4 and I.C.1 to I.C.4, where X, n, R⁵ and R⁶, R⁷, R⁸ and R⁹ have one of the general or preferred meanings given above. Examples of preferred compounds are the individual compounds compiled in the tables below. Moreover, the meanings mentioned below for the individual variables in the tables are per se, independently of the combination in which they are mentioned, a particularly preferred embodiment of the substituents in question.

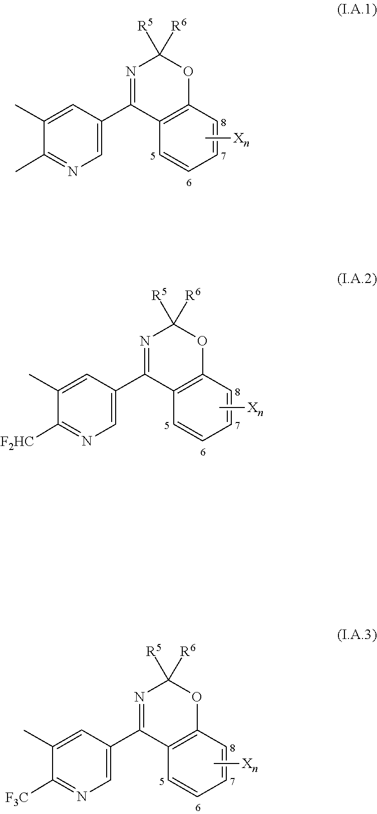

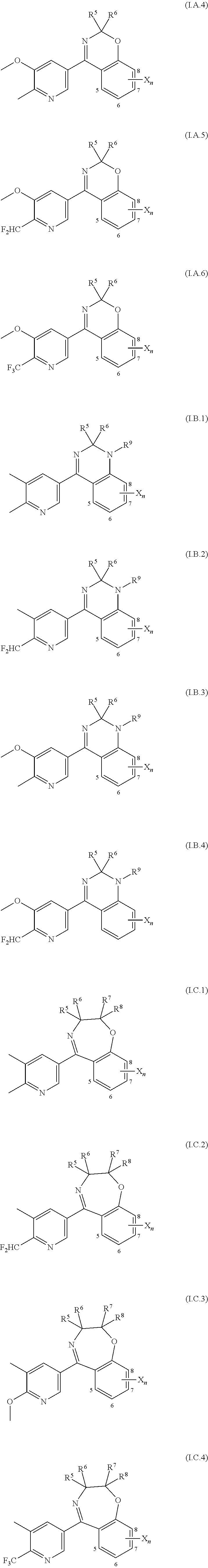

Particularly preferred compounds I.A.1, I.A.2, I.A.3, I.A.4, I.A.5 and I.A.6 are listed in the following tables 1 to 7.

### Table 1

Compounds of the formula I.A. 1, I.A.2, I.A.3, I.A.4, I.A.5, I.A.6 in which n is 0 and the combination of R⁵ and R⁶ for a compound corresponds in each case to one row of Table A

### Table 2

Compounds of the formula I.A.1, I.A.2, I.A.3, I.A.4, I.A.5, I.A.6 in which n is 1, X is 8-F and the combination of R⁵ and R⁶ for a compound corresponds in each case to one row of Table A

### Table 3

Compounds of the formula I.A. 1, I.A.2, I.A.3, I.A.4, I.A.5, I.A.6 in which n is 1, X is 8-Cl and the combination of R⁵ and R⁶ for a compound corresponds in each case to one row of Table A

### Table 4

Compounds of the formula I.A.1, I.A.2, I.A.3, I.A.4, I.A.5, I.A.6 in which n is 1, X is 7-methoxy and the combination of R⁵ and R⁶ for a compound corresponds in each case to one row of Table A

### Table 5

Compounds of the formula I.A.1, I.A.2, I.A.3, I.A.4, I.A.5, I.A.6 in which n is 2, X is 7,8-F₂ and the combination of R⁵ and R⁶ for a compound corresponds in each case to one row of Table A

### Table 6

Compounds of the formula I.A.1, I.A.2, I.A.3, I.A.4, I.A.5, I.A.6 in which n is 2, X is 6,8-F₂ and the combination of R⁵ and R⁶ for a compound corresponds in each case to one row of Table A

### Table 7

Compounds of the formula I.A.1, I.A.2, I.A.3, I.A.4, I.A.5, I.A.6 in which n is 2, X is 5,8-F₂ and the combination of R⁵ and R⁶ for a compound corresponds in each case to one row of Table A

Particularly preferred compounds I.B.1, I.B.2, I.B.3 and I.B.4 are listed in the following tables 8 to 14.

### Table 8

Compounds of the formula I.B.1, I.B.2, I.B.3, I.B.4 in which n is 0 and the combination of R⁵, R⁶ and R⁹ for a compound corresponds in each case to one row of Table B

### Table 9

Compounds of the formula I.B.1, I.B.2, I.B.3, I.B.4 in which n is 1, X is 8-F and the combination of R⁵, R⁶ and R⁹ for a compound corresponds in each case to one row of Table B

### Table 10

Compounds of the formula I.B.1, I.B.2, I.B.3, I.B.4 in which n is 1, X is 8-CI and the combination of R⁵, R⁶ and R⁹ for a compound corresponds in each case to one row of Table B

### Table 11

Compounds of the formula I.B.1, I.B.2, I.B.3, I.B.4 in which n is 1, X is 8-methyl and the combination of R⁵, R⁶ and R⁹ for a compound corresponds in each case to one row of Table B

### Table 12

Compounds of the formula I.B.1, I.B.2, I.B.3, I.B.4 in which n is 1, X is 8-methoxy and the combination of R⁵, R⁶ and R⁹ for a compound corresponds in each case to one row of Table B

### Table 13

Compounds of the formula I.B.1, I.B.2, I.B.3, I.B.4 in which n is 2, X is 7,8-F₂ and the combination of R⁵, R⁶ and R⁹ for a compound corresponds in each case to one row of Table B

### Table 14

Compounds of the formula I.B.1, I.B.2, I.B.3, I.B.4 in which n is 2, X is 7-F, 8-methoxy and the combination of R⁵, R⁶ and R⁹ for a compound corresponds in each case to one row of Table B

Particularly preferred compounds I.C.1, I.C.2, I.C.3 and I.C.4 are listed in the following tables 15 to 21.

### Table 15

Compounds of the formula I.C. 1, I.C.2, I.C.3, I.C.4 in which n is 0 and the combination of R⁵, R⁶, R⁷ and R⁸ for a compound corresponds in each case to one row of Table C

### Table 16

Compounds of the formula I.C.1, I.C.2, I.C.3, I.C.4 in which n is 1, X is 8-F and the combination of R⁵, R⁶, R⁷ and R⁸ for a compound corresponds in each case to one row of Table C

### Table 17

Compounds of the formula I.C.1, I.C.2, I.C.3, I.C.4 in which n is 1, X is 8-CI and the combination of R⁵, R⁶, R⁷ and R⁸ for a compound corresponds in each case to one row of Table C

### Table 18

Compounds of the formula I.C.1, I.C.2, I.C.3, I.C.4 in which n is 1, X is 7-methoxy and the combination of R⁵, R⁶, R⁷ and R⁸ for a compound corresponds in each case to one row of Table C

### Table 19

Compounds of the formula I.C.1, I.C.2, I.C.3, I.C.4 in which n is 2, X is 7,8-F₂ and the combination of R⁵, R⁶, R⁷ and R⁸ for a compound corresponds in each case to one row of Table C

### Table 20

Compounds of the formula I.C.1, I.C.2, I.C.3, I.C.4 in which n is 2, X is 6,8-F₂ and the combination of R⁵, R⁶, R⁷ and R⁸ for a compound corresponds in each case to one row of Table C

### Table 21

Compounds of the formula I.C.1, I.C.2, I.C.3, I.C.4 in which n is 2, X is 5,8-F₂ and the combination of R⁵, R⁶, R⁷ and R⁸ for a compound corresponds in each case to one row of Table C

TABLE A

| No. | R⁵ | R⁶ |
|---|---|---|
| A.1. | Me | Me |
| A.2. | Me | Et |
| A.3. | Et | Et |
| A.4. | iPr | Et |
| A.5. | 2-Bu | Et |
| A.6. | t-Bu | Et |
| A.7. | iBu | Et |
| A.8. | iPt | Et |
| A.9. | CH₂OCH₃ | Et |
| A.10. | Ph | Et |
| A.11. | 2-F—Ph | Et |
| A.12. | 4-F—Ph | Et |
| A.13. | 2,4-F₂—Ph | Et |
| A.14. | 2-Cl—Ph | Et |
| A.15. | 4-Cl—Ph | Et |
| A.16. | Bz | Et |
| A.17. | 2-F—Bz | Et |
| A.18. | 4-F—Bz | Et |
| A.19. | CF₃ | iPr |
| A.20. | Me | iPr |
| A.21. | Et | iPr |
| A.22. | iPr | iPr |
| A.23. | 2-Bu | iPr |
| A.24. | t-Bu | iPr |
| A.25. | iBu | iPr |
| A.26. | iPt | iPr |
| A.27. | CH₂OCH₃ | iPr |
| A.28. | Ph | iPr |
| A.29. | 2-F—Ph | iPr |
| A.30. | 4-F—Ph | iPr |
| A.31. | 2,4-F₂—Ph | iPr |
| A.32. | 2-Cl—Ph | iPr |
| A.33. | 4-Cl—Ph | iPr |
| A.34. | Bz | iPr |
| A.35. | 2-F—Bz | iPr |
| A.36. | 4-F—Bz | iPr |
| A.37. | CF₃ | t-Bu |
| A.38. | Me | t-Bu |
| A.39. | Et | t-Bu |
| A.40. | iPr | t-Bu |
| A.41. | 2-Bu | t-Bu |
| A.42. | t-Bu | t-Bu |
| A.43. | iBu | t-Bu |
| A.44. | iPt | t-Bu |
| A.45. | CH₂OCH₃ | t-Bu |
| A.46. | Ph | t-Bu |
| A.47. | 2-F—Ph | t-Bu |
| A.48. | 4-F—Ph | t-Bu |
| A.49. | 2,4-F₂—Ph | t-Bu |
| A.50. | 2-Cl—Ph | t-Bu |
| A.51. | 4-Cl—Ph | t-Bu |
| A.52. | Bz | t-Bu |
| A.53. | 2-F—Bz | t-Bu |
| A.54. | 4-F—Bz | t-Bu |
| A.55. | CF₃ | iPt |
| A.56. | Me | iPt |
| A.57. | Et | iPt |
| A.58. | iPr | iPt |
| A.59. | 2-Bu | iPt |
| A.60. | t-Bu | iPt |
| A.61. | iBu | iPt |
| A.62. | iPt | iPt |
| A.63. | CH₂OCH₃ | iPt |
| A.64. | Ph | iPt |
| A.65. | 2-F—Ph | iPt |
| A.66. | 4-F—Ph | iPt |
| A.67. | 2,4-F₂—Ph | iPt |
| A.68. | 2-Cl—Ph | iPt |
| A.69. | 4-Cl—Ph | iPt |
| A.70. | Bz | iPt |
| A.71. | 2-F—Bz | iPt |
| A.72. | 4-F—Bz | iPt |
| A.73. | CF₃ | CH₂O-t-Bu |
| A.74. | Me | CH₂O-t-Bu |
| A.75. | Et | CH₂O-t-Bu |
| A.76. | iPr | CH₂O-t-Bu |
| A.77. | 2-Bu | CH₂O-t-Bu |
| A.78. | t-Bu | CH₂O-t-Bu |
| A.79. | iBu | CH₂O-t-Bu |
| A.80. | iPt | CH₂O-t-Bu |
| A.81. | CH₂OCH₃ | CH₂O-t-Bu |
| A.82. | Ph | CH₂O-t-Bu |
| A.83. | 2-F—Ph | CH₂O-t-Bu |
| A.84. | 4-F—Ph | CH₂O-t-Bu |
| A.85. | 2,4-F₂—Ph | CH₂O-t-Bu |
| A.86. | 2-Cl—Ph | CH₂O-t-Bu |
| A.87. | 4-Cl—Ph | CH₂O-t-Bu |
| A.88. | Bz | CH₂O-t-Bu |
| A.89. | 2-F—Bz | CH₂O-t-Bu |
| A.90. | 4-F—Bz | CH₂O-t-Bu |
| A.91. | CF₃ | CH₂OPh |
| A.92. | Me | CH₂OPh |
| A.93. | Et | CH₂OPh |
| A.94. | iPr | CH₂OPh |
| A.95. | 2-Bu | CH₂OPh |
| A.96. | t-Bu | CH₂OPh |
| A.97. | iBu | CH₂OPh |
| A.98. | iPt | CH₂OPh |
| A.99. | CH₂OCH₃ | CH₂OPh |
| A.100. | Ph | CH₂OPh |
| A.101. | 2-F—Ph | CH₂OPh |
| A.102. | 4-F—Ph | CH₂OPh |
| A.103. | 2,4-F₂—Ph | CH₂OPh |
| A.104. | 2-Cl—Ph | CH₂OPh |
| A.105. | 4-Cl—Ph | CH₂OPh |
| A.106. | Bz | CH₂OPh |
| A.107. | 2-F—Bz | CH₂OPh |
| A.108. | 4-F—Bz | CH₂OPh |
| A.109. | CF₃ | iHpt |
| A.110. | Me | iHpt |
| A.111. | Et | iHpt |
| A.112. | iPr | iHpt |
| A.113. | 2-Bu | iHpt |
| A.114. | t-Bu | iHpt |
| A.115. | iBu | iHpt |
| A.116. | iPt | iHpt |
| A.117. | CH₂OCH₃ | iHpt |
| A.118. | Ph | iHpt |
| A.119. | 2-F—Ph | iHpt |
| A.120. | 4-F—Ph | iHpt |
| A.121. | 2,4-F₂—Ph | iHpt |
| A.122. | 2-Cl—Ph | iHpt |
| A.123. | 4-Cl—Ph | iHpt |
| A.124. | Bz | iHpt |
| A.125. | 2-F—Bz | iHpt |
| A.126. | 4-F—Bz | iHpt |
| A.127. | CF₃ | iHx |
| A.128. | Me | iHx |
| A.129. | Et | iHx |
| A.130. | iPr | iHx |
| A.131. | 2-Bu | iHx |
| A.132. | t-Bu | iHx |
| A.133. | iBu | iHx |
| A.134. | iPt | iHx |
| A.135. | CH₂OCH₃ | iHx |
| A.136. | Ph | iHx |
| A.137. | 2-F—Ph | iHx |
| A.138. | 4-F—Ph | iHx |
| A.139. | 2,4-F₂—Ph | iHx |
| A.140. | 2-Cl—Ph | iHx |
| A.141. | 4-Cl—Ph | iHx |
| A.142. | Bz | iHx |
| A.143. | 2-F—Bz | iHx |
| A.144. | 4-F—Bz | iHx |
| A.145. | CF₃ | iDc |
| A.146. | Me | iDc |
| A.147. | Et | iDc |
| A.148. | iPr | iDc |
| A.149. | 2-Bu | iDc |
| A.150. | t-Bu | iDc |
| A.151. | iBu | iDc |
| A.152. | iPt | iDc |
| A.153. | CH₂OCH₃ | iDc |
| A.154. | Ph | iDc |
| A.155. | 2-F—Ph | iDc |
| A.156. | 4-F—Ph | iDc |
| A.157. | 2,4-F₂—Ph | iDc |
| A.158. | 2-Cl—Ph | iDc |
| A.159. | 4-Cl—Ph | iDc |
| A.160. | Bz | iDc |
| A.161. | 2-F—Bz | iDc |
| A.162. | 4-F—Bz | iDc |

| | |
|---|---|
| A.163. | R.1 |
| A.164. | R.2 |
| A.165. | R.3 |
| A.166. | R.4 |
| A.167. | R.5 |
| A.168. | R.6 |
| A.169. | R.7 |
| A.170. | R.8 |
| A.171. | R.9 |
| A.172. | R.10 |
| A.173. | R.11 |
| A.174. | R.12 |
| A.175. | R.13 |
| A.176. | R.14 |
| A.177. | R.15 |
| A.178. | R.16 |
| A.179. | R.17 |
| A.180. | R.18 |

Me = methyl;
Et = ethyl,
n-Pr = n-propyl,
iPr = isopropyl,
n-Bu = n-butyl [(CH₂)₃CH₃],
2-Bu = 2-butyl (sec-butyl; CH(CH₃)CH₂CH₃),
iBu = isobutyl (CH₂CH(CH₃)₂),
t-Bu = tert-butyl (C(CH₃)₃),
iPt = CH₂—C(CH₃)₃,
iHx = CH₂CH₂C(CH₃)₃,
iHpt = CH₂—CH(CH₃)—C(CH₃)₃,
iDc = CH₂—CH(C(CH₃)₃)₂,
OMe = methoxy,
Ph = phenyl,
Bz = benzyl,
2-F—Bz = CH₂—2F—Ph,
4-F—Bz = CH₂—4F—Ph

TABLE B

| No. | R⁵ | R⁶ | R⁹ |
|---|---|---|---|
| B.1. | Me | Me | H |
| B.2. | Me | Me | Me |
| B.3. | Me | Me | Et |
| B.4. | Me | Me | CN |
| B.5. | Me | Me | OMe |
| B.6. | Me | Me | CH₂CN |
| B.7. | Me | Me | CHO |
| B.8. | Me | Me | n-Pr |
| B.9. | Me | Me | t-Bu |
| B.10. | Me | Me | iPt |
| B.11. | Et | Me | H |
| B.12. | Et | Me | Me |
| B.13. | Et | Me | Et |
| B.14. | Et | Me | CN |
| B.15. | Et | Me | OMe |
| B.16. | Et | Me | CH₂CN |
| B.17. | Et | Me | CHO |
| B.18. | Et | Me | n-Pr |
| B.19. | Et | Me | t-Bu |
| B.20. | Et | Me | iPt |
| B.21. | t-Bu | Me | H |
| B.22. | t-Bu | Me | Me |
| B.23. | t-Bu | Me | Et |
| B.24. | t-Bu | Me | CN |
| B.25. | t-Bu | Me | OMe |
| B.26. | t-Bu | Me | CH₂CN |
| B.27. | t-Bu | Me | CHO |
| B.28. | t-Bu | Me | n-Pr |
| B.29. | t-Bu | Me | t-Bu |
| B.30. | t-Bu | Me | iPt |
| B.31. | Et | Et | H |
| B.32. | Et | Et | Me |
| B.33. | Et | Et | Et |
| B.34. | Et | Et | CN |
| B.35. | Et | Et | OMe |
| B.36. | Et | Et | CH₂CN |
| B.37. | Et | Et | CHO |
| B.38. | Et | Et | n-Pr |
| B.39. | Et | Et | t-Bu |
| B.40. | Et | Et | iPt |
| B.41. | t-Bu | Et | H |
| B.42. | t-Bu | Et | Me |
| B.43. | t-Bu | Et | Et |
| B.44. | t-Bu | Et | CN |
| B.45. | t-Bu | Et | OMe |
| B.46. | t-Bu | Et | CH₂CN |
| B.47. | t-Bu | Et | CHO |
| B.48. | t-Bu | Et | n-Pr |
| B.49. | t-Bu | Et | t-Bu |
| B.50. | t-Bu | Et | iPt |
| B.51. | t-Bu | t-Bu | H |
| B.52. | t-Bu | t-Bu | Me |
| B.53. | t-Bu | t-Bu | Et |
| B.54. | t-Bu | t-Bu | CN |
| B.55. | t-Bu | t-Bu | OMe |
| B.56. | t-Bu | t-Bu | CH₂CN |
| B.57. | t-Bu | t-Bu | CHO |
| B.58. | t-Bu | t-Bu | n-Pr |
| B.59. | t-Bu | t-Bu | t-Bu |
| B.60. | t-Bu | t-Bu | iPt |

| | | |
|---|---|---|
| B.61. | R.6 | H |
| B.62. | R.6 | Me |
| B.63. | R.6 | Et |
| B.64. | R.6 | CN |
| B.65. | R.6 | OMe |
| B.66. | R.6 | CH₂CN |
| B.67. | R.6 | CHO |
| B.68. | R.6 | n-Pr |
| B.69. | R.6 | t-Bu |
| B.70. | R.6 | iPt |

TABLE C

| No. | R⁵ | R⁶ | R⁷ | R⁸ |
|---|---|---|---|---|
| C.1. | H | H | H | H |
| C.2. | H | Me | H | H |
| C.3. | Me | Me | H | H |
| C.4. | H | H | Me | H |
| C.5. | H | Me | Me | H |
| C.6. | Me | Me | Me | H |
| C.7. | H | H | Me | Me |
| C.8. | H | Me | Me | Me |
| C.9. | Me | Me | Me | Me |

| | | | |
|---|---|---|---|
| C.10. | R.1 | H | H |
| C.11. | R.2 | H | H |
| C.12. | R.1 | Me | H |
| C.13. | R.2 | Me | H |
| C.14. | R.1 | Me | Me |
| C.15. | R.2 | Me | Me |

| | | | |
|---|---|---|---|
| C.16. | H | H | R.1 |
| C.17. | H | Me | R.1 |
| C.18. | Me | Me | R.1 |
| C.19. | H | H | R.2 |
| C.20. | H | Me | R.2 |
| C.21. | Me | Me | R.2 |

| | | |
|---|---|---|
| C.22. | R.1 | R.1 |
| C.23. | R.2 | R.1 |
| C.24. | R.1 | R.2 |
| C.25. | R.2 | R.2 |

R.1 to R.18 stand for following rings formed by R⁵ and R⁶ together with the carbon atom to which they are bound:

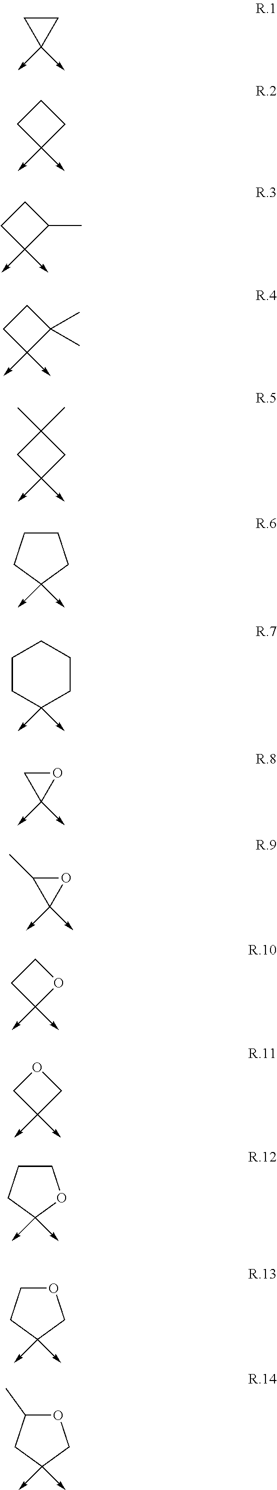

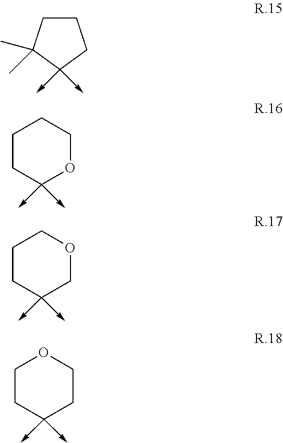

The arrows indicate the attachment point of the carbon ring atom carrying R⁵ and R⁶ to its other two other neighbor atoms (i.e. to N and in case of I.A. 1 to I.A.6 to O, in case of 5 I.B.1 to I.B.4 to NR° and in case of I.C.1 to I.C.4 to the carbon ring atom carrying R⁷ and R⁸).

Alternatively expressed,
in case of R.1 R⁵ and R⁶ form together a bridging group —CH₂CH₂—,
in case of R.2 R⁵ and R⁶ form together a bridging group —CH₂CH₂CH₂—,
in case of R.3 R⁵ and R⁶ form together a bridging group —CH(CH₃) CH₂CH₂—,
in case of R.4 R⁵ and R⁶ form together a bridging group —C(CH₃)₂CH₂CH₂—,
in case of R.5 R⁵ and R⁶ form together a bridging group —CH₂C(CH₃)₂CH₂—,
in case of R.6 R⁵ and R⁶ form together a bridging group —CH₂CH₂CH₂CH₂—,
in case of R.7 R⁵ and R⁶ form together a bridging group —CH₂CH₂CH₂CH₂CH₂—,
in case of R.8 R⁵ and R⁶ form together a bridging group —OCH₂—,
in case of R.9 R⁵ and R⁶ form together a bridging group —OCH(CH₃)—, in case of R.10 R⁵ and R⁶ form together a bridging group —OCH₂CH₂—,
in case of R.11 R⁵ and R⁶ form together a bridging group —CH₂OCH₂—,
in case of R.12 R⁵ and R⁶ form together a bridging group —OCH₂CH₂CH₂—,
in case of R.13 R⁵ and R⁶ form together a bridging group —CH₂OCH₂CH₂—,
in case of R.14 R⁵ and R⁶ form together a bridging group —CH₂OCH(CH₃) CH₂—,
in case of R.15 R⁵ and R⁶ form together a bridging group —CH₂OCH₂C(CH₃)₂—,
in case of R.16 R⁵ and R⁶ form together a bridging group —OCH₂CH₂CH₂CH₂—,
in case of R.17 R⁵ and R⁶ form together a bridging group —CH₂OCH₂CH₂CH₂—,
in case of R.18 R⁵ and R⁶ form together a bridging group —CH₂CH₂OCH₂CH₂—.

Particularly preferred are moreover the compounds used in the examples, the stereoisomers, tautomers, N-oxides agriculturally acceptable salts thereof.

The compounds I are principally known, e.g. from PCT/EP2022/062598, PCT/EP2022/062622 and PCT/EP2022/070790.

As far as the above-identified compounds are new, the invention also relates to these compounds per se, and to the N-oxides, tautomers, stereoisomers or agriculturally acceptable salts thereof.

Compounds I can be prepared as shown in the following schemes, in which, unless otherwise stated, the definition of each variable is as defined above for a compound of formula I. The compounds of the formula I can be prepared according to methods or in analogy to methods that are described in the prior art. The synthesis takes advantage of starting materials that are commercially available or may be prepared according to conventional procedures starting from readily available compounds.

For example, compounds I wherein Y is O (termed l′ in the following) can be prepared by a by palladium-catalyzed Suzuki coupling reaction between a boronic acid derivative 2 and a compound 1 (Z=halide or triflate) using a palladium complex in an organic solvent. It is preferred to conduct the reaction at elevated temperature, preferably between 6° and 160° C., and using 1 to 3 equivalents of boronic acid derivative 2 per 1 equivalent of compound 1, as described in WO 2009/119089.

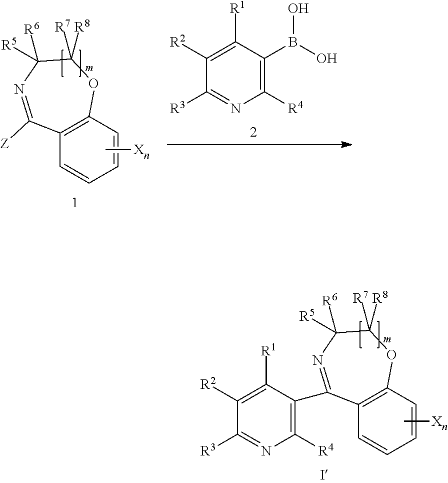

Compounds of the formula 1 wherein Z is a triflate group can be prepared from cyclic amide compound 3 by treatment with trifluoromethylsulfonic acid anhydride in the presence of a base such as pyridine, 2,6-lutidine, 2,3,5-colidine, triethylamine, tributyla-mine, diisopropylethylamine, a tertiary bicyclic amine or amidine, such as 1,4-diazabicyclo [2.2.2]octane, 1,5-diazabicyclo[4.3.0]non-5-ene or 1,8-diazabicyclo[5.4.0]undec-7-ene, or an aromatic amine, such as N,N-dimethylaniline, N,N-diethylaniline or 4-dimethylaminopyridine, in an organic halogenated aliphatic hydrocarbon solvent, such as chloroform, dichloromethane or dichloroethane, as described in WO 2009/119089 and EP2179994B1. Compounds of the formula 1 wherein Z is a halide can be prepared from the cyclic amide compound 3 by treatment with a suitable halogenating agent such as triphenylphosphine and a carbon tetrahalide, triphenylphosphine dichloride, phosgene, oxalyl chloride or thionyl chloride, as described in US 2011/0136782.

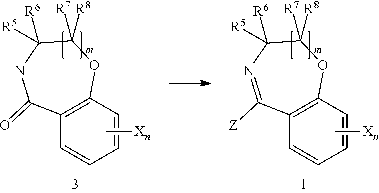

The cyclic amide compounds of the formula 3 wherein m is 0 (termed compounds 3′ in the following) are commercially available or can be prepared from the respective sali-cylic amide 4 by acetal formation with a dimethoxy alkane or a dimethoxy cycloalkane in an organic solvent and in the presence of an acid, like p-toluenesulfonic acid (p-TsOH), pyridinium p-toluenesulfonate, sulfuric acid or acetic acid (as described, for example, in Tetrahedron (2015), 71 (34), 5554-5561, Journal of Organic Chemistry (1981), 46 (16), 3340-2 or Bioorganic & Medicinal Chemistry (2006), 14 (6), 1978-1992). The compounds of the formula 3′ can also be prepared via condensation between sali-cylic amide 4 and ketones 6, catalyzed by secondary amines such as pyrrolidine, mor-pholine, etc. The reactions are best carried out in refluxing benzene or toluene with 10% amine catalyst (see for example J. Org. Chem. 1981, 46, 3340-3342 or Synthesis 1978, 886).

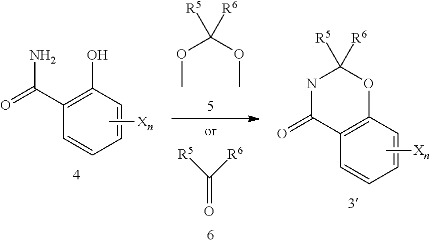

The cyclic amide compounds of the formula 3 wherein m is 1 (termed compounds 3″ in the following) can be prepared by reacting a cyclic acetophenone derivative 7 to an oxime 8, and then carrying out a Beckmann rearrangement to 3″, or alternatively convert-ing the cyclic acetophenone derivative 7 in one step by a Schmidt reaction to yield 3″ . . . . Various variations have been reported for both reactions. The Schmidt reaction can be carried out, for example, by reacting the ketone 7 in sodium azide in the presence of a strong acid, such as concentrated hydrochloric acid, sulfuric acid, trifluoroacetic acid or methane sulfonic acid, in the absence of a solvent or in a solvent such as acetonitrile, chloroform or methylene chloride. In the Beckmann rearrangement, the oxime 8 is reacted with polyphosphoric acid or a trimethylsilyl ester thereof, or is reacted at high temperature with a Lewis acid such as aluminum triiodide or iron (III) chloride-impreg-nated montmorillonite or with thionyl chloride in the absence of solvent or in the presence of a solvent such as acetonitrile. Alternatively, 3″ can be prepared by forming a mesylate or tosylate of the oxime 8 followed by treatment with a base such as aqueous sodium hydroxide solution or treating with a Lewis acid such as diethyl aluminum chloride, as described in Heterocycles 1994, 38 (2), 305-18 or in US 2011/0136782 A1.

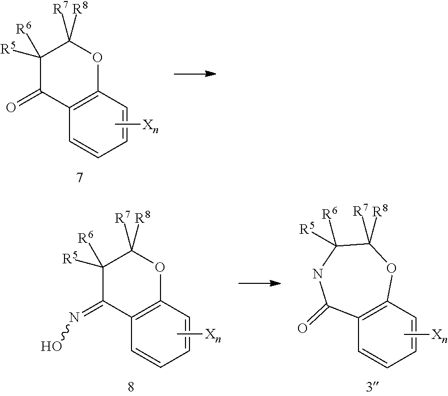

Alternatively, the compound 3″ can be prepared in an one pot reaction by copper (II)-catalyzed Beckmann rearrangement of ketone 7 under mild reaction conditions using hydroxylamine-O-sulfonic acid as aminating agent, as described in Synthesis 2019, 51 (19), 3709-3714.

The cyclic acetophenone derivatives 7 are commercially available or can be prepared starting from 2-hydroxyacetophenone via a classical ring closure reaction using the corresponding ketone in the presence of pyrrolidine, as described in Bioorganic & Medicinal Chemistry (2008), 16 (11), 6124-6130 or in Journal of the Chemical Society, Perkin Transactions 1: Organic and Bio-Organic Chemistry (1995).

Compounds I wherein m is 0 and Y is NR9 (termed I″ in the following) can be prepared from compound 10 by alkylation or acylation in the presence of a base such as potassium or sodium alkoxides or hydride. Dialkyl sulfates can also be used to effect said alkylation or acylation, as described in U.S. Pat. No. 3,625,959. Compounds 10 can be prepared from keto amine compound 9 by reaction with a ketone or aldehyde of the formula 6 in the presence of ammonium acetate. In some cases, the presence of an acid like p-toluenesulfonic acid (p-TsOH), pyridinium p-toluenesulfonate, sulfuric acid or acetic acid improves the yields (see for example Chemistry Select (2018), 3 (32), 9388-9392 and Organic & Biomolecular Chemistry (2003), 1 (2), 367-372).

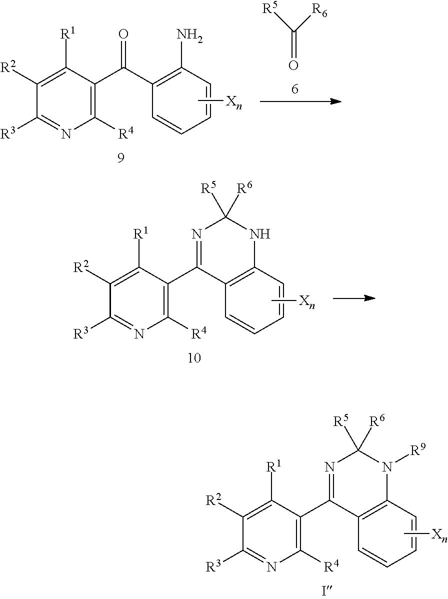

Compounds 9 are commercially available or can be obtained following the general pathway outlined in the following scheme by oxidation of the amino alcohol 14 using for example manganese dioxide, as described in Inorganica Chimica Acta (2012), 382, 72-78, WO 2000/038618, CN107879989 A, or Chinese Science Bulletin 2010, 55 (25), 2817-2819. Compounds 14 can be prepared via catalytic hydrogenation of the respective nitro alcohol 13 using Raney nickel, as described in WO 2000/038618 or in Inorganica Chimica Acta 2012, 382, 72-78. The nitro alcohol 13 can be prepared from 11 by phenyl magnesium bromide-mediated iodine-magnesium exchange in ortho position to NO2 as described by Knochel and coworkers (Angew. Chem., Int. Ed., 2002, 41, 1610), and subsequent addition to commercially available aldehyde 12.

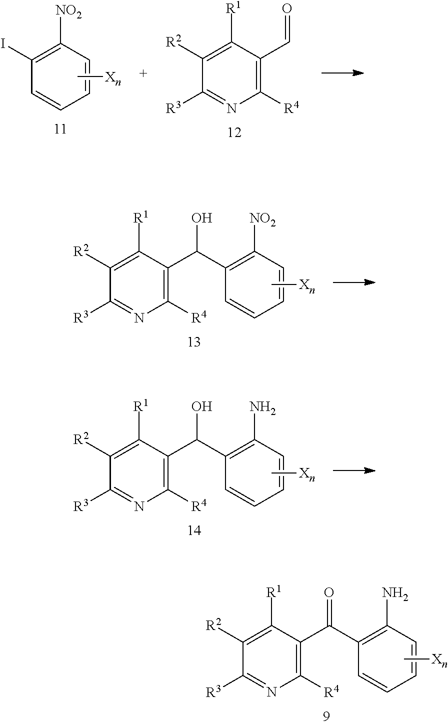

Compounds of the formula I″ wherein R⁹ is alkoxy can be prepared from 13 via following synthetic route, which is characterized by a selective catalytic hydrogenation of the nitro alcohol 13 to the corresponding N-arylhydroxylamines 15 using passivated Raney nickel which was treated by a combined liquid of aqueous ammonia and DMSO, as described in RSC Advances 2020, 28585-28594 or using platinum on carbon (type F 103 RS/W from Degussa), as described in IN1996CH00112.

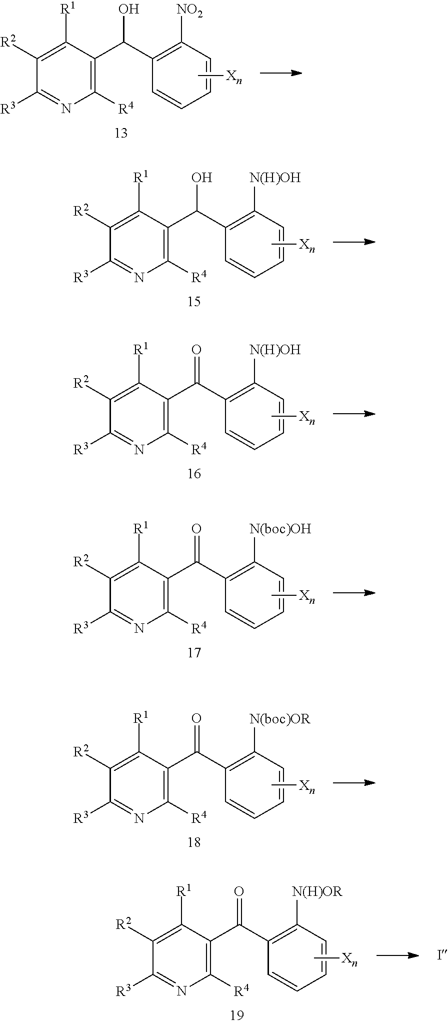

Compounds 16 can be prepared by oxidation of 15 using for example manganese dioxide, as described in Inorganica Chimica Acta 2012, 382, 72-78 and WO 2000/038618. The protected hydroxyl amine 17 can be prepared by methods well known in the literature for amino protecting groups as discussed in Theodora W. Greene's book “Protective Groups in Organic Synthesis”. The above-shown boc protective group is obtained, for example, by reacting with di-tert-butyldicarbonate in an appropriate solvent like DMSO. Compounds 17 can be alkylated using standard bases like LDA, NaH or NaHMDS to deprotonate the hydroxyl amine followed by addition of an alkylating agent with an appropriate leaving group like halide, mesylate, or triflate in an appropriate solvent to provide compounds 18 (see for example CN207973751). The boc protecting group can be removed by any method known in the literature like TFA in methylene chloride to give the compound 19 (see for example WO 2000/038618). Finally, compounds I″, wherein R⁹ is alkoxy can be prepared from 19 by treating with NH₄OAc as described in Chemistry Select, 2018, 3 (32), 9388-9392 or in Organic & Biomolecular Chemistry 2003, 1 (2), 367-372.

The N-oxides of compounds I may be prepared according to conventional oxidation methods, e.g. by treating compounds I with an organic peracid such as metachloroper-benzoic acid (cf. WO 03/64572 or J. Med. Chem. 38 (11), 1892-903, 1995); or with inorganic oxidizing agents such as hydrogen peroxide (cf. J. Heterocyc. Chem. 18 (7), 1305-8, 1981) or oxone (cf. J. Am. Chem. Soc. 123 (25), 5962-5973, 2001). The oxidation may lead to pure mono-N-oxides or to a mixture of different N-oxides, which can be separated by conventional methods such as chromatography.

As a rule, the compounds of formula I including their stereoisomers, salts, and N-oxides, and their precursors in the synthesis process, can be prepared by the methods described above. If individual compounds cannot be prepared via the above-described routes, they can be prepared by derivatization of other compounds I or the respective precursor or by customary modifications of the synthesis routes described. For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

The compounds I and the compositions thereof can be used in crop protection as foliar fungicides, fungicides for seed dressing, and soil fungicides.

The compounds I and the compositions thereof are preferably used in the control of phytopathogenic fungi as defined above or below on various cultivated plants, such as cereals, e. g. wheat, rye, barley, triticale, oats, or rice; beet, e. g. sugar beet or fodder beet; fruits, e. g. pomes (apples, pears, etc.), stone fruits (e.g. plums, peaches, al-monds, cherries), or soft fruits, also called berries (strawberries, raspberries, blackber-ries, gooseberries, etc.); leguminous plants, e. g. lentils, peas, alfalfa, or soybeans; oil plants, e. g. oilseed rape, mustard, olives, sunflowers, coconut, cocoa beans, castor oil plants, oil palms, ground nuts, or soybeans; cucurbits, e. g. squashes, cucumber, or melons; fiber plants, e. g. cotton, flax, hemp, or jute; citrus fruits, e. g. oranges, lemons, grapefruits, or mandarins; vegetables, e. g. spinach, lettuce, asparagus, cabbages, car-rots, onions, tomatoes, potatoes, cucurbits, or paprika; lauraceous plants, e. g. avoca-dos, cinnamon, or camphor; energy and raw material plants, e. g. corn, soybean, oilseed rape, sugar cane, or oil palm; corn; tobacco; nuts; coffee; tea; bananas; vines (table grapes and grape juice grape vines); hop; turf; sweet leaf (also called Stevia); natural rubber plants; or ornamental and forestry plants, e. g. flowers, shrubs, broad-leaved trees, or evergreens (conifers, eucalypts, etc.); on the plant propagation material, such as seeds; and on the crop material of these plants.

More preferably, compounds I and compositions thereof, respectively are used for controlling fungi as defined above or below on field crops, such as potatoes, sugar beets, tobacco, wheat, rye, barley, oats, rice, corn, cotton, soybeans, oilseed rape, leg-umes, sunflowers, coffee or sugar cane; fruits; vines; ornamentals; or vegetables, such as cucumbers, tomatoes, beans or squashes.

The term “plant propagation material” is to be understood to denote all the genera-tive parts of the plant, such as seeds; and vegetative plant materials, such as cuttings and tubers (e. g. potatoes), which can be used for the multiplication of the plant. This includes seeds, roots, fruits, tubers, bulbs, rhizomes, shoots, sprouts and other parts of plants; including seedlings and young plants to be transplanted after germination or after emergence from soil.

Preferably, treatment of plant propagation materials with compounds I and compositions thereof, respectively, is used for controlling fungi as defined above or below on cereals, such as wheat, rye, barley and oats; rice, corn, cotton and soybeans.

The term “cultivated plants” is to be understood as including plants which have been modified by mutagenesis or genetic engineering to provide a new trait to a plant or to modify an already present trait. Mutagenesis includes random mutagenesis using X-rays or mutagenic chemicals, but also targeted mutagenesis to create mutations at a specific locus of a plant genome. Targeted mutagenesis frequently uses oligonucleotides or proteins like CRISPR/Cas, zinc-finger nucleases, TALENs or meganucleases. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant to add a trait or improve or modify a trait. These integrated genes are also referred to as transgenes, while plant comprising such transgenes are referred to as transgenic plants. The process of plant transformation usually produces several transformation events, wich differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought.

Herbicide tolerance has been created by using mutagenesis and genetic engineering. Plants which have been rendered tolerant to acetolactate synthase (ALS) inhibitor herbicides by mutagenesis and breeding are e.g. available under the name Clearfield®. Herbicide tolerance to glyphosate, glufosinate, 2,4-D, dicamba, oxynil herbicides, like bromoxynil and ioxynil, sulfonylurea herbicides, ALS inhibitors and 4-hydroxyphenylpy-ruvate dioxygenase (HPPD) inhibitors, like isoxaflutole and mesotrione, has been created via the use of transgenes.

Transgenes to provide herbicide tolerance traits comprise: for tolerance to glyphosate: cp4 epsps, epsps grg23ace5, mepsps, 2mepsps, gat4601, gat4621, goxv247; for tolerance to glufosinate: pat and bar, for tolerance to 2,4-D: aad-1, aad-12; for tolerance to dicamba: dmo; for tolerance to oxynil herbicies: bxn; for tolerance to sulfonylurea herbicides: zm-hra, csr1-2, gm-hra, S4-HrA; for tolerance to ALS inhibitors: csr1-2;and for tolerance to HPPD inhibitors: hppdPF, W336, avhppd-03.

Transgenic corn events comprising herbicide tolerance genes include, but are not limited to, DAS40278, MON801, MON802, MON809, MON810, MON832, MON87411, MON87419, MON87427, MON88017, MON89034, NK603, GA21, MZHGOJG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC1507 and TC6275. Transgenic soybean events comprising herbicide tolerance genes include, but are not limited to, GTS 40-3-2, MON87705, MON87708, MON87712, MON87769, MON89788, A2704-12, A2704-21, A5547-127, A5547-35, DP356043, DAS44406-6, DAS68416-4,DAS-81419-2, GU262, SYHTØH2, W62, W98, FG72 and CV127. Transgenic cotton events comprising herbicide tolerance genes include, but are not limited to, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40. Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Transgenes to provide insect resistance preferably are toxin genes of Bacillus spp. and synthetic variants thereof, like crylA, crylAb, crylAb-Ac, crylAc, crylA.105, crylF, crylFa2, cry2Ab2, cry2Ae, mcry3A, ecry3.1Ab, cry3Bb1, cry34Ab1, cry35Ab1, cry9C, vip3A (a), vip3Aa20. In addition, transgenes of plant origin, such as genes coding for protease inhibitors, like CpTI and pinll, can be used. A further approach uses transgenes such as dvsnf7 to produce double-stranded RNA in plants.

Transgenic corn events comprising genes for insecticidal proteins or double stranded RNA include, but are not limited to, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098. Transgenic soybean events comprising genes for insecticidal proteins include, but are not limited to, MON87701, MON87751 and DAS-81419. Transgenic cotton events comprising genes for insecticidal proteins include, but are not limited to, SGK321, MON531, MON757, MON1076, MON15985, 31707, 31803, 31807, 31808, 42317, BNLA-601, Event1, COT67B, COT102, T303-3, T304-40, GFM CrylA, GK12, MLS 9124, 281-24-236, 3006-210-23, GHB119 and SGK321.

Cultivated plants with increased yield have been created by using the transgene athb17 (e.g. com event MON87403), or bbx32 (e.g. soybean event MON87712).

Cultivated plants comprising a modified oil content have been created by using the transgenes: gm-fad2-1, Pj.D6D, Nc.Fad3, fad2-1A and fatb1-A (e.g. soybean events 260-05, MON87705 and MON87769).

Tolerance to abiotic conditions, such as drought, has been created by using the transgene cspB (corn event MON87460) and Hahb-4 (soybean event IND-ØØ410-5).

Traits are frequently combined by combining genes in a transformation event or by combining different events during the breeding process resulting in a cultivated plant with stacked traits. Preferred combinations of traits are combinations of herbicide tolerance traits to different groups of herbicides, combinations of insect tolerance to different kind of insects, in particular tolerance to lepidopteran and coleopteran insects, combinations of herbicide tolerance with one or several types of insect resistance, combinations of herbicide tolerance with increased yield as well as combinations of herbicide tolerance and tolerance to abiotic conditions.

Plants comprising singular or stacked traits as well as the genes and events providing these traits are well known in the art. For example, detailed information as to the mutagenized or integrated genes and the respective events are available from web-sites of the organizations “International Service for the Acquisition of Agri-biotech Appli-cations (ISAAA)” (http://www.isaaa.org/gmapprovaldatabase) and the “Center for Envi-ronmental Risk Assessment (CERA)” (http://cera-gmc.org/GMCropDatabase). Further information on specific events and methods to detect them can be found for canola events MS1, MS8, RF3, GT73, MON88302, KK179 in WO01/031042, WO01/041558, WO01/041558, WO02/036831, WO11/153186, WO13/003558; for cotton events MON1445, MON15985, MON531 (MON15985), LLCotton25, MON88913, COT102, 281-24-236, 3006-210-23, COT67B, GHB614, T304-40, GHB119, MON88701, 81910 in WO02/034946, WO02/100163, WO02/100163, WO03/013224, WO04/072235, WO04/039986, WO05/103266, WO05/103266, WO06/128573, WO07/017186, WO08/122406, WO08/151780, WO12/134808, WO13/112527; for corn events GA21, MON810, DLL25, TC1507, MON863, MIR604, LY038, MON88017, 3272, 59122, NK603, MIR162, MON89034, 98140, 32138, MON87460, 5307, 4114, MON87427, DAS40278, MON87411, 33121, MON87403, MON87419 in WO98/044140, U.S. Ser. No. 02/102,582, U.S. Ser. No. 03/126,634, WO04/099447, WO04/011601, WO05/103301, WO05/061720, WO05/059103, WO06/098952, WO06/039376, US2007/292854, WO07/142840, WO07/140256, WO08/112019, WO09/103049, WO09/111263, WO10/077816, WO11/084621, WO11/062904, WO11/022469, WO13/169923, WO14/116854, WO15/053998, WO15/142571; for potato events E12, F10, J3, J55, V11, X17, Y9 in WO14/178910, WO14/178913, WO14/178941, WO14/179276, WO16/183445, WO17/062831, WO17/062825; for rice events LLRICE06, LLRICE601, LLRICE62 in WO00/026345, WO00/026356, WO00/026345; and for soybean events H7-1, MON89788, A2704-12, A5547-127, DP305423, DP356043, MON87701, MON87769, CV127, MON87705, DAS68416-4, MON87708, MON87712, SYHTOH2, DAS81419, DAS81419 x DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The use of compounds I and compositions thereof, respectively, on cultivated plants may result in effects which are specific to a cultivated plant comprising a certain transgene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

The compounds I and compositions thereof are also suitable for controlling harmful microorganisms in the protection of stored products or harvest, and in the protection of materials.

The term “stored products or harvest” is understood to denote natural substances of plant or animal origin and their processed forms for which long-term protection is desired. Stored products of plant origin, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity py-lons and barriers, or in the form of finished articles, such as furniture or objects made from wood. Stored products of animal origin are hides, leather, furs, hairs and alike. Preferably, “stored products” is understood to denote natural substances of plant origin and their processed forms, more preferably fruits and their processed forms, such as pomes, stone fruits, soft fruits and citrus fruits and their processed forms, where application of compounds I and compositions thereof can also prevent disadvantageous effects such as decay, discoloration or mold.

The term “protection of materials” is to be understood to denote the protection of technical and non-living materials, such as adhesives, glues, wood, paper, paperboard, textiles, leather, paint dispersions, plastics, cooling lubricants, fiber, or fabrics against the infestation and destruction by harmful microorganisms, such as fungi and bacteria. When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

The compounds I and compositions thereof, respectively, may be used for improving the health of a plant. The invention also relates to a method for improving plant health by treating a plant, its propagation material, and/or the locus where the plant is growing or is to grow with an effective amount of compounds I and compositions thereof, respectively.

The term “plant health” is to be understood to denote a condition of the plant and/or its products which is determined by several indicators alone or in combination with each other, such as yield (e. g. increased biomass and/or increased content of val-uable ingredients), plant vigor (e. g. improved plant growth and/or greener leaves (“greening effect”)), quality (e. g. improved content or composition of certain ingredients), and tolerance to abiotic and/or biotic stress. The above identified indicators for the health condition of a plant may be interdependent or may result from each other.

The compounds I are employed as such or in form of compositions by treating the fungi, the plants, plant propagation materials, such as seeds; soil, surfaces, materials, or rooms to be protected from fungal attack with a fungicidally effective amount of the active substances. The application can be carried out both before and after the in-fection of the plants, plant propagation materials, such as seeds; soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials, such as seeds, e. g. by dusting, coating, or drenching, amounts of active substance of generally from 0.1 to 1000 g, preferably from 1 to 1000 g, more preferably from 1 to 100 g and most preferably from 5 to 100 g, per 100 kg of plant propagation material (preferably seeds) are required.

An agrochemical composition comprises a fungicidally effective amount of a compound I. The term “fungicidally effective amount” denotes an amount of the composition or of the compounds I, which is sufficient for controlling harmful fungi on cultivated plants or in the protection of stored products or harvest or of materials and which does not result in a substantial damage to the treated plants, the treated stored products or harvest, or to the treated materials. Such an amount can vary in a broad range and is dependent on various factors, such as the fungal species to be controlled, the treated cultivated plant, stored product, harvest or material, the climatic conditions and the specific compound I used.

The user applies the agrochemical composition usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hec-tare of agricultural useful area.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types (see also “Catalogue of pesticide formulation types and international coding system”, Technical Monograph No. 2, 6th Ed. May 2008, CropLife International) are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials, such as seeds (e. g. GF). The compositions are prepared in a known manner, such as described by Mollet and Grubemann, Formulation technology, Wiley VCH, Weinheim, 2001; or by Knowles, New develop-ments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers, and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraf-fin, tetrahydronaphtha¬lene, and alkylated naphthalenes; alcohols, e. g. ethanol, pro-panol, butanol, benzyl alcohol, cyclo-hexanol, glycols; DMSO; ketones, e. g. cyclo¬hexanone; esters, e. g. lactates, carbonates, fatty acid esters, gamma-butyrolac-tone; fatty acids; phosphonates; amines; amides, e. g. N methyl pyrrolidone, fatty acid dimethyl amides; and mixtures thereof.

Suitable solid carriers or fillers are mineral earths, e. g. silicates, silica gels, talc, kao-lins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in Mccutcheon's, Vol. 1: Emulsifiers & Detergents, Mccutcheon's Directories, Glen Rock, USA, 2008 (International Ed. or North American Ed.).

Suitable anionic surfactants are alkali, alkaline earth or ammonium salts of sulf¬onates, sulfates, phosphates, carboxylates, and mixtures thereof. Examples of sulf¬onates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulf¬onates of dodecyl-and tridecylbenzenes, sulfonates of naphthalenes and of alkyl¬naphtha¬lenes, sulfosuccinates, or sulfosuccinamates. Examples of sulfates are sulfates of fatty acids, of oils, of ethoxylated alkylphenols, of alcohols, of ethoxy¬lated alcohols, or of fatty acid esters. Examples of phosphates are phosphate esters. Examples of carboxy¬lates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters, or monoglycerides. Examples of sugar-based surfactants are sorbitans, ethoxylated sorbitans, sucrose and glucose esters, or alkylpoly-glucosides. Examples of polymeric surfactants are home-or copolymers of vinyl pyrrolidone, vinyl alcohols, or vinyl acetate.

Suitable cationic surfactants are quaternary surfactants, for example quaternary am-monium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide, and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound Ion the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives, such as al-kyliso-thiazolinones and benzisothiazolinones.

Suitable anti-freezing agents are ethylene glycol, propylene glycol, urea and glyc-erin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo-and phthalocya-nine colorants).

Suitable tackifiers or binders are polyvinyl pyrrolidones, polyvinyl acetates, polyvinyl alcohols, polyacrylates, biological or synthetic waxes, and cellulose ethers.

The agrochemical compositions generally comprise between 0.01 and 95%, preferably between 0.1 and 90%, more preferably between 1 and 70%, and in particular between 10 and 60%, by weight of active substances (e.g. at least one compound I). The agrochemical compositions generally comprise between 5 and 99.9%, preferably between 10 and 99.9%, more preferably between 30 and 99%, and in particular between 40 and 90%, by weight of at least one auxiliary. The active substances (e.g. compounds I) are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions(ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, soaking, as well as in-furrow application methods. Preferably, compound I or the compositions thereof, respectively, are applied on to the plant propagation material by a method such that germination is not induced, e. g. by seed dressing, pelleting, coating, and dusting.

Various types of oils, wetters, adjuvants, fertilizers, or micronutrients, and further pesticides (e. g. fungicides, growth regulators, herbicides, insecticides, safeners) may be added to the compounds I or the compositions thereof as premix, or, not until imme-diately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial, or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoli-ants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

Biopesticides have been defined as a form of pesticides based on microorganisms (bacteria, fungi, viruses, nematodes, etc.) or natural products (compounds, such as metabolites, proteins, or extracts from biological or other natural sources) (U.S. Envi-ronmental Protection Agency: http://www.epa.gov/pesticides/biopesticides/). Biopesticides fall into two major classes, microbial and biochemical pesticides:
(1) Microbial pesticides consist of bacteria, fungi or viruses (and often include the metabolites that bacteria and fungi produce). Entomopathogenic nematodes are also classified as microbial pesticides, even though they are multicellular.
(2) Biochemical pesticides are naturally occurring substances that control pests or provide other crop protection uses as defined below, but are relatively non-toxic to mammals.

Mixing the compounds I or the compositions comprising them in the use form as fungicides with other fungicides results in many cases in an expansion of the fungicidal spectrum of activity or in a prevention of fungicide resistance development. Furthermore, in many cases, synergistic effects are obtained (synergistic mixtures).

The following list of pesticides II, in conjunction with which the compounds I can be used, is intended to illustrate the possible combinations but does not limit them:

A) Respiration inhibitors
Inhibitors of complex III at Q. site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysas-trobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichloro-phenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famox-adone (A.1.21), fenamidone (A.1.21), methyl-/-[2-[(1,4-dimethyl-5-phenyl-pyrazol-3-yl)oxylmethyl]phenyl]-N-methoxy-carbamate (A.1.22), metyltetraprole (A.1.25), (Z,2E)-5-[1-(2,4-dichlorophenyl) pyrazol-3-yl]-oxy-2-methoxyimino-N,3-dimethyl-pent-3-enamide (A.1.34), (Z,2E)-5-[1-(4-chlorophenyl) pyrazol-3-yl]oxy-2-methoxy-imino-N,3-dimethyl-pent-3-enamide (A.1.35), pyriminostrobin (A.1.36), bifujunzhi (A.1.37), 2-(ortho-((2,5-dimethylphenyl-oxymethylen) phenyl)-3-methoxy-acrylic acid methylester (A.1.38);
inhibitors of complex III at Qi site: cyazofamid (A.2.1), amisulbrom (A.2.2), [(6S,7 R,8 R)-8-benzyl-3-[(3-hydroxy-4-methoxy-pyridine-2-carbonyl)amino]-6-methyl-4,9-dioxo-1,5-dioxonan-7-yl] 2-methylpropanoate (A.2.3), fenpicoxamid (A.2.4), florylpicoxamid (A.2.5), [(1S,2S)-2-(4-fluoro-2-methyl-phenyl)-1,3-dimethyl-butyl](2S)-2-[(3-hydroxy-4-methoxy-pyridine-2-carbonyl)amino]propanoate, [(1S,2S)-2-(2,4-dimethylphenyl)-1,3-dimethyl-butyl](2S)-2-[(3-hydroxy-4-methoxy-pyridine-2-carbonyl)amino]propanoate,
[(1S,2S)-2-(2,4-difluorophenyl)-1,3-dimethyl-butyl](2S)-2-[(3-hydroxy-4-methoxy-pyridine-2-carbonyl)amino]propanoate, [(1S,2S)-2-(2-fluoro-4-methyl-phenyl)-1,3-dimethyl-butyl](2S)-2-[
(3-hydroxy-4-methoxy-pyridine-2-carbonyl)amino]propanoate, [(1S,2S)-2-(4-fluoro-2-methyl-phenyl)-1,3-dimethyl-butyl](2S)-2-[(3-acetoxy-4-methoxy-pyridine-2-carbonyl)amino]propanoate, [(1S,2S)-2-(2,4-dimethylphenyl)-1,3-dimethyl-butyl](2S)-2-[(3-acetoxy-4-methoxy-pyridine-2-carbonyl)amino]propanoate, [(1S,2S)-2-(2,4-difluorophenyl)-1,3-dimethyl-butyl](2S)-2-[(3-acetoxy-4-methoxy-pyridine-2-carbonyl)amino]propanoate, [(1S,2S)-2-(2-fluoro-4-methyl-phenyl)-1,3-dimethyl-butyl](2S)-2-[(3-acetoxy-4-methoxy-pyridine-2-carbonyl)amino]propanoate, [2-[(1S)-2-[(1S,2S)-2-(4-fluoro-2-methyl-phenyl)-1,3-dimethyl-butoxy]-1-methyl-2-oxo-ethyl]carbamoyl]-4-methoxy-3-pyridyl]oxymethyl 2-methylpropanoate, [2-[(1S)-2-[(1S,2S)-2-(2,4-dimethylphenyl)-1,3-dimethyl-butoxy]-1-methyl-2-oxo-ethyl]carbamoyl]-4-methoxy-3-pyridylloxymethyl 2-methylpropanoate, [2-[(1S)-2-[(1S,2S)-2-(2,4-difluorophenyl)-1,3-dimethyl-butoxy]-1-methyl-2-oxo-ethyl]carbamoyl]-4-methoxy-3-pyridyl]oxymethyl 2-methylpropanoate, [2-[(1S)-2-[(1S,2S)-2-(2-fluoro-4-methyl-phenyl)-1,3-dimethyl-butoxy]-1-methyl-2-oxo-ethyl]carbamoyl]-4-methoxy-3-pyridyl]oxymethyl 2-methylpropanoate, [(1S,2S)-1-methyl-2-(o-tolyl) propyl](2S)-2-[(3-acetoxy-4-methoxy-pyridine-2-carbonyl)amino]propanoate, [(1S,2S)-1-methyl-2-(o-tolyl) propyl](2S)-2-[(4-methoxy-3-propanoyloxy-pyridine-2-carbonyl) amino]propanoate, [(1S,2S)-1-methyl-2-(o-tolyl) propyl](2S)-2-[(3-hydroxy-4-methoxy-pyridine-2-carbonyl)amino]propanoate, [4-methoxy-2-[[(1S)-1-methyl-2-[(1S,2S)-1-methyl-2-(o-tolyl) propoxy]-2-oxo-ethyl]carbamoyl]-3-pyridyl]2-methylpropanoate, [(1S,2S)-2-(2,4-dimethylphenyl)-1-methyl-propyl](2S)-2-[(3-acetoxy-4-methoxy-pyridine-2-car-bonyl)amino]propanoate, [2-[[(1S)-2-[(1S,2S)-2-(2,4-dimethylphenyl)-1-methyl-propoxy]-1-methyl-2-oxo-ethyl]carbamoyl]-4-methoxy-3-pyridyl]2-methylpropanoate,
[(1S,2S)-2-(2,4-dimethylphenyl)-1-methyl-propyl](2S)-2-[(3-hydroxy-4-methoxy-pyri-dine-2-carbonyl)amino]propanoate, [(1S,2S)-2-(2,6-dimethylphenyl)-1-methyl-propyl](2S)-2-[(3-acetoxy-4-methoxy-pyridine-2-carbonyl)amino]propanoate, [2-[(1S)-2-[(1S,2S)-2-(2,6-dimethylphenyl)-1-methyl-propoxy]-1-methyl-2-oxo-ethyl]carbamoyl]-4-methoxy-3-pyridyl]2-methylpropanoate, [(1S,2S)-2-(2,6-dimethylphenyl)-1-methyl-propyl](2S)-2-[(3-hydroxy-4-methoxy-pyridine-2-carbonyl)amino]propanoate, [(1S,2S)-2-[4-fluoro-2-(trifluoromethyl)phenyl]-1-methyl-propyl](2S)-2-[(3-acetoxy-4-methoxy-pyridine-2-carbonyl)amino]propanoate, [2-[(1S)-2-[(1S,2S)-2-[4-fluoro-2-(trifluoromethyl)phenyl]-1-methyl-propoxy]-1-methyl-2-oxo-ethyl]carbamoyl]-4-methoxy-3-pyridyl]2-methylpropanoate, [(1S,2S)-2-[4-fluoro-2-(trifluoro-methyl)phenyl]-1-methyl-propyl](2S)-2-[(3-hydroxy-4-methoxy-pyridine-2-carbonyl)-amino]propanoate, [(1S,2S)-2-(4-fluoro-2-methyl-phenyl)-1-methyl-propyl](2S)-2-[(3-acetoxy-4-methoxy-pyridine-2-carbonyl)amino]propanoate, [2-[[(1S)-2-[(1S,2S)-2-(4-fluoro-2-methyl-phenyl)-1-methyl-propoxy]-1-methyl-2-oxo-ethyl]carbamoyl]-4-methoxy-3-pyridyl]2-methylpropanoate, [(1S,2S)-2-(4-fluoro-2-methyl-phenyl)-1-methyl-propyl](2S)-2-[(3-hydroxy-4-methoxy-pyridine-2-carbonyl)amino]propanoate, [(1S,2S)-1-methyl-2-[2-(trifluoromethyl)phenyl]propyl](2S)-2-[(3-acetoxy-4-methoxy-pyridine-2-carbonyl)amino]propanoate, [4-methoxy-2-[(1S)-1-methyl-2-[(1S,2S)-1-methyl-2-[2-(trifluoromethyl)phenyl]propoxy]-2-oxo-ethyl]carbamoyl]-3-pyridyl]2-methylpropanoate, [(1S,2S)-1-methyl-2-[2-(trifluoromethyl)phenyl]propyl](2S)-2-[(3-hydroxy-4-methoxy-pyridine-2-carbonyl)amino]propanoate, [(1S,2S)-2-(4-fluoro-2,6-dimethyl-phenyl)-1-methyl-propyl](2S)-2-[(3-acetoxy-4-methoxy-pyridine-2-carbonyl)amino]propanoate, [2-[[(1S)-2-[(1S,2S)-2-(4-fluoro-2,6-dimethyl-phenyl)-1-methyl-propoxy]-1-methyl-2-oxo-ethyl]carbamoyl]-4-methoxy-3-pyridyl]2-methylpropanoate, [(1S,2S)-2-(4-fluoro-2,6-dimethyl-phenyl)-1-methyl-propyl](2S)-2-[(3-hydroxy-4-methoxy-pyridine-2-carbonyl)amino]propanoate;
inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), the cloftalam (A.3.20), thifluzamide (A.3.21), inpyrfluxam (A.3.22), pyrapropoyne (A.3.23), fluindapyr (A.3.28), N-[2-[2-chloro-4-(trifluoromethyl) phenoxy]phenyl]-3-(difluoromethyl)-5-fluoro-1-methyl-pyrazole-4-carboxamide (A.3.29), methyl (E)-2-[2-[(5-cyano-2-methyl-phenoxy) methyl]phenyl]-3-methoxy-prop-2-enoate (A.3.30), isoflucypram (A.3.31), 2-(difluoromethyl)-N-(1,1,3-trimethyl-indan-4-yl)pyridine-3-carboxamide (A.3.32), 2-(difluoromethyl)-N-[(3R)-1, 1,3-trimethylindan-4-yl]pyridine-3-carboxamide (A.3.33), 2-(difluoromethyl)-N-(3-ethyl-1,1-dimethyl-indan-4-yl) pyri-dine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]pyridine-3-carboxamide (A.3.37), 2-(difluoromethyl)-N-(3-isobu-tyl-1,1-dimethyl-indan-4-yl)pyridine-3-carboxamide (A.3.38), 2-(difluoromethyl)-N-[(3R)-3-isobutyl-1,1-dimethyl-indan-4-yl]pyridine-3-carboxamide (A.3.39);
other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);

### B) Sterol Biosynthesis Inhibitors (SBI Fungicides)

C14 demethylase inhibitors: triazoles: azaconazole (B.1.1), bitertanol (B.1.2), bro-muconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), flu-quinconazole (B.1.10), flusilazole (B.1.11), flutriafol (B.1.12), hexaconazole (B.1.13), imibenconazole (B.1.14), ipconazole (B.1.15), metconazole (B.1.17), my-clobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy)phenyl]-2-pyridyl]propan-2-ol (B.1.32), 4-[6-[2-(2,4-difluorophenyl)-1,1-difluoro-2-hydroxy-3-(5-sulfanyl-1,2,4-triazol-1-yl) propyl]-3-pyridyl]oxy]benzonitrile (B.1.33), ipfentrifluconazole (B.1.37), mefentrifluconazole (B.1.38), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines, piperazines: fenarimol (B.1.49), pyrifenox (B.1.50), triforine (B.1.51), [3-(4-chloro-2-fluoro-phenyl)-5-(2,4-difluorophenyl) isoxazol-4-yl]-(3-pyridyl) methanol (B.1.52), 4-[6-[2-(2,4-difluorophenyl)-1,1-difluoro-2-hydroxy-3-(1,2,4-triazol-1-yl) propyl]-3-pyridyl]oxy]benzonitrile (B.1.53), 2-[6-(4-bromophenoxy)-2-(trifluoromethyl)-3-pyridyl]-1-(1,2,4-triazol-1-yl) propan-2-ol (B.1.54), 2-[6-(4-chlorophenoxy)-2-(trifluoro-methyl)-3-pyridyl]-1-(1,2,4-triazol-1-yl) propan-2-ol (B.1.55);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piper-alin (B.2.7), spiroxamine (B.2.8);
Inhibitors of 3-keto reductase: fenhexamid (B.3.1);
Other Sterol biosynthesis inhibitors: chlorphenomizole (B.4.1);

### C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
other nucleic acid synthesis inhibitors: hymexazole (C.2.1), octhilinone (C.2.2), ox-olinic acid (C.2.3), bupirimate (C.2.4), 5-fluorocytosine (C.2.5), 5-fluoro-2-(p-tol-ylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);

### D) Inhibitors of Cell Division and Cytoskeleton

tubulin inhibitors: benomyl (D.1.1), carbendazim (D.1.2), fuberidazole (D1.3), thia-bendazole (D.1.4), thiophanate-methyl (D.1.5), pyridachlometyl (D.1.6), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl) butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluoro-phenyl)-N-(2-chloro-6-fluoro-phenyl)-2,5-dimethyl-pyrazol-3-amine (D.1.16);
other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7), phenamacril (D.2.8);

### E) Inhibitors of Amino Acid and Protein Synthesis

methionine synthesis inhibitors: cyprodinil (E.1.1), mepanipyrim (E.1.2), pyrime-thanil (E.1.3);
protein synthesis inhibitors: blasticidin-S(E.2.1), kasugamycin (E.2.2), kasugamycin hydrochloride-hydrate (E.2.3), mildiomycin (E.2.4), streptomycin (E.2.5), oxytetracy-clin (E.2.6); 20

### F) Signal Transduction Inhibitors

MAP/histidine kinase inhibitors: fluoroimid (F.1.1), iprodione (F.1.2), procymidone (F.1.3), vinclozolin (F.1.4), fludioxonil (F.1.5);
G protein inhibitors: quinoxyfen (F.2.1);
   G) Lipid and membrane synthesis inhibitors
Phospholipid biosynthesis inhibitors: edifenphos (G.1.1), iprobenfos (G.1.2), pyrazo-phos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7), zinc thiazole (G.2.8); 30
phospholipid biosynthesis and cell wall deposition: dimethomorph (G.3.1), flumorph (G.3.2), mandipropamid (G.3.3), pyrimorph (G.3.4), benthiavalicarb (G.3.5), iprovali-carb (G.3.6), valifenalate (G.3.7);
compounds affecting cell membrane permeability and fatty acides: propamocarb (G.4.1);
inhibitors of oxysterol binding protein: oxathiapiprolin (G.5.1), fluoxapiprolin (G.5.3), 4-[1-[2-[3-(difluoromethyl)-5-methyl-pyrazol-1-yl]acetyl]-4-piperidyl]-N-tetralin-1-yl-pyridine-2-carboxamide (G.5.4), 4-[1-[2-[3,5-bis (difluoromethyl) pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl) pyrazol-1-yl]acetyl]-4-piperidyl]-N-tetralin-1-yl-pyridine-2-carboxamide (G.5.6), 4-[1-[2-[5-cyclopropyl-3-(difluoromethyl) pyrazol-1-yl]acetyl]-4-piperidyl]-N-tetralin-1-yl-pyridine-2-carboxamide (G.5.7), 4-[1-[2-[5-methyl-3-(trifluoro-methyl) pyrazol-1-yl]acetyl]-4-piperidyl]-N-tetralin-1-yl-pyridine-2-carboxamide (G.5.8), 4-[1-[2-[5-(difluoromethyl)-3-(trifluoromethyl) pyrazol-1-yl]acetyl]-4-piperidyl]-N-tetralin-1-yl-pyridine-2-carboxamide (G.5.9), 4-[1-[2-[3,5-bis(trifluoromethyl) pyra-zol-1-yl]acetyl]-4-piperidyl]-N-tetralin-1-yl-pyridine-2-carboxamide (G.5.10), (4-[1-[2-[5-cyclopropyl-3-(trifluoromethyl) pyrazol-1-yl]acetyl]-4-piperidyl]-/-tetralin-1-yl-pyri-dine-2-carboxamide (G.5.11);
   H) Inhibitors with Multi Site Action
inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
thio-and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris (albesilate) (H.4.8), dithianon (H.4.9), 2,6-di-methyl-1 H,5H-[1,4]dithiino [2,3-c: 5,6-c′]dipyrrole-1,3,5,7 (2H,6 H)-tetraone (H.4.10);

### I) Cell Wall Synthesis Inhibitors

inhibitors of glucan synthesis: validamycin (1.1.1), polyoxin B (1.1.2);
melanin synthesis inhibitors: pyroquilon (1.2.1), tricyclazole (1.2.2), carpropamid (1.2.3), dicyclomet (1.2.4), fenoxanil (1.2.5);

### J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-dimethoxyphenyl) thiadiazole-5-carboxamide (J.1.10);

### K) Unknown Mode of Action

bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitro-pan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), ni-trothal-isopropyl (K.1.20), tolprocarb (K.1.21), oxin-copper (K.1.22), proquinazid (K.1.23), tebufloquin (K.1.24), tecloftalam (K.1.25), triazoxide (K.1.26), N′ (4-(4-chloro-3-trifluoromethyl-phenoxy)-2,5-dimethyl-phenyl)-N-ethyl-N-methyl formamidine (K.1.27), N′ (4-(4-fluoro-3-trifluoromethyl-phenoxy)-2,5-dimethyl-phenyl)-N-ethyl-N-methyl formamidine (K.1.28), N′ [4-[3-[(4-chlorophenyl)methyl]-1,2,4-thiadiazol-5-yl]oxy]-2,5-dimethyl-phenyl]-N-ethyl-N-methyl-formamidine (K.1.29), N′ (5-bromo-6-indan-2-yloxy-2-methyl-3-pyridyl)-N-ethyl-N-methyl-formamidine (K.1.30), N′ [5-bromo-6-[1-(3,5-difluorophenyl) ethoxy]-2-methyl-3-pyridyl]-N-ethyl-N-methyl-formamidine (K.1.31), N′ [5-bromo-6-(4-isopropylcyclohexoxy)-2-methyl-3-pyridyl]-N-ethyl-N-methyl-formamidine (K.1.32), N′ [5-bromo-2-methyl-6-(1-phenylethoxy)-3-pyridyl]-N-ethyl-N-methyl-formamidine (K.1.33), N′ (2-methyl-5-trifluo-romethyl-4-(3-trimethylsilanyl-propoxy)-phenyl)-N-ethyl-N-methyl formamidine (K.1.34), N′ (5-difluoromethyl-2-methyl-4-(3-trimethylsilanyl-propoxy)-phenyl)-N ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrim-idin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[(Z)-[(1-methylte-trazol-5-yl)-phenyl-methylene]amino]oxymethyl]-2-pyridyl]carbamate (K.1.42), but-3-ynyl N-[6-[(Z)-[(1-methyltetrazol-5-yl)-phenyl-methylene]amino]oxymethyl]-2-pyridyl]carbamate (K.1.43), ipflufenoquin (K.1.44), quinofumelin (K.1.47), benziothi-azolinone (K.1.48), bromothalonil (K.1.49), 2-(6-benzyl-2-pyridyl) quinazoline (K.1.50), 2-[6-(3-fluoro-4-methoxy-phenyl)-5-methyl-2-pyridyl]quinazoline (K.1.51), dichlobentiazox (K.1.52), N′ (2,5-dimethyl-4-phenoxy-phenyl)-N-ethyl-N-methyl-formamidine (K.1.53), aminopyrifen (K.1.54), fluopimomide (K.1.55), N-[5-bromo-2-methyl-6-(1-methyl-2-propoxy-ethoxy)-3-pyridyl]-N-ethyl-N-methyl-formamidine (K. 1.56), N′-[4-(4,5-dichlorothiazol-2-yl)oxy-2,5-dimethyl-phenyl]-N-ethyl-N-methyl-formamidine (K.1.57), N-(2-fluorophenyl)-4-[5-(trifluoromethyl)-1,2,4-oxadiazol-3-yl]benzamide (K.1.58), N-methyl-4-[5-(trifluoromethyl)-1,2,4-oxadiazol-3-yl]benzene-carbothioamide (K.1.59);

### L) Biopesticides

L1) Microbial pesticides with fungicidal, bactericidal, viricidal and/or plant defense activator activity: Ampelomyces quisqualis, Aspergillus flavus, Aureobasidium pullulans, Bacillus altitudinis, B. amyloliquefaciens, B. amyloliquefaciens ssp. plantarum (also referred to as B. velezensis), B. megaterium, B. mojavensis, B. mycoides, B. pumilus, B. simplex, B. solisalsi, B. subtilis, B. subtilis var. am-yloliquefaciens, B. velezensis, Candida oleophila, C. saitoana, Clavibacter michiganensis (bacteriophages), Coniothyrium minitans, Cryphonectria para-sitica, Cryptococcus albidus, Dilophosphora alopecuri, Fusarium oxysporum, Clonostachys roseaf. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus alvei, Paenibacillus epiphyticus, P. polymyxa, Pantoea vagans, Penicillium bilaiae, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperelloides, T. asperellum, T. atroviride, T. fertile, T. gamsii, T. harmatum, T. harzianum, T. polysporum, T. stromaticum, T. virens, T. viride, Typhula phacorrhiza, Ulocladium oudemansii, Verticillium dahlia, zucchini yellow mosaic virus (avirulent strain);
L2) Biochemical pesticides with fungicidal, bactericidal, viricidal and/or plant defense activator activity: harpin protein, Reynoutria sachalinensis extract;
L3) Microbial pesticides with insecticidal, acaricidal, molluscidal and/or nematicidal activity: Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniartii, Burkholderia spp., Chromobacterium subtsugae, Cydia pomonella granulovirus (CpGV), Cryptophlebia leucotreta granulovirus (CrleGV), Flavobacterium spp., Helicoverpa armigera nucleopolyhedrovirus (HearNPV), Helicoverpa zea nucleopolyhedrovirus (HzNPV), Helicoverpa zea single capsid nucleopolyhedrovirus (HzSNPV), Heterorhabditis bacteriophora, Isaria fumosorosea, Lecanicillium longisporum, L. muscarium, Metarhizium anisopliae, M. anisopliae var. anisopliae, M. anisopliae var. acridum, Nomuraea rileyi, Paecilomyces fumosoroseus, P. lilacinus, Paenibacillus popilliae, Pasteuria spp., P. nishizawae, P. penetrans, P. ramosa, P. thomea, P. usgae, Pseudomonas fluorescens, Spodoptera littoralis nucleopolyhedrovirus (SpliNPV), Steinernema carpocapsae, S. feltiae, S. kraussei, Streptomyces galbus, S. microflavus,
L4) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadeca-dien-1-ol, (R)-1-octen-3-ol, pentatermanone, (E,Z,Z)-3,8,11-tetradecatrienyl acetate, (Z,E)-9,12-tetradecadien-1-yl acetate, (Z)-7-tetradecen-2-one, (Z)-9-tetradecen-1-yl acetate, (Z)-11-tetradecenal, (Z)-11-tetradecen-1-ol, extract of Chenopodium ambrosiodes, Neem oil, Quillay extract;
L5) Microbial pesticides with plant stress reducing, plant growth regulator, plant growth promoting and/or yield enhancing activity: Azospirillum amazonense, A. brasilense, A. lipoferum, A. irakense, A. halopraeferens, Bradyrhizobium spp., B. elkanii, B. japonicum, B. liaoningense, B. lupini, Delftia acidovorans, Glomus intraradices, Mesorhizobium spp., Rhizobium leguminosarum bv. phaseoli, R. I. bv. trifolii, R. I. bv. viciae, R. tropici, Sinorhizobium meliloti,
   O) Insecticides from Classes O.1 to 0.29
O.1 Acetylcholine esterase (AChE) inhibitors: aldicarb, alanycarb, bendiocarb, benfura-carb, butocarboxim, butoxycarboxim, carbaryl, carbofuran, carbosulfan, ethiofen-carb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate; acephate, azamethiphos, azinphos-ethyl, azinphosmethyl, ca-dusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, mal-athion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sul-fotep, tebupirimfos, temephos, terbufos, tetrachlorvinphos, thiometon, triazophos, trichlorfon, vamidothion;
O.2 GABA-gated chloride channel antagonists: endosulfan, chlordane; ethiprole, fipronil, flufiprole, pyrafluprole, pyriprole;
O.3 Sodium channel modulators: acrinathrin, allethrin, d-cis-trans allethrin, d-trans allethrin, bifenthrin, kappa-bifenthrin, bioallethrin, bioallethrin S-cyclopentenyl, bio-resmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfen-valerate, etofenprox, fenpropathrin, fenvalerate, flucythrinate, flumethrin, tau-fluvali-nate, halfenprox, heptafluthrin, imiprothrin, meperfluthrin, metofluthrin, momfluorothrin, epsilon-momfluorothrin, permethrin, phenothrin, prallethrin, profluthrin, pyre-thrin (pyrethrum), resmethrin, silafluofen, tefluthrin, kappa-tefluthrin, tetra-methylfluthrin, tetramethrin, tralomethrin, transfluthrin; DDT, methoxychlor;
O.4 Nicotinic acetylcholine receptor (nAChR) agonists: acetamiprid, clothianidin, cy-cloxaprid, dinotefuran, imidacloprid, nitenpyram, thiacloprid, thiamethoxam; 4,5-di-hydro-N-nitro-1-(2-oxiranylmethyl)-1 H-imidazol-2-amine, (2E)-1-[(6-chloropyridin-3-yl)methyl]-N′nitro-2-pentylidenehydrazinecarboximidamide; 1-[(6-chloropyridin-3-yl)methyl]-7-methyl-8-nitro-5-propoxy-1,2,3,5,6,7-hexahydroimidazo[1,2-a]pyridine; nicotine; sulfoxaflor, flupyradifurone, triflumezopyrim, (3R)-3-(2-chlorothiazol-5-yl)-8-methyl-5-oxo-6-phenyl-2,3-dihydrothiazolo[3,2-a]pyrimidin-8-ium-7-olate, (3S)-3-(6-chloro-3-pyridyl)-8-methyl-5-oxo-6-phenyl-2,3-dihydrothiazolo[3,2-a]pyrimidin-8-ium-7-olate, (3S)-8-methyl-5-oxo-6-phenyl-3-pyrimidin-5-yl-2,3-dihydrothiazolo[3,2-a]py-rimidin-8-ium-7-olate, (3R)-3-(2-chlorothiazol-5-yl)-8-methyl-5-oxo-6-[3-(trifluoromethyl)phenyl]-2,3-dihydrothiazolo[3,2-a]pyrimidin-8-ium-7-olate; (3R)-3-(2-chlorothia-zol-5-yl)-6-(3,5-dichlorophenyl)-8-methyl-5-oxo-2,3-dihydrothiazolo[3,2-a]pyrimidin-8-ium-7-olate, (3R)-3-(2-chlorothiazol-5-yl)-8-ethyl-5-oxo-6-phenyl-2,3-dihydrothiazolo [3,2-a]pyrimidin-8-ium-7-olate;
O.5 Nicotinic acetylcholine receptor allosteric activators: spinosad, spinetoram;
O.6 Chloride channel activators: abamectin, emamectin benzoate, ivermectin, lepimec-tin, milbemectin;
O.7 Juvenile hormone mimics: hydroprene, kinoprene, methoprene; fenoxycarb, pyriproxyfen;
O.8 miscellaneous non-specific (multi-site) inhibitors: methyl bromide and other alkyl halides; chloropicrin, sulfuryl fluoride, borax, tartar emetic;
O.9 Chordotonal organ TRPV channel modulators: pymetrozine, pyrifluquinazon;
O.10 Mite growth inhibitors: clofentezine, hexythiazox, diflovidazin; etoxazole;
O.11 Microbial disruptors of insect midgut membranes: Bacillus thuringiensis, Bacillus sphaericus and the insecticdal proteins they produce: Bacillus thuringiensis subsp. israelensis, Bacillus sphaericus, Bacillus thuringiensis subsp. aizawai, Bacillus thuringiensis subsp. kurstaki, Bacillus thuringiensis subsp. tenebrionis, the Bt crop proteins: CrylAb, CrylAc, CrylFa, Cry2Ab, mCry3A, Cry3Ab, Cry3Bb, Cry34/35Ab1;
O.12 Inhibitors of mitochondrial ATP synthase: diafenthiuron; azocyclotin, cyhexatin, fenbutatin oxide, propargite, tetradifon;
O.13 Uncouplers of oxidative phosphorylation via disruption of the proton gradient: chlorfenapyr, DNOC, sulfluramid;
O.14 Nicotinic acetylcholine receptor (nAChR) channel blockers: bensultap, cartap hydrochloride, thiocyclam, thiosultap sodium;
O.15 Inhibitors of the chitin biosynthesis type 0: bistrifluron, chlorfluazuron, difluben-zuron, flucycloxuron, flufenoxuron, hexaflumuron, lufenuron, novaluron, noviflumuron, teflubenzuron, triflumuron;
O.16 Inhibitors of the chitin biosynthesis type 1: buprofezin;
O.17 Moulting disruptors: cyromazine;
O.18 Ecdyson receptor agonists: methoxyfenozide, tebufenozide, halofenozide, fufeno-zide, chromafenozide;
0.19 Octopamin receptor agonists: amitraz;
O.20 Mitochondrial complex III electron transport inhibitors: hydramethylnon, acequi-nocyl, fluacrypyrim, bifenazate;
O.21 Mitochondrial complex I electron transport inhibitors: fenazaquin, fenpyroximate, pyrimidifen, pyridaben, tebufenpyrad, tolfenpyrad; rotenone;
O.22 Voltage-dependent sodium channel blockers: indoxacarb, metaflumizone, 2-[2-(4-cyanophenyl)-1-[3-(trifluoromethyl)phenyl]ethylidene]-N-[4-(difluoromethoxy) phenyl]-hydrazinecarboxamide, N-(3-chloro-2-methylphenyl)-2-[(4-chlorophenyl)-[4-[methyl (methylsulfonyl) amino]phenyl]methylene]-hydrazinecarboxamide;
O.23 Inhibitors of the of acetyl CoA carboxylase: spirodiclofen, spiromesifen, spirotetra-mat, spiropidion;
O.24 Mitochondrial complex IV electron transport inhibitors: aluminium phosphide, calcium phosphide, phosphine, zinc phosphide, cyanide;
O.25 Mitochondrial complex II electron transport inhibitors: cyenopyrafen, cyflumetofen;
O.26 Ryanodine receptor-modulators: flubendiamide, chlorantraniliprole, cyan-traniliprole, cyclaniliprole, tetraniliprole; (R)-3-chloro-M-{2-methyl-4-[1,2,2,2-tetra-fluoro-1-(trifluoromethyl)ethyl]phenyl}-/2-(1-methyl-2-methylsulfonylethyl) phthalamide, (S)-3-chloro-M-{2-methyl-4-[1,2,2,2-tetrafluoro-1-(trifluo-romethyl)ethyl]phenyl}-N-(1-methyl-2-methylsulfonylethyl) phthalamide, methyl-2-[3,5-dibromo-2- ({[3-bromo-1-(3-chloropyridin-2-yl)-1H-pyrazol-5-yl]carbonyl}-amino) benzoyl]-1,2-dimethylhydrazinecarboxylate; N-[4,6-dichloro-2-[(diethyl-lambda-4-sulfanylidene) carbamoyl]-phenyl]-2-(3-chloro-2-pyridyl)-5-(trifluoromethyl) pyrazole-3-carboxamide; N-[4-chloro-2-[(diethyl-lambda-4-sulfanylidene)carbamoyl]-6-methyl-phenyl]-2-(3-chloro-2-pyridyl)-5-(trifluoromethyl) pyrazole-3-carboxamide; N-[4-chloro-2-[(di-2-propyl-lambda-4-sulfanylidene) carbamoyl]-6-methyl-phenyl]-2-(3-chloro-2-pyridyl)-5-(trifluoromethyl) pyrazole-3-carboxamide;/-[4,6-di-chloro-2-[(di-2-propyl-lambda-4-sulfanylidene) carbamoyl]-phenyl]-2-(3-chloro-2-pyridyl)-5-(trifluoromethyl) pyrazole-3-carboxamide; N-[4,6-dibromo-2-[(diethyl-lambda-4-sulfanylidene) carbamoyl]-phenyl]-2-(3-chloro-2-pyridyl)-5-(trifluoromethyl) pyrazole-3-carboxamide; N-[2-(5-amino-1,3,4-thiadiazol-2-yl)-4-chloro-6-methylphenyl]-3-bromo-1-(3-chloro-2-pyridinyl)-1 H-pyrazole-5-carboxamide; 3-chloro-1-(3-chloro-2-pyridinyl)-N-[2,4-dichloro-6-[(1-cyano-1-methylethyl) amino]car-bonyl]phenyl]-1 H-pyrazole-5-carboxamide; tetrachlorantraniliprole; N-[4-chloro-2-[[(1,1-dimethylethyl) amino]carbonyl]-6-methylphenyl]-1-(3-chloro-2-pyridinyl)-3-(fluo-romethoxy)-1 H-pyrazole-5-carboxamide; cyhalodiamide;
O.27: Chordotonal organ modulators-undefined target site: flonicamid;
O.28. insecticidal compounds of unknown or uncertain mode of action: afidopyropen, afoxolaner, azadirachtin, amidoflumet, benzoximate, broflanilide, bromopropylate, chinomethionat, cryolite, dicloromezotiaz, dicofol, flufenerim, flometoquin, fluensul-fone, fluhexafon, fluopyram, fluralaner, metoxadiazone, piperonyl butoxide, pyflu-bumide, pyridalyl, tioxazafen, 11-(4-chloro-2,6-dimethylphenyl)-12-hydroxy-1,4-di-oxa-9-azadispiro[4.2.4.2]-tetradec-11-en-10-one, 3-(4′-fluoro-2,4-dimethylbiphenyl-3-yl)-4-hydroxy-8-oxa-1-azaspiro[4.5]dec-3-en-2-one, 1-[2-fluoro-4-methyl-5-[(2,2,2-trifluoroethyl) sulfinyl]phenyl]-3-(trifluoromethyl)-1 H-1,2,4-triazole-5-amine, Bacillus firmus I-1582; flupyrimin; fluazaindolizine; 4-[5-(3,5-dichlorophenyl)-5-(trifluoromethyl)-4 H-isoxazol-3-yl]-2-methyl-N-(1-oxothietan-3-yl)benzamide ; fluxametamide; 5-[3-[2,6-dichloro-4-(3,3-dichloroallyloxy) phenoxy]propoxy]-1 H-pyrazole; 4-cyano-N-[2-cyano-5-[2,6-dibromo-4-[1,2,2,3,3,3-hexafluoro-1-(trifluoromethyl) propyl]phenyl]carbamoyl]phenyl]-2-methyl-benzamide; 4-cyano-3-[(4-cyano-2-methyl-benzoyl) amino]-N-[2,6-dichloro-4-[1,2,2,3,3,3-hexafluoro-1-(trifluoromethyl) propyl]phenyl]-2-fluoro-benzamide; N-[5-[2-chloro-6-cyano-4-[1,2,2,3,3,3-hexafluoro-1-(trifluo-romethyl) propyl]phenyl]carbamoyl]-2-cyano-phenyl]-4-cyano-2-methyl-benzamide; N-[5-[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; N-[5-[2-bromo-6-chloro-4-[1,2,2,3,3,3-hexafluoro-1-(trifluoromethyl) propyl]phenyl]carbamoyl]-2-cy-ano-phenyl]-4-cyano-2-methyl-benzamide; 4-cyano-N-[2-cyano-5-[2,6-dichloro-4-[1,2,2,3,3,3-hexafluoro-1-(trifluoromethyl) propyl]phenyl]carbamoyl]phenyl]-2-methyl-benzamide; 4-cyano-N-[2-cyano-5-[2,6-dichloro-4-[1,2,2,2-tetrafluoro-1-(trifluoromethyl)ethyl]phenyl]carbamoyl]phenyl]-2-methyl-benzamide; N-[5-[2-bromo-6-chloro-4-[1,2,2,2-tetrafluoro-1-(trifluoromethyl)ethyl]phenyl]carbamoyl]-2-cyano-phenyl]-4-cy-ano-2-methyl-benzamide; 2-(1,3-dioxan-2-yl)-6-[2-(3-pyridinyl)-5-thiazolyl]-pyridine; 2-[6-[2-(5-fluoro-3-pyridinyl)-5-thiazolyl]-2-pyridinyl]-pyrimidine; 2-[6-[2-(3-pyridinyl)-5-thiazolyl]-2-pyridinyl]-pyrimidine; N-methylsulfonyl-6-[2-(3-pyridyl) thiazol-5-yl]pyri-dine-2-carboxamide; N-methylsulfonyl-6-[2-(3-pyridyl) thiazol-5-yl]pyridine-2-carboxamide; 1-[(6-chloro-3-pyridinyl)methyl]-1,2,3,5,6,7-hexahydro-5-methoxy-7-methyl-8-nitro-imidazo[1,2-a]pyridine; 1-[(6-chloropyridin-3-yl)methyl]-7-methyl-8-nitro-1,2,3,5,6,7-hexahydroimidazo[1,2-a]pyridin-5-ol; 1-isopropyl-N,5-dimethyl-N-pyri-dazin-4-yl-pyrazole-4-carboxamide; 1-(1,2-dimethylpropyl)-N-ethyl-5-methyl-N-pyri-dazin-4-yl-pyrazole-4-carboxamide; N,5-dimethyl-N-pyridazin-4-yl-1-(2,2,2-trifluoro-1-methyl-ethyl) pyrazole-4-carboxamide; 1-[1-(1-cyanocyclopropyl)ethyl]-N-ethyl-5-methyl-N-pyridazin-4-yl-pyrazole-4-carboxamide; N-ethyl-1-(2-fluoro-1-methyl-propyl)-5-methyl-N-pyridazin-4-yl-pyrazole-4-carboxamide; 1-(1,2-dimethylpropyl)-N,5-dimethyl-N-pyridazin-4-yl-pyrazole-4-carboxamide; 1-[1-(1-cyanocyclopropyl)ethyl]-N,5-dimethyl-N-pyridazin-4-yl-pyrazole-4-carboxamide; N-methyl-1-(2-fluoro-1-methyl-propyl]-5-methyl-N-pyridazin-4-yl-pyrazole-4-carboxamide; 1-(4,4-difluorocyclo-hexyl)-N-ethyl-5-methyl-N-pyridazin-4-yl-pyrazole-4-carboxamide; 1-(4,4-difluorocy-clohexyl)-N,5-dimethyl-N-pyridazin-4-yl-pyrazole-4-carboxamide, N-(1-methylethyl)-2-(3-pyridinyl)-2H-indazole-4-carboxamide; N-cyclopropyl-2-(3-pyridinyl)-2H-inda-zole-4-carboxamide; N-cyclohexyl-2-(3-pyridinyl)-2H-indazole-4-carboxamide; 2-(3-pyridinyl)-N-(2,2,2-trifluoroethyl)-2H-indazole-4-carboxamide; 2-(3-pyridinyl)-N-[(tet-rahydro-2-furanyl)methyl]-2H-indazole-5-carboxamide; methyl 2-[2-(3-pyridinyl)-2H-indazol-5-yl]carbonyl]hydrazinecarboxylate; N-[(2,2-difluorocyclopropyl)methyl]-2-(3-pyridinyl)-2H-indazole-5-carboxamide; N-(2,2-difluoropropyl)-2-(3-pyridinyl)-2H-inda-zole-5-carboxamide; 2-(3-pyridinyl)-N-(2-pyrimidinylmethyl)-2H-indazole-5-carboxamide; N-[(5-methyl-2-pyrazinyl)methyl]-2-(3-pyridinyl)-2H-indazole-5-carboxamide, tyclopyrazoflor; sarolaner, lotilaner, N-[4-chloro-3-[(phenylmethyl) amino]car-bonyl]phenyl]-1-methyl-3-(1, 1,2,2,2-pentafluoroethyl)-4-(trifluoromethyl)-1H-pyra-zole-5-carboxamide; 2-(3-ethylsulfonyl-2-pyridyl)-3-methyl-6-(trifluoromethyl) imidazo [4,5-b]pyridine, 2-[3-ethylsulfonyl-5-(trifluoromethyl)-2-pyridyl]-3-methyl-6-(trifluo-romethyl) imidazo[4,5-b]pyridine, isocycloseram, N-[4-chloro-3-(cyclopropylcar-bamoyl)phenyl]-2-methyl-5-(1,1,2,2,2-pentafluoroethyl)-4-(trifluoromethyl) pyrazole-3-carboxamide, N-[4-chloro-3-[(1-cyanocyclopropyl) carbamoyl]phenyl]-2-methyl-5-(1, 1,2,2,2-pentafluoroethyl)-4-(trifluoromethyl) pyrazole-3-carboxamide; acyn-onapyr; benzpyrimoxan; tigolaner; chloro-N-(1-cyanocyclopropyl)-5-[1-[2-methyl-5-(1, 1,2,2,2-pentafluoroethyl)-4-(trifluoromethyl) pyrazol-3-yl]pyrazol-4-yl]benzamide, oxazosulfyl, [(2S,3R,4 R,5 S,6 S)-3,5-dimethoxy-6-methyl-4-propoxy-tetrahydropyran-2-yl]-N-[4-[1-[4-(trifluoromethoxy)phenyl]-1,2,4-triazol-3-yl]phenyl]carbamate, [(2S,3 R,4 R,5 S,6 S)-3,4,5-trimethoxy-6-methyl-tetrahydropyran-2-yl]N-[4-[1-[4-(tri-fluoromethoxy)phenyl]-1,2,4-triazol-3-yl]phenyl]carbamate, [(2S,3 R,4 R,5 S,6 S)-3,5-dimethoxy-6-methyl-4-propoxy-tetrahydropyran-2-yl]-N-[4-[1-[4-(1,1,2,2,2-pentafluo-roethoxy)phenyl]-1,2,4-triazol-3-yl]phenyl]carbamate, [(2S,3 R,4 R,5 S,6 S)-3,4,5-tri-methoxy-6-methyl-tetrahydropyran-2-yl]-N-[4-[1-[4-(1,1,2,2,2-pentafluoroethoxy) phenyl]-1,2,4-triazol-3-yl]phenyl]carbamate, (2Z)-3-(2-isopropylphenyl)-2-[(E)-[4-[1-[4-(1,1,2,2,2-pentafluoroethoxy)phenyl]-1,2,4-triazol-3-yl]phenyl]methylenehydra-zono]thiazolidin-4-one; 2-(6-chloro-3-ethylsulfonyl-imidazo[1,2-a]pyridin-2-yl)-3-methyl-6-(trifluoromethyl) imidazo[4,5-b]pyridine, 2-(6-bromo-3-ethylsulfonyl-imidazo [1,2-a]pyridin-2-yl)-3-methyl-6-(trifluoromethyl) imidazo[4,5-b]pyridine, 2-(3-ethyl-sulfonyl-6-iodo-imidazo[1,2-a]pyridin-2-yl)-3-methyl-6-(trifluoromethyl) imidazo-[4,5-b]pyridine, 2-[3-ethylsulfonyl-6-(trifluoromethyl) imidazo[1,2-a]pyridin-2-yl]-3-methyl-6-(trifluoromethyl) imidazo[4,5-b]pyridine, 2-(7-chloro-3-ethylsulfonyl-imidazo [1,2-a]pyridin-2-yl)-3-methyl-6-(trifluoromethyl) imidazo[4,5-b]pyridine, 2-(3-ethyl-sulfonyl-7-iodo-imidazo[1,2-a]pyridin-2-yl)-3-methyl-6-(trifluoromethyl) imidazo-[4,5-b]pyridine, 3-ethylsulfonyl-6-iodo-2-[3-methyl-6-(trifluoromethyl) imidazo[4,5-b]pyridin-2-yl]imidazo[1,2-a]pyridine-8-carbonitrile, 2-[3-ethylsulfonyl-8-fluoro-6-(trifluoromethyl) imidazo[1,2-a]pyridin-2-yl]-3-methyl-6-(trifluoromethyl) imidazo[4,5-b]pyridine, 2-[3-ethylsulfonyl-7-(trifluoromethyl) imidazo[1,2-a]pyridin-2-yl]-3-methyl-6-(trifluoromethylsulfinyl) imidazo[4,5-b]pyridine, 2-[3-ethylsulfonyl-7-(trifluoromethyl) imidazo[1,2-a]pyridin-2-yl]-3-methyl-6-(trifluoromethyl) imidazo[4,5-c]pyridine, 2-(6-bromo-3-ethylsulfonyl-imidazo[1,2-a]pyridin-2-yl)-6-(trifluoromethyl) pyrazolo[4,3-c]pyridine.

The active substances referred to as component 2, their preparation and their activity e. g. against harmful fungi is known (cf.: http://www.alanwood.net/pesticides/); these substances are commercially available. The compounds described by IUPAC nomen-clature, their preparation and their pesticidal activity are also known (cf. Can. J. Plant Sci. 48 (6), 587-94, 1968; EP-A 141 317; EP-A 152 031; EP-A 226 917; EP-A 243 970;EP-A 256 503; EP-A 428 941; EP-A 532 022; EP-A 1 028 125; EP-A 1 035 122;EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024,CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441, WO 16/156241, WO 16/162265). Some compounds are identified by their CAS Registry Number which is separated by hyphens into three parts, the first consisting from two up to seven digits, the second consisting of two digits, and the third consisting of a single digit.

According to the invention, the solid material (dry matter) of the biopesticides (with the exception of oils such as Neem oil) are considered as active components (e. g. to be obtained after drying or evaporation of the extraction or suspension medium in case of liquid formulations of the microbial pesticides). The weight ratios and percentages used for a biological extract such as Quillay extract are based on the total weight of the dry content (solid material) of the respective extract(s).

The total weight ratios of compositions comprising at least one microbial pesticide in the form of viable microbial cells including dormant forms, can be determined using the amount of CFU of the respective microorganism to calculate the total weight of the respective active component with the following equation that 1×10¹⁰ CFU equals one gram of total weight of the respective active component. Colony forming unit is measure of viable microbial cells. In addition, CFU may also be understood as the number of (juvenile) individual nematodes in case of nematode biopesticides, such as Steinernema feltiae.

In the binary mixtures the weight ratio of the component 1) and the component 2)generally depends from the properties of the components used, usually it is in the range of from 1:10,000 to 10,000:1, often from 1:100 to 100:1, regularly from 1:50 to 50:1, preferably from 1:20 to 20:1, more preferably from 1:10 to 10:1, even more preferably from 1:4 to 4:1 and in particular from 1:2 to 2:1. According to further embodiments, the weight ratio of the component 1) and the component 2) usually is in the range of from 1000:1 to 1:1, often from 100:1 to 1:1, regularly from 50:1 to 1:1, preferably from 20:1 to 1:1, more preferably from 10:1 to 1:1, even more preferably from 4:1 to 1:1 and in particular from 2:1 to 1:1. According to further embodiments, the weight ratio of the component 1) and the component 2) usually is in the range of from 20,000:1 to 1:10, often from 10,000:1 to 1:1, regularly from 5,000:1 to 5:1, preferably from 5,000:1 to 10:1, more preferably from 2,000:1 to 30:1, even more preferably from 2,000:1 to 100:1 and in particular from 1,000:1 to 100:1. According to further embodiments, the weight ratio of the component 1) and the component 2) usually is in the range of from 1:1 to 1:1000, often from 1:1 to 1:100, regularly from 1:1 to 1:50, preferably from 1:1 to 1:20, more preferably from 1:1 to 1:10, even more preferably from 1:1 to 1:4 and in particular from 1:1 to 1:2. According to further embodiments, the weight ratio of the component 1) and the component 2) usually is in the range of from 10:1 to 1:20,000, often from 1:1 to 1:10,000, regularly from 1:5 to 1:5,000, preferably from 1:10 to 1:5,000, more preferably from 1:30 to 1:2,000, even more preferably from 1:100 to 1:2,000 to and in particular from 1:100 to 1:1,000.

In the ternary mixtures, i.e. compositions comprising the component 1) and component 2) and a compound III (component 3), the weight ratio of component 1) and component 2) depends from the properties of the active substances used, usually it is in the range of from 1:100 to 100:1, regularly from 1:50 to 50:1, preferably from 1:20 to 20:1, more preferably from 1:10 to 10:1 and in particular from 1:4 to 4:1, and the weight ratio of component 1) and component 3) usually it is in the range of from 1:100 to 100:1, regularly from 1:50 to 50:1, preferably from 1:20 to 20:1, more preferably from 1:10 to 10:1 and in particular from 1:4 to 4:1. Any further active components are, if desired, added in a ratio of from 20:1 to 1:20 to the component 1). These ratios are also suitable for mixtures applied by seed treatment.

When mixtures comprising microbial pesticides are employed in crop protection, the application rates range from 1×10⁶ to 5×10¹⁶ (or more) CFU/ha, preferably from 1 ×10⁸ to 1×10¹³ CFU/ha, and even more preferably from 1×10⁹ to 5×10¹⁵ CFU/ha and in particular from 1×10¹² to 5×10¹⁴ CFU/ha. In the case of nematodes as microbial pesticides (e. g. Steinernema feltiae), the application rates regularly range from 1×10⁵ to 1×10¹² (or more), preferably from 1×10⁸ to 1×10¹¹, more preferably from 5×10⁸ to 1×10¹⁰ individuals (e. g. in the form of eggs, juvenile or any other live stages, preferably in an infetive juvenile stage) per ha.

When mixtures comprising microbial pesticides are employed in seed treatment, the application rates generally range from 1×10⁶ to 1×10¹² (or more) CFU/seed, preferably from 1×10⁶ to 1×10⁹ CFU/seed. Furthermore, the application rates with respect to seed treatment generally range from 1×10⁷ to 1×10¹⁴ (or more) CFU per 100 kg of seed, preferably from 1×10⁹ to 1×10¹² CFU per 100 kg of seed.

Preference is given to mixtures comprising as component 2) at least one active substance selected from inhibitors of complex III at Qₒ site in group A), more preferably selected from compounds (A.1.1), (A.1.4), (A.1.8), (A.1.9), (A.1.10), (A.1.12), (A.1.13), (A.1.14), (A.1.17), (A.1.21), (A.1.25), (A.1.34) and (A.1.35); particularly selected from (A.1.1), (A.1.4), (A.1.8), (A.1.9), (A.1.13), (A.1.14), (A.1.17), (A.1.25), (A.1.34) and (A.1.35).

Preference is also given to mixtures comprising as component 2) at least one active substance selected from inhibitors of complex III at Qᵢ site in group A), more preferably selected from compounds (A.2.1), (A.2.3) and (A.2.4); particularly selected from (A.2.3)and (A.2.4).

Preference is also given to mixtures comprising as component 2) at least one active substance selected from inhibitors of complex II in group A), more preferably selected from compounds (A.3.2), (A.3.3), (A.3.4), (A.3.7), (A.3.9), (A.3.11), (A.3.12), (A.3.15), (A.3.16), (A.3.17), (A.3.18), (A.3.19), (A.3.20), (A.3.21), (A.3.22), (A.3.23), (A.3.28), (A.3.31), (A.3.32), (A.3.33), (A.3.34), (A.3.35), (A.3.36), (A.3.37), (A.3.38) and (A.3.39); particularly selected from (A.3.2), (A.3.3), (A.3.4), (A.3.7), (A.3.9), (A.3.12), (A.3.15), (A.3.17), (A.3.19), (A.3.22), (A.3.23), (A.3.31), (A.3.32), (A.3.33), (A.3.34), (A.3.35), (A.3.36), (A.3.37), (A.3.38) and (A.3.39).

Preference is also given to mixtures comprising as component 2) at least one active substance selected from other respiration nhibitors in group A), more preferably selected from compounds (A.4.5) and (A.4.11); in particular (A.4.11).

Preference is also given to mixtures comprising as component 2) at least one active substance selected from C14 demethylase inhibitors in group B), more preferably selected from compounds (B.1.4), (B.1.5), (B.1.8), (B.1.10), (B.1.11), (B.1.12), (B.1.13), (B.1.17), (B.1.18), (B.1.21), (B.1.22), (B.1.23), (B.1.25), (B.1.26), (B.1.29), (B.1.34), (B.1.37), (B.1.38), (B.1.43), (B.1.46), (B.1.53), (B.1.54) and (B.1.55); particularly selected from (B.1.5), (B.1.8), (B.1.10), (B.1.17), (B.1.22), (B.1.23), (B.1.25), (B.1.33), (B.1.34), (B.1.37), (B.138), (B.1.43) and (B.1.46).

Preference is also given to mixtures comprising as component 2) at least one active substance selected from Delta14-reductase inhibitors in group B), more preferably selected from compounds (B.2.4), (B.2.5), (B.2.6) and (B.2.8); in particular (B.2.4).

Preference is also given to mixtures comprising as component 2) at least one active substance selected from phenylamides and acyl amino acid fungicides in group C), more preferably selected from compounds (C.1.1), (C.1.2), (C.1.4) and (C.1.5); particularly selected from (C.1.1) and (C.1.4).

Preference is also given to mixtures comprising as component 2) at least one active substance selected from other nucleic acid synthesis inhibitors in group C), more preferably selected from compounds (C.2.6), (C.2.7) and (C.2.8).

Preference is also given to mixtures comprising as component 2) at least one active substance selected from group D), more preferably selected from compounds (D.1.1), (D.1.2), (D.1.5), (D.2.4) and (D.2.6); particularly selected from (D.1.2), (D.1.5) and (D.2.6).

Preference is also given to mixtures comprising as component 2) at least one active substance selected from group E), more preferably selected from compounds (E.1.1), (E.1.3), (E.2.2) and (E.2.3); in particular (E.1.3).

Preference is also given to mixtures comprising as component 2) at least one active substance selected from group F), more preferably selected from compounds (F.1.2), (F.1.4) and (F.1.5).

Preference is also given to mixtures comprising as component 2) at least one active substance selected from group G), more preferably selected from compounds (G.3.1), (G.3.3), (G.3.6), (G.5.1), (G.5.3), (G.5.4), (G.5.5), G.5.6), G.5.7), (G.5.8), (G.5.9), (G.5.10) and (G.5.11); particularly selected from (G.3.1), (G.5.1) and (G.5.3).

Preference is also given to mixtures comprising as component 2) at least one active substance selected from group H), more preferably selected from compounds (H.2.2), (H.2.3), (H.2.5), (H.2.7), (H.2.8), (H.3.2), (H.3.4), (H.3.5), (H.4.9) and (H.4.10); particularly selected from (H.2.2), (H.2.5), (H.3.2), (H.4.9) and (H.4.10).

Preference is also given to mixtures comprising as component 2) at least one active substance selected from group I), more preferably selected from compounds (1.2.2)and (1.2.5).

Preference is also given to mixtures comprising as component 2) at least one active substance selected from group J), more preferably selected from compounds (J.1.2), (J.1.5), (J.1.8), (J.1.11) and (J.1.12); in particular (J.1.5).

Preference is also given to mixtures comprising as component 2) at least one active substance selected from group K), more preferably selected from compounds (K.1.41), (K.1.42), (K.1.44), (K.1.47), (K.1.57), (K.1.58) and (K.1.59); particularly selected from (K.1.41), (K.1.44), (K.1.47), (K.1.57), (K.1.58) and (K.1.59).

The biopesticides from group L1) and/or L2) may also have insecticidal, acaricidal, molluscidal, pheromone, nematicidal, plant stress reducing, plant growth regulator, plant growth promoting and/or yield enhancing activity. The biopesticides from group L3) and/or L4) may also have fungicidal, bactericidal, viricidal, plant defense activator, plant stress reducing, plant growth regulator, plant growth promoting and/or yield enhancing activity. The biopesticides from group L5) may also have fungicidal, bactericidal, viricidal, plant defense activator, insecticidal, acaricidal, molluscidal, pheromone and/or nematicidal activity.

The microbial pesticides, in particular those from groups L1), L3) and L5), embrace not only the isolated, pure cultures of the respective microorganism as defined herein, but also its cell-free extract, its suspension in a whole broth culture and a metabolite-containing culture medium or a purified metabolite obtained from a whole broth culture of the microorganism.

Many of these biopesticides have been deposited under deposition numbers mentioned herein (the prefices such as ATCC or DSM refer to the acronym of the respective culture collection, for details see e. g. here: http://www.wfcc.info/ccinfo/collec-tion/by_acronym/), are referred to in literature, registered and/or are commercially available: mixtures of Aureobasidium pullulans DSM 14940 and DSM 14941 isolated in 1989 in Konstanz, Germany (e. g. blastospores in BlossomProtect® from bio-ferm GmbH, Austria), Azospirillum brasilense Sp245 originally isolated in wheat reagion of South Brazil (Passo Fundo) at least prior to 1980 (BR 11005; e. g. GELFIX® Gramíneas from BASF Agricultural Specialties Ltd., Brazil), A. brasilense strains Ab-V5 and Ab-V6 (e. g. in AzoMax from Novozymes BioAg Produtos papra Agricultura Ltda., Quattro Barras, Brazil or Simbiose-Maíz® from Simbiose-Agro, Brazil; Plant Soil 331, 413-425, 2010), Bacillus amyloliquefaciens strain AP-188 (NRRL B-50615 and B-50331; U.S. Pat. No. 8,445,255); B. amyloliquefaciens ssp. plantarum strains formerly also some-times referred to as B. subtilis, recently together with B. methylotrophicus, and B. velezensis classified as B. velezensis (Int. J. Syst. Evol. Microbiol. 66, 1212-1217, 2016): B. a. ssp. plantarum or B. velezensis D747 isolated from air in Kikugawa-shi, Japan (US20130236522 A1; FERM BP-8234; e. g. Double Nickel™ 55 WDG from Certis LLC, USA), B. a. ssp. plantarum or B. velezensis FZB24 isolated from soil in Brandenburg, Germany (also called SB3615; DSM 96-2; J. Plant Dis. Prot. 105, 181-197, 1998; e. g. Taegro® from Novozyme Biologicals, Inc., USA), B. a. ssp. plantarum or B. velezensis FZB42 isolated from soil in Brandenburg, Germany (DSM 23117; J. Plant Dis. Prot. 105, 181-197, 1998; e. g. RhizoVital® 42 from AbiTEP GmbH, Germany), B. a. ssp. plantarum or B. velezensis MBI600 isolated from faba bean in Sutton Bonington, Nottinghamshire, U.K. at least before 1988 (also called 1430; NRRL B-50595;US 2012/0149571 A1; e. g. Integral® from BASF Corp., USA), B. a. ssp. plantarum or B. velezensis QST-713 isolated from peach orchard in 1995 in California, U.S.A. (NRRL B-21661; e. g. Serenade® MAX from Bayer Crop Science LP, USA), B. a. ssp. plantarum or B. velezensis TJ1000 isolated in 1992 in South Dakoda, U.S.A. (also called 1BE; ATCC BAA-390; CA 2471555 A1; e. g. QuickRoots™ from TJ Technolo-gies, Watertown, SD, USA); B. firmus CNCM I-1582, a variant of parental strain EIP-N1 (CNCM I-1556) isolated from soil of central plain area of Israel (WO 2009/126473, U.S. Pat. No. 6,406,690; e. g. Votivo® from Bayer CropScience LP, USA), B. pumilus GHA 180 isolated from apple tree rhizosphere in Mexico (IDAC 260707-01; e. g. PRO-MIX® BX from Premier Horticulture, Quebec, Canada), B. pumilus INR-7 otherwise referred to as BU-F22 and BU-F33 isolated at least before 1993 from cucumber infested by Erwinia tracheiphila (NRRL B-50185, NRRL B-50153; U.S. Pat. No. 8,445,255), B. pumilus KFP9F isolated from the rhizosphere of grasses in South Africa at least before 2008 (NRRL B-50754; WO 2014/029697; e. g. BAC-UP or FUSION-P from BASF Agricultural Specialities (Pty) Ltd., South Africa), B. pumilus QST 2808 was isolated from soil collected in Pohnpei, Federated States of Micronesia, in 1998 (NRRL B-30087; e. g. Sonata® or Ballad® Plus from Bayer Crop Science LP, USA), B. simplex ABU 288 (NRRL B-50304; U.S. Pat. No. 8,445,255), B. subtilis FB17 also called UD 1022 or UD10-22 isolated from red beet roots in North America (ATCC PTA-11857; System. Appl. Microbiol. 27, 372-379, 2004; US 2010/0260735; WO 2011/109395); B. thuringiensis ssp. aizawai ABTS-1857 isolated from soil taken from a lawn in Ephraim, Wisconsin, U.S.A., in 1987 (also called ABG-6346; ATCC SD-1372; e. g. XenTari® from BioFa AG, Münsingen, Germany), B. t. ssp. kurstaki ABTS-351 identical to HD-1 isolated in 1967 from diseased Pink Bollworm black larvae in Brownsville, Texas, U.S.A. (ATCC SD-1275; e. g. Dipel® DF from Valent BioSciences, IL, USA), B. t. ssp. kurstaki SB4 isolated from E. saccha-rina larval cadavers (NRRL B-50753; e. g. Beta Pro® from BASF Agricultural Specialities (Pty) Ltd., South Africa), B. t. ssp. tenebrionis NB-176-1, a mutant of strain NB-125, a wild type strain isolated in 1982 from a dead pupa of the beetle Tenebrio molitor (DSM 5480; EP 585 215 B1; e. g. Novodor® from Valent BioSciences, Switzerland), Beauveria bassiana GHA (ATCC 74250; e. g. BotaniGard® 22WGP from Laverlam Int. Corp., USA), B. bassiana JW-1 (ATCC 74040; e. g. Naturalis® from CBC (Europe) S.r.l., Italy), B. bassiana PPRI 5339 isolated from the larva of the tortoise beetle Conchyloctenia punctata (NRRL 50757; e. g. BroadBand® from BASF Agricultural Specialities (Pty) Ltd., South Africa), Bradyrhizobium elkanii strains SEMIA 5019 (also called 29W) isolated in Rio de Janeiro, Brazil and SEMIA 587 isolated in 1967 in the State of Rio Grande do Sul, from an area previously inoculated with a North American isolate, and used in commercial inoculants since 1968 (Appl. Environ. Microbiol. 73 (8), 2635, 2007; e. g. GELFIX 5 from BASF Agricultural Specialties Ltd., Brazil), B. japonicum 532c isolated from Wisconsin field in U.S.A. (Nitragin 61A152; Can. J. Plant. Sci. 70, 661-666, 1990; e. g. in Rhizoflo®, Histick®, Hicoat® Super from BASF Agricultural Specialties Ltd., Canada), B. japonicum E-109 variant of strain USDA 138 (INTA E109, SEMIA 5085; Eur. J. Soil Biol. 45, 28-35, 2009; Biol. Fertil. Soils 47, 81-89, 2011); B. japonicum strains deposited at SEMIA known from Appl. Environ. Microbiol. 73 (8), 2635, 2007: SEMIA 5079 isolated from soil in Cerrados region, Brazil by Embrapa-Cerrados used in commercial inoculants since 1992 (CPAC 15; e. g. GELFIX 5 or ADHERE 60 from BASF Agricultural Specialties Ltd., Brazil), B. japonicum SEMIA 5080 obtained under lab condtions by Embrapa-Cerrados in Brazil and used in commercial inoculants since 1992, being a natural variant of SEMIA 586 (CB1809) originally isolated in U.S.A. (CPAC 7; e. g. GELFIX 5 or ADHERE 60 from BASF Agricultural Specialties Ltd., Brazil); Burkholderia sp. A396 isolated from soil in Nikko, Japan, in 2008 (NRRL B-50319; WO 2013/032693; Marrone Bio Innovations, Inc., USA), Coniothyrium minitans CON/M/91-08 isolated from oilseed rape (WO 1996/021358; DSM 9660; e. g. Contans® WG, Intercept® WG from Bayer CropScience AG, Germany), harpin (alpha-beta) protein (Science 257, 85-88, 1992; e. g. Messenger™ or HARP—N-Tek from Plant Health Care plc, U.K.), Helicoverpa armigera nucleopolyhedrovirus (HearNPV) (J. In-vertebrate Pathol. 107, 112-126, 2011; e. g. Helicovex® from Adermatt Biocontrol, Switzerland; Diplomata® from Koppert, Brazil; Vivus@ Max from AgBiTech Pty Ltd., Queensland, Australia), Helicoverpa zea single capsid nucleopolyhedrovirus (HzSNPV) (e. g. Gemstar® from Certis LLC, USA), Helicoverpa zea nucleopolyhedrovirus ABA-NPV-U (e. g. Heligen® from AgBiTech Pty Ltd., Queensland, Australia), Heterorhabditis bacteriophora (e. g. Nemasys® G from BASF Agricultural Specialities Limited, UK), Isaria fumosorosea Apopka-97 isolated from mealy bug on gynura in Apopka, Florida, U.S.A. (ATCC 20874; Biocontrol Science Technol. 22 (7), 747-761, 2012; e. g. PFR-97™ or PreFeRal® from Certis LLC, USA), Metarhizium anisopliae var. anisopliae F52 also called 275 or V275 isolated from codling moth in Austria (DSM 3884, ATCC 90448; e. g. Met52® Novozymes Biologicals BioAg Group, Canada), Metschnikowia fructicola 277 isolated from grapes in the central part of Israel (U.S. Pat. No. 6,994,849; NRRL Y-30752; e. g. formerly Shemer® from Agrogreen, Israel), Paecilomyces ilacinus 251 isolated from infected nematode eggs in the Philippines (AGAL 89/030550; WO1991/02051; Crop Protection 27, 352-361, 2008; e. g. BioAct®from Bayer CropScience AG, Germany and MeloCon® from Certis, USA), Paenibacillus alvei NAS6G6 isolated from the rhizosphere of grasses in South Africa at least before 2008 (WO 2014/029697; NRRL B-50755; e.g. BAC-UP from BASF Agricultural Specialities (Pty) Ltd., South Africa), Paenibacillus strains isolated from soil samples from a variety of European locations including Germany: P. epiphyticus Lu17015 (WO 2016/020371;DSM 26971), P. polymyxa ssp. plantarum Lu16774 (WO 2016/020371; DSM 26969), P. p. ssp. plantarum strain Lu17007 (WO 2016/020371; DSM 26970); Pasteuria nishizawae Pn1 isolated from a soybean field in the mid-2000s in Illinois, U.S.A. (ATCC SD-5833; Federal Register 76 (22), 5808 Feb. 2, 2011; e.g. Clariva™ PN from Syngenta Crop Protection, LLC, USA), Penicillium bilaiae (also called P. bilaii) strains ATCC 18309 (=ATCC 74319), ATCC 20851 and/or ATCC 22348 (=ATCC 74318)originally isolated from soil in Alberta, Canada (Fertilizer Res. 39, 97-103, 1994; Can. J. Plant Sci. 78 (1), 91-102, 1998; U.S. Pat. No. 5,026,417, WO 1995/017806; e. g. Jump Start®, Provide® from Novozymes Biologicals BioAg Group, Canada), Reynoutria sachalinensis extract (EP 0307510 B1; e. g. Regalia® SC from Marrone BioInnovations, Da-vis, CA, USA or Milsana® from BioFa AG, Germany), Steinernema carpocapsae (e. g. Millenium® from BASF Agricultural Specialities Limited, UK), S. feltiae (e. g. Nemashield® from BioWorks, Inc., USA; Nemasys® from BASF Agricultural Specialities Limited, UK), Streptomyces microflavus NRRL B-50550 (WO 2014/124369; Bayer CropScience, Germany), Trichoderma asperelloides JM41R isolated in South Africa (NRRL 50759; also referred to as T. fertile, e. g. Trichoplus® from BASF Agricultural Specialities (Pty) Ltd., South Africa), T. harzianum T-22 also called KRL-AG2 (ATCC 20847; BioControl 57, 687-696, 2012; e. g. Plantshield® from BioWorks Inc., USA or SabrEx™ from Advanced Biological Marketing Inc., Van Wert, OH, USA).

According to another embodiment of the mixtures, the at least one pesticide II is selected from the groups L1) to L5):
L1) Microbial pesticides with fungicidal, bactericidal, viricidal and/or plant defense activator activity: Aureobasidium pullulans DSM 14940 and DSM 14941 (L1.1), Bacillus amyloliquefaciens AP-188 (L.1.2), B. amyloliquefaciens ssp. plantarum D747 (L.1.3), B. amyloliquefaciens ssp. plantarum FZB24 (L.1.4), B. amyloliquefaciens ssp. plantarum FZB42 (L.1.5), B. amyloliquefaciens ssp. plantarum MBI600 (L. 1.6), B. amyloliquefaciens ssp. plantarum QST-713 (L.1.7), B. amyloliquefaciens ssp. plantarum TJ1000 (L.1.8), B. pumilus GB34 (L.1.9), B. pumilus GHA 180 (L.1.10), B. pumilus INR-7 (L.1.11), B. pumilus KFP9F (L.1.12), B. pumilus QST 2808 (L.1.13), B. simplex ABU 288 (L.1.14), B. subtilis FB17 (L.1.15), Coniothyrium minitans CON/M/91-08 (L.1.16), Metschnikowia fructicola NRRL Y-30752 (L.1.17), Paenibacillus alvei NAS6G6 (L.1.18), P. epiphyticus Lu17015 (L.1.25), P. polymyxa ssp. plantarum Lu16774 (L.1.26), P. p. ssp. plantarum strain Lu17007 (L.1.27), Penicillium bilaiae ATCC 22348 (L.1.19), P. bilaiae ATCC 20851 (L.1.20), Penicillium bilaiae ATCC 18309 (L.1.21), Streptomyces microflavus NRRL B-50550 (L.1.22), Trichoderma asperelloides JM41R (L.1.23), T. harzianum T-22 (L.1.24);
L2) Biochemical pesticides with fungicidal, bactericidal, viricidal and/or plant defense activator activity: harpin protein (L.2.1), Reynoutria sachalinensis extract (L.2.2);
L3) Microbial pesticides with insecticidal, acaricidal, molluscidal and/or nematicidal activity: Bacillus firmus I-1582 (L.3.1); B. thuringiensis ssp. aizawai ABTS-1857 (L.3.2), B. t. ssp. kurstaki ABTS-351 (L.3.3), B. t. ssp. kurstaki SB4 (L.3.4), B. t. ssp. tenebrionis NB-176-1 (L.3.5), Beauveria bassiana GHA (L.3.6), B. bassiana JW-1 (L.3.7), B. bassiana PPRI 5339 (L.3.8), Burkholderia sp. A396 (L.3.9), Helicoverpa armigera nucleopolyhedrovirus (HearNPV) (L.3.10), Helicoverpa zea nucleopolyhedrovirus (HzNPV) ABA-NPV-U (L.3.11), Helicoverpa zea single capsid nucleopolyhedrovirus (HzSNPV) (L.3.12), Heterohabditis bacteriophora (L.3.13), Isaria fumosorosea Apopka-97 (L.3.14), Metarhizium anisopliae var. anisopliae F52 (L.3.15), Paecilomyces lilacinus 251 (L.3.16), Pasteuria nishizawae Pn1 (L.3.17), Steinernema carpocapsae (L.3.18), S. feltiae (L.3.19);
L4) Biochemical pesticides with insecticidal, acaricidal, molluscidal, pheromone and/or nematicidal activity: cis-jasmone (L.4.1), methyl jasmonate (L.4.2), Quillay extract (L.4.3);
L5) Microbial pesticides with plant stress reducing, plant growth regulator, plant growth promoting and/or yield enhancing activity: Azospirillum brasilense Ab-V5 and Ab-V6 (L.5.1), A. brasilense Sp245 (L.5.2), Bradyrhizobium elkanii SEMIA 587 (L.5.3), B. elkanii SEMIA 5019 (L.5.4), B. japonicum 532c (L.5.5), B. japonicum E-109 (L.5.6), B. japonicum SEMIA 5079 (L.5.7), B. japonicum SEMIA 5080 (L.5.8).

The present invention furthermore relates to agrochemical compositions comprising a mixture of at least one compound I (component 1) and at least one biopesticide selected from the group L) (component 2), in particular at least one biopesticide selected from the groups L1) and L2), as described above, and if desired at least one suitable auxiliary.

The present invention furthermore relates to agrochemical compositions comprising a mixture of of at least one compound I (component 1) and at least one biopesticide selected from the group L) (component 2), in particular at least one biopesticide selected from the groups L3) and L4), as described above, and if desired at least one suitable auxiliary.

Preference is also given to mixtures comprising as pesticide II (component 2) a biopesticide selected from the groups L1), L3) and L5), preferably selected from strains denoted above as (L.1.2), (L.1.3), (L.1.4), (L.1.5), (L.1.6), (L.1.7), (L.1.8), (L.1.10), (L.1.11), (L.1.12), (L.1.13), (L.1.14), (L.1.15), (L.1.17), (L.1.18), (L.1.19), (L.1.20), (L.1.21), (L.1.25), (L.1.26), (L.1.27), (L.3.1); (L.3.9), (L.3.16), (L.3.17), (L.5.1), (L.5.2), (L.5.3), (L.5.4), (L.5.5), (L.5.6), (L.5.7), (L.5.8); (L.4.2), and (L.4.1); even more preferably selected from (L.1.2), (L.1.6), (L.1.7), (L.1.8), (L.1.11), (L.1.12), (L.1.13), (L.1.14), (L.1.15), (L.1.18), (L.1.19), (L.1.20), (L.1.21), (L.3.1); (L.3.9), (L.3.16), (L.3.17), (L.5.1), (L.5.2), (L.5.5), (L.5.6); (L.4.2), and (L.4.1). These mixtures are particularly suitable for treatment of propagation materials, i. e. seed treatment purposes and likewise for soil treatment. These seed treatment mixtures are particularly suitable for crops such as cereals, corn and leguminous plants such as soybean.

Preference is also given to mixtures comprising as pesticide II (component 2) a biopesticide selected from the groups L1), L3) and L5), preferably selected from strains denoted above as (L1.1), (L.1.2), (L.1.3), (L.1.6), (L.1.7), (L.1.9), (L.1.11), (L.1.12), (L.1.13), (L.1.14), (L.1.15), (L.1.17), (L.1.18), (L.1.22), (L.1.23), (L.1.24), (L.1.25), (L.1.26), (L.1.27), (L.2.2); (L.3.2), (L.3.3), (L.3.4), (L.3.5), (L.3.6), (L.3.7), (L.3.8), (L.3.10), (L.3.11), (L.3.12), (L.3.13), (L.3.14), (L.3.15), (L.3.18), (L.3.19); (L.4.2), even more preferably selected from (L.1.2), (L.1.7), (L.1.11), (L.1.13), (L.1.14), (L.1.15), (L.1.18), (L.1.23), (L.3.3), (L.3.4), (L.3.6), (L.3.7), (L.3.8), (L.3.10), (L.3.11), (L.3.12), (L.3.15), and (L.4.2). These mixtures are particularly suitable for foliar treatment of cultivated plants, preferably of vegetables, fruits, vines, cereals, corn, and leguminous crops such as soybeans.

The compositions comprising mixtures of active ingredients can be prepared by usual means, e. g. by the means given for the compositions of compounds I.

When living microorganisms, such as pesticides II from groups L1), L3) and L5), form part of the compositions, such compositions can be prepared by usual means (e. g. H. D. Burges: Formulation of Micobial Biopesticides, Springer, 1998; WO 2008/002371, U.S. Pat. Nos. 6,955,912, 5,422,107).

As explained above, the invention also relates to agrochemical compositions comprising at least one compound of formula I, as defined above, an N-oxide or an agriculturally acceptable salt thereof and an auxiliary for the use for combating dihydroorotate-dehydrogenase inhibitor resistant phytopathogenic fungi as described above. Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers, and binders. Suitable solvents, carriers etc. are those described above. In a particular embodiment, the composition additionally comprises at least one further active substance. Suitable further active substances correspond to those described above, especially to those of classes A) to O).

### b) Particular Biochemical Embodiments of the Claimed Use

According to a further embodiment the present invention relates to the use of a compound of the above-mentioned formula I, wherein the DHODH inhibitor resistant phytopathogenic fungus contains at least one, as for example 1, 2, 3, 4, 5, 6, 7, 8, 9 or 10, particularly 1 to 5, more particularly 1 or 2, resistance-inducing mutations in its endogenous DHODH gene, in particular resulting in at least one difference, as for example substitution, deletion, insertion, inversion, in the amino acid sequence of the mutated DHODH protein relative to the non-mutated DHODH protein.

The DHODH inhibitor resistant phytopathogenic fungus may be selected from the group of basidiomycetes, ascomycetes and oomycetes.

In particular, the DHODH inhibitor resistant phytopathogenic fungus is selected from a genus of the group of Alternaria, Bipolaris, Botrytis, Cercospora, Colletotrichum, Corynespora, Fusarium, Plenodomus, Parastagonospora, Monographella, Pseudocer-cospora, Pyricularia, Pyrenophora, Ramularia, Rhynchosporium, Sclerotinia, Zymosep-toria, and Venturia, more particularly from the genus Botrytis, Pyricularia, Sclerotinia, and Zymoseptoria.

More particularly, the DHODH inhibitor resistant phytopathogenic fungus is selected from a species of the group of Alternaria alternata, Alternaria solani, Bipolaris sorokiniana, Botrytis cinerea, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monilinia laxa, Monographella nivale, Pseudocercospora fijiensis, Pyricularia oryzae, Pyrenophora teres, Pyrenophora tritici-repentis, Ramularia collo-cygni, Rhynchosporium secalis, Sclerotinia sclerotiorum, Zymoseptoria tritici, and Venturia inaequalis. more particularly from the species Botrytis cinerea, Pyricularia oryzae, Sclerotinia sclerotiorum and Zymoseptoria tritici.

According to another embodiment of the claimed use the DHODH inhibitor resistant phytopathogenic fungus contains at least one, as for example 1, 2, 3, 4, 5, 6, 7, 8, 9 or 10, particularly 1 to 5, more particularly 1 or 2, resistance-inducing mutation in the amino acid sequence of its endogenous non-mutated DHODH enzyme in at least one sequence portion corresponding to one of the following non-mutated consensus sequence motifs (CM) 1 to 6:
a) X1 X2 X3 X4 X5 X6 X7 R
   (Consensus Motif 1) (SEQ ID NO:52)
   wherein X₁ to X₇ independently of each other represent any natural, proteinogenic amino acid residue, and in particular have the following meanings:
   X₁=W, F, Y, H or Del, wherein Del designates a missing amino acid residue
   X₂=L, A, I, For V
   X₃=V, L, S or A
   X₄=V, T or P
   X₅=P, A or M
   X₆=A, L, V, I, Tor F
   X₇=L, V, M or I
b) D X₂ E X₄ A H X₇ X₈ G
   (Consensus Motif 2) (SEQ ID NO:53)
   wherein X₂ to X8 independently of each other represent any natural, proteinogenic amino acid residue, and in particular have the following meanings:
   X₂=A, G or P
   X₄=D, E
   X₇=H or Q
   X₈=V, A, F, S or M
c) L X₂ X₃ X₄ G X₆ X₇ X₈ V E
   (Consensus Motif 3) (SEQ ID NO:54)wherein
   X₂ to X₈ independently of each other represent any natural, proteinogenic amino acid residue, and in particular have the following meanings:
   X₂=F, M or L
   X₃=E, A, D or S
   X₄=L, V or M
   X₆=P or A
   X₇=A or G
   X₈₌₁ or V
d) X₁ Q X₃ G N X₆ X₇ PR X₁₀ F R
   (Consensus Motif 4) (SEQ ID NO:55)wherein
   X₁ to X₁₀ independently of each other represent any natural, proteinogenic amino acid residue, and in particular have the following meanings:
   X₁=P or A
   X₃=D, E, P, A, L or K
   X₆=P or D
   X₇=K, R or Q
   X₁₀=V or M
e) NRYGX5NS
   (Consensus Motif 5) (SEQ ID NO:56)
   wherein
   X₅ represents any natural, proteinogenic amino acid residue, and in particular L or F
f) X₁ X₂ X₃ Y G G X₇ GT X₁₀ X₁₁ R X₁₃ K
   (Consensus Motif 6) (SEQ ID NO:57)
   wherein X₁ to X₁₃ independently of each other represent any natural, proteinogenic amino acid residue, and in particular have the following meanings:
   X₁=A, G, S or K
   X₂=M, L, R or I
   X₃=I, V, M or T
   X₇=A, S, V or P
   X₁₀=I or V
   X₁₁=T, G or S
   X₁₃=I, M or V

In particular, the order of the consensus motifs (CM) 1 to 6 within the respective amino acid sequence is, from the N- to the C-terminus: CM1-CM2-CM3-CM4-CM5-CM6, wherein “-” represents a peptidic linkage or, more particularly and independently of each other a monopeptide, oligopeptide or polypeptide bridge, as for example formed by one or more, as for example about 1 to 800, about 1 to 600, about 1 to 300 or about 2 to 70 or about 2 to 10 amino acid residues, as for example derivable from the alignment of FIG. 1.

According to another embodiment of the claimed use the endogenous non-mutated DHODH enzyme is selected from enzymes comprising at least one amino acid sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49 or 51 or a sequence having a degree of sequence identity of at least 70%, 75%, 80%, 85%, 90%, 95%, 96%, 97%, 98%, 99%, 99.5% or 99.9% to anyone of said sequences, while retaining DHODH enzyme activity.

According to a particular embodiment of the claimed use the DHODH inhibitor resistant phytopathogenic fungus is of the species Botrytis cinerea and contains at least one, as for example 1, 2, 3, 4, 5, 6, 7, 8, 9 or 10, particularly 1, 2, 3 or 4, more particularly 1 or 2, and most particularly 1 resistance-inducing mutation in at least, particularly 1, 2 or 3, more particularly 1 or 2, and most particularly 1 of the following non-mutated sequence motifs (M) 1 to 6 of its non-mutated DHODH comprising an amino acid sequence of SEQ ID NO:1, or a sequence having a degree of sequence identity of at least 70%, 75%, 80%, 85%, 90%, 95%, 96%, 97%, 98%, 99%, 99.5% or 99.9% thereto:
a) WLVVPALR (Motif 1) (SEQ ID NO:58) corresponding to amino acid residues 91 to 98 of SEQ ID NO:1
b) DAEDAHHVG (Motif 2) (SEQ ID NO:59) corresponding to amino acid resi-dues 103 to 111 of SEQ ID NO:1
c) LFALGPAIVE (Motif 3) (SEQ ID NO:60) corresponding to amino acid residues 169 to 178 of SEQ ID NO:1
d) AQDGNPKPRVFR (Motif 4) (SEQ ID NO:61) corresponding to amino acid residues 186 to 197 of SEQ ID NO:1
e) NRYGLNS (Motif 5) SEQ ID NO:62 corresponding to amino acid residues 206 to 212 of SEQ ID NO:1
f) AMVYGGAGTITRIK (Motif 6) SEQ ID NO:63 corresponding to amino acid res-idues 492 to 505 of SEQ ID NO:1

In particular, the order of the motifs (M) 1 to 6 within the respective amino acid sequence is, from the N- to the C-terminus: M1-M2-M3-M4-M5-M6, wherein “-” repre-sents a peptide linkage or, more particularly independently of each other a monopep-tide, oligopeptide or polypeptide bridge, as for example formed by one or more, as for example about 1 to 800, about 1 to 600, about 1 to 300 or about 2 to 70 or about 2 to 10 amino acid residues, as for example derivable from the alignment of FIG. 1.

According to a more particular use of the invention the DHODH inhibitor resistant phy-topathogenic fungus of the species Botrytis cinerea contains:
at least one resistance-inducing mutation in Motif 4 (SEQ ID NO:61) of SEQ ID NO:1;or
a combination of at least one resistance-inducing mutation in (Motif 4) (SEQ ID NO:61)and at least one resistance-inducing mutation in Motif 6 (SEQ ID NO:63) each of SEQ ID NO: 1; or
a combination of at least one resistance-inducing mutation in Motif 4 (SEQ ID NO:61)and at least one resistance-inducing mutation in Motif 2 (SEQ ID NO:59) each of SEQ ID NO: 1.

According to a very particular use of the invention the DHODH inhibitor resistant phytopathogenic fungus is of the species Botrytis cinerea and contains at least one resistance-inducing mutation in the non-mutated sequence of SEQ ID NO:1 or in a sequence which is at least 70% identical thereto, wherein the mutant is selected from:
a) the single mutants
   V94X, in particular F, I, L, D, A, G, E or M, more particularly I or L
   A104X, in particular T, P, S, D, V, G or E, more particularly D
   A107X, in particular T, P, S, D, V, G or E, more particularly T or V
   H108X, in particular Q, P, R, L, D, Y or N, more particularly P Del109,
   G111X, in particular E, A, V, R, D, C, W or S, more particularly R, V or E
   L172X, in particular F, I, S, V, H, P, R, Q or M, more particularly F
   V195X, in particular F, I, L, D, A, G, E or M, more particularly F, E or D, especially F
   Y208X, in particular S, C, F, N, H or D, more particularly H
   V494X, in particular F, I, L, D, A, G, E or M, more particularly G
   G497X, in particular E, A, V, R, D, C, W or S, more particularly V
      wherein
      Del designates a deletion of an amino acid residue and
      X represents any natural, proteinogenic, in particular any natural, proteinogenic resistance-inducing amino acid residue; and
b) multiple mutants
   containing a combination of at least 2 of the resistance inducing single mutations as defined above under a),
   in particular double mutants selected from
   V195X/A107X, more particularly V195F/A107V and V195F/A107T; and
   V195X/V494X, more particularly V195F/V494G
      wherein
      X represents any natural, proteinogenic, in particular any natural, proteinogenic resistance-inducing amino acid residue.

According to still another embodiment of the use of the invention the DHODH inhibitor resistant phytopathogenic fungus is different from Botrytis cinerea and contains at least one resistance-inducing mutation in the endogenous DHODH amino acid sequence, in an amino acid sequence position analogous to that of a mutant of the non-mutated Botrytis cinerea DHODH amino acid sequence of SEQ ID NO:1 as defined above.

In particular, the DHODH inhibitor resistant phytopathogenic fungus different from Botrytis cinerea is selected from a species selected from Alternaria alternata, Alternaria solani, Bipolaris sorokiniana, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monographella nivale, Monilinia laxa, Pseudocercospora fijiensis, Pyricularia oryzae, Pyrenophora teres, Pyrenophora tritici-repentis, Ramularia collo-cygni, Rhynchosporium secalis, Sclerotinia sclerotiorum, Zymoseptoria tritici, and Venturia inaequalis.

Mutants of different fungal species analogous to the particular Botrytis cinerea mutants as described above can be easily derived from a sequence alignment of the respective non-mutated, endogenous DHODH enzymes of Botrytis cinerea and the other fungal species and identifying corresponding analogous positions to be mutated therein. An example of a suitable alignment is depicted in FIG. 1 of the specification.

Accordingly, the following non-limiting examples of analogous mutants shall be given:
a) the V195X-analogous mutants selected from
   V181X mutants of the Alternaria alternata DHODH having a non-mutated amino acid sequence of SEQ ID NO: 3
   V187X mutants of the Alternaria solani DHODH having a non-mutated amino acid sequence of SEQ ID NO: 5
   V186X mutants of the Bipolaris sorokiniana DHODH having a non-mutated amino acid sequence of SEQ ID NO: 11
   V221X mutants of the Cercospora sojina DHODH having a non-mutated amino acid sequence of SEQ ID NO: 9
   V221X mutants of the Cercospora beticola DHODH having a non-mutated amino acid sequence of SEQ ID NO: 7
   V185X mutants of the Colletotrichum graminicola DHODH having a non-mutated amino acid sequence of SEQ ID NO: 13
   V183X mutants of the Colletotrichum obriculare DHODH having a non-mutated amino acid sequence of SEQ ID NO: 15
   V178X mutants of the Corynespora cassicola DHODH having a non-mutated amino acid sequence of SEQ ID NO: 17
   V179X mutants of the Fusarium culmorum DHODH having a non-mutated amino acid sequence of SEQ ID NO: 19
   V179X mutants of the Fusarium graminearum DHODH having a non-mutated amino acid sequence of SEQ ID NO: 23
   V179X mutants of the Fusarium oxysporum f. sp. Lycopersici DHODH having a non-mutated amino acid sequence of SEQ ID NO: 21
   V544X mutants of the Plenodomus lingam DHODH having a non-mutated amino acid sequence of SEQ ID NO: 25
   V181X mutants of the Parastagonospora nodorum DHODH having a non-mutated amino acid sequence of SEQ ID NO: 27
   M192X mutants of the Monographella nivale DHODH having a non-mutated amino acid sequence of SEQ ID NO: 29
   V176X mutants of the Monilinia laxa DHODH having a non-mutated amino acid sequence of SEQ ID NO: 49
   V139X mutants of the Pseudocercospora fijiensis DHODH having a non-mutated amino acid sequence of SEQ ID NO: 31
   V184X mutants of the Pyricularia oryzae DHODH having a non-mutated amino acid sequence of SEQ ID NO: 33
   V175X mutants of the Pyrenophora teres DHODH having a non-mutated amino acid sequence of SEQ ID NO: 35
   V175X mutants of the Pyrenophora tritici-repentis DHODH having a non-mutated amino acid sequence of SEQ ID NO: 37
   V198X mutants of the Ramularia collo-cygni DHODH having a non-mutated amino acid sequence of SEQ ID NO: 39
   V185X mutants of the Rhynchosporium secalis DHODH having a non-mutated amino acid sequence of SEQ ID NO: 41
   V176X mutants of the Sclerotinia sclerotiorum DHODH having a non-mutated amino acid sequence of SEQ ID NO: 43
   V246X mutants of the Zymoseptoria tritici DHODH having a non-mutated amino acid sequence of SEQ ID NO: 45 and
   V188X mutants of the Venturia inaequalis DHODH having a non-mutated amino acid sequence of SEQ ID NO: 47.
b) the A107X-analogous mutants are selected from
   A99X mutants of the P. oryzae DHODH having a non-mutated amino acid sequence of SEQ ID NO: 33
   A160X mutants of the Z. tritici DHODH having a non-mutated amino acid sequence of SEQ ID NO: 45

Further analogous mutants of DHODHs from Alternaria alternata, Alternaria solani, Bipolaris sorokiniana, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parstagonospora nodorum, Monographella nivale, Monilinia laxa, Pseudocercospora fijiensis, Pyrenophora teres, Pyrenophora tritici-repentis, Ramularia collo-cygni, Rhynchosporium secalis, Sclerotinia sclerotiorum, and Venturia inaequalis, can be easily derived for this sequence position from the sequence alignment depicted in FIG. 1.
c) the H108X-analogous mutants are selected from
   H161X mutants of the Z. tritici DHODH having a non-mutated amino acid sequence of SEQ ID NO: 45.

Further analogous mutants of DHODHs from Alternaria alternata, Alternaria solani, Bipolaris sorokiniana, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monographella nivale, Monilinia laxa, Pseudocercospora fijiensis, Pyricularia oryzae, Pyrenophora teres, Pyrenophora tritici-repentis, Ramularia collo-cygni, Rhynchosporium secalis, Sclerotinia sclerotiorum, and Venturia inaequalis, can be easily derived for this sequence position from the sequence alignment depicted in FIG. 1.
d) the Y208X-analogous mutants are selected from
   Y197X mutants of the P. oryzae DHODH having a non-mutated amino acid sequence of SEQ ID NO: 33

Further analogous mutants of DHODHs from Alternaria alternata, Alternaria solani, Bi-polaris sorokiniana, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monographella nivalie, Monilinia laxa, Pseudocercospora fijiensis, Pyricularia oryzae, Pyrenophora teres, Pyrenophora tritici-repentis, Ramularia collo-cygni, Rhynchosporium secalis, Sclerotinia sclerotiorum, and Venturia inaequalis, can be easily derived for this sequence position from the sequence alignment depicted in FIG. 1.

### c) Particular Embodiments Concerning Enzyme Mutants of the Invention

The present invention also relates to mutated DHODH inhibitor resistant DHODH enzyme as defined herein above.

In particular the present invention relates to DHODH inhibitor resistant DHODH enzyme mutants, which are obtainable from a phytopathogenic fungus that contains at least one, as for example 1, 2, 3, 4, 5, 6, 7, 8, 9 or 10, particularly 1 to 5, more particularly 1 or 2, resistance-inducing mutations in its endogenous DHODH gene, in particular resulting in at least one difference, as for example substitution, deletion, insertion, inversion, in the amino acid sequence of the mutated DHODH protein relative to the non-mutated DHODH protein.

Such mutant enzymes are obtainable from DHODH inhibitor resistant phytopathogenic fungus selected from the group of basidiomycetes, ascomycetes and oomycetes.

In particular, such mutant enzymes are obtainable from a DHODH inhibitor resistant phytopathogenic fungus, selected from the genus Alternaria, Bipolaris, Botrytis, Cercospora, Colletotrichum, Corynespora, Fusarium, Plenodomus, Parastagonospora, Monographella, Pseudocercospora, Pyricularia, Pyrenophora, Ramularia, Rhynchosporium, Sclerotinia, Zymoseptoria, and Venturia, more particularly from the genus Botrytis, Pyricularia, Sclerotinia, and Zymoseptorias, and more particularly, from a fungal species selected from Alternaria altemata, Alternaria solani, Bipolaris sorokiniana, Botrytis cinerea, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monilinia laxa, Monographella nivale, Pseudocercospora fijiensis, Pyricularia oryzae, Pyrenophora teres, Pyrenophora tritici-repentis, Ramularia collocygni, Rhynchosporium secalis, Sclerotinia sclerotiorum, Zymoseptoria tritici, and Venturia inaequalis. more particularly from the species, Botrytis cinerea, Pyricularia oryzae, Sclerotinia sclerotiorum and Zymoseptoria tritici.

According to a more embodiment the DHODH mutant is, for example, obtainable from an DHODH inhibitor resistant phytopathogenic fungus which contains at least one, as for example 1, 2, 3, 4, 5, 6, 7, 8, 9 or 10, particularly 1 to 5, more particularly 1 or 2, resistance-inducing mutation in at least one sequence portion corresponding to one of the following non-mutated consensus sequence motifs (CM) 1 to 6 of its endogenous non-mutated amino acid sequence
a) X₁ X₂ X₃ X₄ X₅ X₆ X₇ R
   (Consensus Motif 1) (SEQ ID NO:52)wherein
   X₁ to X₇ independently of each other represent any natural, proteinogenic amino acid residue, and in particular have the following meanings:
   X₁=W, F, Y, H or Del, wherein Del designates a missing amino acid residue
   X₂=L, A, I, F or V
   X₃=V, L, S or A
   X₄=V, T or P
   X₅=P, A or M
   X₆=A, L, V, I, Tor F
   X₇=L, V, M or I
b) D X₂ E X₄ A H X₇ X₈ G
   (Consensus Motif 2) (SEQ ID NO:53)
   wherein X² to X₈ independently of each other represent any natural, proteinogenic amino acid residue, and in particular have the following meanings:
   X₂=A, G or P
   X₄=D, E
   X₇=H or Q
   X₈=V, A, F, S or M
c) L X₂ X₃ X₄ G X₆ X₇ X₈ V E
   (Consensus Motif 3) (SEQ ID NO:54)
   wherein X² to X₈ independently of each other represent any natural, proteinogenic amino acid residue, and in particular have the following meanings:
   X₂=F, M or L
   X₃=E, A, D or S
   X₄=L, V or M
   X₆=Por A
   X₇=A or G
   X₈=I or V
d) X₁ Q X₃ G N X₆ X₇ P R X₁₀ F R
   (Consensus Motif 4) (SEQ ID NO:55)
   wherein X₁ to X₁₀ independently of each other represent any natural, proteinogenic amino acid residue, and in particular have the following meanings:
   X₁=P or A
   X₃=D, E, P, A, L or K
   X₆=P or D
   X₇=K, R or Q
   X₁₀=V or M
e) NRYGX5NS
   (Consensus Motif 5) (SEQ ID NO:56)
   wherein X₅=any natural, proteinogenic amino acid residue, and in particular L or F
f) X₁ X₂ X₃ Y GG X₇ G T X₁₀ X₁₁ R X₁₃ K
   (Consensus Motif 6) (SEQ ID NO:57)
   wherein X₁ to X₁₃ independently of each other represent any natural, proteinogenic amino acid residue, and in particular have the following meanings:
   X₁=A, G, S or K
   X₂=M, L, R or I
   X₃=I, V, M or T
   X₇=A, S, V or P
   X₁₀₌₁ or V
   X₁₁=T, G or S
   X₁₃=I, M or V

In particular, the order of the consensus motifs (CM) 1 to 6 within the respective amino acid sequence is, from the N -to the C-terminus: CM1-CM2-CM3-CM4-CM5-CM6, wherein “-” represents a peptidic linkage or, more particularly independently of each other aminopeptide, oligopeptide or polypeptide bridge, as for example formed by one or more, as for example about 1 to 800, about 1 to 600, about 1 to 300 or about 2 to 70 or about 2 to 10 amino acid residues, as for example derivable from the alignment of FIG. 1.

According to another more particular embodiment, said endogenous non-mutated DHODH enzyme is selected from enzymes comprising at least one amino acid sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49 or 51, or a sequence having a degree of sequence identity of at least 70%, 75%, 80%, 85%, 90%, 95%, 96%, 97%, 98%, 99%, 99.5% or 99.9% to anyone of said sequences, while retaining DHODH enzyme activity.

According to a still more particular embodiment, the DHODH mutant is for example obtainable from a DHODH inhibitor resistant phytopathogenic fungus of the species Botrytis cinerea. The mutant contains at least one, particularly 1, 2, 3 or 4, more particularly 1 or 2, and most particularly 1 resistance-inducing mutation in at least, particularly 1, 2 or 3, more particularly 1 or 2, and most particularly 1 of the following non-mutated sequence motifs of the non-mutated DHODH comprising an amino acid sequence of SEQ ID NO:1, or a sequence having a degree of sequence identity of at least 70%, 75%, 80%, 85%, 90%, 95%, 96%, 97%, 98%, 99%, 99.5% or 99.9% thereto:
a) WLVVPALR (Motif 1) (SEQ ID NO:58) corresponding to amino acid residues 91 to 98 of SEQ ID NO:1
b) DAEDAHHVG (Motif 2) (SEQ ID NO:59) corresponding to amino acid resi-dues 103 to 111 of SEQ ID NO:1
c) LFALGPAIVE (Motif 3) (SEQ ID NO:60) corresponding to amino acid residues 169 to 178 of SEQ ID NO:1
d) AQDGNPKPRVFR (Motif 4) (SEQ ID NO:61) corresponding to amino acid residues 186 to 197 of SEQ ID NO:1
e) NRYGLNS (Motif 5) SEQ ID NO:62 corresponding to amino acid residues 206 to 212 of SEQ ID NO:1
f) AMVYGGAGTITRIK (Motif 6) SEQ ID NO:63 corresponding to amino acid res-idues 492 to 505 of SEQ ID NO:1

In particular, the order of the motifs (M) 1 to 6 within the respective amino acid sequence is, from the N- to the C-terminus: M1-M2-M3-M4-M5-M6, wherein “-” repre-sents a peptidic linkage or, more particularly independently of each other a monopep-tide, oligopeptide or polypeptide bridge, as for example formed by one or more, as for example about 1 to 800, about 1 to 600, about 1 to 300 or about 2 to 70 or about 2 to 10 amino acid residues, as for example derivable from the alignment of FIG. 1.

According to a very particular embodiment of the invention the DHODH mutant is for example obtainable from an inhibitor resistant phytopathogenic fungus of the species Botrytis cinerea. It contains
at least one resistance-inducing mutation in Motif 4 (SEQ ID NO:61) of SEQ ID NO:1;
or
a combination of at least one resistance-inducing mutation in Motif 4 (SEQ ID NO:61)and at least one resistance-inducing mutation in Motif 6 (SEQ ID NO:63) each of SEQ ID NO: 1; or
a combination of at least one resistance-inducing mutation in Motif 4 (SEQ ID NO:61)and at least one resistance-inducing mutation in Motif 2 (SEQ ID NO:59) each of SEQ ID NO: 1.

According to a very particular embodiment the DHODH mutant is for example obtainable from inhibitor resistant phytopathogenic fungus is of the species Botrytis cinerea. The mutant contains at least one resistance-inducing mutation in the non-mutated sequence of SEQ ID NO:1 or in a sequence which is at least 70%, 75%, 80%, 85%, 90%, 95%, 96%, 97%, 98%, 99%, 99.5% or 99.9% identical thereto, wherein the mutant is selected from
a) the single mutants
   V94X, in particular F, I, L, D, A, G, E or M, more particularly I or L
   A104X, in particular T, P, S, D, V, G or E, more particularly D
   A107X, in particular T, P, S, D, V, G or E, more particularly T or V
   H108X, in particular Q, P, R, L, D, Y or N, more particularly P
   Del109,
   G111X, in particular E, A, V, R, D, C, W or S, more particularly R, V or E
   L172X, in particular F, I, S, V, H, P, R, Q or M, more particularly F
   V195X, in particular F, I, L, D, A, G, E or M, more particularly F, E or D, especially F
   Y208X, in particular S, C, F, N, H or D, more particularly H
   V494X, in particular F, I, L, D, A, G, E or M, more particularly G
   G497X, in particular E, A, V, R, D, C, W or S, more particularly V
      wherein
      Del designates a deletion of an amino acid residue and
      X represents any natural, proteinogenic, in particular any natural, proteinogenic resistance-inducing amino acid residue; and
b) multiple mutants
   containing a combination of at least 2 of the resistance inducing single mutations as defined above under a),
   in particular double mutants selected from
   V195X/A107X, more particularly V195F/A107V and V195F/A107T; and
   V195X/V494X, more particularly V195F/V494G
      wherein
      X represents any natural, proteinogenic, in particular any natural, proteinogenic resistance-inducing amino acid residue.

According to still another embodiment further DHODH mutants are provided are different from mutants as obtainable from Botrytis cinerea. Such further mutants contain at least one resistance-inducing mutation in the endogenous DHODH amino acid sequence, in an amino acid sequence position analogous to that of a mutant of the non-mutated Botrytis cinerea DHODH amino acid sequence of SEQ ID NO:1 as defined above.

In particular such further DHODH different from that obtainable from Botrytis cinerea can be derived from endogenous DHODHs of fungal species selected from Alternaria alternata, Alternaria solani, Bipolaris sorokiniana, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monographella nivale, Monilinia laxa, Pseudocercospora fijiensis, Pyricularia oryzae, Pyrenophora teres, Pyrenophora tritici-repentis, Ramularia collo-cygni, Rhynchosporium secalis, Sclerotinia sclerotiorum, Zymoseptoria tritici, and Venturia inaequalis.

DHODH mutants of different fungal species but analogous to the particular Botrytis ci- nerea mutants as described above can be easily derived from a sequence alignment of the respective non-mutated, endogenous DHODH enzymes of Botrytis cinerea and the non-mutated DHODH of other fungal species and by identifying corresponding analogous positions to be mutated therein. An example of a suitable alignment is depicted in FIG. 1 of the specification.

Accordingly, the following non-limiting examples of analogous mutants shall be given:
a) the V195X-analogous mutants selected from
   V181X mutants of the Alternaria alternata DHODH having a non-mutated amino acid sequence of SEQ ID NO: 3
   V187X mutants of the Alternaria solani DHODH having a non-mutated amino acid sequence of SEQ ID NO: 5
   V186X mutants of the Bipolaris sorokiniana DHODH having a non-mutated amino acid sequence of SEQ ID NO: 11
   V221X mutants of the Cercospora sojina DHODH having a non-mutated amino acid sequence of SEQ ID NO: 9
   V221X mutants of the Cercospora beticola DHODH having a non-mutated amino acid sequence of SEQ ID NO: 7
   V185X mutants of the Colletotrichum graminicola DHODH having a non-mutated amino acid sequence of SEQ ID NO: 13
   V183X mutants of the Colletotrichum obriculare DHODH having a non-mutated amino acid sequence of SEQ ID NO: 15
   V178X mutants of the Corynespora cassicola DHODH having a non-mutated amino acid sequence of SEQ ID NO: 17
   V179X mutants of the Fusarium culmorum DHODH having a non-mutated amino acid sequence of SEQ ID NO: 19
   V179X mutants of the Fusarium graminearum DHODH having a non-mutated amino acid sequence of SEQ ID NO: 23
   V179X mutants of the Fusarium oxysporum f. sp. Lycopersici DHODH having a non-mutated amino acid sequence of SEQ ID NO: 21
   V544X mutants of the Plenodomus lingam DHODH having a non-mutated amino acid sequence of SEQ ID NO: 25
   V181X mutants of the Parastagonospora nodorum DHODH having a non-mutated amino acid sequence of SEQ ID NO: 27
   M192X mutants of the Monographella nivalis DHODH having a non-mutated amino acid sequence of SEQ ID NO: 29
   V176X mutants of the Monilinia laxa DHODH having a non-mutated amino acid sequence of SEQ ID NO: 49
   V139X mutants of the Pseudocercospora fijiensis DHODH having a non-mutated amino acid sequence of SEQ ID NO: 31
   V184X mutants of the Pyricularia oryzae DHODH having a non-mutated amino acid sequence of SEQ ID NO: 33
   V175X mutants of the Pyrenophora teres DHODH having a non-mutated amino acid sequence of SEQ ID NO: 35
   V175X mutants of the Pyrenophora tritici-repentis DHODH having a non-mutated amino acid sequence of SEQ ID NO: 37
   V198X mutants of the Ramularia collo-cygni DHODH having a non-mutated amino acid sequence of SEQ ID NO: 39
   V185X mutants of the Rhynchosporium secalis DHODH having a non-mutated amino acid sequence of SEQ ID NO: 41
   V176X mutants of the Sclerotinia sclerotiorum DHODH having a non-mutated amino acid sequence of SEQ ID NO: 43
   V246X mutants of the Zymoseptoria tritici DHODH having a non-mutated amino acid sequence of SEQ ID NO: 45 and
   V188X mutants of the Venturia inaequalis DHODH having a non-mutated amino acid sequence of SEQ ID NO: 47.
b) the A107X-analogous mutants are selected from
   A99X mutants of the P. oryzae DHODH having a non-mutated amino acid sequence of SEQ ID NO: 33
   A160X mutants of the Z. tritici DHODH having a non-mutated amino acid sequence of SEQ ID NO: 45

Further analogous mutants of DHODHs from Alternaria alternata, Alternaria solani, Bipolaris sorokiniana, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monographella nivale, Monilinia laxa, Pseudocercospora fijiensis,, Pyrenophora teres, Pyrenophora tritici-repentis, Ramularia collo-cygni, Rhynchosporium secalis, Sclerotinia sclerotiorum, and Venturia inaequalis, can be easily derived for this particular sequence from the sequence alignment depicted in FIG. 1.
c) the H108X-analogous mutants are selected from
   H161X mutants of the Z. tritici DHODH having a non-mutated amino acid sequence of SEQ ID NO: 45.

Further analogous mutants of DHODHs from Alternaria alternata, Alternaria solani, Bipolaris sorokiniana, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monographella nivale, Monilinia laxa, Pseudocercospora fijiensis, Pyricularia oryzae, Pyrenophora teres, Pyrenophora tritici-repentis, Ramularia collo-cygni, Rhynchosporium secalis, Sclerotinia sclerotiorum, and Venturia inaequalis, can be easily derived for this particular sequence from the sequence alignment depicted in FIG. 1.
d) the Y208X-analogous mutants are selected from
   Y197X mutants of the P. oryzae DHODH having a non-mutated amino acid sequence of SEQ ID NO: 45

Further analogous mutants of DHODHs from Alternaria alternata, Alternaria solani, Bipolaris sorokiniana, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monographella nivalis, Monilinia laxa, Pseudocercospora fijiensis, Pyricularia oryzae, Pyrenophora teres, Pyrenophora tritici-repentis, Ramularia collo-cygni, Rhynchosporium secalis, Sclerotinia sclerotiorum, and Venturia inaequalis, can be easily derived for this particular sequence from the sequence alignment depicted in FIG. 1.

While the above-mentioned embodiments have been described on the basis of the DHODH enzyme of Botrytis cinerea in its long version according to SEQ ID NO: 1, the enzyme also exists in a short version, wherein an amino terminal portion of 19 residues is missing and which is described herein as SEQ ID NO:51. In this short version, for example, position V195 of the long version would correspond to V176. The numbering of the other particular sequence positions would be changed accordingly.

A further embodiment of the invention relates to a nucleic acid molecule comprising at least one nucleotide sequence encoding a mutated DHODH enzyme as defined above, in particular selected from mutant nucleic acid sequences derived from nucleotide sequences according to SEQ ID NO: 2, 4, 6, 8, 10, 12, 14, 16, 18, 20, 22, 24, 26, 28, 30, 32, 34, 36, 38, 40, 42, 44, 46, 48, or 50 and sequences having at least 70%, 75%, 80%, 85%, 90%, 95%, 96%, 97%, 98%, 99%, 99.5% or 99.9% sequence identity to anyone of said sequences, comprising at least one mutation in the nucleotide sequence which encodes said mutated DHODH enzyme.

Still another embodiment of the invention relates to an expression construct comprising at least one nucleic acid molecule comprising at least one nucleotide sequence encoding a mutated DHODH enzyme as defined above, in particular selected from mutant nucleic acid sequences derived from nucleotide sequences according to SEQ ID NO: 2, 4, 6, 8, 10, 12, 14, 16, 18, 20, 22, 24, 26, 28, 30, 32, 34, 36, 38, 40, 42, 44, 46, 48, or 50 and sequences having at least 70%, 75%, 80%, 85%, 90%, 95%, 96%, 97%, 98%, 99%, 99.5% or 99.9% sequence identity to anyone of said sequences, comprises-ing at least one mutation in the nucleotide sequence which encodes said mutated DHODH enzyme.

Another embodiment of the invention provides a vector comprising at least one nucleic acid molecule comprising at least one nucleotide sequence encoding a mutated DHODH enzyme as defined above, in particular selected from mutant nucleic acid sequences derived from nucleotide sequences according to SEQ ID NO: 2, 4, 6, 8, 10, 12, 14, 16, 18, 20, 22, 24, 26, 28, 30, 32, 34, 36, 38, 40, 42, 44, 46, 48, or 50 and sequences having at least 70%, 75%, 80%, 85%, 90%, 95%, 96%, 97%, 98%, 99%, 99.5% or 99.9% sequence identity to anyone of said sequences, comprising at least one mutation in the nucleotide sequence which encodes said mutated DHODH enzyme, or at least one expression construct comprising at least one such coding nucleotide sequence.

Another embodiment of the invention provides a natural or non-natural, recombinant microorganism, host or host cell comprising at least one such nucleic acid molecule, or at least one such expression construct or at least one vector, each comprising at least one nucleic acid molecule comprising at least one nucleotide sequence encoding a mutated DHODH enzyme as defined above, in particular selected from mutant nucleic acid sequences derived from nucleotide sequences according to SEQ ID NO: 2, 4, 6, 8, 10, 12, 14, 16, 18, 20, 22, 24, 26, 28, 30, 32, 34, 36, 38, 40, 42, 44, 46, 48, or 50 and sequences having at least 70%, 75%, 80%, 85%, 90%, 95%, 96%, 97%, 98%, 99%, 99.5% or 99.9% sequence identity to anyone of said sequences

In another embodiment the present invention provides a method for combatting a phy-topathogenic fungus is selected from the genus Alternaria, Bipolaris, Botrytis, Cercospora, Colletotrichum, Corynespora, Fusarium, Plenodomus, Parastagonospora, Monographella, Pseudocercospora, Pyricularia, Pyrenophora, Ramularia, Rhynchosporium, Sclerotinia, Zymoseptoria, and Venturia, more particularly from the genus Botrytis, Pyricularia, Sclerotinia, and Zymoseptoria, more particularly, a phytopathogenic fungus is selected from the species Alternaria alternata, Alternaria solani, Bipolaris sorokiniana, Botrytis cinerea, Cercospora sojina, Cercospora beticola, Colletotrichum graminicola, Colletotrichum obriculare, Corynespora cassicola, Fusarium culmorum, Fusarium graminearum, Fusarium oxysporum f. sp. Lycopersici, Plenodomus lingam, Parastagonospora nodorum, Monilinia laxa, Monographella nivale, Pseudocercospora fijiensis, Pyricularia oryzae, Pyrenophora teres, Pyrenophora tritici-repentis, Ramularia collo-cygni, Rhynchosporium secalis, Sclerotinia sclerotiorum, Zymoseptoria tritici, and Venturia inaequalis, more particularly from the species, Botrytis cinerea, Pyricularia oryzae, Sclerotinia sclerotiorum and Zymoseptoria tritici, comprising:
treating the phytopathogenic fungus or the materials, plants, the soil or seeds that are at risk of being diseased from said phytopathogenic fungus with an effective amount of at least one compound of formula I as defined herein above or a composition comprises-ing it.

More particularly said method comprises:
c) identifying said phytopathogenic fungus, or the materials, plants, the soil or seeds that are at risk of being diseased from said phytopathogenic fungus; and
d) treating said fungus or the materials, plants, the soil or seeds with an effective amount of at least one compound of formula I as defined herein above or a composition comprising it.

Still another embodiment of the invention relates to a screening method for identifying DHODH inhibitor compounds suitable for combatting DHODH inhibitor resistant phytopathogenic fungi, which method comprises contraction a DHODH inhibitor resistant phytopathogenic fungus or spores thereof capable of expressing a DHODH enzyme mutant as defined above with at least one chemical or biochemical molecule suspected to exert inhibiting activity on such fungus, cultivating the fungus or spores thereof in the presence of said at least one chemical or biochemical molecule, and determining the ability of the fungal culture to grow.

d) Further more general biochemical embodiments

### 1. Polypeptides of the Invention

In this context the following definitions apply:

The generic terms “polypeptide” or “peptide”, which may be used interchangeably, refer to a natural or synthetic linear chain or sequence of consecutive, peptidically linked amino acid residues, comprising about 10 to up to more than 1.000 residues. Short chain polypeptides with up to 30 residues are also designated as “oligopeptides”.

The term “protein” refers to a macromolecular structure consisting of one or more polypeptides. The amino acid sequence of its polypeptide(s) represents the “primary structure” of the protein. The amino acid sequence also predetermines the “secondary structure” of the protein by the formation of special structural elements, such as alpha-helical and beta-sheet structures formed within a polypeptide chain. The arrangement of a plurality of such secondary structural elements defines the “tertiary structure” or spatial arrangement of the protein. If a protein comprises more than one polypeptide chains said chains are spatially arranged forming the “quaternary structure” of the protein. A correct spacial arrangement or “folding” of the protein is prerequisite of protein function. Denaturation or unfolding destroys protein function. If such destruction is reversible, protein function may be restored by refolding.

A typical protein function referred to herein is an “enzyme function”, i.e. the protein acts as biocatalyst on a substrate, for example a chemical compound, and cata-lyzes the conversion of said substrate to a product. An enzyme may show a high or low degree of substrate and/or product specificity.

A “polypeptide” referred to herein as having a particular “activity” thus implicitly refers to a correctly folded protein showing the indicated activity, as for example a specific enzyme activity.

Thus, unless otherwise indicated the term “polypeptide” also encompasses the terms “protein” and “enzyme”.

Similarly, the term “polypeptide fragment” encompasses the terms “protein fragment” and “enzyme fragment”.

The term “isolated polypeptide” refers to an amino acid sequence that is removed from its natural environment by any method or combination of methods known in the art and includes recombinant, biochemical and synthetic methods.

The present invention also relates to “functional equivalents” (also designated as “analogs” or “functional mutations”) of the polypeptides specifically described herein.

For example, “functional equivalents” refer to polypeptides, which, in a test used for determining an enzymatic activity referred to herein, as in particular DHODH activity, display at least a 1 to 10%, or at least 20%, or at least 50%, or at least 75%, or at least 90% higher or lower activity, as that of the polypeptides specifically described herein.

“Functional equivalents”, according to the invention, also cover particular mutants, which, in at least one sequence position of an amino acid sequences stated herein, have an amino acid that is different from that concretely stated one, but never-theless possess one of the aforementioned biological activities, as for example enzyme activity. “Functional equivalents” thus comprise mutants obtainable by one or more, like 1 to 20, in particular 1 to 15 or 5 to 10 amino acid additions, substitutions, in particular conservative substitutions, deletions and/or inversions, where the stated changes can occur in any sequence position, provided they lead to a mutant with the profile of properties according to the invention. Functional equivalence is in particular also provided if the activity patterns coincide qualitatively between the mutant and the unchanged polypeptide, i.e. if, for example, interaction with the same agonist or antagonist or substrate, however at a different rate, (i.e. expressed by a EC₅₀ or IC₅₀ value or any other parameter suitable in the present technical field) is observed. Examples of suitable (conservative) amino acid substitutions are shown in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described herein, as well as “functional derivatives” and “salts” of the polypeptides.

“Precursors” are in that case natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. Salts of acid addition, for example salts with inorganic acids, such as hydrochloric acid or sulfuric acid and salts with organic acids, such as acetic acid and oxalic acid, are also covered by the invention.

“Functional derivatives” of polypeptides according to the invention can also be produced on functional amino acid side groups or at their N-terminal or C-terminal end using known techniques. Such derivatives comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxyl groups, produced by reaction with acyl groups.

“Functional equivalents” naturally also comprise polypeptides that can be obtained from other organisms, as well as naturally occurring variants. For example, ar-eas of homologous sequence regions can be established by sequence comparison, and equivalent polypeptides can be determined on the basis of the concrete parameters of the invention.

“Functional equivalents” also comprise “fragments”, like individual domains or sequence motifs, of the polypeptides according to the invention, or N-and or C-termi-nally truncated forms, which may or may not display the desired biological function. Particularly such “fragments” retain the desired biological function at least qualitatively.

“Functional equivalents” are, moreover, fusion proteins, which have one of the polypeptide sequences stated herein or functional equivalents derived there from and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

“Functional equivalents” which are also comprised in accordance with the invention are homologs to the specifically disclosed polypeptides. These have at least 60%, particularly at least 75%, in particular at least 80 or 85%, such as, for example, 90, 91, 92, 93, 94, 95, 96, 97, 98 or 99%, homology (or identity) to one of the specifically disclosed amino acid sequences, calculated by the algorithm of Pearson and Lip-man, Proc. Natl. Acad, Sci. (USA) 85 (8), 1988, 2444-2448. A homology or identity, expressed as a percentage, of a homologous polypeptide according to the invention means in particular an identity, expressed as a percentage, of the amino acid residues based on the total length of one of the amino acid sequences described specifically herein.

The identity data, expressed as a percentage, may also be determined with the aid of BLAST alignments, algorithm blastp (protein-protein BLAST), or by applying the Clustal settings specified herein below.

In the case of a possible protein glycosylation, “functional equivalents” according to the invention comprise polypeptides as described herein in deglycosylated or gly-cosylated form as well as modified forms that can be obtained by altering the glycosylation pattern.

Functional equivalents or homologues of the polypeptides according to the invention can be produced by mutagenesis, e.g. by point mutation, lengthening or shortening of the protein or as described in more detail below.

Functional equivalents or homologs of the polypeptides according to the invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are a great many methods that can be used for the production of databases of potential homologues from a degenerated oligonucleotide sequence. Chemical synthesis of a degenerated gene sequence can be carried out in an automatic DNA synthesizer, and the synthetic gene can then be li-gated in a suitable expression vector. The use of a degenerated genome makes it possible to supply all sequences in a mixture, which code for the desired set of potential protein sequences. Methods of synthesis of degenerated oligonucleotides are known toa person skilled in the art.

In the prior art, several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologues according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologues.

An embodiment provided herein provides orthologs and paralogs of polypeptides disclosed herein as well as methods for identifying and isolating such orthologs and paralogs. A definition of the terms “ortholog” and “paralog” is given below and applies to amino acid and nucleic acid sequences.

The polypeptides of the invention include all active forms, including active sub-sequences, e.g., catalytic domains or active sites, of an enzyme of the invention. In one aspect, the invention provides catalytic domains or active sites as set forth below. In one aspect, the invention provides a peptide or polypeptide comprising or consisting of an active site domain as predicted through use of a database such as Pfam (http://pfam.wustl.edu/hmmsearch.shtml) (which is a large collection of multiple sequence alignments and hidden Markov models covering many common protein families, The Pfam protein families database, A. Bateman, E. Birney, L. Cerruti, R. Durbin, L. Etwiller, S. R. Eddy, S. Griffiths-Jones, K. L. Howe, M. Marshall, and E. L. L. Sonnhammer, Nucleic Acids Research, 30 (1): 276-280, 2002) or equivalent, as for example InterPro and SMART databases (http://www.ebi.ac.uk/interpro/scan.html, http://smart.embl-heidelberg.de/).

The invention also encompasses a “polypeptide variant” having the desired activity, wherein the variant polypeptide is selected from an amino acid sequence having at least 50%, 55%, 60%, 65%, 70%, 75%, 80%, 85%, 90%, 91%, 92%, 93%, 94%, 95%, 96%, 97%, 98% or 99%, sequence identity to a specific, in particular natural, amino acid sequence as referred to by a specific SEQ ID NO and contains at least one substitution modification relative said SEQ ID NO.

### 2. Nucleic Acids and Constructs

### 2. 1 Nucleic Acids

In this context the following definitions apply:

The terms “nucleic acid sequence,” “nucleic acid,” “nucleic acid molecule” and “polynucleotide” are used interchangeably meaning a sequence of nucleotides. A nucleic acid sequence may be a single-stranded or double-stranded deoxyribonucleotide, or ribonucleotide of any length, and include coding and non-coding sequences of agene, exons, introns, sense and antisense complimentary sequences, genomic DNA, cDNA, miRNA, siRNA, mRNA, rRNA, tRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference of thymine (T) being replaced by uracil (U). The term “nucleotide sequence” should also be understood as comprising a polynucleotide molecule or an oligonucleotide molecule in the form of a separate fragment or as a component of a larger nucleic acid.

An “isolated nucleic acid” or “isolated nucleic acid sequence” relates to a nucleic acid or nucleic acid sequence that is in an environment different from that in which the nucleic acid or nucleic acid sequence naturally occurs and can include those that are substantially free from contaminating endogenous material.

The term “naturally-occurring” as used herein as applied to a nucleic acid refers to a nucleic acid that is found in a cell of an organism in nature and which has not been intentionally modified by a human in the laboratory.

A “fragment” of a polynucleotide or nucleic acid sequence refers to contiguous nucleotides that is particularly at least 15 bp, at least 30 bp, at least 40 bp, at least 50 bp and/or at least 60 bp in length of the polynucleotide of an embodiment herein. Particularly the fragment of a polynucleotide comprises at least 25, more particularly at least 50, more particularly at least 75, more particularly at least 100, more particularly at least 150, more particularly at least 200, more particularly at least 300, more particularly at least 400, more particularly at least 500, more particularly at least 600, more particularly at least 700, more particularly at least 800, more particularly at least 900, more particularly at least 1000 contiguous nucleotides of the polynucleotide of an embodiment herein. Without being limited, the fragment of the polynucleotides herein may be used as a PCR primer, and/or as a probe, or for antisense gene silencing or RNAi.

As used herein, the term “hybridization” or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein below. Appropriate hybridization conditions can also be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

“Recombinant nucleic acid sequences” are nucleic acid sequences that result from the use of laboratory methods (for example, molecular cloning) to bring together genetic material from more than on source, creating or modifying a nucleic acid sequence that does not occur naturally and would not be otherwise found in biological organisms.

“Recombinant DNA technology” refers to molecular biology procedures to pre-pare a recombinant nucleic acid sequence as described, for instance, in Laboratory Manuals edited by Weigel and Glazebrook, 2002, Cold Spring Harbor Lab Press; and Sambrook et al., 1989, Cold Spring Harbor, NY, Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising, e.g., sequences involved in translation initiation, a coding region of cDNA or genomic DNA, introns, exons, and/or a 3′non-translated sequence comprising, e.g., transcription termination sites.

“Polycistronic” refers to nucleic acid molecules, in particular mRNAs, that can encode more than one polypeptide separately within the same nucleic acid molecule

A “chimeric gene” refers to any gene which is not normally found in nature in a species, in particular, a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription). The term “chimeric gene” also includes genes obtained through the combination of portions of one or more coding sequences to produce a new gene.

A “3′ UTR” or “3′ non-translated sequence” (also referred to as “3′ untranslated region,” or “3′end”) refers to the nucleic acid sequence found downstream of the coding sequence of a gene, which comprises, for example, a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly (A) tail may be added, which is involved in the transport of the mRNA to the site of translation, e.g., cytoplasm.

The term “primer” refers to a short nucleic acid sequence that is hybridized to a template nucleic acid sequence and is used for polymerization of a nucleic acid sequence complementary to the template.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

The invention also relates to nucleic acid sequences that code for polypeptides as defined herein.

In particular, the invention also relates to nucleic acid sequences (single-stranded and double-stranded DNA and RNA sequences, e.g. cDNA, genomic DNA and mRNA), coding for one of the above polypeptides and their functional equivalents, which can be obtained for example using artificial nucleotide analogs.

The invention relates both to isolated nucleic acid molecules, which code for polypeptides according to the invention or biologically active segments thereof, and to nucleic acid fragments, which can be used for example as hybridization probes or primers for identifying or amplifying coding nucleic acids according to the invention.

The present invention also relates to nucleic acids with a certain degree of “identity” to the sequences specifically disclosed herein. “Identity” between two nucleic acids means identity of the nucleotides, in each case over the entire length of the nucleic acid.

The “identity” between two nucleotide sequences (the same applies to peptide or amino acid sequences) is a function of the number of nucleotide residues (or amino acid residues) or that are identical in the two sequences when an alignment of these two sequences has been generated. Identical residues are defined as residues that are the same in the two sequences in a given position of the alignment. The percentage of sequence identity, as used herein, is calculated from the optimal alignment by taking the number of residues identical between two sequences dividing it by the total number of residues in the shortest sequence and multiplying by 100. The optimal alignment is the alignment in which the percentage of identity is the highest possible. Gaps may be introduced into one or both sequences in one or more positions of the alignment to obtain the optimal alignment. These gaps are then taken into account as non-identical residues for the calculation of the percentage of sequence identity. Alignment for the purpose of determining the percentage of amino acid or nucleic acid sequence identity can be achieved in various ways using computer programs and for instance publicly available computer programs available on the world wide web.

Particularly, the BLAST program (Tatiana et al, FEMS Microbiol Lett., 1999, 174:247-250, 1999) set to the default parameters, available from the National Center for Biotechnology Information (NCBI) website at ncbi.nlm.nih.gov/BLAST/bl2seq/wblast2.cgi, can be used to obtain an optimal alignment of protein or nucleic acid sequences and to calculate the percentage of sequence identity.

In another example the identity may be calculated by means of the Vector NTI Suite 7.1 program of the company Informax (USA) employing the Clustal Method (Hig-gins DG, Sharp PM. ((1989))) with the following settings:

### Multiple Alignment Parameters:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

### Pairwise Alignment Parameter:

| FAST algorithm | on |
|---|---|
| K-tuple size | 1 |
| Gap penalty | 3 |
| Window size | 5 |
| Number of best diagonals | 5 |

Alternatively the identity may be determined according to Chenna, et al. (2003), the web page: http://www.ebi.ac.uk/Tools/clustalw/index.html #and the following settings

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

All the nucleic acid sequences mentioned herein (single-stranded and double-stranded DNA and RNA sequences, for example cDNA and mRNA) can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al. (1989), see below.

The nucleic acid molecules according to the invention can in addition contain non-translated sequences from the 3′ and/or 5′ end of the coding genetic region.

The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Such probes or primers generally comprise a nucleotide sequence region which hybridizes under “stringent” conditions (as defined herein elsewhere) on at least about 12, particularly at least about 25, for example about 40, 50 or 75 successive nucleotides of a sense strand of a nucleic acid sequence according to the invention or of a corresponding antisense strand.

“Homologous” sequences include orthologous or paralogous sequences. Methods of identifying orthologs or paralogs including phylogenetic methods, sequence similarity and hybridization methods are known in the art and are described herein.

“Paralogs” result from gene duplication that gives rise to two or more genes with similar sequences and similar functions. Paralogs typically cluster together and are formed by duplications of genes within related plant species. Paralogs are found in groups of similar genes using pair-wise Blast analysis or during phylogenetic analysis of gene families using programs such as CLUSTAL. In paralogs, consensus sequences can be identified characteristic to sequences within related genes and having similar functions of the genes.

“Orthologs”, or orthologous sequences, are sequences similar to each other because they are found in species that descended from a common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in host cells or organisms, such as plants or microorganisms, overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making the host cells, organism such as plants or microorganisms producing enzymes of the invention.

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

A nucleic acid molecule according to the invention can be recovered by means of standard techniques of molecular biology and the sequence information supplied according to the invention. For example, cDNA can be isolated from a suitable cDNA library, using one of the concretely disclosed complete sequences or a segment thereof as hybridization probe and standard hybridization techniques (as described for example in Sambrook, (1989)).

In addition, a nucleic acid molecule comprising one of the disclosed sequences or a segment thereof, can be isolated by the polymerase chain reaction, using the oligonucleotide primers that were constructed on the basis of this sequence. The nucleic acid amplified in this way can be cloned in a suitable vector and can be characterized by DNA sequencing. The oligonucleotides according to the invention can also be produced by standard methods of synthesis, e.g. using an automatic DNA synthesizer.

Nucleic acid sequences according to the invention or derivatives thereof, homologues or parts of these sequences, can for example be isolated by usual hybridization techniques or the PCR technique from other bacteria, e.g. via genomic or cDNA libraries. These DNA sequences hybridize in standard conditions with the sequences according to the invention.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These “standard conditions” vary depending on the nucleic acid used (oligonucleotide, longer fragment or complete sequence) or depending on which type of nucleic acid—DNA or RNA—is used for hybridization. For example, the melting temperatures for DNA: DNA hybrids are approx. 10° C. lower than those of DNA: RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA: DNA hybrids are 0.1×SSC and temperatures between about 20° C. to 45° C., particularly between about 30° C. to 45° C. For DNA: RNA hybrids the hybridization conditions are advantageously 0.1×SSC and temperatures between about 30° C. to 55° C., particularly between about 45° C. to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., 1989, and can be calculated using formulae that are known by a person skilled in the art, for example depending on the length of the nucleic acids, the type of hybrids or the G+C content. A person skilled in the art can obtain further information on hybridization from the following textbooks: Ausubel et al. (eds), (1985), Brown (ed) (1991).

“Hybridization” can in particular be carried out under stringent conditions. Such hybridization conditions are for example described in Sambrook (1989), or in Current Protocols in Molecular Biology, John Wiley & Sons, N.Y. (1989), 6.3.1-6.3.6.

As used herein, the term hybridization or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other. Definitions of low stringency, moderate, and high stringency hybridization conditions are provided herein.

Appropriate hybridization conditions can be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

As used herein, defined conditions of low stringency are as follows. Filters containing DNA are pretreated for 6 h at 40° C. in a solution containing 35% formamide, 5x SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20x106 32P-labeled probe is used. Filters are incubated in hybridization mixture for 18-20 h at 40° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of moderate stringency are as follows. Filters containing DNA are pretreated for 7 h at 50° C. in a solution containing 35% forma-mide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20x106 32P-labeled probe is used. Filters are incubated in hybridization mixture for 30 h at 50° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

As used herein, defined conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 μg/ml denatured salmon sperm DNA and 5-20x106 cpm of 32P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes.

Other conditions of low, moderate, and high stringency well known in the art (e.g., as employed for cross-species hybridizations) may be used if the above conditions are in-appropriate (e.g., as employed for cross-species hybridizations).

A detection kit for nucleic acid sequences encoding a polypeptide of the invention may include primers and/or probes specific for nucleic acid sequences encoding the polypeptide, and an associated protocol to use the primers and/or probes to detect nucleic acid sequences encoding the polypeptide in a sample. Such detection kits may be used to determine whether a plant, organism, microorganism or cell has been modified, i.e., transformed with a sequence encoding the polypeptide.

To test a function of variant DNA sequences according to an embodiment herein, the sequence of interest is operably linked to a selectable or screenable marker gene and expression of said reporter gene is tested in transient expression assays, for example, with microorganisms or with protoplasts or in stably transformed plants.

The invention also relates to derivatives of the concretely disclosed or derivable nucleic acid sequences.

Thus, further nucleic acid sequences according to the invention can be derived from the sequences specifically disclosed herein and can differ from it by one or more, like 1 to 20, in particular 1 to 15 or 5 to 10 additions, substitutions, insertions or deletions of one or several (like for example 1 to 10) nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism.

According to a particular embodiment of the invention variant nucleic acids may be prepared in order to adapt its nucleotide sequence to a specific expression system. For example, bacterial expression systems are known to more efficiently express polypeptides if amino acids are encoded by particular codons. Due to the degeneracy of the genetic code, more than one codon may encode the same amino acid sequence, multiple nucleic acid sequences can code for the same protein or polypeptide, all these DNA sequences being encompassed by an embodiment herein. Where appropriate, the nucleic acid sequences encoding the polypeptides described herein may be optimized for increased expression in the host cell. For example, nucleic acids of an embodiment herein may be synthesized using codons particular to a host for improved expression.

The invention also encompasses naturally occurring variants, e.g. splicing variants or allelic variants, of the sequences described therein.

Allelic variants may have at least 60% homology at the level of the derived amino acid, particularly at least 80% homology, quite especially particularly at least 90% homology over the entire sequence range (regarding homology at the amino acid level, reference should be made to the details given above for the polypeptides). Advantageously, the homologies can be higher over partial regions of the sequences.

The invention also relates to sequences that can be obtained by conservative nucleotide substitutions (i.e. as a result thereof the amino acid in question is replaced by an amino acid of the same charge, size, polarity and/or solubility).

The invention also relates to the molecules derived from the concretely disclosed nucleic acids by sequence polymorphisms. Such genetic polymorphisms may exist in cells from different populations or within a population due to natural allelic variation. Allelic variants may also include functional equivalents. These natural variations usually produce a variance of 1 to 5% in the nucleotide sequence of a gene. Said polymorphisms may lead to changes in the amino acid sequence of the polypeptides disclosed herein. Allelic variants may also include functional equivalents.

Furthermore, derivatives are also to be understood to be homologs of the nucleic acid sequences according to the invention, for example animal, plant, fungal or bacterial homologs, shortened sequences, single-stranded DNA or RNA of the coding and noncoding DNA sequence. For example, homologs have, at the DNA level, a homology of at least 40%, particularly of at least 60%, especially particularly of at least 70%, quite especially particularly of at least 80% over the entire DNA region given in a sequence specifically disclosed herein.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

### 2.2 Constructs for Expressing Polypeptides of the Invention

In this context the following definitions apply:

“Expression of a gene” encompasses “heterologous expression” and “over-expression” and involves transcription of the gene and translation of the mRNA into a protein. Overexpression refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

An “expression vector” as used herein includes any linear or circular recombinant vector including but not limited to viral vectors, bacteriophages and plasmids. The skilled person is capable of selecting a suitable vector according to the expression system. In one embodiment, the expression vector includes the nucleic acid of an embodiment herein operably linked to at least one “regulatory sequence”, which controls transcription, translation, initiation and termination, such as a transcriptional promoter, operator or enhancer, or an mRNA ribosomal binding site and, optionally, including at least one selection marker. Nucleotide sequences are “operably linked” when the regulatory sequence functionally relates to the nucleic acid of an embodiment herein.

An “expression system” as used herein encompasses any combination of nucleic acid molecules required for the expression of one, or the co-expression of two or more polypeptides either in vivo of a given expression host, or in vitro. The respective coding sequences may either be located on a single nucleic acid molecule or vector, as for example a vector containing multiple cloning sites, or on a polycistronic nucleic acid, or may be distributed over two or more physically distinct vectors. As a particular example there may be mentioned an operon comprising a promotor sequence, one or more operator sequences and one or more structural genes each encoding an enzyme as described herein

As used herein, the terms “amplifying” and “amplification” refer to the use of any suitable amplification methodology for generating or detecting recombinant of naturally expressed nucleic acid, as described in detail, below. For example, the invention provides methods and reagents (e.g., specific degenerate oligonucleotide primer pairs, oligo dT primer) for amplifying (e.g., by polymerase chain reaction, PCR) naturally expressed (e.g., genomic DNA or mRNA) or recombinant (e.g., cDNA) nucleic acids of the invention in vivo, ex vivo or in vitro.

“Regulatory sequence” refers to a nucleic acid sequence that determines expression level of the nucleic acid sequences of an embodiment herein and is capable of regulating the rate of transcription of the nucleic acid sequence operably linked to the regulatory sequence. Regulatory sequences comprise promoters, enhancers, transcription factors, promoter elements and the like.

A “promoter”, a “nucleic acid with promoter activity” or a “promoter sequence” is understood as meaning, in accordance with the invention, a nucleic acid which, when functionally linked to a nucleic acid to be transcribed, regulates the transcription of said nucleic acid. “Promoter” in particular refers to a nucleic acid sequence that controls the expression of a coding sequence by providing a binding site for RNA polymerase and other factors required for proper transcription including without limitation transcription factor binding sites, repressor and activator protein binding sites. The meaning of the term promoter also includes the term “promoter regulatory sequence”. Promoter regulatory sequences may include upstream and downstream elements that may influences transcription, RNA processing or stability of the associated coding nucleic acid sequence. Promoters include naturally-derived and synthetic sequences. The coding nucleic acid sequences is usually located downstream of the promoter with respect to the direction of the transcription starting at the transcription initiation site.

In this context, a “functional” or “operative” linkage is understood as meaning for example the sequential arrangement of one of the nucleic acids with a regulatory sequence. For example the sequence with promoter activity and of a nucleic acid sequence to be transcribed and optionally further regulatory elements, for example nucleic acid sequences which ensure the transcription of nucleic acids, and for example a terminator, are linked in such a way that each of the regulatory elements can perform its function upon transcription of the nucleic acid sequence. This does not necessarily require a direct linkage in the chemical sense. Genetic control sequences, for example enhancer sequences, can even exert their function on the target sequence from more remote positions or even from other DNA molecules. Preferred arrangements are those in which the nucleic acid sequence to be transcribed is positioned behind (i.e. at the 3′-end of) the promoter sequence so that the two sequences are joined together cova-lently. The distance between the promoter sequence and the nucleic acid sequence to be expressed recombinantly can be smaller than 200 base pairs, or smaller than 100 base pairs or smaller than 50 base pairs.

In addition to promoters and terminator, the following may be mentioned as examples of other regulatory elements: targeting sequences, enhancers, polyadenylation signals, selectable markers, amplification signals, replication origins and the like. Suitable regulatory sequences are described, for example, in Goeddel, Gene Expression Technology: Methods in Enzymology 185, Academic Press, San Diego, CA (1990).

The term “constitutive promoter” refers to an unregulated promoter that allows for continual transcription of the nucleic acid sequence it is operably linked to.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous. The nucleotide sequence associated with the promoter sequence may be of homologous or heterologous origin with respect to the plant to be transformed. The sequence also may be entirely or partially synthetic. Regardless of the origin, the nucleic acid sequence associated with the promoter sequence will be expressed or silenced in accordance with promoter properties to which it is linked after binding to the polypeptide of an embodiment herein. The associated nucleic acid may code for a protein that is desired to be expressed or suppressed throughout the organism at all times or, alternatively, at a specific time or in specific tissues, cells, or cell compartment. Such nucleotide sequences particularly encode proteins conferring desirable phenotypic traits to the host cells or organism altered or transformed therewith. More particularly, the associated nucleotide sequence leads to the production of the product or products of interest as herein defined in the cell or organism. Particularly, the nucleotide sequence encodes a polypeptide having an enzyme activity as herein defined.

The nucleotide sequence as described herein above may be part of an “expression cassette”. The terms “expression cassette” and “expression construct” are used synonymously. The (particularly recombinant) expression construct contains a nucleotide sequence which encodes a polypeptide according to the invention and which is under genetic control of regulatory nucleic acid sequences.

In a process applied according to the invention, the expression cassette may be part of an “expression vector”, in particular of a recombinant expression vector.

An “expression unit” is understood as meaning, in accordance with the invention, a nucleic acid with expression activity which comprises a promoter as defined herein and, after functional linkage with a nucleic acid to be expressed or a gene, regulates the expression, i.e. the transcription and the translation of said nucleic acid or said gene. It is therefore in this connection also referred to as a “regulatory nucleic acid sequence”. In addition to the promoter, other regulatory elements, for example enhancers, can also be present.

An “expression cassette” or “expression construct” is understood as meaning, in accordance with the invention, an expression unit which is functionally linked to the nucleic acid to be expressed or the gene to be expressed. In contrast to an expression unit, an expression cassette therefore comprises not only nucleic acid sequences which regulate transcription and translation, but also the nucleic acid sequences that are to be expressed as protein as a result of transcription and translation.

The terms “expression” or “overexpression” describe, in the context of the invention, the production or increase in intracellular activity of one or more polypeptides in a microorganism, which are encoded by the corresponding DNA. To this end, it is possible for example to introduce a gene into an organism, replace an existing gene with another gene, increase the copy number of the gene(s), use a strong promoter or use a gene which encodes for a corresponding polypeptide with a high activity; optionally, these measures can be combined.

Particularly such constructs according to the invention comprise a promoter 5′-upstream of the respective coding sequence and a terminator sequence 3′-downstream and optionally other usual regulatory elements, in each case in operative linkage with the coding sequence.

Nucleic acid constructs according to the invention comprise in particular a sequence coding for a polypeptide for example derived from the amino acid related SEQ ID NOs as described therein or the reverse complement thereof, or derivatives and homologs thereof and which have been linked operatively or functionally with one or more regulatory signals, advantageously for controlling, for example increasing, gene expression.

In addition to these regulatory sequences, the natural regulation of these sequences may still be present before the actual structural genes and optionally may have been genetically modified so that the natural regulation has been switched off and expression of the genes has been enhanced. The nucleic acid construct may, however, also be of simpler construction, i.e. no additional regulatory signals have been inserted before the coding sequence and the natural promoter, with its regulation, has not been removed. Instead, the natural regulatory sequence is mutated such that regulation no longer takes place and the gene expression is increased.

A preferred nucleic acid construct advantageously also comprises one or more of the already mentioned “enhancer” sequences in functional linkage with the promoter, which sequences make possible an enhanced expression of the nucleic acid sequence. Additional advantageous sequences may also be inserted at the 3′-end of the DNA sequences, such as further regulatory elements or terminators. One or more copies of the nucleic acids according to the invention may be present in a construct. In the construct, other markers, such as genes which complement auxotrophisms or antibiotic resistances, may also optionally be present so as to select for the construct.

Examples of suitable regulatory sequences are present in promoters such as cos, tac, trp, tet, trp-tet, Ipp, lac, Ipp-lac, lacla, 17, T5, T3, gal, trc, ara, rhaP (rhaP-BAD) SP6, lambda-PR or in the lambda-PL promoter, and these are advantageously employed in Gram-negative bacteria. Further advantageous regulatory sequences are present for example in the Gram-positive promoters amy and SPO2, in the yeast or fungal promoters ADC1, MFalpha, AC, P-60, CYC1, GAPDH, TEF, rp28, ADH. Artificial promoters may also be used for regulation.

For expression in a host organism, the nucleic acid construct is inserted advantageously into a vector such as, for example, a plasmid or a phage, which makes possible optimal expression of the genes in the host. Vectors are also understood as meaning, in addition to plasmids and phages, all the other vectors which are known to the skilled worker, that is to say for example viruses such as SV40, CMV, baculovirus and adenovirus, transposons, IS elements, phasmids, cosmids and linear or circular DNA or artificial chromosomes. These vectors are capable of replicating autonomously in the host organism or else chromosomally. These vectors are a further development of the invention. Binary or cpo-integration vectors are also applicable.

Suitable plasmids are, for example, in E. coli pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCI, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The abovementioned plasmids are a small selection of the plasmids which are possi-ble. Further plasmids are well known to the skilled worker and can be found for example in the book Cloning Vectors (Eds. Pouwels P. H. et al. Elsevier, Amsterdam-New York-Oxford, 1985, ISBN 0 444 904018).

In a further development of the vector, the vector which comprises the nucleic acid construct according to the invention or the nucleic acid according to the invention can advantageously also be introduced into the microorganisms in the form of a linear DNA and integrated into the host organism's genome via heterologous or homologous recombination. This linear DNA can consist of a linearized vector such as a plasmid or only of the nucleic acid construct or the nucleic acid according to the invention.

For optimal expression of heterologous genes in organisms, it is advantageous to modify the nucleic acid sequences to match the specific “codon usage” used in the organism. The “codon usage” can be determined readily by computer evaluations of other, known genes of the organism in question.

An expression cassette according to the invention is generated by fusing a suitable promoter to a suitable coding nucleotide sequence and a terminator or polyadenylation signal. Customary recombination and cloning techniques are used for this pur-pose, as are described, for example, in T. Maniatis, E. F. Fritsch and J. Sambrook, Molecular Cloning: A Laboratory Manual, Cold Spring Harbor Laboratory, Cold Spring Harbor, NY (1989) and in T. J. Silhavy, M. L. Berman and L. W. Enquist, Experiments with Gene Fusions, Cold Spring Harbor Laboratory, Cold Spring Harbor, NY (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which makes possible optimal expression of the genes in the host. Vectors are well known to the skilled worker and can be found for example in “cloning vectors” (Pouwels P. H. et al., Ed., Elsevier, Amsterdam-New York-Oxford, 1985).

An alternative embodiment of an embodiment herein provides a method to “al-ter gene expression” in a host cell. For instance, the polynucleotide of an embodiment herein may be enhanced or overexpressed or induced in certain contexts (e.g. upon exposure to certain temperatures or culture conditions) in a host cell or host organism.

Alteration of expression of a polynucleotide provided herein may also result in ectopic expression which is a different expression pattern in an altered and in a control or wild-type organism. Alteration of expression occurs from interactions of polypeptide of an embodiment herein with exogenous or endogenous modulators, or as a result of chemical modification of the polypeptide. The term also refers to an altered expression pattern of the polynucleotide of an embodiment herein which is altered below the detection level or completely suppressed activity.

In one embodiment, provided herein is also an isolated, recombinant or synthetic polynucleotide encoding a polypeptide or variant polypeptide provided herein.

In one embodiment, several polypeptide encoding nucleic acid sequences are co-expressed in a single host, particularly under control of different promoters. In another embodiment, several polypeptide encoding nucleic acid sequences can be present on a single transformation vector or be co-transformed at the same time using separate vectors and selecting transformants comprising both chimeric genes. Similarly, one or polypeptide encoding genes may be expressed in a single plant, cell, microorganism or organism together with other chimeric genes.

### EXPERIMENTAL SECTION

### Examples

### A. Synthesis

### 1. Preparation of compounds I.a

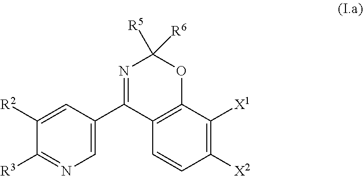

1.1 Preparation of 4-[6-(difluoromethyl)-5-methyl-3-pyridyl]spiro[1,3-benzoxazine-2,1′-cyclobutane](example compound 25 in Table I)

1.1a) Preparation of spiro[3H-1,3-benzoxazine-2,1′-cyclobutane]-4-one p-Toluene sulfonic acid (98 mg, 0.2 eq) was added to a suspension of 3-fluoro-2-hydroxy-benzamide (400 mg, 1 eq) and cyclobutanone (542 mg, 3 eq) in toluene (30 ml), and the mixture was heated at reflux with azeotropic removal of water for 12 h. The reaction solution was cooled and concentrated in vacuo, and the resultant residue was diluted with ethyl acetate, washed successively with 2 N HCl, water and brine, and dried over anhydrous magnesium sulfate. Removal of solvent in vacuo afforded the titled compound (526 mg) as a brown powder.

¹H NMR (400 MHZ, CDCl₃): δ [ppm]: 8.02-7.86 (m, 1H), 7.47 (ddd, J=8.3, 7.3, 1.7 Hz, 1H), 7.10 (td, J=7.5, 1.1 Hz, 1H), 7.01 (ddd, J=8.3, 1.1, 0.5 Hz, 1H), 6.62 (s, 1H), 2.65-2.50 (m, 2H), 2.34 (ddtt, J=12.5, 6.4, 3.1, 1.5 Hz, 2H), 2.07-1.91 (m, 1H), 1.84 (dtt, J=11.6, 9.3, 6.4 Hz, 1H).

### 1.1b) Preparation of spiro[1,3-benzoxazine-2,1′-cyclobutane]-4-yl trifluoro-methanesul-fonate

Trifluoromethanesulfonic anhydride (7.8 g, 2.5 eq) and 2,6-lutidine (2.38 g, 2 eq) were added dropwise to a suspension of spiro[3H-1,3-benzoxazine-2,1′-cyclobutane]-4-one (2.1 g, 1 eq) in dichloromethane (120 mL) under cooling at −78° C., and the mixture was stirred at the same tem-perature for 1.0 hour. The reaction mixture stirred for 20 min at 0° C., poured into ice water, and the solution was extracted with dichloromethane. The organic layer was washed successively with a saturated aqueous solution of sodium bicarbonate and brine, dried over anhydrous magnesium sulfate, and concentrated in vacuo to give the titled compound (1.8 g) as a brown oil. The title compound was used directly without further purification.

### 1.1c) Preparation of 4-[6-(difluoromethyl)-5-methyl-3-pyridyl]spiro[1,3-benzoxazine-2,1′-cyclobutane]

[6-(difluoromethyl)-5-methyl-3-pyridyl]boronic acid (572 mg, 1.1 eq), potassium carbonate (1.54 g, 4 eq), water (2 ml) and dichlorobis(triphenylphosphine) palladium (II) (391 mg, 0.2 eq) were added to a solution of spiro[1,3-benzoxazine-2,1′-cyclobutane]-4-yl trifluoromethanesulfonate (900 mg, 1 eq) in dimethoxyethane (10 mL), and the mixture was stirred under argon atmosphere at 80° C. for 2.5 hours. After cooling, the reaction solution was diluted with ethyl acetate, and the solution was washed successively with water and brine, dried over anhydrous magnesium sulfate, and concentrated in vacuo. The crude product was purified by High Performance Liquid Chromatography on silica gel (HPLC-column Kinetex XB C_{18 1,7}u (50×2.1 mm); eluent: acetonitrile/water (gradient from 5:95 to 100:0 in 1.5 min at 60° C., flow gradient from 0.8 to 1.0 ml/min in 1.5 min) to give the titled compound (97 mg) as a yellow-brown oil. ¹H NMR (400 MHZ, CDCl₃): δ [ppm]: 8.67-8.60 (m, 1H), 7.90-7.84 (m, 1H), 7.42 (td, J=7.8, 1.6 Hz, 1H), 7.12 (dd, J=7.7, 1.6 Hz, 1H), 7.02 (d, J=8.2 Hz, 1H), 6.98-6.93 (m, 1H), 6.75 (t, J=54.5 Hz, 1H), 2.59 (s, 3H), 2.57-2.46 (m, 4H), 1.99 (dddd, J=25.7, 15.6, 7.8, 4.1 Hz, 2H).

The compounds listed in Table I were prepared in an analogous manner.

TABLE I

| Ex.- | | | HPLC Rₜ | |
|---|---|---|---|---|
| No. | Structure | Description | (min)* | LC-MS |
| Ex-1 | 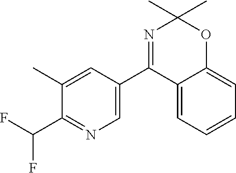 [SMILES: Cc1cc(C2=NC(C)(C)Oc3ccccc32)cnc1C(F)F] | Cpd. I.a wherein R² = Me, R³ = CHF₂, R⁵, R⁶ = Me, X¹, X² = H | 1.22 | 303.0 |
| Ex-2 | 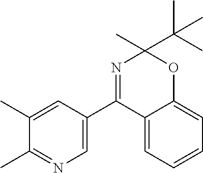 [SMILES: Cc1cc(C2=NC(C)(C(C)(C)C)Oc3ccccc32)cnc1C] | Cpd. I.a wherein R², R³ = Me, R⁵ = Me, R⁶ = t-Bu, X¹, X² = H | 1.03 | 309.0 |
| Ex-3 | 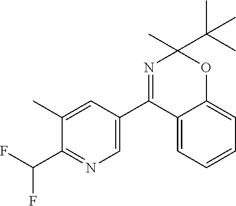 [SMILES: Cc1cc(C2=NC(C)(C(C)(C)C)Oc3ccccc32)cnc1C(F)F] | Cpd. I.a wherein R² = Me, R³ = CHF₂, R⁵ = Me, R⁶ = t-Bu, X¹, X² = H | 1.49 | 345.0 |
| Ex-4 | 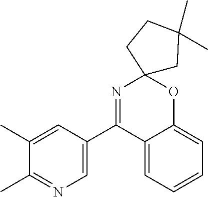 | Cpd. I.a wherein R², R³ = Me, R⁵, R⁶ form together with C atom to which they are bound 3,3-dimethylcyclopentan-1,1- diyl, X¹, X² = H | 0.999 | 321.0 |
| Ex-5 | 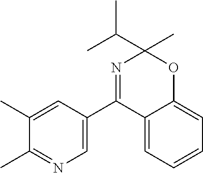 [SMILES: Cc1cc(C2=NC(C)(C(C)C)Oc3ccccc32)cnc1C] | Cpd. I.a wherein R², R³ = Me, R⁵ = Me, R⁶ = iPr, X¹, X² = H | 0.921 | 295.2 |
| Ex-6 | 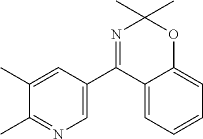 [SMILES: Cc1cc(C2=NC(C)(C)Oc3ccccc32)cnc1C] | Cpd. I.a wherein R², R³ = Me, R⁵, R⁶ = Me, X¹, X² = H | 0.783 | 267.2 |
| Ex-7 | 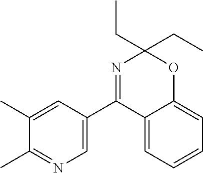 [SMILES: CCC1(CC)N=C(c2cnc(C)c(C)c2)c2ccccc2O1] | Cpd. I.a wherein R², R³ = Me, R⁵, R⁶ = Et, X¹, X² = H | 0.906 | 295.0 |
| Ex-8 | 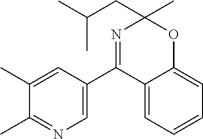 [SMILES: Cc1cc(C2=NC(C)(CC(C)C)Oc3ccccc32)cnc1C] | Cpd. I.a wherein R², R³ = Me, R⁵ = Me, R⁶ = iBu, X¹, X² = H | 0.976 | 309.3 |
| Ex-9 | 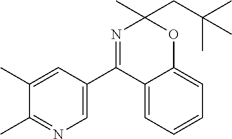 [SMILES: Cc1cc(C2=NC(C)(CC(C)(C)C)Oc3ccccc32)cnc1C] | Cpd. I.a wherein R², R³ = Me, R⁵ = Me, R⁶ = CH₂C(CH₃)₃, X¹, X² = H | 1.019 | 323.3 |
| Ex-10 | 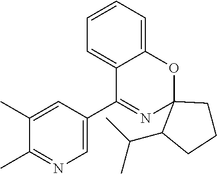 [SMILES: Cc1cc(C2=NC3(CCCC3C(C)C)Oc3ccccc32)cnc1C] | Cpd. I.a wherein R², R³ = Me, R⁵, R⁶ form together with C atom to which they are bound 2-isopropylcyclopentan-1,1-diyl, X¹, X² = H | 1.053 | 335.0 |
| Ex-11 | 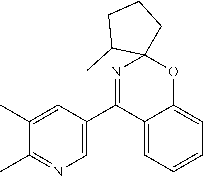 [SMILES: Cc1cc(C2=NC3(CCCC3C)Oc3ccccc32)cnc1C] | Cpd. I.a wherein R², R³ = Me, R⁵, R⁶ form together with C atom to which they are bound 2-methylcyclopentan-1,1-diyl, X¹, X² = H | 0.951 | 307.0 |
| Ex-12 | 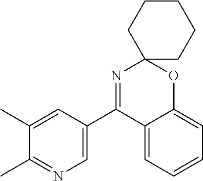 [SMILES: Cc1cc(C2=NC3(CCCCC3)Oc3ccccc32)cnc1C] | Cpd. I.a wherein R², R³ = Me, R⁵, R⁶ form together with C atom to which they are bound cyclohexan-1,1-diyl, X¹, X² = H | 0.93 | 307.1 |
| Ex-13 | 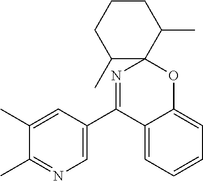 [SMILES: Cc1cc(C2=NC3(Oc4ccccc42)C(C)CCCC3C)cnc1C] | Cpd. I.a wherein R², R³ = Me, R⁵, R⁶ form together with C atom to which they are bound 2,6-dimethylcyclohexan-1,1-diyl, X¹, X² = H | 1.047 | 335.3 |
| Ex-14 | 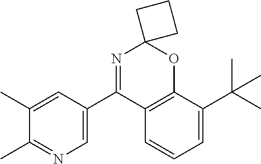 [SMILES: Cc1cc(C2=NC3(CCC3)Oc3c2cccc3C(C)(C)C)cnc1C] | Cpd. I.a wherein R², R³ = Me, R⁵, R⁶ form together with C atom to which they are bound cyclobutan-1,1-diyl, X¹ = t-Bu, X² = H | 1.046 | 335.3 |
| Ex-15 | 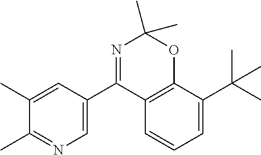 [SMILES: Cc1cc(C2=NC(C)(C)Oc3c2cccc3C(C)(C)C)cnc1C] | Cpd. I.a wherein R², R³ = Me, R⁵, R⁶ = Me, X¹ = t-Bu, X² = H | 1.014 | 323.2 |
| Ex-16 | 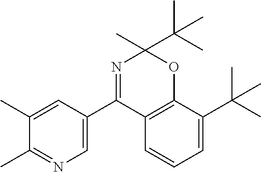 [SMILES: Cc1cc(C2=NC(C)(C(C)(C)C)Oc3c2cccc3C(C)(C)C)cnc1C] | Cpd. I.a wherein R², R³ = Me, R⁵ = Me, R⁶ t-Bu, X¹ = t-Bu, X² = H | 1.216 | 365.1 |
| Ex-17 | 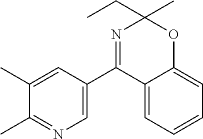 [SMILES: CCC1(C)N=C(c2cnc(C)c(C)c2)c2ccccc2O1] | Cpd. I.a wherein R², R³ = Me, R⁵ = Me, R⁶ = Et, X¹, X² = H | 0.852 | 281.0 |
| Ex-18 | 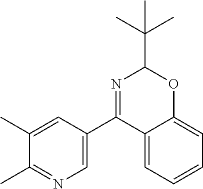 [SMILES: Cc1cc(C2=NC(C(C)(C)C)Oc3ccccc32)cnc1C] | Cpd. I.a wherein R², R³ = Me, R⁵ = H R⁶ = t-Bu, X¹, X² = H | 0.982 | 294.9 |
| Ex-19 | 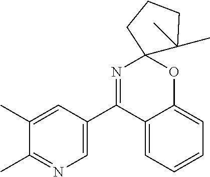 | Cpd. I.a wherein R², R³ = Me, R⁵, R⁶ form together with C atom to which they are bound 2,2-dimethylcyclopentan-1,1- diyl, X¹, X² = H | 1.02 | 321.2 |
| Ex-20 | 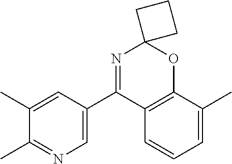 | Cpd. I.a wherein R², R³ = Me, R⁵, R⁶ form together with C atom to which they are bound cyclobutan-1,1-diyl, X¹ = Me, X² = H | 0.905 | 293.1 |
| Ex-21 | 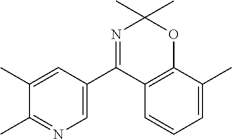 | Cpd. I.a wherein R², R³ = Me, R⁵, R⁶ = Me, X¹ = Me, X² = H | 0.859 | 281.1 |
| Ex-22 | 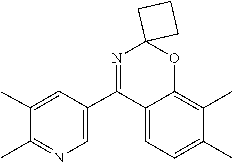 | Cpd. I.a wherein R², R³ = Me, R⁵, R⁶ form together with C atom to which they are bound cyclobutan-1,1-diyl, X¹, X² = Me | 0.946 | 306.9 |
| Ex-23 | 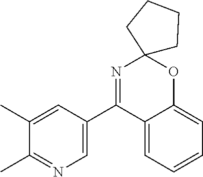 [SMILES: Cc1cc(C2=NC3(CCCC3)Oc3ccccc32)cnc1C] | Cpd. I.a wherein R², R³ = Me, R⁵, R⁶ form together with C atom to which they are bound cyclopentan-1,1-diyl, X¹, X² = H | 0.888 | 293.0 |
| Ex-24 | 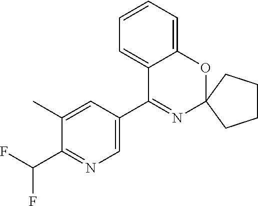 [SMILES: Cc1cc(C2=NC3(CCCC3)Oc3ccccc32)cnc1C(F)F] | Cpd. I.a wherein R² = Me, R³ = CHF₂, R⁵, R⁶ form together with C atom to which they are bound cyclopentan-1,1-diyl, X¹, X² = H | 1.327 | 329.0 |
| Ex-25 | 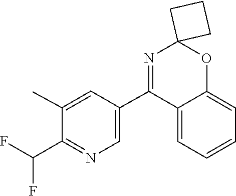 [SMILES: Cc1cc(C2=NC3(CCC3)Oc3ccccc32)cnc1C(F)F] | Cpd. I.a wherein R² = Me, R³ = CHF₂, R⁵, R⁶ form together with C atom to which they are bound cyclobutan-1,1-diyl, X¹, X² = H | 1.275 | 315.2 |
| Ex-26 | 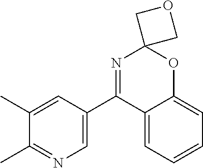 [SMILES: Cc1cc(C2=NC3(COC3)Oc3ccccc32)cnc1C] | Cpd. I.a wherein R², R³ = Me, R⁵, R⁶ form together with C atom to which they are bound oxetan-3,3-diyl, X¹, X² = H | 0.721 | 281.0 |
| Ex-27 | 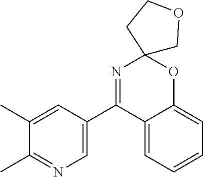 [SMILES: Cc1cc(C2=NC3(CCOC3)Oc3ccccc32)cnc1C] | Cpd. I.a wherein R², R³ = Me, R⁵, R⁶ form together with C atom to which they are bound tetrahydrofuran-3,3-diyl, X¹, X² = H | 0.732 | 295.1 |
| Ex-28 | 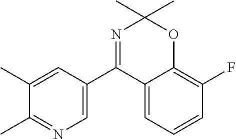 [SMILES: Cc1cc(C2=NC(C)(C)Oc3c(F)cccc32)cnc1C] | Cpd. I.a wherein R², R³ = Me, R⁵, R⁶ = Me, X¹ = F, X² = H | 0.81 | 285.1 |
| Ex-29 | 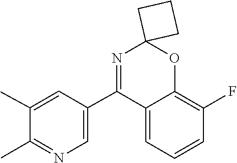 [SMILES: Cc1cc(C2=NC3(CCC3)Oc3c(F)cccc32)cnc1C] | Cpd. I.a wherein R², R³ = Me, R⁵, R⁶ form together with C atom to which they are bound cyclobutan-1,1-diyl, X¹ = F, X² = H | 0.853 | 297.2 |
| Ex-30 | 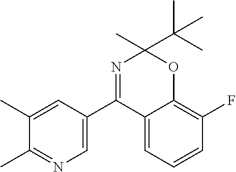 [SMILES: Cc1cc(C2=NC(C)(C(C)(C)C)Oc3c(F)cccc32)cnc1C] | Cpd. I.a wherein R², R³ = Me, R⁵ = Me, R⁶ = t-Bu, X¹ = F, X² = H | 1.031 | 327.1 |
| Ex-31 | 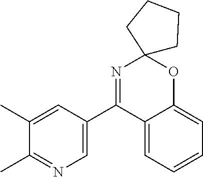 [SMILES: Cc1cc(C2=NC3(CCCC3)Oc3ccccc32)cnc1C] | Cpd. I.a wherein R², R³ = Me, R⁵, R⁶ form together with C atom to which they are bound cyclopentan-1,1-diyl, X¹, X² = H | 0.986 | 319.0 |
| Ex-32 | 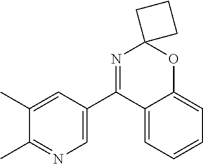 [SMILES: Cc1cc(C2=NC3(CCC3)Oc3ccccc32)cnc1C] | Cpd. I.a wherein R², R³ = Me, R⁵, R⁶ form together with C atom to which they are bound cyclobutan-1,1-diyl, X¹, X² = H | 0.829 | 277.0 |
| Ex-33 | 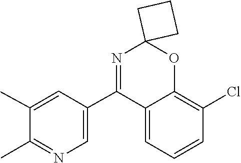 [SMILES: Cc1cc(C2=NC3(CCC3)Oc3c(Cl)cccc32)cnc1C] | Cpd. I.a wherein R², R³ = Me, R⁵, R⁶ form together with C atom to which they are bound cyclobutan-1,1-diyl, X¹ = F, X² = H | 0.934 | 313.0 |
| Ex-34 | 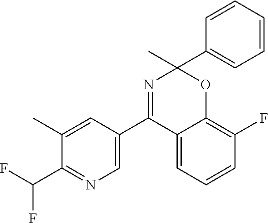 [SMILES: Cc1cc(C2=NC(C)(c3ccccc3)Oc3c(F)cccc32)cnc1C(F)F] | Cpd. I.a wherein R² = Me, R³ = CHF₂, R⁵ = Me, R⁶ = Ph, X¹ = F, X² = H | 1.379 | 382.9 |
| Ex-35 | 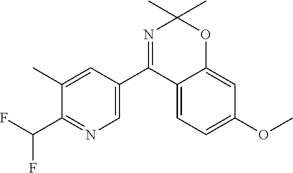 [SMILES: COc1ccc2c(c1)OC(C)(C)N=C2c1cnc(C(F)F)c(C)c1] | Cpd. I.a wherein R² = Me, R³ = CHF₂, R⁵, R⁶ = Me, X¹ = H, X² = OMe | 1.036 | 333.1 |
| Ex-36 | 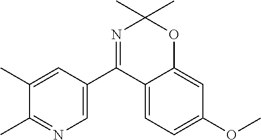 | Cpd. I.a wherein R², R³ = Me, R⁵, R⁶ = Me, X¹ = H, X² = OMe | 0.795 | 297.2 |
| Ex-37 | 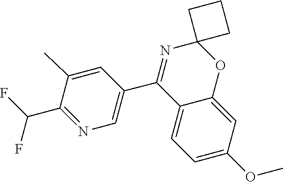 [SMILES: COc1ccc2c(c1)OC1(CCC1)N=C2c1cnc(C(F)F)c(C)c1] | Cpd. I.a wherein R² = Me, R³ = CHF₂, R⁵, R⁶ form together with C atom to which they are bound cyclobutan-1,1-diyl, X¹ = H, X² = OMe | 1.138 | 345.1 |
| Ex-38 | 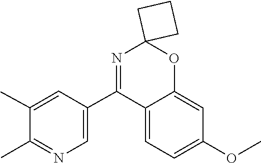 | Cpd. I.a wherein R², R³ = Me, R⁵, R⁶ form together with C atom to which they are bound cyclobutan-1,1-diyl, X¹ = H, X² = OMe | 1.138 | 309.1 |

*HPLC: High Performance Liquid Chromatography;
HPLC-column Kinetex XB C18 1,7 μ (50 × 2,1 mm); eluent:
acetonitrile/water + 0.1% trifluoroacetic acid (gradient from 5:95 to 100:
0 in 1.5 min at 60° C., flow gradient from 0.8 to 1.0 ml/min in 1.5 min).
Rₜ: retention time in minutes.

### 2. Preparation of compounds I.b

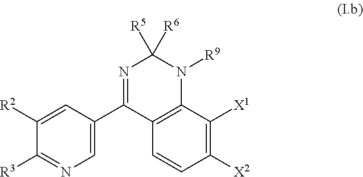

### 2.1 Preparation of 4-[6-(difluoromethyl)-5-methyl-3-pyridyl]-8-fluoro-2,2-dimethyl-1H-quinazoline (example compound 44 in Table II)

### 2.1a) Preparation of 1-[6-(difluoromethyl)-5-methyl-3-pyridyl]-(3-fluoro-2-nitro-phenyl) methanol

PhMgBr solution in THF (3M) (1.9 mL, 5.6 mmol) was added dropwise at −78° C. under N₂ to the solution of 1-fluoro-3-iodo-2-nitro benzene (1440 mg, 5.3 mmol) in THF (2 mL). The mixture was stirred at −78° C. for 1 h. Then the solution of 6-(difluoromethyl)-5-methyl-pyridine-3-carbaldehyde (600 mg, 3.5 mmol) in THF (10 mL) was added dropwise at −78° C. under N₂ to the mixture, and the mixture was stirred at −78° C. for 3 h. The mixture was quenched with ice water (50 mL), and the solution was extracted with EtOAc (15 mL×3). The combined organic layer was washed successively with brine, dried over anhydrous magnesium sulfate, and concentrated in vacuo to give the titled compound as a yellow solid.

¹H NMR (400 MHZ, DMSO): δ [ppm]: 8.41 (d, J=1.5 Hz, 1H), 7.74-7.65 (m, 2H), 7.60 -7.51 (m, 1H), 7.48 (d, J=7.9 Hz, 1H), 7.16-6.85 (m, 1H), 6.72 (d, J=4.8 Hz, 1H), 6.01 (d, J=4.6 Hz, 1H), 2.42 (s, 3H)

### 2.1b) Preparation of (2-amino-3-fluoro-phenyl)-[6-(difluoromethyl)-5-methyl-3-pyridyl]methanol

To mixture of [6-(difluoromethyl)-5-methyl-3-pyridyl]-(3-fluoro-2-nitro-phenyl) methanol (550 mg, 16 mmol) and Raney Ni (110 mg) in EtOH (10 mL) was stirred at 20° C. for 1 h under H₂ (15 PSi). The mixture was filtered, and the filtrate was concentrated in vacuo. The residue was triturated with (PE: EtOAc=10:1) to give the title compound as a yellow solid.

¹H NMR (400 MHZ, CDCl₃): u [ppm]: 8.44 (s, 1H), 7.60 (s, 1H), 7.01 (ddd, J=1.2, 8.2, 10.7 Hz, 1H), 6.85-6.55 (m, 3H), 5.94 (br s, 1H), 4.15 (br s, 2H), 2.88 (br d, J=3.5 Hz, 1H), 2.51 (s, 3H)

### 2.1c) Preparation of (2-amino-3-fluoro-phenyl)-[6-(difluoromethyl)-5-methyl-3-pyridyl]methanone

To a solution of (2-amino-3-fluoro-phenyl)-[6-(difluoromethyl)-5-methyl-3-pyridyl]methanol (450 mg, 1.6 mmol) in DMF (10 mL) was added at 20° C. K2CO₃ (442 mg, 3.2 mmol) and CuCl (31 mg, 0.32 mmol) and the mixture was stirred at 65° C. for 16 h under O2 (15 PSi). The mixture was poured into ice water (30 mL) and filtered. The filtrate was extracted with EtOAc (30 mL×3), washed with brine (15 mL), dried over anhydrous magnesium sulfate, and concentrated in vacuo. The crude product was purified by liquid chromatography on silica gel (PE:EtOAc=5:1) to give the title compound as a yellow oil.

¹H NMR (400 MHZ, CDCl₃): δ [ppm]: 8.65 (s, 1H), 7.85 (s, 1H), 7.23-7.16 (m, 2H), 6.90-6.61 (m, 1H), 6.58 (dt, J=4.9, 8.0 Hz, 1H), 6.29 (br s, 2H), 2.60 (s, 3H)

### 2.1d) Preparation of 4-[6-(difluoromethyl)-5-methyl-3-pyridyl]-8-fluoro-2,2-dimethyl-1H-quinazoline

The mixture of (2-amino-3-fluoro-phenyl)-[6-(difluoromethyl)-5-methyl-3-pyridyl]methanone (200 mg, 0.7 mmol) and NH₄OAc (539 mg, 7.0 mmol) in acetone (7 mL) was stirred at 65° C. for 4 h. The mixture was poured into ice water (15 mL), extracted with EtOAc (10 mL×3), washed with brine (15 mL), dried over anhydrous magnesium sulfate, and concentrated in vacuo. The crude product was purified by liquid chromatography on silica gel (PE:EtOAc=3:1) to give the title compound as a yellow solid.

¹H NMR (400 MHZ, CDCl₃): δ [ppm]: 8.55 (s, 1H), 7.77 (s, 1H), 7.08 (ddd, J=1.1, 8.2, 10.4 Hz, 1H), 6.93-6.52 (m, 3H), 4.25 (br s, 1H), 2.57 (s, 3H), 1.60 (s, 6H)

The compounds listed in Table II were prepared in an analogous manner.

TABLE II

| Ex.- | | | HPLC Rₜ | m.p. | |
|---|---|---|---|---|---|
| No. | Structure | Description | (min)* | [° C.] | LC-MS |
| Ex- 39 | 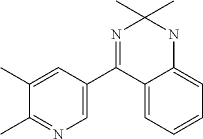 [SMILES: Cc1cc(C2=NC(C)(C)Nc3ccccc32)cnc1C] | Cpd. I.b wherein R², R³ = Me, R⁵, R⁶ = Me, R⁹ = H, X¹, X² = H | | | 265.3 |
| Ex- 40 | 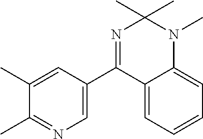 | Cpd. I.b wherein R², R³ = Me, R⁵, R⁶ = Me, R⁹ = Me, X¹, X² = H | | | 279.3 |
| Ex- 41 | 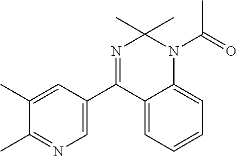 [SMILES: CC(=O)N1c2ccccc2C(c2cnc(C)c(C)c2)=NC1(C)C] | Cpd. I.b wherein R², R³ = Me, R⁵, R⁶ = Me, R⁹ = —C(═O)Me, X¹, X² = H | | | 307.3 |
| Ex- 42 | 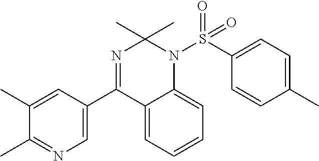 | Cpd. I.b wherein R², R³ = Me, R⁵, R⁶ = Me, R⁹ = S(O)₂-(4-methylphenyl), X¹, X² = H | 0.937 | 161 | 420.3 |
| Ex- 43 | 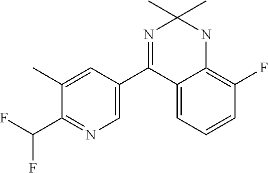 [SMILES: Cc1cc(C2=NC(C)(C)Nc3c(F)cccc32)cnc1C(F)F] | Cpd. I.b wherein R² = Me, R³ = CHF₂, R⁵, R⁶ = Me, R⁹ = H, X¹ = F, X² = H | 0.812 | 95 | 320.0 |
| Ex- 44 | 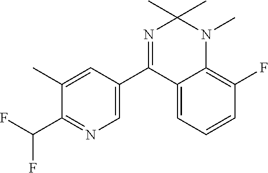 [SMILES: Cc1cc(C2=NC(C)(C)N(C)c3c(F)cccc32)cnc1C(F)F] | Cpd. I.b wherein R² = Me, R³ = CHF₂, R⁵, R⁶ = Me, R⁹ = Me, X¹ = F, X² = H | 0.872 | | 334.0 |
| Ex- 45 | 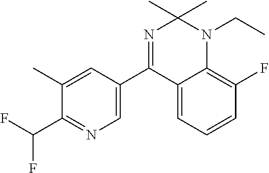 [SMILES: CCN1c2c(F)cccc2C(c2cnc(C(F)F)c(C)c2)=NC1(C)C] | Cpd. I.b wherein R² = Me, R³ = CHF₂, R⁵, R⁶ = Me, R⁹ = Et, X¹ = F, X² = H | 0.925 | | 348.0 |

*HPLC: High Performance Liquid Chromatography;
HPLC-column Kinetex XB C18 1,7 μ (50 × 2,1 mm); eluent:
acetonitrile/water + 0.1% trifluoro-acetic acid (gradient from 5:95 to 100:
0 in 1.5 min at 60° C., flow gradient from 0.8 to 1.0 ml/min in 1.5 min).
Rₜ: retention time in minutes.

### 3. Preparation of compounds I.c

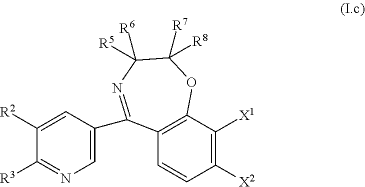

### 3.1 Preparation of 5-[6-(difluoromethyl)-5-methyl-3-pyridyl]-2,2,3-trimethyl-3H-1,4-ben-zoxazepine (example compound 58 in Table III)

### 3.1a) Preparation of 2,2,3-trimethylchroman-4-one oxime

Hydroxylamine hydrochloride (72.87 g, 3 eq) was added to a solution of 2,2,3-trimethylchroman-4-one (1 eq, 66.5g) in pyridine (423 ml, 15 eq) and the reaction mixture was stirred for 18 h at 85° C. The reaction solution was poured into water (1000 ml), and the solution was extracted with heptane, washed successively with water and brine, and dried over anhydrous magnesium sulfate. Removal of solvent in vacuo afforded the titled compound (66.6) as a brown powder. The title compound was used directly without further purification.

¹H NMR (400 MHZ, CDCl₃): δ [ppm]: 9.15 (s, 1H), 7.75 (dd, J=7.9, 1.7 Hz, 1H), 7.26 (dd, J=7.1, 1.5 Hz, 1H), 6.91 (ddd, J=8.2, 7.2, 1.2 Hz, 1H), 6.86 (dd, J=8.3, 1.2 Hz, 1H), 3.39 (q, J=7.0 Hz, 1H), 1.45 (s, 3H), 1.27 (s, 3H), 1.13 (d, J=7.0 Hz, 3H).

3.1b) Preparation of 2,2,3-trimethyl-3,4-dihydro-1,4-benzoxazepin-5-one 2,2,3-Trimethylchroman-4-one oxime (66 g, 1 eq) was added to thionyl chloride (80, 3.5 eq) at a temperature below 30° C., and the reaction mixture was stirred at 50° C. for 17 hours. After removal of thionyl chloride in vacuo the residue was poured into 1,4-dioxan (500 ml) and water (200 ml), and stirred for 1 h at 80° C. After removal of 1,4-dioxan in vacuo the resultant residue was extracted with ethyl acetate, washed successively with water and brine, and dried over anhydrous magnesium sulfate. Removal of solvent in vacuo the crude product was purified by flash chromatography on silica gel using hep-tane/MTBE as eluent to give the titled compound (25.4 g) as a with powder. ¹H NMR (400 MHZ, CDCl₃): d [ppm]: 7.74 (dd, J=7.7, 1.8 Hz, 1H), 7.44 (td, J=7.7, 1.8 Hz, 1H), 7.20 (td, J=7.5, 1.1 Hz, 1H), 6.98 (dd, J=8.1, 1.1 Hz, 1H), 6.44 (s, 1H), 3.37 (qd, J=6.9, 5.2 Hz, 1H), 1.39 (s, 3H), 1.29 (s, 3H), 1.20 (d, J=6.9 Hz, 3H).

### 3.1c) Preparation of 5-chloro-2,2,3-trimethyl-3H-1,4-benzoxazepine

A mixture of 2,2,3-trimethyl-3,4-dihydro-1,4-benzoxazepin-5-one (10 g, 1 eq) with phos-phoryl chloride (100 ml) and 11.67g phosphorus (V) chloride (1.15 eq) was stirred Heat for 2 h at 110° C. After cooling, the reaction solution concentrated in vacuo, diluted with dichloromethane, washed twice with saturated sodium carbonate solution, and dried over anhydrous magnesium sulfate. Removal of solvent in vacuo afforded the crude product (10.8 g). The title compound was used directly without further purification.

### 3.1d) Preparation of 5-[6-(difluoromethyl)-5-methyl-3-pyridyl]-2,2,3-trimethyl-3H-1,4-benzoxazepine

[6-(Difluoromethyl)-5-methyl-3-pyridyl]boronic acid (3.11 g, 1.2 eq), potassium carbonate (3.83 g, 2 eq), silver oxide (1.61, 0.5 eq) and dichlorobis(triphenylphosphine)-palladium (II) (490 mg, 0.05 eq) were added to a solution of 5-chloro-2,2,3-trimethyl-3H-1,4-benzoxazepine (3.1 g, 1 eq) in dry tetrahydrofuran (57 mL), and the mixture was stirred under argon atmosphere at 80° C. for 18 hours. After cooling, the reaction solution was diluted with ethyl acetate, and the solution was washed successively with water and brine, dried over anhydrous magnesium sulfate, and concentrated in vacuo. The crude product was purified by High Performance Liquid Chromatography on silica RP-18 using acetonitrile/water as eluent to give the titled compound (1.5 g) as a white powder.

¹H NMR (400 MHZ, CDCL): d [ppm]: 8.58 (d, J=1.9 Hz, 1H), 7.92 (8, 1H), 7.46 (t, J=7.7 Hz, 1H), 7.18 (td, J=7.5, 1.2 Hz, 1H), 7.09 (td, J=7.7, 1.4 Hz, 2H), 6.73 (t, J=54.5 Hz, 1H), 3.28 (d, J=6.8 Hz, 1H), 2.54 (d, J=2.2 Hz, 3H), 1.59 (s, 3H), 1.45 (s, 3H), 1.39 (8, 3H).

The compounds listed in Table III were prepared in an analogous manner.

TABLE III

| Ex .- | | | | HPLC Rₜ | m.p. |
|---|---|---|---|---|---|
| No. | Structure | Description | LC-MS | (min)* | [° C.] |
| Ex- 46 | 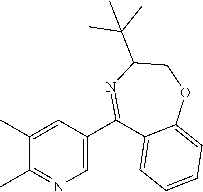 [SMILES: Cc1cc(C2=NC(C(C)(C)C)COc3ccccc32)cnc1C] | Cpd. I.c wherein R², R³ = Me, R⁵ = H, R⁶ = t-Bu, R⁷, R⁸ = H, X¹, X² = H | 309.2 | 0.968 | |
| Ex- 47 | 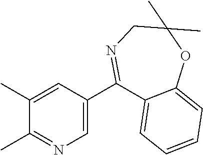 [SMILES: Cc1cc(C2=NCC(C)(C)Oc3ccccc32)cnc1C] | Cpd. I.c wherein R², R³ = Me, R⁵, R⁶ = H, R⁷, R⁸ = Me, X¹, X² = H | 281.2 | 0.75 | 82 |
| Ex- 48 | 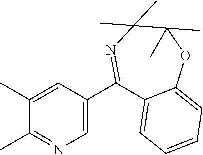 | Cpd. I.c wherein R², R³, R⁵, R⁶, R⁷, R⁸ = Me, X¹, X² = H | 309.3 | 0.789 | 100 |
| Ex- 49 | 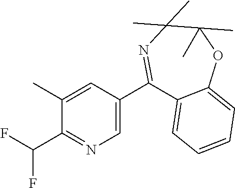 | Cpd. I.c wherein R₂ = Me, R₃ = CHF₂, R₅, R₆, R₇, R₈ = Me, X¹, X² = H | 345.0 | 0.887 | 108.5 |
| Ex- 50 | 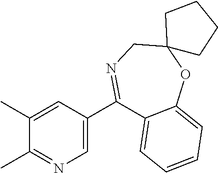 [SMILES: Cc1cc(C2=NCC3(CCCC3)Oc3ccccc32)cnc1C] | Cpd. I.c wherein R², R³ = Me, R⁵, R⁶ = H, R⁷, R⁸ form together with C atom to which they are bound cyclo- pentan-1,1-diyl, X¹, X² = H | 307.2 | 0.841 | |
| Ex 51 | 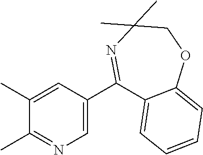 [SMILES: Cc1cc(C2=NC(C)(C)COc3ccccc32)cnc1C] | Cpd. I.c wherein R², R³ = Me, R⁵, R⁶ = Me, R⁷, R⁸ = H, X¹, X² = H | 281.0 | 0.684 | 116 |
| Ex- 52 | 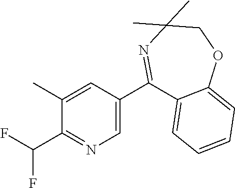 [SMILES: Cc1cc(C2=NC(C)(C)COc3ccccc32)cnc1C(F)F] | Cpd. I.c wherein R² = Me, R³ = CHF₂, R⁵, R⁶ = Me, R⁷, R⁸ = H, X¹, X² = H | 317.2 | 0.794 | 106 |
| Ex- 53 | 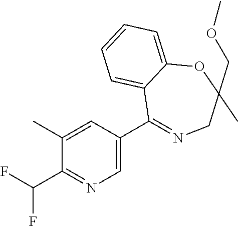 [SMILES: COCC1(C)CN=C(c2cnc(C(F)F)c(C)c2)c2ccccc2O1] | Cpd. I.c wherein R² = Me, R³ = CHF², R⁵, R⁶ = H, R⁷ = Me R⁸ = CH₂OCH₃, X¹, X² = H | 346.9 | 0.883 | |
| Ex- 54 | 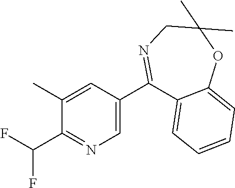 [SMILES: Cc1cc(C2=NCC(C)(C)Oc3ccccc32)cnc1C(F)F] | Cpd. I.c wherein R² = Me, R³ = CHF₂, R⁵, R⁶ = H, R⁷, R⁸ = Me, X¹, X² = H | 317.2 | 0.86 | |
| Ex- 55 | 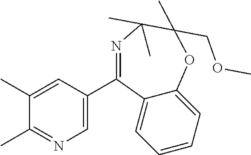 | Cpd. I.c wherein R², R³ = Me, R⁵, R⁶ = Me, R⁷ = Me, R⁸ = CH₂OCH₃, X¹, X² = H | 339.3 | 0.796 | 119 |
| Ex- 56 | 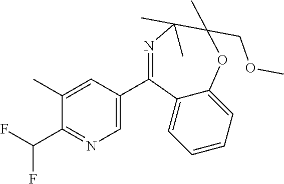 | Cpd. I.c wherein R² = Me, R³ = CHF₂, R⁵, R⁶ = Me, R⁷ = Me, R⁸ = CH₂OCH₃, X¹, X² = H | 375.3 | 0.893 | 111 |
| Ex- 57 | 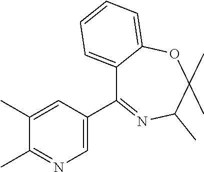 | Cpd. I.c wherein R², R³ = Me, R⁵, R⁶ = Me, R⁷ = Me, R⁸ = H, X¹, X² = H | 295.2 | 0.798 | 140 |
| Ex- 58 | 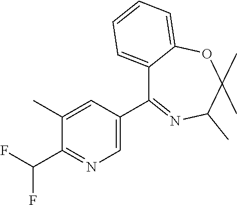 [SMILES: Cc1cc(C2=NC(C)C(C)(C)Oc3ccccc32)cnc1C(F)F] | Cpd. I.c wherein R² = Me, R³ = CHF₂, R⁵, R⁶ = Me, R⁷ = Me, R⁸ = H, X¹, X² = H | 331.2 | 0.906 | 120 |
| Ex- 59 | 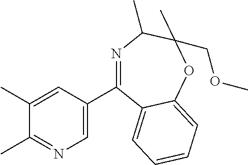 | Cpd. I.c wherein R², R³ = Me, R⁵ = Me, R⁶ = H, R⁷ = Me, R⁸ = CH2OCH3, X¹, X² = H | 325.1 | 0.797 | 173 |
| Ex- 60 | 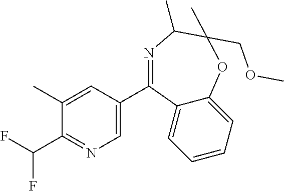 [SMILES: COCC1(C)Oc2ccccc2C(c2cnc(C(F)F)c(C)c2)=NC1C] | Cpd. I.c wherein R² = Me, R³ = CHF₂, R⁵ = Me, R⁶ = H, R⁷ = Me, R⁸ = CH₂OCH₃, X¹, X² = H | 361.2 | 0.927 | 153 |
| Ex- 61 | 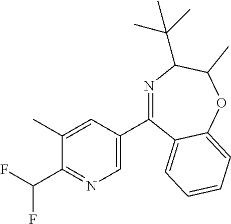 [SMILES: Cc1cc(C2=NC(C(C)(C)C)C(C)Oc3ccccc32)cnc1C(F)F] | Cpd. I.c wherein R² = Me, R³ = CHF₂, R⁵ = H, R⁶ = t-Bu, R⁷ = Me, R⁸ = H, X¹, X² = H | 359.3 | 1.132 | |
| Ex- 62 | 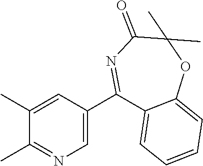 [SMILES: Cc1cc(C2=NC(=O)C(C)(C)Oc3ccccc32)cnc1C] | Cpd. I.c wherein R², R³ = Me, R⁵ and R⁶ form together ═O (oxo), R⁷, R⁸ = Me, X¹, X² = H | 295.2 | 0.84 | |
| Ex- 63 | 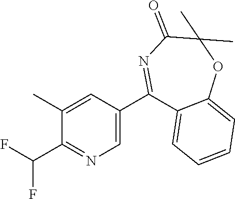 [SMILES: Cc1cc(C2=NC(=O)C(C)(C)Oc3ccccc32)cnc1C(F)F] | Cpd. I.c wherein R² = Me, R³ = CHF₂, R⁵ and R⁶ form together ═O (oxo), R⁷, R⁸ = Me, X¹, X² = H | 331.2 | 1.224 | 119 |
| Ex- 64 | 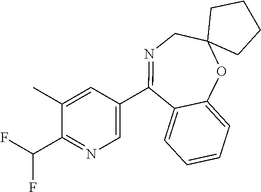 [SMILES: Cc1cc(C2=NCC3(CCCC3)Oc3ccccc32)cnc1C(F)F] | Cpd. I.c wherein R² = Me, R³ = CHF₂, R⁵, R⁶ = H, R⁷, R⁸ form together with C atom to which they are bound cyclopentan-1,1-diyl, X¹, X² = H | 343.1 | 0.962 | |

B. Fungicidal activity

### I. General

### Sensitivity Tests in Microtiter Format

The active substances were formulated separately as a stock solution in dimethyl sulfoxide (DMSO) at a concentration of 10 000 ppm.

### Selection of Resistant Strains

The active substances were formulated separately as a stock solution in dimethyl sulfoxide (DMSO) at a concentration of 10 000 ppm.

Spontaneous mutants were selected by plating out B. cinerea spores (in the range of 10⁷-10⁸) on DOB agar plates (3.4 g/l YNB (Becton Dickinson), 10 g/l Ammoniumsulfat, 40 g/l Glucose, 20 g/l Agar), amended with different lethal concentrations of active ingredient. After 7-14 days growing colonies were transferred to DOB agar plates, amended with the same lethal concentrations of active ingredient and then further propagated on Oatmeal agar medium (50 g/l Oatmeal agar (Sigma), 15 g/l Agar (Difco) for sensitivity testing.

### Detection of Mutations

Mutations in the target gene of resistant strains can be detected on well know nucleic acid level methods, e.g. Sanger sequencing and Pyrosequencing.

### II. Examples

1. Example 1: Activity Against Botrytis cinerea in the Microtiter Test

### 1.1 General Procedure for the Antifungal Activity Determination

Fungal strains used:
a) Botrytis cinerea (sensitive, wild-type)
b) Various mutants of Botrytis cinerea

The spores of Botrytis cinerea were harvested with a cotton swab from an Oatmeal agar plate (50 g/l Oatmealagar (Sigma, Art.O-3506), 15 g/l agar, (Difco) and this is dipped in 3 ml of double concentrated DOB-medium (3.4 g/l YNB (Becton Dickinson), 10 g/l Ammoniumsulfat, 40 g/l Glucose, adjusted to pH 7) and the spore suspension is adjusted to a spore density of 3.2×10⁴ spores/ml. The compound solutions were diluted from stock solution in (dimethylsulfoxide) DMSO in several steps with sterile deionized water before use. High test concentrations were 50 mg/L (ppm) or 31 mg/L (ppm). Compound solutions were transferred into empty microplates and filled up with an equal amount of spore suspension.

The antifungal activity was determined by measuring the turbidity of a culture in 96-well microplates in the presence of test compounds after 7 days incubation at 620 nm. A blank value for each concentration (growth media+compound, without spores) are subtracted from the spore suspension. The relative antifungal activity was calculated by comparing the effect of the test compounds with the effect of a DMSO control The results are shown in Table IV (for compounds of general formula I.A), VI (for compounds of general formula I.B), and VIII (for compounds of general formula I.C).

A compound showing an IC₅₀ value for the mutant strain IC₅₀ (MUT) equal to or lower than the IC₅₀ value observed for the wild-type strain IC₅₀ (WT) is considered particularly active (“AA”) as DHODH inhibitor, and consequently as particularly useful for combatting the respective B. cinerea mutant strain.

A compound showing an IC₅₀ (MUT) significantly below 31 mg/L and an IC₅₀ (MUT) to IC₅₀ (WT) ratio being in a range of 1 to 100 is considered active (“A”) as DHODH inhibitor, and consequently as useful for combatting the respective B. cinerea mutant strain.

A compound showing an IC₅₀ (MUT) above the IC₅₀ (WT) and showing an IC₅₀ (MUT) to IC₅₀ (WT) ratio being in a range above 100 is considered unsuitable (“I”) as DHODH inhibitor.

The absolute IC₅₀-values (concentration of test compound resulting in 50% inhibition of fungal growth) were calculated from the resulting dose-response for each compound and strain.

The results are reported in Table V (for compounds of general formula I.A), Table VII (for compounds of general formula I.B) and Table IX (for compounds of general formula I.C).

The initial concentration of the test compounds and several steps of dilution allowed IC₅₀-values from 0.0032 to 50 mg/L (ppm) (1:5 dilution) or 0.0001 to 31 mg/L (ppm) (1:4 dilution) to be assessed s shown in the tables.

### 1.2 Results

### 1.2.1 Results for Compounds of General Formula I.A

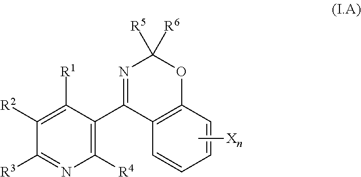

### 1.2.1.1 Relative Antifungal Activity for Compounds of General Formula I.A

TABLE IV
Relative antifungal activity expressed as IC₅₀ values (ppm) obtained for
compounds of general formula I.A according to the present invention

| | | Biological data for the compounds of | |
|---|---|---|---|
| No. | Structure | general formula I.A (IC₅₀ in ppm) | |
| 1 | 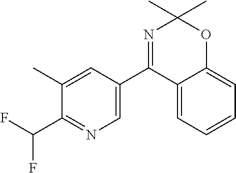 [SMILES: Cc1cc(C2=NC(C)(C)Oc3ccccc32)cnc1C(F)F] | H108P G111E G111V V195F | A A A AA |
| 2 | 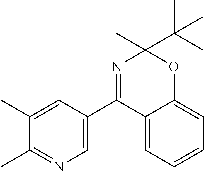 [SMILES: Cc1cc(C2=NC(C)(C(C)(C)C)Oc3ccccc32)cnc1C] | H108P G111E G111V V195F | A A A AA |
| 4 | 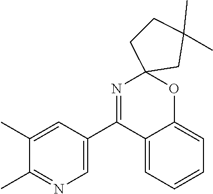 | H108P G111E G111V V195F | A A A A |
| 5 | 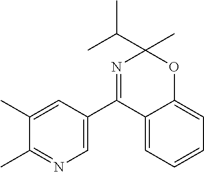 [SMILES: Cc1cc(C2=NC(C)(C(C)C)Oc3ccccc32)cnc1C] | H108P G111E G111V V195F Y208H | AA A A AA A |
| 6 | 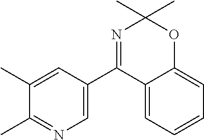 [SMILES: Cc1cc(C2=NC(C)(C)Oc3ccccc32)cnc1C] | H108P G111E G111V V195F | AA A A AA |
| 7 | 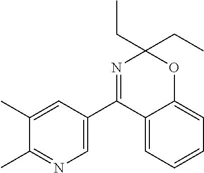 [SMILES: CCC1(CC)N=C(c2cnc(C)c(C)c2)c2ccccc2O1] | H108P G111E G111V V195F | AA A A AA |
| 8 | 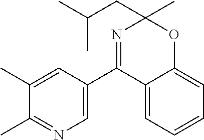 [SMILES: Cc1cc(C2=NC(C)(CC(C)C)Oc3ccccc32)cnc1C] | H108P G111E G111V V195F Y208H | A A AA AA A |
| 9 | 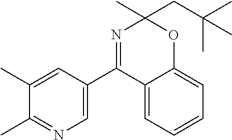 [SMILES: Cc1cc(C2=NC(C)(CC(C)(C)C)Oc3ccccc32)cnc1C] | H108P G111V V195F Y208H | A A AA A |
| 10 | 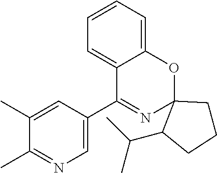 [SMILES: Cc1cc(C2=NC3(CCCC3C(C)C)Oc3ccccc32)cnc1C] | H108P G111V V195F Y208H | A A AA A |
| 11 | 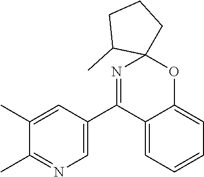 [SMILES: Cc1cc(C2=NC3(CCCC3C)Oc3ccccc32)cnc1C] | H108P G111E G111V V195F | AA A AA AA |
| 12 | 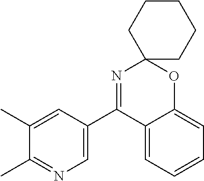 [SMILES: Cc1cc(C2=NC3(CCCCC3)Oc3ccccc32)cnc1C] | H108P G111E G111V V195F | A A A AA |
| 13 | 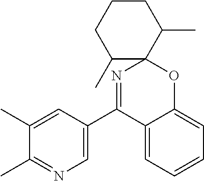 [SMILES: Cc1cc(C2=NC3(Oc4ccccc42)C(C)CCCC3C)cnc1C] | H108P G111E G111V V195F Y208H | AA A AA AA AA |
| 14 | 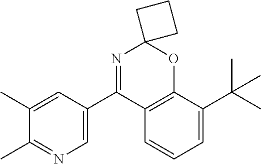 [SMILES: Cc1cc(C2=NC3(CCC3)Oc3c2cccc3C(C)(C)C)cnc1C] | H108P G111E G111V V195F Y208H | AA AA AA AA AA |
| 15 | 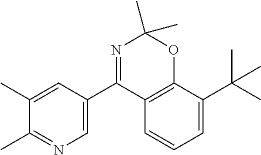 [SMILES: Cc1cc(C2=NC(C)(C)Oc3c2cccc3C(C)(C)C)cnc1C] | H108P G111V V195F | AA AA AA |
| 16 | 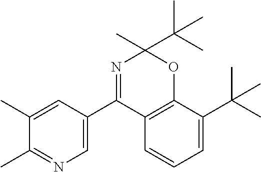 [SMILES: Cc1cc(C2=NC(C)(C(C)(C)C)Oc3c2cccc3C(C)(C)C)cnc1C] | V195F | AA |
| 17 | 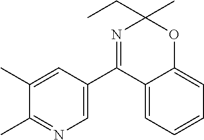 [SMILES: CCC1(C)N=C(c2cnc(C)c(C)c2)c2ccccc2O1] | H108P G111V V195F Y208H | A A AA A |
| 18 | 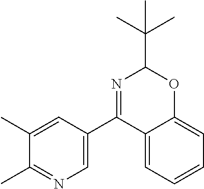 [SMILES: Cc1cc(C2=NC(C(C)(C)C)Oc3ccccc32)cnc1C] | H108P G111E G111V V195F | AA AA AA AA |
| 19 | 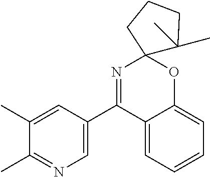 | H108P G111V V195F Y208H | A A AA A |
| 20 | 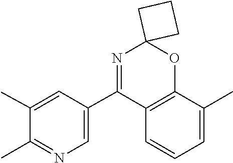 | H108P G111E G111V V195F Y208H | AA A AA AA A |
| 21 | 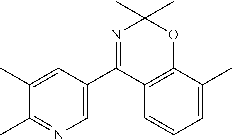 | H108P G111E G111V V195F Y208H | AA A AA AA A |
| 22 | 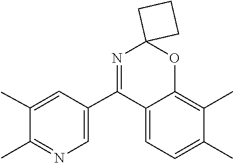 | H108P G111E G111V V195F Y208H | AA A AA AA A |
| 23 | 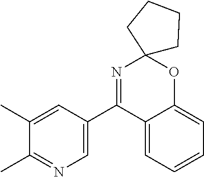 [SMILES: Cc1cc(C2=NC3(CCCC3)Oc3ccccc32)cnc1C] | H108P G111V V195F | A A AA |
| 26 | 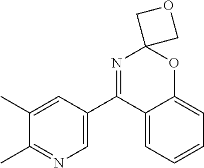 [SMILES: Cc1cc(C2=NC3(COC3)Oc3ccccc32)cnc1C] | H108P G111E G111V V195F | AA AA AA AA |
| 27 | 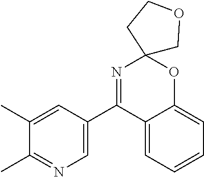 [SMILES: Cc1cc(C2=NC3(CCOC3)Oc3ccccc32)cnc1C] | H108P G111E G111V V195F Y208H | AA A AA AA AA |
| 28 | 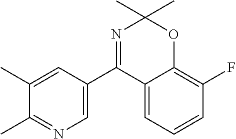 [SMILES: Cc1cc(C2=NC(C)(C)Oc3c(F)cccc32)cnc1C] | H108P G111E G111V V195F Y208H | A A A AA A |
| 30 | 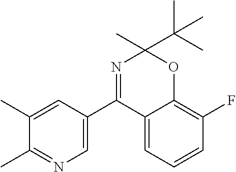 [SMILES: Cc1cc(C2=NC(C)(C(C)(C)C)Oc3c(F)cccc32)cnc1C] | H108P G111V V195F | A A AA |
| 31 | 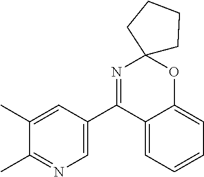 [SMILES: Cc1cc(C2=NC3(CCCC3)Oc3ccccc32)cnc1C] | H108P G111V V195F | A A AA |
| 33 | 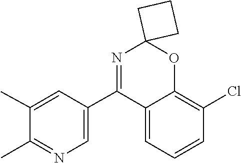 [SMILES: Cc1cc(C2=NC3(CCC3)Oc3c(Cl)cccc32)cnc1C] | H108P G111E G111V V195F Y208H | AA A AA AA A |
| 36 | 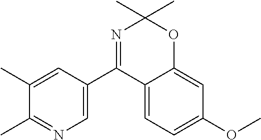 | H108P G111E G111V V195F | AA A AA AA |
| 38 | 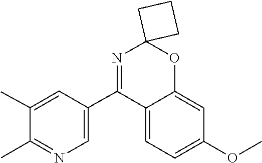 | H108P G111E G111V V195F Y208H | AA A AA AA A |

1.2.1.2 Absolute antifungal activity for compounds of general formula I.A

The following data show a comparison between the absolute ICH obtained when subjecting a set of compounds of general formula I.A and comparative compounds (as shown in Table V) to the screening activity test following the general procedure as outlined above.

TABLE V
Absolute IC₅₀ (ppm) obtained for compounds of general formula I.A according to the present invention

| | | Biological data | | | Biological data | |
|---|---|---|---|---|---|---|
| | | for the compounds | | | for the | |
| | | of general | | | comparative | |
| [symbol] | | formula I.A | | | compounds | |
| No. | Structure | (IC₅₀ in ppm) | | Comparative compounds | (IC₅₀ in ppm) | |
| 3 | 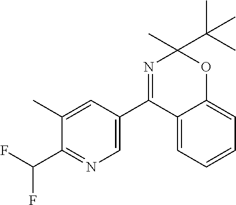 [SMILES: Cc1cc(C2=NC(C)(C(C)(C)C)Oc3ccccc32)cnc1C(F)F] | wild type H108P G111E G111V V195F | 0.19 2.54 2.81 3.17 0.75 | 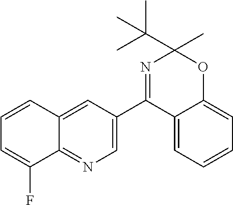 [SMILES: CC(C)(C)C1(C)N=C(c2cnc3c(F)cccc3c2)c2ccccc2O1] | wild type V195F | 0.07 >31 |
| 24 | 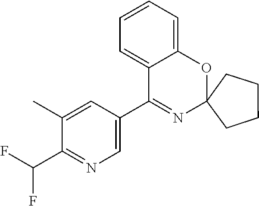 [SMILES: Cc1cc(C2=NC3(CCCC3)Oc3ccccc32)cnc1C(F)F] | wild type H108P G111E G111V V195F | 0.35 0.98 15.31 1.31 0.69 | 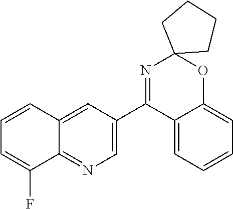 [SMILES: Fc1cccc2cc(C3=NC4(CCCC4)Oc4ccccc43)cnc12] | wild type V195F | 0.05 10.91 |
| 25 | 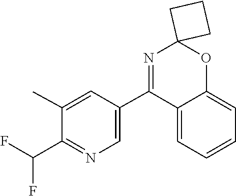 [SMILES: Cc1cc(C2=NC3(CCC3)Oc3ccccc32)cnc1C(F)F] | wild type H108P G111V V195F Y208H | 0.05 0.02 0.03 0.08 0.28 | 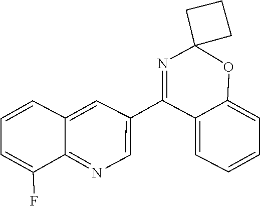 | wild type H108P G111V V195F Y208H | 0.03 0.17 0.14 16.41 16.00 |
| 29 | 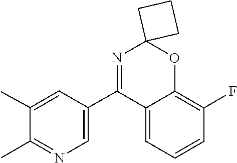 [SMILES: Cc1cc(C2=NC3(CCC3)Oc3c(F)cccc32)cnc1C] | wild type H108P G111E G111V V195F Y208H | 0.02 0.02 2.34 0.04 0.02 0.93 | 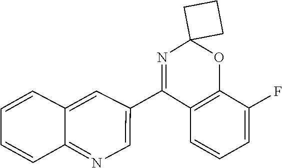 [SMILES: Fc1cccc2c1OC1(CCC1)N=C2c1cnc2ccccc2c1] | wild type H108P G111E G111V V195F Y208H | 0.03 0.11 9.48 0.14 24.15 1.59 |
| 32 | 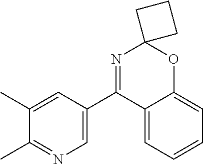 [SMILES: Cc1cc(C2=NC3(CCC3)Oc3ccccc32)cnc1C] | wild type H108P G111E G111V V195F Y208H | 0.12 0.07 2.34 2.85 0.13 1.23 | 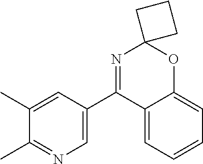 [SMILES: Cc1cc(C2=NC3(CCC3)Oc3ccccc32)cnc1C] | wild type H108P G111E G111V V195F Y208H | 0.11 0.25 10.94 0.25 2.99 1.43 |
| 34 | 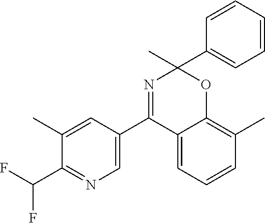 [SMILES: Cc1cc(C2=NC(C)(c3ccccc3)Oc3c(C)cccc32)cnc1C(F)F] | wild type H108P G111V V195F Y208H | 0.08 0.53 0.77 0.09 1.19 | 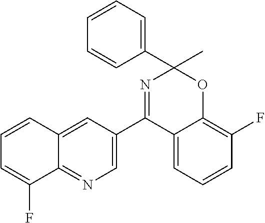 [SMILES: CC1(c2ccccc2)N=C(c2cnc3c(F)cccc3c2)c2cccc(F)c2O1] | wild type V195F | 0.04 15.53 |
| 35 | 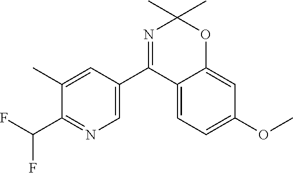 [SMILES: COc1ccc2c(c1)OC(C)(C)N=C2c1cnc(C(F)F)c(C)c1] | wild type H108P G111E G111V V195F Y208H | 0.02 0.29 2.70 0.72 0.05 2.02 | 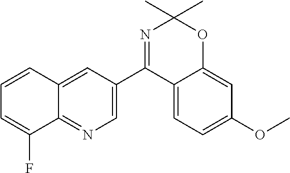 [SMILES: COc1ccc2c(c1)OC(C)(C)N=C2c1cnc2c(F)cccc2c1] | wild type V195F | 0.01 5.28 |
| 37 | 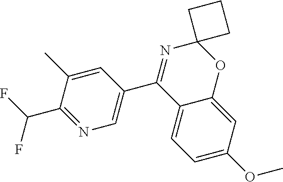 [SMILES: COc1ccc2c(c1)OC1(CCC1)N=C2c1cnc(C(F)F)c(C)c1] | wild type H108P G111E G111V V195F | 0.03 0.06 0.54 0.09 0.11 | 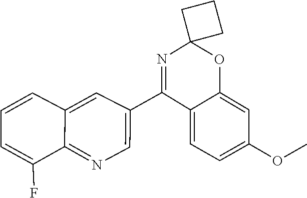 | wild type H108P G111E G111V V195F | 0.03 0.65 1.10 0.74 19.41 |

[symbol]

indicates data missing or illegible when filed

From the above data it is evident that the use of the set of compounds of general formula I.A leads to a noticeable improvement in terms of antifungal activity

### 1.2.2 Results for Compounds of General Formula I.B

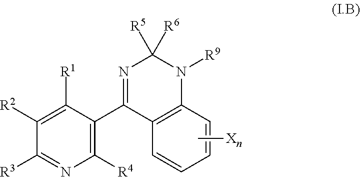

### 1.2.2.1 Relative Antifungal Activity for Compounds of General Formula I.B

TABLE VI
Relative antifungal activity expressed as IC₅₀ (ppm) obtained for compounds of
general formula I.B according to the present invention.

| | | Biological data for the | |
|---|---|---|---|
| | | compounds of general | |
| No. | Structure | formula I.B (IC₅₀ in ppm) | |
| 39 | 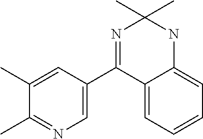 [SMILES: Cc1cc(C2=NC(C)(C)Nc3ccccc32)cnc1C] | H108P G111E G111V V195F | AA AA AA AA |
| 40 | 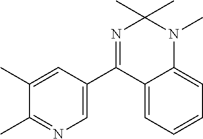 | H108P G111E G111V V195F Y208H | A AA A AA A |
| 41 | 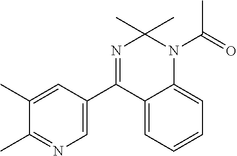 [SMILES: CC(=O)N1c2ccccc2C(c2cnc(C)c(C)c2)=NC1(C)C] | H108P G111V V195F | A A AA |
| 42 | 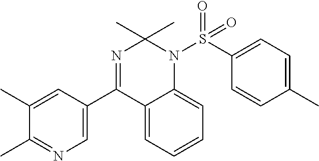 | H108P G111E G111V V195F | AA AA AA AA |
| 43 | 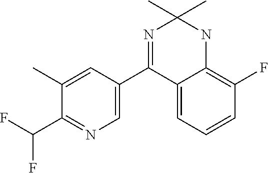 [SMILES: Cc1cc(C2=NC(C)(C)Nc3c(F)cccc32)cnc1C(F)F] | H108P G111E G111V V195F Y208H | A A AA AA A |

### 1.2.2.2 Absolute Antifungal Activity for Compounds of General Formula I.B

The following data show a comparison between the absolute ICs obtained when subjecting a set of compounds of general formula I.B and comparative compounds (as shown in table VII) to the screening activity test according to the general procedure as outlined above.

TABLE VII
Absolute IC₅₀ (ppm) obtained for compounds of general formula I.B according to the present invention.

| | | Biological data for the | | | | |
|---|---|---|---|---|---|---|
| | | compounds of general | | | Biological data for the | |
| [symbol] | | formula | | | comparative compounds | |
| No. | Structure | I.B (IC₅₀ in ppm) | | Comparative compounds | (IC₅₀ in ppm) | |
| 44 | 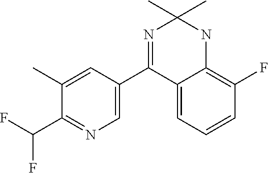 [SMILES: Cc1cc(C2=NC(C)(C)Nc3c(F)cccc32)cnc1C(F)F] | wild type H108P G111E G111V V195F Y208H | 0.01 0.50 0.08 0.73 0.03 1.64 | 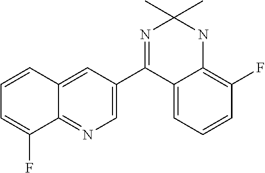 [SMILES: CC1(C)N=C(c2cnc3c(F)cccc3c2)c2cccc(F)c2N1] | wild type V195F | 0.005 4.42 |
| 45 | 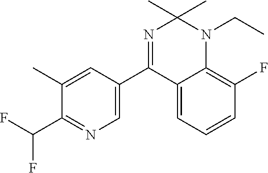 [SMILES: CCN1c2c(F)cccc2C(c2cnc(C(F)F)c(C)c2)=NC1(C)C] | wild type H108P G111E G111V V195F Y208H | 0.01 0.4 0.57 0.96 0.02 0.27 | 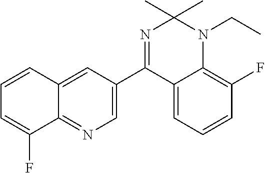 [SMILES: CCN1c2c(F)cccc2C(c2cnc3c(F)cccc3c2)=NC1(C)C] | wild type V195F | 0.02 >31 |

[symbol]

indicates data missing or illegible when filed

### 1.2.3 Results for Compounds of General Formula I.C

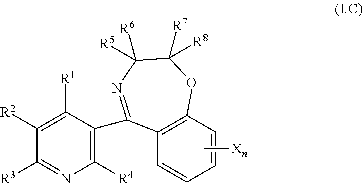

### 1.2.3.1 Relative Antifungal Activity for Compounds of General Formula I.C

TABLE VIII
Relative antifungal activity expressed as IC₅₀ (ppm) obtained for compounds of
general formula I.C according to the present invention.

| | | Biological data for the compounds | |
|---|---|---|---|
| | | of general formula I.C | |
| No. | Structure | (IC₅₀ in ppm) | |
| 46 | 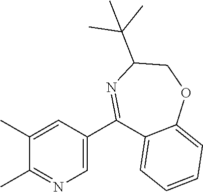 [SMILES: Cc1cc(C2=NC(C(C)(C)C)COc3ccccc32)cnc1C] | H108P G111E G111V V195F Y208H | AA A AA AA A |
| 48 | 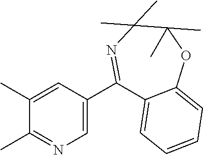 | V195F | AA |
| 50 | 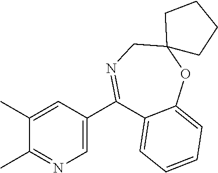 [SMILES: Cc1cc(C2=NCC3(CCCC3)Oc3ccccc32)cnc1C] | V195F | AA |
| 51 | 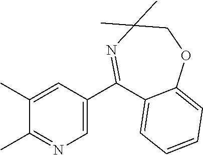 [SMILES: Cc1cc(C2=NC(C)(C)COc3ccccc32)cnc1C] | H108P G111V V195F | AA AA AA |
| 52 | 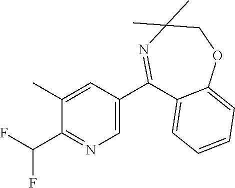 [SMILES: Cc1cc(C2=NC(C)(C)COc3ccccc32)cnc1C(F)F] | H108P G111E G111V V195F | AA A AA AA |
| 53 | 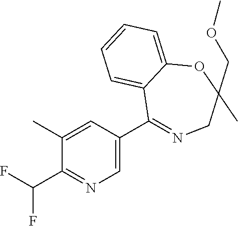 [SMILES: COCC1(C)CN=C(c2cnc(C(F)F)c(C)c2)c2ccccc2O1] | H108P G111V V195F Y208H | A A AA A |
| 54 | 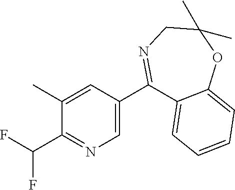 [SMILES: Cc1cc(C2=NCC(C)(C)Oc3ccccc32)cnc1C(F)F] | H108P G111E G111V V195F Y208H | A A A AA A |
| 55 | 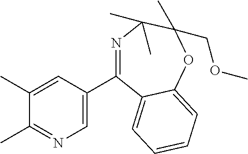 | H108P G111V V195F Y208H | A A AA A |
| 56 | 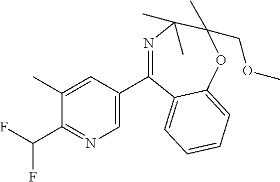 | H108P V195F Y208H | A AA A |
| 57 | 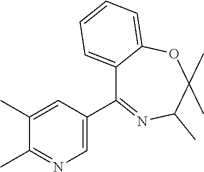 | H108P G111V V195F Y208H | A A AA A |
| 58 | 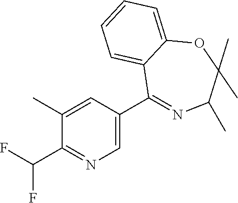 [SMILES: Cc1cc(C2=NC(C)C(C)(C)Oc3ccccc32)cnc1C(F)F] | H108P G111E G111V V195F Y208H | A A A AA A |
| 59 | 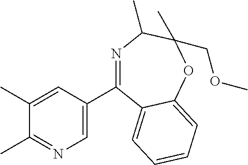 | H108P G111E G111V V195F Y208H | A A A AA A |
| 60 | 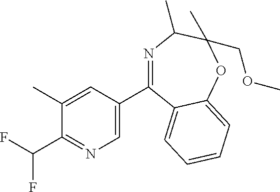 [SMILES: COCC1(C)Oc2ccccc2C(c2cnc(C(F)F)c(C)c2)=NC1C] | H108P G111E G111V V195F Y208H | AA A AA AA AA |
| 61 | 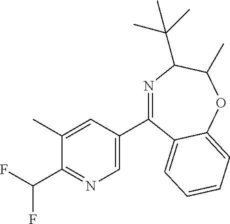 [SMILES: Cc1cc(C2=NC(C(C)(C)C)C(C)Oc3ccccc32)cnc1C(F)F] | H108P G111V V195F Y208H | AA A AA A |
| 62 | 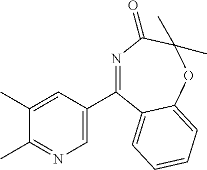 | H108P G111E G111V V195F | AA A A AA |
| 63 | 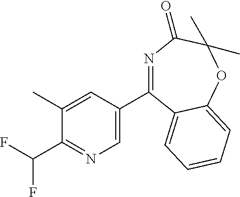 [SMILES: Cc1cc(C2=NC(=O)C(C)(C)Oc3ccccc32)cnc1C(F)F] | H108P G111V V195F Y208H | AA AA AA A |
| 64 | 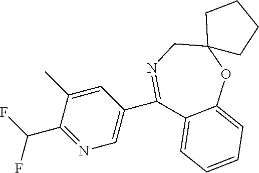 [SMILES: Cc1cc(C2=NCC3(CCCC3)Oc3ccccc32)cnc1C(F)F] | H108P G111E G111V V195F | A A A AA |

### 1.2.3.2Absolute Antifungal Activity for Compounds of General Formula I.C

The following data show a comparison between the absolute ICH obtained when subjecting a set of compounds of general formula I.C and comparative compounds (as shown in table IX) to the screening activity test according to the general procedure as outlined above

TABLE IX
Absolute IC₅₀ (ppm) obtained for compounds of general formula I.C according to the present invention.

| | | Biological | | | | |
|---|---|---|---|---|---|---|
| | | data for the | | | Biological data | |
| | | compounds | | | for the | |
| | | of general | | | comparative | |
| | | formula I.C | | | compounds | |
| No. | Structure | (IC₅₀ in ppm) | | Comparative compounds | (IC₅₀ in ppm) | |
| 47 | 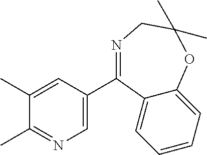 | wild type H108P G111E G111V V195F Y208H | 0.04 0.83 2.47 0.55 0.02 1.90 | 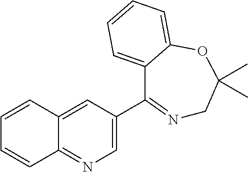 [SMILES: CC1(C)CN=C(c2cnc3ccccc3c2)c2ccccc2O1] | wild type V195F | 0.001 19.54 |
| 49 | 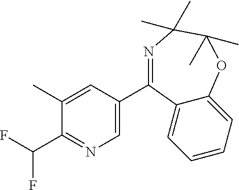 | wild type H108P G111E G111V V195F Y208H | 0.01 0.32 2.35 0.70 0.01 0.97 | 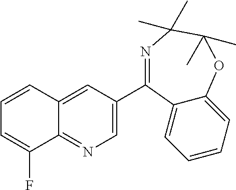 | wild type V195F | 0.001 2.84 |

### 2. Example 2: Activity Against Sclerotinia sclerotiorum in Tissue Culture Plate Assay with Mycelial Plugs

### 2.1 General Procedure of the Antifungal Activity Determination

Fungal strains used:
a) Sclerotinia sclerotiorum (sensitive, wild-type)
b) Mutants of Sclerotinia sclerotiorum

Pure cultures are grown on DOB-agar medium, (3.4 g/l YNB (Becton Dickinson), 10 g/l Ammoniumsulfat, 40 g/l Glucose) for 5-7 days at 18° C. Mycelial plugs (6 mm diame-ter) taken from the margin of the colonies are used as inoculum for the sensitivity test. 500 μl fungicide-amended DOB medium (3.4 g/l YNB (Becton Dickinson), 10 g/l Ammoniumsulfat, 40 g/l Glucose, adjusted to pH 7) are added to each well of 24-well tissue culture plates. Each well is then inoculated with a mycelial plug, with the myce-lium surface up. Two replicate wells are used for each isolate and fungicide concentration. Growth is assed after 7 days incubation at 18° C. in the dark by estimating % growth in the well. IC₅₀-values (concentration of test compound resulting in 50% inhibition of fungal growth) were calculated from the resulting dose-response for each compound and strain.

### 2.2 Results

### 2.2.1 Absolute Antifungal Activity for Compounds of Formula I.a and Comparative Compounds

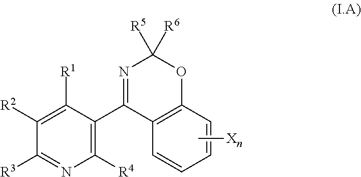

The results are shown in Table X.

TABLE X
Absolute IC₅₀ (ppm) obtained for compounds of general formula I.A according to the present
invention and respective comparative compounds

| | | Data for | | | | |
|---|---|---|---|---|---|---|
| | | compounds of | | | Data for | |
| | | general | | | comparative | |
| | | Formul I.A | | | compounds | |
| No | Structure | (IC50 in ppm) | | Comparative compound | (IC50 in ppm) | |
| Ex-3 | 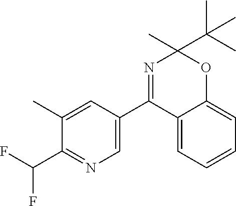 [SMILES: Cc1cc(C2=NC(C)(C(C)(C)C)Oc3ccccc32)cnc1C(F)F] | wild typ [symbol] V182F | 0.893 1.058 | 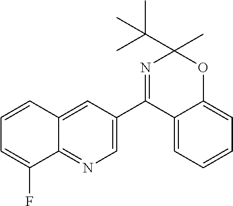 [SMILES: CC(C)(C)C1(C)N=C(c2cnc3c(F)cccc3c2)c2ccccc2O1] | wild typ [symbol] V182F | 0.137 >50 |
| Ex-34 | 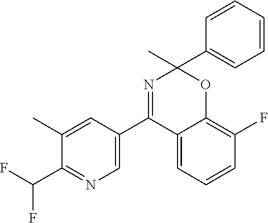 [SMILES: Cc1cc(C2=NC(C)(c3ccccc3)Oc3c(F)cccc32)cnc1C(F)F] | wild typ [symbol] V182F | 1.796 0.251 | 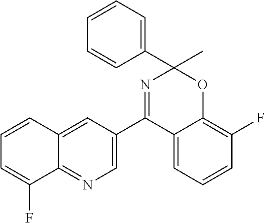 [SMILES: CC1(c2ccccc2)N=C(c2cnc3c(F)cccc3c2)c2cccc(F)c2O1] | wild typ [symbol] V18F | 0.154 >50 |
| Ex-35 | 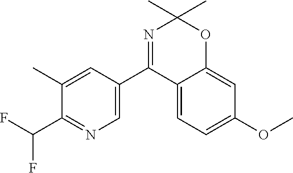 [SMILES: COc1ccc2c(c1)OC(C)(C)N=C2c1cnc(C(F)F)c(C)c1] | wild typ [symbol] V182F | 0.468 0.836 | 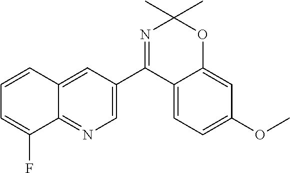 [SMILES: COc1ccc2c(c1)OC(C)(C)N=C2c1cnc2c(F)cccc2c1] | wild typ [symbol] V182F | 0.06 >50 |

[symbol]

indicates data missing or illegible when filed

### 2.2.2 Absolute Antifungal Activity for Compounds of Formula I.B and Comparative Compounds

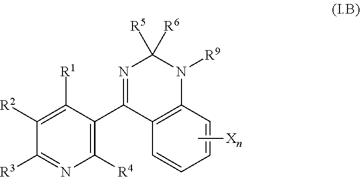

The results are shown in Table XI.

TABLE XI
Absolute IC₅₀ (ppm) obtained for compounds of general formula I.B according to the present
invention and respective comparative compounds.

| | | Data for | | | Data for | |
|---|---|---|---|---|---|---|
| | | compounds of general | | | comparative compounds | |
| | | Formul I.B | | | (IC50 in | |
| No | Structure | (IC50 in ppm) | | Comparative compound | in ppm) | |
| EX-6 | 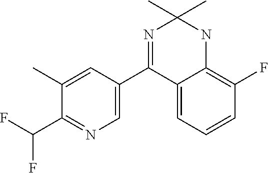 [SMILES: Cc1cc(C2=NC(C)(C)Nc3c(F)cccc32)cnc1C(F)F] | Wild typ [symbol] V182F | 0.08 0.06 | 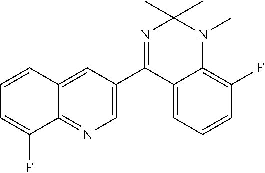 [SMILES: CN1c2c(F)cccc2C(c2cnc3c(F)cccc3c2)=NC1(C)C] | Wild typ [symbol] V182F | 0.03 >50 |
| EX-7 | 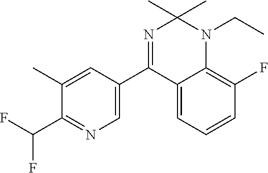 [SMILES: CCN1c2c(F)cccc2C(c2cnc(C(F)F)c(C)c2)=NC1(C)C] | Wild typ [symbol] V182F | 0.23 1.392 | 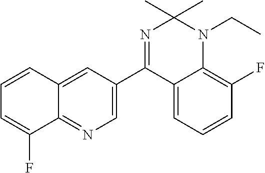 [SMILES: CCN1c2c(F)cccc2C(c2cnc3c(F)cccc3c2)=NC1(C)C] | Wild typ [symbol] V182F | 0.03 >50 |

[symbol]

indicates data missing or illegible when filed

2.2.3 Absolute antifungal activity for compounds of Formula I.C and comparative compounds

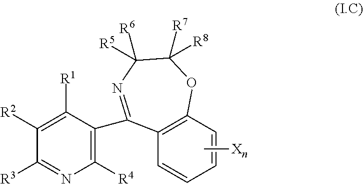

The results are shown in Table XII.

TABLE XII
Absolute IC₅₀ (ppm) obtained for compounds of general formula I.C according to the present
invention and respective comparative compounds.

| | | Data for | | | Data for | |
|---|---|---|---|---|---|---|
| | | compounds of | | | comparative | |
| | | general | | | compounds | |
| | | Formul I.B | | | (IC50 in | |
| No | Structure | (IC50 in ppm) | | Comparative compound | in ppm) | |
| 4 | 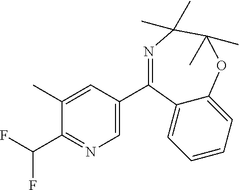 | wild typ [symbol] V182F | 8.454 24.342 | 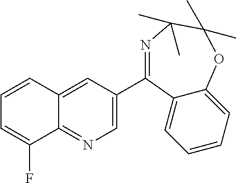 | wild typ [symbol] V182F | 1.995 >50 |

[symbol]

indicates data missing or illegible when filed

### Summary of Sequence:

| SEQ | | | |
|---|---|---|---|
| ID NO | Description | Source | Type |
| 1 | BOTRCI_DHODH_EMR82015.1 | Botrytis cinerea | AA |
| 2 | BOTRCI_DHODH_cDNA_KB708057.1_30538- | Artificial | NA |
| | 30546, 30690-32158, 32250-32319 | | |
| 3 | ALTEAL_DHODH_XP_018389332 | Alternaria altemata | AA |
| 4 | ALTEAL_DHODH_cDNA_XM_018536128.1 | Artificial | NA |
| 5 | ALTESO_DHODH_no_accession | Alternaria solani | AA |
| 6 | ALTESO_DHODH_cDNA_CP022024 | Artificial | NA |
| 7 | CERCBE_DHODH_XP_023451964 | Cercospora beticola | AA |
| 8 | CERCBE_DHODH_NT_cDNA_XM_023596659.1 | Artificial | NA |
| 9 | CERSCO_DHODH_no_accession | Cercospora sojina | AA |
| 10 | CERSCO_DHODH_cDNA_CP036211.1_684093- | Artificial | NA |
| | 686351 | | |
| 11 | COCHSA_DHODH_XP_007699125 | Bipolaris sorokiniana | AA |
| 12 | COCHSA_DHODH_cDNA_XM_007700935.1 | Artificial | NA |
| 13 | COLLGR_DHODH_XP_008100212 | Colletotrichum graminicola | AA |
| 14 | COLLGR_DHODH_cDNA_XM_008102021 | Artificial | NA |
| 15 | COLLLA_DHODH_AA_TDZ24597.1 | Colletotrichum obriculare | AA |
| 16 | COLLLA_DHODH_cDNA_AMCV02000004.1 | Artificial | NA |
| 17 | CORYCA_DHODH_PSN63766.1 | Corynespora cassicola | AA |
| 18 | CORYCA_DHODH_cDNA_JAQF01000000 | Artificial | NA |
| 19 | FUSACU_DHODH_PTD01677.1 | Fusarium culmorum | AA |
| 20 | FUSACU_DHODH_cDNA_LT598662 | Artificial | NA |
| 21 | FUSAOX_DHODH_XP_018245138 | Fusarium oxysporum | AA |
| 22 | FUSAOX_DHODH_cDNA_XM_018387299.1 | Artificial | NA |
| 23 | GIBBZE_DHODH_XP_011328027.1 | Fusarium graminearum | AA |
| 24 | GIBBZE_DHODH_cDNA_XM_011329725.1 | Artificial | NA |
| 25 | LEPTMA_DHODH_XP_003834159.1 | Plenodomus lingam | AA |
| 26 | LEPTMA_DHODH_cDNA_XM_003834111.1 | Artificial | NA |
| 27 | LEPTNO_DHODH_XP_001803781.1 | Parastagonospora nodorum | AA |
| 28 | LEPTNO_DHODH_cDNA_XM_001803729.1 | Artificial | NA |
| 29 | MONGNI_DHODH_in_house | Monographella nivalis | AA |
| 30 | MONGNI_DHODH_cDNA_inhouse | Artificial | NA |
| 31 | MYCOFI_DHODH_XP_007930122.1 | Pseudocercospora fijiensis | AA |
| 32 | MYCOFI_DHODH_cDNA_XM_007931931.1 | Artificial | NA |
| 33 | PYRIOR_DHODH_XP_003719157 | Pyricularia oryzae | AA |
| 34 | PYRIOR_DHODH_cDNA_XM_003719109.1 | Artificial | NA |
| 35 | PYRNTE_DHODH_EFQ86357.1 | Pyrenophora teres | AA |
| 36 | PYRNTE_DHODH_cDNA_GL537441.1: | Artificial | NA |
| | c28889-27234 | | |
| 37 | PYRNTR_DHODH_KAA8622403.1 | Pyrenophora triticirepentis | AA |
| 38 | PYRNTR_DHODH_cDNA_SAXQ01000002.1_{—} | Artificial | NA |
| | c685824-684169 | | |
| 39 | RAMUCC_DHODH_XP_023626237 | Ramularia collo-cygni | AA |
| 40 | RAMUCC_DHODH_cDNA_XM_023770469.1 | Artificial | NA |
| 41 | RHYNSE_DHODH_CZT49653.1 | Rhynchosporium secalis | AA |
| 42 | RHYNSE_DHODH_cDNA_FJVC01000391.135744- | Artificial | NA |
| | 37378 | | |
| 43 | SCLESC_DHODH_APA13900.1 | Sclerotinia sclerotiorum | AA |
| 44 | SCLESC_DHODH_cDNA_CP017824.1_2042520- | Artificial | NA |
| | 2043950 | | |
| 45 | SEPTTR_DHODH_XP_003852344.1 | Zymoseptoria tritici | AA |
| 46 | SEPTTR_DHODH_cDNA_XM_003852296.1 | Artificial | NA |
| 47 | VENTIN_DHODH_KAE9964670.1 | Venturia inaequalis | AA |
| 48 | VENTIN_DHODH_cDNA_WNWS01000666.1 | Artificial | NA |
| 49 | MONILA_DHODH_AA_KAB8292478.1 | Monilinia laxa | AA |
| 50 | MONILA_DHODH_cDNA_VIGI01000013.1 | Artificial | NA |
| 51 | BOTRCI_DHODH_XP_024553432 | Botrytis cinerea | AA |
| 52 | Consensus Motif 1 | Artificial | AA |
| 53 | Consensus Motif 2 | Artificial | AA |
| 54 | Consensus Motif 3 | Artificial | AA |
| 55 | Consensus Motif 4 | Artificial | AA |
| 56 | Consensus Motif 5 | Artificial | AA |
| 57 | Consensus Motif 6 | Artificial | AA |
| 58 | Motif 1 | Artificial | AA |
| 59 | Motif 2 | Artificial | AA |
| 60 | Motif 3 | Artificial | AA |
| 61 | Motif 4 | Artificial | AA |
| 62 | Motif 5 | Artificial | AA |
| 63 | Motif 6 | Artificial | AA |

AA = amino acid
NA = nucleic acid

The disclosure of any document as cited herein above is incorporated by reference

1. A method for combatting dihydroorotate-dehydrogenase (DHODH) inhibitor-resistant phytopathogenic fungi comprising:
treating the phytopathogenic fungi or materials, plants, soil or seeds that are at risk of being diseased from the phytopathogenic fungi with an effective amount of at least one compound of formula I 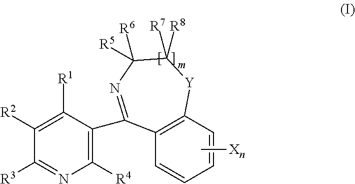
where
R¹ is hydrogen, halogen, CN, C₁-C₄-alkyl or C₁-C₄-haloalkyl;
R² is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₁-C₆-alkoxy, C₂-C₆-alkenyloxy or C₂-C₆-alkynyloxy;
R³ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₁-C₆-alkoxy, C₂-C₆-alkenyloxy or C₂-C₆-alkynyloxy;
R⁴ is hydrogen, halogen, CN, C₁-C₄-alkyl or C₁-C₄-haloalkyl;
R⁵ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the 8 last-mentioned aliphatic or aromatic radicals are unsubstituted or carry 1, 2 or 3 substituents R^{5a}; where each R^{5a} is independently halogen, CN, C₁-C₆-alkyl, C₁-C₆-haloalkyl or C₁-C₆-alkoxy;
R⁶ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the 8 last-mentioned aliphatic or aromatic radicals are unsubstituted or carry 1, 2 or 3 substituents R^{6a}; where
each R^{6a} is independently halogen, CN, C₁-C₆-alkyl, C₁-C₆-haloalkyl or C₁-C₆-alkoxy;
or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing 1, 2 or 3 heteroatoms selected from O and S as ring members; where the carbocyclic or heterocyclic ring is unsubstituted or carries 1, 2 or 3 substituents R⁵⁶; where
each R⁵⁶ is independently halogen, C₁-C₆-alkyl or C₁-C₆-haloalkyl;
R⁷ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the 8 last-mentioned aliphatic or aromatic radicals are unsubstituted or carry 1, 2 or 3 substituents R^{7a}; where
each R^{7a} is independently halogen, CN, C₁-C₆-alkyl, C₁-C₆-haloalkyl or C₁-C₆-alkoxy;
R⁸ is hydrogen, halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the 8 last-mentioned aliphatic or aromatic radicals are unsubstituted or carry 1, 2 or 3 substituents R^{8a}; where each R^{8a} is independently halogen, CN, C₁-C₆-alkyl, C₁-C₆-haloalkyl or C₁-C₆-alkoxy;
or
R⁷ and R⁸, together with the carbon atom to which they are bound, form a 3-, 4-, 5- or 6-membered saturated carbocyclic ring or a 3-, 4-, 5- or 6-membered saturated heterocyclic ring containing 1, 2 or 3 heteroatoms selected from O and S as ring members;
each X is independently halogen, CN, C₁-C₆-alkyl, C₁-C₆-haloalkyl, C₂-C₆-alkenyl, C₂-C₆-alkynyl, C₃-C₆-cycloalkyl, C₁-C₆-alkoxy or C₁-C₆-haloalkoxy;
Y is O if m is 1; and is O or NR⁹ if m is 0;
where
R⁹ is hydrogen, CN, CH₂CN, CH(CH₃)CN, CH(═O), —C(═O)C₁-C₆-alkyl, —C(═O)C₂-C₆-alkenyl, —C(═O)C₂-C₆-alkynyl, —C(═O)C₃-C₆-cycloalkyl, —C(═O)—N(H) C₁-C₄-alkyl, —C(═O)—N(C₁-C₄-alkyl)₂, C₁-C₆-alkyl, C₁-C₆-alkoxy, C₁-C₄-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl,
—S(═O)₂-R^{9a}, five- or six-membered heteroaryl, aryl or benzyl; wherein heteroaryl contains 1,2 or 3 heteroatoms selected from N, O and S as ring members; wherein aryl and the phenyl ring in benzyl are unsubstituted or carry 1, 2, 3, 4 or 5 substituents selected from the group consisting of CN, halogen, OH, C₁-C₄-alkyl, C₁-C₄-haloalkyl, C₁-C₄-alkoxy and C₁-C₄-haloalkoxy;
wherein
R^{9a} is C₁-C₆-alkyl, C₁-C₆-haloalkyl, C₂-C₆-alkenyl, C₂-C₆-haloalkenyl, C₂-C₆-alkynyl, C₂-C₆-haloalkynyl, phenyl or benzyl, where the phenyl ring in the two last-mentioned radicals is unsubstituted or carries 1, 2 or 3 substituents each independently selected from the group consisting of halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl;
m is 0 or 1; and
n is 0, 1, 2 or 3;
or an N-oxide, a tautomer, a stereoisomer or an agriculturally acceptable salt thereof.

2. The method according to claim 1, wherein one, two, three or all four of following conditions a), b), c) and/or d) apply:
a) R¹ and R⁴ are hydrogen;
b) R² and R³, independently of each other, are C₁-C₄-alkyl or C₁-C₄-haloalkyl;
c) R⁵ and R⁶, independently of each other, are hydrogen, C₁-C₆-alkyl or phenyl;
or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶; where each R⁵⁶ is independently C₁-C₄-alkyl;
d) each X is independently halogen, CN, C₁-C₆-alkyl, C₁-C₆-haloalkyl, C₁-C₆-alkoxy or C₁-C₆-haloalkoxy.

3. The method according to claim 1, wherein
R⁷ and R⁸, independently of each other, are hydrogen, C₁-C₄-alkyl or C₁-C₄-alkoxy-C₁-C₄-alkyl;
or
R⁷ and R⁸, together with the carbon atom to which they are bound, form a 3-, 4-, 5- or 6-membered saturated carbocyclic ring.

4. The method according to claim 1, wherein R⁹ is hydrogen, —CH (═O), —C(═O)C₁-C₆-alkyl, —C(═O)C₂-C₆-alkenyl, —C(═O)C₂-C₆-alkynyl, —C(═O)C₃-C₆-cycloalkyl, C₁-C₆-alkyl, C₁-C₄-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, —S(═O)₂-R^{9a}, five- or six-membered heteroaryl or phenyl; wherein heteroaryl contains 1, 2 or 3 heteroatoms selected from N, O and S as ring members; wherein phenyl is unsubstituted or carries 1, 2, 3, 4 or 5 substituents selected from the group consisting of CN, halogen, OH, C₁-C₄-alkyl, C₁-C₄-haloalkyl, C₁-C₄-alkoxy and C₁-C₄-haloalkoxy;
wherein R^{9a} is C₁-C₆-alkyl, C₁-C₆-haloalkyl or phenyl, where the phenyl ring is unsubstituted or carries 1, 2 or 3 C₁-C₄-alkyl substituents.

5. The method according to claim 1, wherein
R¹ is hydrogen;
R² is C₁-C₄-alkyl;
R³ is C₁-C₄-alkyl or C₁-C₄-haloalkyl;
R⁴ is hydrogen;
R⁵ is hydrogen, C₁-C₆-alkyl or phenyl;
R⁶ is hydrogen, C₁-C₆-alkyl or phenyl;
or
R⁵ and R⁶ form together an oxo group (═O);
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶; where
each R⁵⁶ is independently C₁-C₄-alkyl;
R⁷ is hydrogen, C₁-C₄-alkyl or C₁-C₄-alkoxy-C₁-C₄-alkyl;
R⁸ is hydrogen, C₁-C₄-alkyl or C₁-C₄-alkoxy-C₁-C₄-alkyl;
or
R⁷ and R⁸, together with the carbon atom to which they are bound, form a 3-, 4-, 5- or 6-membered saturated carbocyclic ring;
each X is independently halogen, C₁-C₆-alkyl or C₁-C₆-alkoxy; and
R⁹ is hydrogen, C₁-C₄-alkyl, —C(═O)C₁-C₃-alkyl or —S(═O)₂-R^{9a};
wherein
R^{9a} is C₁-C₆-alkyl, C₁-C₆-haloalkyl or phenyl, where the phenyl ring is unsubstituted or carries 1, 2 or 3 C₁-C₄-alkyl substituents.

6. The method according to claim 1, wherein
R¹ is hydrogen;
R² is C₁-C₄-alkyl;
R³ is C₁-C₄-alkyl or C₁-C₄-haloalkyl;
R⁴ is hydrogen;
R⁵ is hydrogen, C₁-C₆-alkyl or phenyl;
R⁶ is C₁-C₆-alkyl or phenyl;
or
R⁵ and R⁶, together with the carbon atom to which they are bound, form a 3-, 4-, 5- or 6-membered saturated carbocyclic ring or a 3-, 4-, 5- or 6-membered saturated heterocyclic ring containing one oxygen atom as ring member; where the carbocyclic or heterocyclic ring is unsubstituted or carries 1, 2 or 3 substituents R⁵⁶; where
each R⁵⁶ is independently C₁-C₄-alkyl;
each X is independently halogen, C₁-C₆-alkyl or C₁-C₆-alkoxy;
Y is O; and
m is 0; and m
n is 0, 1 or 2;
or
R¹ is hydrogen;
R² is C₁-C₄-alkyl;
R³ is C₁-C₄-alkyl or C₁-C₄-haloalkyl;
R⁴ is hydrogen;
R⁵ is C₁-C₆-alkyl;
R⁶ is C₁-C₆-alkyl;
each X is independently halogen;
Y is NR⁹;
R⁹ is hydrogen, C₁-C₄-alkyl, —C(═O)C₁-C₃-alkyl or —S(═O)₂-R^{9a};
wherein
R^{9a} is phenyl, where the phenyl ring is unsubstituted or carries 1, 2 or 3 C₁-C₄-alkyl substituents;
m is 0; and
n is 0, 1 or 2;
or
R¹ is hydrogen;
R² is C₁-C₄-alkyl;
R³ is C₁-C₄-alkyl or C₁-C₄-haloalkyl;
R⁴ is hydrogen;
R⁵ is hydrogen or C₁-C₆-alkyl;
R⁶ is hydrogen or C₁-C₆-alkyl;
or
R⁵ and R⁶ form together an oxo group (═O);
R⁷ is hydrogen, C₁-C₄-alkyl or C₁-C₄-alkoxy-C₁-C₄-alkyl;
R⁸ is hydrogen, C₁-C₄-alkyl or C₁-C₄-alkoxy-C₁-C₄-alkyl;
or
R⁷ and R⁸, together with the carbon atom to which they are bound, form a 3-, 4-, 5- or 6-membered saturated carbocyclic ring;
Y is O;
m is 1; and
n is 0.

7. The method according to claim 1, wherein the DHODH inhibitor resistant phytopathogenic fungus contains at least one resistance-inducing mutation in its endogenous DHODH gene resulting in at least one difference in the amino acid sequence of the mutated DHODH protein relative to the non-mutated DHODH protein.

8. The method according to claim 1, wherein the DHODH inhibitor resistant phytopathogenic fungus is selected from the genus Alternaria, Bipolaris, Botrytis, Cercospora, Colletotrichum, Corynespora, Fusarium, Plenodomus, Parastagonospora, Monographella, Pseudocercospora, Pyricularia, Pyrenophora, Ramularia, Rhynchosporium, Sclerotinia, Zymoseptoria, and Venturia.

9. The method according to claim 1, wherein the DHODH inhibitor resistant phytopathogenic fungus contains at least one resistance-inducing mutation in the amino acid sequence of its endogenous DHODH enzyme in at least one sequence portion corresponding to one of the following non-mutated consensus sequence motifs:
a) X₁ X₂ X₃ X₄ X₅ X₆ X₇ R
(Consensus Motif 1) (SEQ ID NO:52)
wherein X₁ to X₇ independently of each other represent any natural amino acid residue,
b) D X₂ E X₄ A H X₇ X₈ G
(Consensus Motif 2) (SEQ ID NO:53)
wherein X₂ to X₈ independently of each other represent any natural amino acid residue,
c) L X₂ X₃ X₄ G X₆ X₇ X₈ V E
(Consensus Motif 3) (SEQ ID NO:54)
wherein X² to X₈ independently of each other represent any natural amino acid residue,
X₇=A or G
X₈=I or V
d) X₁ Q X₃ G N X₆ X₇ P R X₁₀ F R
(Consensus Motif 4) (SEQ ID NO:55)
wherein X₁ to X₁₀ independently of each other represent any natural amino acid residue,
e) NRYG X₅ NS
(Consensus Motif 5) (SEQ ID NO:56)
wherein X₅=any natural amino acid residue, and in particular L or F
f) X₁ X₂ X₃ Y G G X₇ G T X₁₀ X₁₁ R X₁₃ K
(Consensus Motif 6) (SEQ ID NO:57)
wherein X₁ to X₁₃ independently of each other represent any natural amino acid residue.

10. The method according to claim 9, wherein the endogenous DHODH enzyme is selected from enzymes comprising at least one amino acid sequence selected from SEQ ID NOs: 1, 3, 5, 7, 9, 11, 13, 15, 17, 19, 21, 23, 25, 27, 29, 31, 33, 35, 37, 39, 41, 43, 45, 47, 49 or 51 or a sequence having a degree of sequence identity of at least 70% to anyone of said sequences.

11. The method according to claim 1, wherein the DHODH inhibitor resistant phytopathogenic fungus is of the species Botrytis cinerea and contains at least one resistance-inducing mutation in at least one of the following non-mutated sequence motifs of its non-mutated DHODH comprising an amino acid sequence of SEQ ID NO: 1
a) WLVVPALR (Motif 1) (SEQ ID NO:58) corresponding to amino acid residues 91 to 98 of SEQ ID NO:1
b) DAEDAHHVG (Motif 2) (SEQ ID NO:59) corresponding to amino acid residues 103 to 111 of SEQ ID NO:1
c) LFALGPAIVE (Motif 3) (SEQ ID NO:60) corresponding to amino acid residues 169 to 178 of SEQ ID NO:1
d) AQDGNPKPRVFR (Motif 4) (SEQ ID NO:61) corresponding to amino acid residues 186 to 197 of SEQ ID NO:1
e) NRYGLNS (Motif 5) SEQ ID NO:62 corresponding to amino acid residues 206 to 212 of SEQ ID NO:1
f) AMVYGGAGTITRIK (Motif 6) SEQ ID NO:63 corresponding to amino acid residues 492 to 505 of SEQ ID NO:1;
wherein the DHODH inhibitor resistant phytopathogenic fungus is of the species Botrytis cinerea and contains at least one resistance-inducing mutation in the non-mutated sequence of SEQ ID NO:1 or in a sequence which is at least 70% identical thereto, wherein the mutant is selected from
a) the single mutants
V94X,
A104X,
A107X,
H108X,
Del109,
G111X,
L172X,
V195X,
Y208X,
V494X,
G497X,
wherein
Del designates a deletion of an amino acid residue and
X represents any natural amino acid residue; and
b) multiple mutants
containing a combination of at least 2 of the resistance inducing single mutations as defined above under a),
wherein
X represents any natural amino acid residue.

12. The method according to claim 1, wherein the DHODH inhibitor resistant phytopathogenic fungus is different from Botrytis cinerea and contains at least one resistance-inducing mutation in an amino acid sequence position analogous to that of a mutant of the non-mutated Botrytis cinerea DHODH amino acid sequence of SEQ ID NO: 1 as defined in claim 11.

13. A mutated DHODH inhibitor resistant DHODH enzyme comprising at least one resistance-inducing mutation in its endogenous DHODH gene resulting in at least one difference in the amino acid sequence of the mutated DHODH protein relative to the non-mutated DHODH protein.

14. A nucleic acid molecule comprising at least one nucleotide sequence encoding a mutated DHODH enzyme as defined in claim 13.

15. The method of claim 1, comprising:
a) identifying the phytopathogenic fungi, or the materials, plants, the soil or seeds that are at risk of being diseased from phytopathogenic fungi,
and
b) treating said fungi or the materials, plants, the soil or seeds with an effective amount of at least one compound of formula I or an N-oxide, a tautomer, a stereoisomer or an agriculturally acceptable salt thereof or with an effective amount of a composition comprising at least one compound of formula I or an N-oxide, a tautomer, a stereoisomer or an agriculturally acceptable salt thereof.

16. The method according to claim 1, wherein the DHODH inhibitor resistant phytopathogenic fungus contains at least one resistance-inducing mutation in the amino acid sequence of its endogenous DHODH enzyme in at least one sequence portion corresponding to one of the following non-mutated consensus sequence motifs:
a) X₁ X₂ X₃ X₄ X₅ X₆ X₇ R
(Consensus Motif 1) (SEQ ID NO:52)
wherein X₁ to X₇ have the following meanings
X₁=W, F, Y, H or Del, wherein Del designates a missing amino acid residue
X₂=L, A, I, For V
X₃=V, L, S or A
X₄=V, T or P
X₅=P, A or M
X₆=A, L, V, I, T or F
X₇=L, V, M or I
b) D X₂ E X₄ A H X₇ X₈ G
(Consensus Motif 2) (SEQ ID NO:53)
wherein X² to X₈ have the following meanings
X₂=A, G or P
X₄=D, E
X₇=H or Q
X₈=V, A, F, S or Mc)
L X₂ X₃ X₄ G X₆ X₇ X₈ V E
(Consensus Motif 3) (SEQ ID NO:54)
wherein X₂ to X₈ have the following meanings
X₂=F, M or L
X₃=E, A, D or S
X₄=L, V or M
X₆=P or A
X₇=A or G
X₈=I or V
d) X₁ Q X₃ G N X₆ X₇ P R X₁₀ F R
(Consensus Motif 4) (SEQ ID NO:55)
wherein X₁ to X₁₀ have the following meanings
X₁=P or A
X₃=D, E, P, A, L or K
X₆=P or D
X₇=K, R or Q
X₁₀=V or M
e) N R Y G X₅N S
(Consensus Motif 5) (SEQ ID NO:56)
wherein X₅=L or F
f) X₁ X₂ X₃ Y G G X₇G T X₁₀ X₁₁ R X₁₃ K
(Consensus Motif 6) (SEQ ID NO:57)
wherein X₁ to X₁₃ have the following meanings
X₁=A, G, S or K
X₂=M, L, R or I
X₃=I, V, M or T
X₇=A, S, V or P
X₁₀=I or V
X₁₁=T, G or S
X₁₃=I, M or V.

17. The method according to claim 11, wherein the DHODH inhibitor resistant phytopathogenic fungus contains at least one resistance-inducing mutation in the non-mutated sequence of SEQ ID NO:1 or in a sequence which is at least 70% identical thereto, wherein the mutant is selected from
a) the single mutants
V94F, I, L, D, A, G, E or M,
A104T, P, S, D, V, G or E,
A107T, P, S, D, V, G or E,
H108Q, P, R, L, D, Y or N,
Del109,
G111E, A, V, R, D, C, W or S,
L172F, I, S, V, H, P, R, Q or M,
V195F, I, L, D, A, G, E or M,
Y208S, C, F, N, H or D,
V494F, I, L, D, A, G, E or M,
G497E, A, V, R, D, C, W or S,
wherein
Del designates a deletion of an amino acid residue
and
b) multiple mutants
containing a combination of at least 2 of the resistance inducing single mutations as defined above under a).

18. The nucleic acid molecule of claim 14, wherein the at least one nucleotide sequence encoding the mutated DHODH enzyme is selected from mutant nucleic acid sequences derived from nucleotide sequences according to SEQ ID NO: 2, 4, 6, 8, 10, 12, 14, 16, 18, 20, 22, 24, 26, 28, 30, 32, 34, 36, 38, 40, 42, 44, 46, 48, or 50 and sequences having at least 70% sequence identity to anyone of said sequences, comprising at least one mutation in the nucleotide sequence which encodes said mutated DHODH enzyme.
